(19) United States
(12) Patent Application Publication
(10) Pub. No.: US 2025/0332233 A1
(43) Pub. Date: Oct. 30, 2025

(54) REPLICANT / STAV FOR DISEASE TREATMENT AND METHODS OF USE

(71) Applicant: Glen N. Barber, Columbus, OH (US)

(72) Inventor: Glen N. Barber, Columbus, OH (US)

(21) Appl. No.: 19/187,765

(22) Filed: Apr. 23, 2025

Related U.S. Application Data
(63) Continuation of application No. PCT/US25/25960, filed on Jan. 1, 0001.
(60) Provisional application No. 63/638,337, filed on Apr. 24, 2024.
(60) Provisional application No. 63/792,880, filed on Apr. 22, 2025.

(51) Int. Cl. A61K 39/00; A61K 9/51; A61K 38/21

(52) U.S. Cl. CPC A61K 39/0005; A61K 9/5123; A61K 38/21; A61K 2039/53

(57) ABSTRACT

Activation of STimulator of INterferon Genes (STING) triggers cytokine production and facilitates tumor antigen cross-presentation. In an embodiment of the present invention, STING-dependent innate immune signaling pathway activators (STAVs) together with Replicants including mRNA adapted to express an antigen can be delivered to antigen presenting cells (APC's) using lipid nanoparticle formulations. In various embodiments of the present invention, the range of cancers amenable to STAV/Replicant therapy can be extended using a non-cell-based nanoparticle strategy that effectively delivers the STAV/Replicant into the Tumor Micro Environment (TME) to potently generate anti-tumor cytotoxic T cell activity together with humoral immune responses. The STAV/Replicant formulations can be introduced into solid tumors present in the subject. Alternatively, the STAV/Replicant can be introduced through direct inoculation, intramuscularly, or intravenously. The lipid nanoparticles stick to the tumor cells and are co-phagocytosed to activate STING in APC's.

This application is a continuation under 35 U.S.C. section 111(a) of (i) PCT Application No.: PCT/US25/25960 entitled ‘REPLICANT/STAV FOR DISEASE TREATMENT AND METHODS OF USE’, filed Apr. 23, 2025 which claims the priority benefit of (ii) U.S. Provisional Patent Application No. 63/792,880, entitled ‘A REPLICANT PLUS A STAV IN A LIPID NANOPARTICLE VEHICLE FOR DISEASE TREATMENT AND METHODS OF USE’, filed Apr. 22, 2025, and (iii) U.S. Provisional Patent Application No. 63/638,337, entitled ‘LIPID NANOPARTICLES FOR DELIVERY OF REPLICANTS/STING-DEPENDENT ADJUVANTS AND METHODS OF USE’, Apr. 24, 2024, which applications (i)-(iii) are herein incorporated by reference in their entireties and for all purposes.

### REFERENCE TO A ‘SEQUENCE LISTING,’ A TABLE, OR A COMPUTER PROGRAM LISTING APPENDIX SUBMITTED AS AN ASCII TEXT FILE

The Sequence Listing written in file STMM-01017US2_ST26.xml, created Apr. 24, 2025, 695,076 bytes, machine format IBM-PC, MS-Windows operating system, is hereby incorporated by reference in its entirety and for all purposes.

Embodiments of the invention relate to compositions and methods for modulating innate and adaptive immunity in a subject and/or for the treatment of an immune-related disorder, cancer, autoimmunity, treating and preventing infections with the combination of Sting Dependent Adjuvants and Replicants.

Cellular innate immune sensors, such as STING, have evolved to detect microbial infection of the cell. STimulator of Interferon Genes (STING) is activated by cyclic dinucleotides (CDN's) such as cyclic di-GMP and cyclic-di-AMP secreted by intracellular bacteria following infection. Alternatively, STING can be activated by cyclic GMP-AMP (cGAMP) generated by a cellular cGAMP synthase cGAS (MB21D1) after association with aberrant cytosolic dsDNA species, which can include microbial DNA or self-DNA leaked from the nucleus. Association with CDN's enables STING to activate the transcription factors IRF3 and NF-κB which stimulate the production of type I interferon (IFN) and pro-inflammatory cytokines, which facilitate adaptive immunity. Aside from being critical for the protection against microbial infection, STING signaling has been shown to be important for facilitating anti-tumor T cell activity. Regulation of the immune system to facilitate robust anti-tumor cytotoxic T cell responses is proving to be a powerful approach for the effective treatment of a variety of cancers.

HTLV-1 affects about 10-20 million people worldwide. HTLV-1 is endemic in southwest Japan, the southern United States, central Australia, the Caribbean, sub-Saharan Africa, parts of South America, particularly Brazil and Peru and equatorial Africa, and the middle East. South Florida (containing Miami and Broward counties) due to its close proximity to the Caribbean, has a large population of immigrants from HTLV-1 endemic areas, therefore ATLL is commonly encountered in this geographic area.

Adult T cell leukemia (ATLL) is a rare fast-growing T-cell lymphoma that can be found in the blood, lymph nodes, skin, or multiple areas of the body. ATLL was first described as a distinct clinical entity in 1979 and its association with HTLV-1 was reported shortly thereafter. The majority of infected HTLV-1 carriers are asymptomatic for their lifetime, however, an estimated 5% of HTLV-1 positive individuals will develop ATL or 2% into HAM/TSP after prolonged latency periods. Despite the relatively low penetrance of HTLV-1 associated diseases, HTLV-1 is a major problem in endemic communities as there are no effective treatment options for either ATL or HAM/TSP afflicted individuals. ATLL can present in multiple forms and is generally sub-classified into four subtypes. Lymphoma and acute ATLL are the two most aggressive variants where patients usually present with a high tumor burden and hypercalcemia. The chronic and smoldering forms of ATLL have a more indolent course, although they often progress to the more malignant forms of the disease. ATLL carries a dismal prognosis, and is generally incurable with conventional chemotherapy alone. In a Japanese study of 1,594 patients with ATLL treated with modern aggressive therapies between 2000 and 2009, the median survival (MS) times were 8.3 and 10.6 months for acute and lymphomatous types respectively.

Acute Lymphoblastic Leukemia (ALL) is a blood cancer that affects lymphoblasts in the bone marrow and blood. However, ALL can spread to other parts of the body. ALL is more common in children than adults, and is an aggressive cancer. ALL is the least common type of acute leukemia in adults although it is the most common in the pediatric population. Adult patients have a relatively poor prognosis as compared to children and young adults, who can be cured with intensive chemotherapy.

The epidermal growth factor receptor (EGFR) is a protein on the surface of cells that binds to epidermal growth factor (EGF). The binding mechanism triggers signaling within the cell that alters cell growth, cell division, and cell survival. Mutations in the EGFR gene resulting in increased EGFR expression, lead to cancer development and progression.

cGMP regulates a number of cellular processes and plays an important role in maintaining intestinal homeostasis. GUCY2C is expressed on the luminal surfaces of the intestinal epithelium and in certain types of hypothalamic neurons. Transmembrane 4 L six family member 5 (TM4SF5), is a tetraspanin protein involved in cell migration, cell invasion, and tumor cell growth. TM4SF5 acts as a sensor for lysosomal arginine levels, regulating mTORC1 signaling and impacting cellular metabolism. TM4SF5 is implicated in fibrosis, cancer, and various other diseases.

Trophoblast glycoprotein (5T4) is a human protein encoded by a TPBG gene. 5T4 is an N-glycosylated transmembrane 72 kDa glycoprotein antigen expressed in a number of carcinomas. Guanylate cyclase 2C (GUCY2C), is a transmembrane protein that acts as a receptor for guanylin, uroguanylin, and the heat-stable enterotoxin produced by some bacteria. GUCY2C is a receptor that, when activated, triggers the production of cyclic guanosine monophosphate (cGMP). MLANA, is a small, transmembrane protein primarily expressed in melanocytes, retinal pigment epithelium, and melanoma cells, but not in normal healthy tissue. MLANA is involved in the stability of the GPR143 protein and the trafficking and processing of PMEL protein which are each involved in melanosome formation and stability. MLANA's expression is often associated with melanomas. MLANA is a useful marker for melanomas. Mucin 1 (MUC1), is a transmembrane glycoprotein that plays a role in both normal and cancerous cells. In healthy cells, it acts as a protective barrier, lubricating, and moisturizing epithelial surfaces. If MUC1 is overexpressed or aberrantly glycosylated it results in tumor progression in the epithelium. Tyrosinase-related protein 1 (TYRP1), is a gene product primarily found in melanocytes, the cells responsible for producing melanin, the pigment that gives skin, hair, and eyes their color. TYRP1 plays a crucial role in melanin synthesis and is also involved in organelles within melanocytes producing melanin.

Mitotic Centromere-Associated Kinesin (MCAK) is a microtubule depolymerase, helping to regulate the formation of the mitotic spindle, corrects attachment of chromosomes to the spindle microtubules and depolymerizes microtubules, which are essential steps for chromosome movement and cell division. Accordingly, MCAK plays a crucial role in cell division, and mitosis. MCAK is present throughout a cell, concentrated at the centromeres, kinetochores, and spindle poles. Deregulation of MCAK results in cancer cell growth, and metastasis. Carcinoembryonic antigen (CEA), is a cell surface glycoprotein that plays a role in cell adhesion. CEA is upregulated in many cancers, including lung, pancreatic, and breast cancers, and is implicated in tumor progression, migration, and proliferation. Accordingly, CEA can be used as a marker for a number of malignancies, including colorectal cancer and non-small-cell lung cancer.

Cytomegalovirus (CMV), Epstein-Barr virus (EBV), and Human Herpesvirus-8 (HHV-8) and Human Papillomavirus (HPV) are all viral infections that are sexually transmitted by skin-to-skin contact or bodily fluids. HPV can lead to genital warts and/or cancer. CMV, EBV, and HHV-8 belong to the human herpesvirus family. EBV and CMV can both cause mononucleosis, but have distinct clinical presentations. EBV establishes permanent infections in humans and can lead to lymphomas. CMV in humans (β-herpesvirus-5 or HHV-5), can cause a mononucleosis-like syndrome with prolonged fevers and systemic symptoms and has been linked to hematological and autoimmune disorders. HHV-8 causes Kaposi sarcoma (a vascular malignancy) and B cell lymphoproliferative diseases such as primary effusion lymphoma and multicentric Castleman disease.

### SUMMARY OF THE INVENTION

Tumor cells are notoriously non-immunogenic through their ability to mimic the properties of normal cells which have naturally evolved to avoid activating the immune system following cell death and phagocytosis. In an embodiment of the present invention, a new approach overcomes this obstacle and makes previously immuno-evasive, inert tumor cells highly immunogenic. This has been achieved by developing DNase-resistant nucleic acid-based STING-dependent adjuvants or activators, referred to as STAVs (dsDNA species of approximate length 76 nucleotides) as activators of the STING-dependent innate immune signaling pathway in combination with RNA which generates a humoral immune response against the tumor cells. In an embodiment of the present invention, tumor cells loaded with STAVs and RNA nucleic acid encoding an antigen (such as ssRNA, RNA, mRNA, self-replicating RNA's or DNA/RNA chimeras or ssDNA or dsDNA) renders non-immunogenic cells immunogenic. In an embodiment of the present invention, the tumor cells loaded with STAVs (i.e., dsDNA), and an RNA encoding an antigen directed to a disease (microbial, self or tumor specific antigen) are able to stimulate antigen presenting cells (APCs) in vitro and in vivo, in trans. In an embodiment of the present invention, the tumor cells loaded with STAVs, and DNA encoding mRNA, where the RNA encodes an antigen directed to a disease (microbial, self or tumor specific antigen) are able to stimulate antigen presenting cells (APCs) in vitro and in vivo, in trans. In an alternative embodiment of the present invention, the tumor cells loaded with STAVs, and RNA (DNA) directed to a disease with IFN type I are able to stimulate antigen presenting cells (APCs) in vitro and in vivo.

In an embodiment of the present invention, a subject is injected intramuscularly or intravenously with a LNP comprising a STAV, and a Replicant adapted to express an OVA peptide (SIINFEKL, SEQ ID NO:34). In an alternative embodiment of the present invention, subject is injected intramuscularly or intravenously with a LNP comprising a STAV, and the Replicant adapted to express the OVA peptide (SIINFEKL, SEQ ID NO:34) and boosted every 3 weeks to generate cytotoxic CD8+ T cell (CTL) activity specific against infected/diseased cells including cancer cells.

In an embodiment of the present invention, tumor cells loaded with STAVs, RNA directed to for example HTLV-1 TAX (TAX) and/or HTLV-1 basic leucine zipper factor (HBZ) can be used to treat autologous aggressive leukemia cells (ATLL, AML, and ALL) concomitant with a personalized dendritic cell (DCs) vaccine. In an alternative embodiment of the present invention, tumor cells loaded with STAVs, and mRNA directed to for example TAX and/or HBZ can be used to generate a vaccine for ATLL, AML, and ALL. In an embodiment of the present invention, the tumor cells loaded with STAVs, with mRNA directed to TAX and/or HBZ, and IFN type I are able to stimulate antigen presenting cells (APCs) to generate immunity in adult T cell leukemia (ATLL) infected patients.

In an embodiment of the present invention, tumor cells loaded with STAVs, RNA directed to for example HTLV1 gp62 envelope glycoprotein (gp62G) and/or TAX and/or HBZ can be used to treat autologous aggressive leukemia cells (ATLL, AML, and ALL) concomitant with a personalized dendritic cell (DCs) vaccine. In an alternative embodiment of the present invention, tumor cells loaded with STAVs, and mRNA directed to for example gp62G, and/or HBZ and/or TAX can be used to generate a vaccine for ATLL, AML, and ALL. In an embodiment of the present invention, the tumor cells loaded with STAVs, with mRNA directed to IFN type I and/or gp62G, and/or HBZ and/or TAX are able to stimulate antigen presenting cells (APCs) to generate immunity in adult T cell leukemia (ATLL) infected patients. In an embodiment of the present invention, the tumor cells loaded with STAVs, with mRNA directed to IFN type I and gp62G-HBZΔ1-TAXΔ2 are able to stimulate antigen presenting cells (APCs) to generate immunity in adult T cell leukemia (ATLL) infected patients, see U.S. patent application Ser. No. 17/603,331 entitled ‘A Recombinant HTLV1 Vaccine’, inventor Glen N. Barber, filed Oct. 12, 2021 and which application is herein incorporated by reference in its entirety and for all purposes. In various embodiments of the invention, the treatment can be in vivo or ex vivo.

In an embodiment of the present invention, tumor cells loaded with STAVs, RNA directed to for example HTLV1 gp62 envelope glycoprotein (gp62G) can be used to treat autologous aggressive leukemia cells (ATLL, AML, and ALL) concomitant with a personalized dendritic cell (DCs) vaccine. In an alternative embodiment of the present invention, tumor cells loaded with STAVs, and mRNA directed to for example gp62G can be used to generate a vaccine for ATLL, AML, and ALL. In an embodiment of the present invention, the tumor cells loaded with STAVs, with mRNA directed to IFN type I and/or gp62G are able to stimulate antigen presenting cells (APCs) to generate immunity in adult T cell leukemia (ATLL) infected patients. In an embodiment of the present invention, the tumor cells loaded with STAVs, with mRNA directed to IFN type I and gp62G are able to stimulate antigen presenting cells (APCs) to generate immunity in adult T cell leukemia (ATLL) infected patients.

In an embodiment of the present invention, the tumor cells loaded with STAVs, with RNA directed to tumor-associated antigens (TAAs) and tumor-specific antigens (TSAs), also called neoantigens including head and neck cancers genes (such as HPV E6, E7), with or without cytokines such as IFN type I are able to stimulate antigen presenting cells (APCs) to generate an immune response to patients suffering from, for example, head and neck cancer. In an embodiment of the present invention, the tumor cells loaded with STAVs, with mRNA directed to melanoma tumor antigens TYRP-1/2 Melan-A-1/2 (melanoma antigen recognized by T cells 1), with IFN type I are able to stimulate antigen presenting cells (APCs) to generate an immune response to melanoma patients. In an embodiment of the present invention, the tumor cells loaded with STAVs, with mRNA encoding tumor antigens associated with colorectal cancer (CRC), for example Carcinoembryonic antigen (CEA), mucin 1 (MUC-1), epidermal growth factor receptor (EGFR), vascular endothelial growth factor receptor 1 and 2 (VEGFR1, VEGFR2), transmembrane 4 superfamily member 5 protein (TM4SF5), survivin, mitotic centromere-associated kinesin (MCAK), guanylyl cyclase C (GUCY2C), and 5T4, with or without IFN type I are able to stimulate antigen presenting cells (APCs) to generate an immune response to CRC patients.

Custom tumor antigens specific to patients can include novel TSAs. TSAs that include Custom tumor antigens specific to a patient can be identified using a three-step algorithm: 1) identifying somatic mutations or productions in DNA or messenger RNA (mRNA) sequences; 2) evaluating the affinity and presentation of MHC I/II molecules with new peptides; 3) determining whether new epitopes can stimulate T-cell proliferation and related immune responses. By improving the algorithm and exploring subtype-specific antigens, potential antigen targets of cancer vaccines can be found. This improved algorithm can lay a foundation for subsequent vaccine preparation. In an embodiment of the present invention, the tumor cells loaded with STAVs, with RNA directed to coronavirus spike protein, with or without IFN type I are able to stimulate antigen presenting cells (APCs) to generate an immune response to protect patients from coronavirus infection.

The present application provides the combination of STAVs+RNA (directed to a specific disease) as therapeutic agents in the generation of a vaccine to the specific disease, treatment or prevention of the specific disease such as cancer, coronavirus, inflammation, and other immunological disorders. In an alternative embodiment of the present application, the combination of STAVs+RNA (directed to a specific disease) and IFN type I act as therapeutic agents in the generation of a vaccine to the specific disease, treatment or prevention of the specific disease such as cancer, coronavirus, inflammation, and other immunological disorders.

### BRIEF DESCRIPTION OF THE DRAWINGS

Various embodiments of the present invention will be described in detail based on the following Figures, where:

FIG. 1A shows an immunoblot of total protein production probed with a gp62 antibody (11) and β-actin antibody (10) in HEK293T cells 24 hours after transfecting with either Mock (7), 2 μg gp62 mRNA (17), 5 μg gp62 mRNA (18), or 10 μg gp62 mRNA (19), using a DMRIE-C liposomal transfection reagent, according to an embodiment of the present invention;

FIG. 1B shows an immunoblot of total protein production probed with a gp62 antibody (11) and β-actin antibody (10) in HEK293T cells 32 hours after transfecting with either Mock (7), 2 μg gp62 mRNA (17), 5 μg gp62 mRNA (18), or 10 μg gp62 mRNA (19), using a DMRIE-C liposomal transfection reagent, according to an embodiment of the present invention;

FIG. 2A shows an immunoblot of total protein production probed with a gp62 antibody (11) and β-actin antibody (10) in HEK293T cells 20 hours after transfecting with either Mock (7), 2 μg gp62 mRNA (17), or 5 μg gp62 mRNA (18), using a DMRIE-C liposomal transfection reagent, according to an embodiment of the present invention;

FIG. 2B shows an immunoblot of total protein production probed with a gp62 antibody (11) and β-actin antibody (10) in HEK293T cells 20 hours after transfecting with either Mock (7), or 5 μg gp62 mRNA (18), using lipofectamine 2000 liposomal transfection reagent, according to an embodiment of the present invention;

FIG. 2C shows an immunoblot of total protein production probed with a gp62 antibody (11) and β-actin antibody (10) in HEK293T cells 30 hours after transfecting with either Mock (7), 2 μg gp62 mRNA (17), or 5 μg gp62 mRNA (18), using a DMRIE-C liposomal transfection reagent, according to an embodiment of the present invention;

FIG. 2D shows an immunoblot of total protein production probed with a gp62 antibody (11) and β-actin antibody (10) in HEK293T cells 30 hours after transfecting with either Mock (7), or 5 μg gp62 mRNA (18), using lipofectamine 2000 liposomal transfection reagent, according to an embodiment of the present invention;

FIG. 3A shows an immunoblot of total protein production (cells are lysed for protein extraction) probed with a HBZ antibody (13), a Tax antibody (12) and β-actin antibody (10) in HEK293T cells 36 hours after transfecting with either Liposomal transfection reagent alone (8), empty vector (9), HBZ-Tax mRNA (30), HBZ mRNA (31), Tax mRNA (32), using lipofectamine 2000 liposomal transfection reagent, according to an embodiment of the present invention;

FIG. 3B shows an immunoblot of total protein production (cells are lysed for protein extraction) probed with a HBZ antibody (13), a Tax antibody (12) and β-actin antibody (10) in HEK293T cells 48 hours after transfecting with either HBZ-Tax (R114D) mRNA (33), HBZ-Tax (R114D) with the N-terminal endoplasmic reticulum (ER) signalling peptide1 of human BiP (UniprotKB ID: P11021) mRNA (34), HBZ-Tax (R114D) with the N-terminal endoplasmic reticulum (ER) signalling peptide of human BiP mRNA with ER retention sequence (35), HBZ-Tax (R114D) with the N-terminal endoplasmic reticulum (ER) signalling peptide2 of human BiP mRNA (36), HBZ-Tax (R114D) with the N-terminal endoplasmic reticulum (ER) signalling peptide2 of human BiP mRNA with ER retention sequence (37), Tax mRNA (32), HBZ-Tax (R114D) with the Mitochondrial Targeting signal from human COX411 (UniprotKB ID: H3BNI5) mRNA (38), or Tax mRNA (32), HBZ-Tax (R114D) with the Myrisoylation/palmitoylation site of human Lyn kinase (UniProt ID: P07948.3) mRNA (39), using lipofectamine 2000 liposomal transfection reagent, according to an embodiment of the present invention;

FIG. 4A shows an immunoblot of total protein production (cells are lysed for protein extraction) probed with ovalbumin antibody (15) and β-actin antibody (10) in HEK293T cells 6 hours after reagents (2 g/mL) in a lipo-nano-particle (LNP) are delivered with either Mock (7), empty vector (9), OVA mRNA (41), Replicon (42), OVA mRNA+Replicon (2:1) (43), OVA mRNA+Replicon (1:2) (44), according to an embodiment of the present invention;

FIG. 4B shows an immunoblot of total protein production (cells are lysed for protein extraction) probed with ovalbumin antibody (15) and β-actin antibody (10) in HEK293T cells 24 hours after reagents (2 g/mL) in a lipo-nano-particle (LNP) are delivered with either empty vector (9), OVA mRNA (41), Replicon (42), OVA mRNA+Replicon (2:1) (43), OVA mRNA+Replicon (1:2) (44), according to an embodiment of the present invention;

FIG. 5A shows a schematic representation of IFN-β production in HEK293T cells irradiated with UV (120 mJ/cm²) after 6 hours, incubated for 24 hours fed to WT macrophages where either the HEK293T cells are treated with Mock (7), UV light only (6), empty LNP (9), OVA mRNA (41), Replicon (42), OVA mRNA+Replicon (2:1) (43), OVA mRNA+Replicon (1:2) (44), according to an embodiment of the present invention;

FIG. 5B shows a schematic representation of CXCL10 production in HEK293T cells irradiated with UV (120 mJ/cm²) after 6 hours, incubated for 24 hours fed to WT macrophages where either the HEK293T cells are treated with Mock (7), UV light only (6), empty LNP (9), OVA mRNA (41), Replicon (42), OVA mRNA+Replicon (2:1) (43), OVA mRNA+Replicon (1:2) (44), according to an embodiment of the present invention;

FIG. 5C shows a schematic representation of CCL5 production in HEK293T cells irradiated with UV (120 mJ/cm²) after 6 hours, incubated for 24 hours fed to WT macrophages where either the HEK293T cells are treated with Mock (7), UV light only (6), empty LNP (9), OVA mRNA (41), Replicon (42), OVA mRNA+Replicon (2:1) (43), OVA mRNA+Replicon (1:2) (44), according to an embodiment of the present invention;

FIG. 6 shows an ELISPOT representation of IFN-γ production in C57/BL6 mice at 5/6 weeks (n=4 per group) intramuscularly injected with PBS or 10 μg of different LNP conditions (for (9), (41), (42), (43) and (44)) per mouse and boosted twice every 3 weeks (where 10 days after the 2nd boost, mice are sacrificed and their spleen collected to measure cytotoxic CD8+ T cell (CTL) activity specific against OVA peptide (SIINFEKL, SEQ ID NO:34) where PBS (7), empty LNP (9), OVA mRNA (41), STAV1 (42), OVA mRNA+STAV1 (2:1) (43), OVA mRNA+STAV1 (1:2) (44), and VSV-OVA (Positive control) (45), according to an embodiment of the present invention;

FIG. 7A a flow diagram showing the protocol for i.t. administration of STAVs and Replicant (mRNA directed to HPV, melanoma or CRC antigens), according to an embodiment of the present invention;

FIG. 7B a flow diagram showing the protocol for i.t. administration of STAVs and mRNA Replicant (mRNA directed to HPV, melanoma or CRC antigens) and i.t. administration of IFN, according to an embodiment of the present invention;

FIG. 7C a flow diagram showing the protocol for i.t. administration of STAVs and Replicant (mRNA directed to TAX and/or HBZ antigens), according to an embodiment of the present invention; and

FIG. 7D a flow diagram showing the protocol for i.t. administration of STAVs and Replicant (mRNA directed to TAX and/or HBZ antigens) and i.t. administration of IFN type I, according to an embodiment of the present invention.

### DETAILED DESCRIPTION OF THE INVENTION

Definitions of certain terms that are used hereinafter include:

The transitional term ‘comprising’ is synonymous with ‘including,’ ‘containing,’ or ‘characterized by,’ is inclusive or open-ended and does not exclude additional, unrecited elements or method steps.

The transitional phrase ‘consisting of’ excludes any element, step, or ingredient not specified in the claim, but does not exclude additional components or steps that are unrelated to the invention such as impurities ordinarily associated with a composition.

The transitional phrase ‘consisting essentially of’ limits the scope of a claim to the specified materials or steps and those that do not materially affect the basic and novel characteristic(s) of the claimed invention.

The term ‘RNA’ encompasses both messenger RNA and non mRNA (e.g., genomic RNA).

The term ‘Replicant’ means RNA which when inserted into a vector is capable of encoding a protein product of a gene (e.g., ssRNA, RNA, mRNA, self-replicating RNA's, DNA/RNA chimeras, ssDNA or dsDNA). A person of ordinary skill in the art would understand that a vector comprising a Replicant, where e.g., the Replicant comprises at least a first mRNA encoding an OVA antigen (SEQ ID NO:48), can be adapted to express the OVA antigen. A person of ordinary skill in the art would understand that the DNA sequence can be converted to an RNA sequence or vice versa. Further, a person of ordinary skill in the art would understand that the DNA or RNA sequence can be converted to a protein sequence.

The term ‘Percent Complimentary’ and the like refer in the usual and customary sense the degree of identity between complimentary bases. If e.g., all possible base pairs are formed, the Percent Complimentary is 100 percent. The region of comparison to determine Percent Complimentary for two nucleic strands that are of differing length is limited to the length of the shorter strand.

The term ‘Vaccine’ refers in the usual and customary sense to a composition to protect a mammal from a disease and also to an immunotherapeutic used to harnesses the mammal's own immune system to fight the disease. A vaccine can involve boosting the immune system in a general way or training it to specifically target diseased or infected cells. A vaccine may be administered to a mammal who has never been exposed to the disease, a mammal who has been infected by the disease, or a mammal infected and debilitated by the disease.

The term ‘STAV’ means dsDNA. In an embodiment of the present invention, a STAV species is of approximate length 76 nucleotides, where approximately means plus or minus twenty (20) percent. In an alternative embodiment of the present invention, a STAV species is of approximate length 60 nucleotides, where approximately means plus or minus twenty (20) percent. In another embodiment of the present invention, a STAV species is of approximate length 90 nucleotides, where approximately means plus or minus twenty (20) percent.

The phrase ‘STing Dependent AdjuVants’ or ‘STAVs’ refers to dsDNA between forty (40) and ninety (90) nucleobases, where the STAV is an innate immune activator of STING despite being ‘immunologically inert’ and ‘functionally inert’, see PCT Application No. PCT/US22/034796 entitled ‘STING DEPENDENT ADJUVANTS’, inventor Glen N. Barber, filed Jun. 23, 2022 and U.S. Utility patent application Ser. No. 18/176,406, filed Feb. 28, 2023, LIPID NANOPARTICLES FOR DELIVERY OF STING-DEPENDENT ADJUVANTS, by Glen N. Barber which applications are herein incorporated by reference in their entireties and for all purposes. With reference to a STAV, ‘immunologically inert’ means that there is no significant humoral response. In this context, a humoral response involves antibodies produced by B cells. With reference to a STAV, ‘functionally inert’ means that there is no mechanism for transcription of the DNA, whether that involves the absence of a region to initiate transcription of the DNA and/or the sequence of the DNA is such that a person of ordinary skill in the art can understand that the protein product is not physiologically relevant (e.g. a STAV1, SEQ ID NO:24 comprises a dsDNA where a first strand comprises eighty (80) percent or more of A nucleotides and a second strand SEQ ID NO:25 comprises eighty (80) percent or more of T nucleotides can generate a polyphenylalanine protein which is not physiologically relevant. Alternatively, a first SEQ ID NO:28 strand comprises eighty (80) percent or more of alternating A nucleotides and T nucleotides can generate a polytyrosine protein which is not physiologically relevant). The term ‘functionally inert’ does not preclude that the STAV has a biological activity to activate STING. A STAV can optionally comprise where each strand of DNA comprises at least one (1) exonuclease resistant phosphorothioate backbone moiety (ps) at the 5′ end and at least one (1) ps at the 3′ end. In an embodiment of the invention, the STAV compositions of the present invention comprise at least three modifications which confers increased or enhanced stability to the STAVs, including, for example, improved resistance to nuclease digestion in vivo. In an embodiment, the STAV compositions of the present invention have undergone a chemical or biological modification to render them more stable. Exemplary modifications to the STAVs include the modification of a base, for example, the chemical modification of a base.

The STAV compositions of the invention are useful for the treatment of cancer, inflammation and other disorders. The term ‘therapeutic levels’ refers to levels of STAVs above normal physiological levels, or the levels in the subject prior to administration of the STAV composition. As provided herein, the compositions include a transfer vehicle. As used herein, the term ‘transfer vehicle’ can include any of the standard pharmaceutical carriers, diluents, excipients and the like which are generally intended for use in connection with the administration of biologically active agents, including nucleic acids. The compositions and in particular the transfer vehicles described herein are capable of delivering STAVs to the target cell. In embodiments, the transfer vehicle is a lipid nanoparticle (LNP).

The term ‘LNP’ means a lipid nanoparticle. A LNP represents a particle made from one or more lipids (e.g., cationic lipids, non-cationic lipids, conjugated lipids) and/or a sterol that prevents aggregation of the nanoparticle. A LNP can be used to deliver, a STAV and a vector containing a Replicant (i.e., one or more RNA), where the STAVs and the vector are encapsulated within the LNP. In an embodiment of the present invention, the LNPs used were GenVoy-ILM (Precision Nanosystems, Vancouver, Canada) based composition (<200 nm particle size, <0.2 PDI, >70% encapsulation efficiency and 0.1 mg/mL concentration).

In an embodiment of the present invention, DSPC, cholesterol, MC3 and DMG-PEG 2000 or combinations thereof can be used to generate the LNP to be combined with STAV1+RNA directed to a disease to target cells, STAV2+RNA directed to the disease to target cells or STAV3+RNA directed to the disease to target cells, where the diameter of the spherical LNPs can be approximately 88 nm, where approximately means+−10 nm. In an embodiment of the present invention, the cholesterol can be between 35-45% of the LNP composition. In an embodiment of the present invention, the LNP comprises a DSPC, cholesterol, an MC3-like lipid and a PEG-conjugated lipid. The phospholipid and cholesterol promote stability and structural integrity of the LNP. The ionizable lipid promotes electrostatic interaction with the negatively charged nucleic acids and assists intracellular delivery. The polymer-conjugated lipid improves solubility of the LNP in serum, and circulation by preventing the particles from aggregating, while retaining good biocompatibility and having good tolerance characteristics. In an embodiment of the present invention, the STAVs were composed of 76 bp of dsDNA modified with ps to block exonuclease activity, encapsulated at a nitrogen to phosphate mole ratio of approximately 6 (where approximately means plus or minus one). In an embodiment of the present invention, the LNP containing the STAVs+RNA directed to a disease to target cells can be approximately 100 nm in size, where approximately means plus or minus ten (10) percent. In an embodiment of the present invention, the STAVS+RNA directed to a disease to target cells are approximately 50% encapsulated in the LNPs. In this range approximately means plus or minus twenty (20) percent. In an alternative embodiment of the present invention, the STAVS are approximately 75% encapsulated in the LNPs. In this range approximately means plus or minus ten (10) percent. In another embodiment of the present invention, the STAVS+RNA directed to a disease to target cells are at least approximately 90% encapsulated in the LNPs. In this range approximately means plus or minus five (5) percent. In another alternative embodiment of the present invention, the STAVS+RNA directed to a disease to target cells are approximately 98% encapsulated in the LNPs. In this range approximately means plus or minus one (1) percent. In an embodiment of the present invention, the STAVS+RNA directed to a disease to target cells are approximately 98% encapsulated in the LNPs, at a concentration of dsDNA in the LNP in PBS of 0.2 mg/mL. LNP can be extremely useful for systemic applications, as they can exhibit extended circulation lifetimes following i.v. injection, they can accumulate at distal sites, and they can deliver the STAVs+RNA at sites distal to the site of administration.

The methods of the invention provide for optional co-delivery of one or more unique STAVs and RNA directed to a disease to target cells, for example, by combining two unique STAVs and RNA directed to a disease into a single transfer vehicle. In an embodiment of the present invention, a therapeutic first STAV and RNA directed to a disease, and a therapeutic second STAV and RNA directed to the disease, can be formulated in a single transfer vehicle and administered. In an alternative embodiment of the present invention, a therapeutic first STAV, with RNA directed to a disease and IFN type I, and a therapeutic second STAV, with RNA directed to the disease and IFN type I, can be formulated in a single transfer vehicle and administered. The present invention also contemplates co-delivery and/or co-administration of a therapeutic first STAV and RNA directed to a disease and subsequently delivery of IFN type I, and a second STAV and RNA directed to the disease and subsequently delivery of IFN type I to facilitate and/or enhance the function or delivery of one or both the therapeutic first STAV and the therapeutic second STAV.

Retrieved tumor cells transfected with STAVs activate APCs in trans and can generate potent anti-tumor T cell activity. Immunocompetent mice bearing metastatic tumors can be treated with STAV ‘loaded’ tumor cells after reinfusion and inoculation. Select leukemias, such as acute myeloid leukemia (AML), acute lymphocytic leukemia (ALL) and adult T cell leukemia (ATLL) can theoretically be amenable to treatment with STAVs. Further, the range of cancers can be extended to include melanomas and cutaneous T cell lymphomas. The i.t. inoculation of melanoma tumors (B16) in immunocompetent mice can be used to generate effective anti-tumor CTL activity and cause tumor regression. However, in situations where it is not feasible to retrieve sufficient tumor cells to carry out the transfection with STAVs for re-infusion, the STAV based approach may not be applicable.

In an embodiment of the present invention, the direct introduction of the STAVs into the tumor microenvironment (TME) can represent a significant advance. Further, in various embodiments of the present invention, the range of cancers amenable to STAV therapy can be extended using a non-cell based LNP strategy that effectively delivers high concentrations of STAVs+RNA into the TME to potently generate anti-tumor cytotoxic T cell activity. In an embodiment of the present invention, the tumor regression generated by STAVs+RNA can be augmented by co-delivery of checkpoint inhibitors.

In an embodiment of the present invention, data indicates that STAVs+RNA are a potent anti-tumor therapy that suppresses the growth of localized tumors (B16 melanoma model in C57/BL6 mice). In an embodiment of the present invention, the tumor regression effect was greatly augmented with the synergistic addition of checkpoint inhibitors. In an embodiment of the present invention, the activation of STING signaling in APC's is a main mechanism of generating anti-tumor T cell activity and is capable of overcoming resistance to checkpoint therapy. In an embodiment of the present invention, the benefit of STAVs+RNA over small drug agonists is that the procedure mimics the normal process of antigen cross-presentation, is non-toxic, simple, and inexpensive.

For any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

### Synthesis and Evaluation of STAVs

### STAVs Synthesized to Determine Optimum Length and Sequence

A:T30ES (SEQ ID NO:1, SEQ ID NO:2); A:T50ES (SEQ ID NO:3, SEQ ID NO:4); A:T60ES (SEQ ID NO:5, SEQ ID NO:6); A:T70ES 25 (SEQ ID NO:7, SEQ ID NO:8); A:T80ES (SEQ ID NO:9, SEQ ID NO:10); A:T90ES (SEQ ID NO:11, SEQ ID NO:12); and A:T100ES (SEQ ID NO:13, SEQ ID NO:14); A:T110ES (SEQ ID NO:15, SEQ ID NO:16); GC30ES (SEQ ID NO:17); GC50ES (SEQ ID No:18); GC60ES (SEQ ID NO:19); GC70ES (SEQ ID NO:20); GC80ES (SEQ ID NO:21); GC90ES 37 (SEQ ID NO:22); GC100ES (SEQ ID NO:23); PolyACTG76ES (SEQ ID NO:30), PolyCAGT76ES (SEQ ID NO:31), HSV RL2 intron-S(SEQ ID NO:32), HSV RL2 intron-AS (SEQ ID NO:33); polyA90ES-FAM (SEQ ID NO:35), polyT90ES (SEQ ID NO:36).

STAV1 = polyA76ES (SEQ ID NO: 24) +
polyT76ES (SEQ ID NO: 25)
PolyA76ES is
(SEQ ID NO: 24)
A(ps)A(ps)A(ps)AAAAAAAAAAAAAAAAAAAAAAAAAAAAAAAAAAA
AAAAAAAAAAAAAAAAAAAAAAAAAAAAAAAAAAA(ps)A(ps)A(ps)
A,
PolyT76ES
(SEQ ID NO: 25)
T(ps)T(ps)T(ps)TTTTTTTTTTTTTTTTTTTTTTTTTTTTTTTTTTT
TTTTTTTTTTTTTTTTTTTTTTTTTTTTTTTTTTT(ps)T(ps)T(ps)
T.
STAV2 = polyAC76ES (SEQ ID NO: 26) +
polyTG76ES (SEQ ID NO: 27)
PolyAC76ES is
(SEQ ID NO: 26)
A(ps)C(ps)A(ps)CACACACACACACACACACACACACACACACACAC
ACACACACACACACACACACACACACACACACACA(ps)C(ps)A(ps)
C,
PolyTG76ES
(SEQ ID NO: 27)
G(ps)T(ps)G(ps)TGTGTGTGTGTGTGTGTGTGTGTGTGTGTGTGTGT
GTGTGTGTGTGTGTGTGTGTGTGTGTGTGTGTGTG(ps)T(ps)G(ps)
T.
STAV3 = polyAT76ES (SEQ ID NO: 37) +
polyTA76ES (SEQ ID NO: 38)
PolyAT:TA76ES is
(SEQ ID NO: 37)
A(ps)T(ps)T(ps)AATTAATTAATTAATTAATTAATTAATTAATTAAT
TAATTAATTAATTAATTAATTAATTAATTAATTAA(ps)T(ps)T(ps)
A,
PolyTA:AT76ES
(SEQ ID NO: 38)
T(ps)A(ps)A(ps)TTAATTAATTAATTAATTAATTAATTAATTAATTA
ATTAATTAATTAATTAATTAATTAATTAATTAATT(ps)A(ps)A(ps)
T.
STAV4 = (SEQ ID NO: 39) + (SEQ ID NO: 40)
(SEQ ID NO: 39)
A(ps)C(ps)T(ps)GACTGACTGACTGACTGACTGACTGACTGACTGAC
TGACTGACTGACTGACTGACTGACTGACTGACTGA(ps)C(ps)T(ps)
G,
(SEQ ID NO: 40)
C(ps)A(ps)G(ps)TCAGTCAGTCAGTCAGTCAGTCAGTCAGTCAGTCA
GTCAGTCAGTCAGTCAGTCAGTCAGTCAGTCAGTC(ps)A(ps)G(ps)
T.
STAV5 = (SEQ ID NO: 41) + (SEQ ID NO: 42)
(SEQ ID NO: 41)
G(ps)A(ps)C(ps)CCTATCGATACAGGGCACGGGGTCGAACTGTTGGG
TTTCGCCATGGTACCCCCTGCATTTATATAGCCAG(ps)A(ps)C(ps)
C,
(SEQ ID NO: 42)
G(ps)G(ps)T(ps)CTGGCTATATAAATGCAGGGGGTACCATGGCGAAA
CCCAACAGTTCGACCCCGTGCCCTGTATCGATAGG(ps)G(ps)T(ps)
C.
STAV6 = (SEQ ID NO: 43) + (SEQ ID NO: 44)
(SEQ ID NO: 43)
A(ps)A(ps)A(ps)AAAAAAAAAAAAAAAAAAAAAAAAAAAAAAAAAAA
AAAAAAAAAAAAAAAAAAAAAAAAAAAAAAAAAAAAAAAAAAAAAAAAA
(ps)A(ps)A(ps)A,
(SEQ ID NO: 44)
T(ps)T(ps)T(ps)TTTTTTTTTTTTTTTTTTTTTTTTTTTTTTTTTTT
TTTTTTTTTTTTTTTTTTTTTTTTTTTTTTTTTTTTTTTTTTTTTTTTT
(ps)T(ps)T(ps)T.
STAV7 = (SEQ ID NO: 45) + (SEQ ID NO: 46)
(SEQ ID NO: 45)
G(ps)C(ps)G(ps)CGCGCGCGCGCGCGCGCGCGCGCGCGCGCGCGCGC
GCGCGCGCGCGCGCGCGCGCGCGCGCGCGCGCGCGCGCGCGCGCGCGCG
(ps)C(ps)G(ps)C,
(SEQ ID NO: 46)
C(ps)G(ps)C(ps)GCGCGCGCGCGCGCGCGCGCGCGCGCGCGCGCGCG
CGCGCGCGCGCGCGCGCGCGCGCGCGCGCGCGCGCGCGCGCGCGCGCGC
(ps)G(ps)C(ps)G.

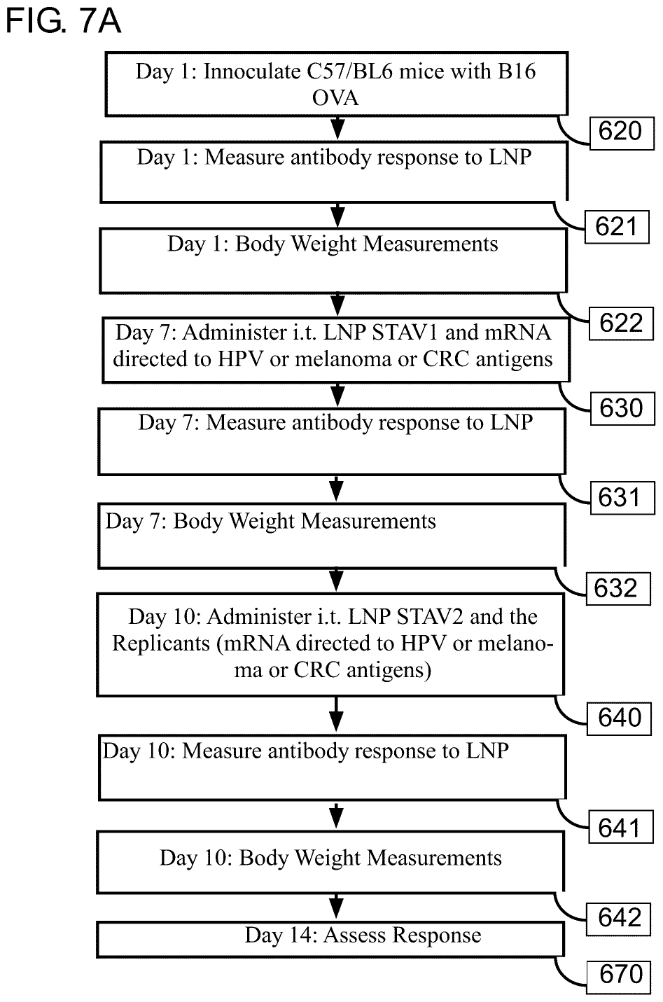

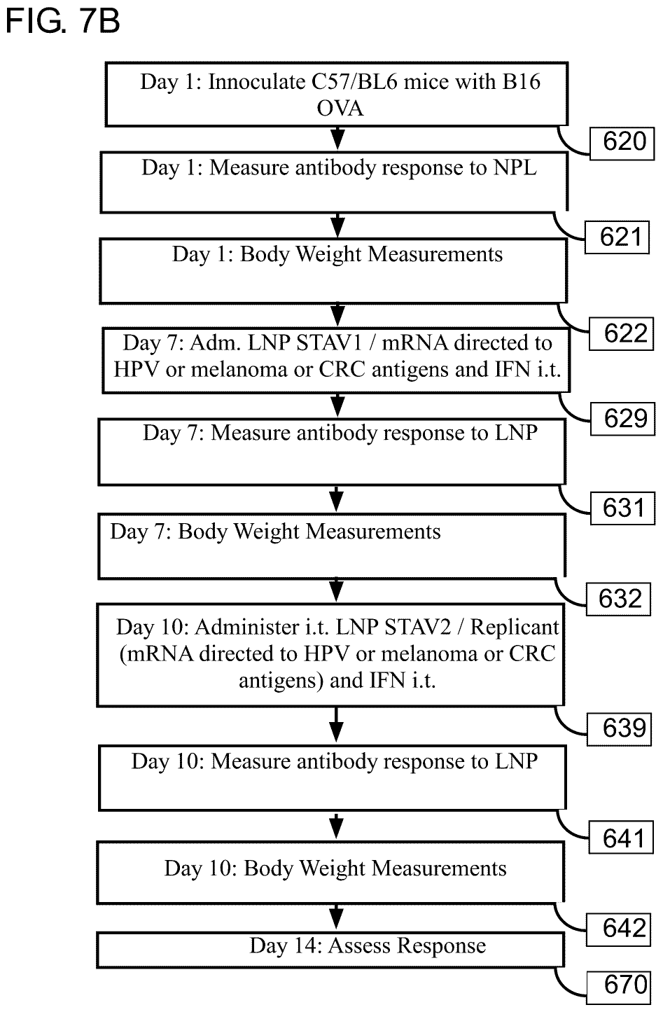

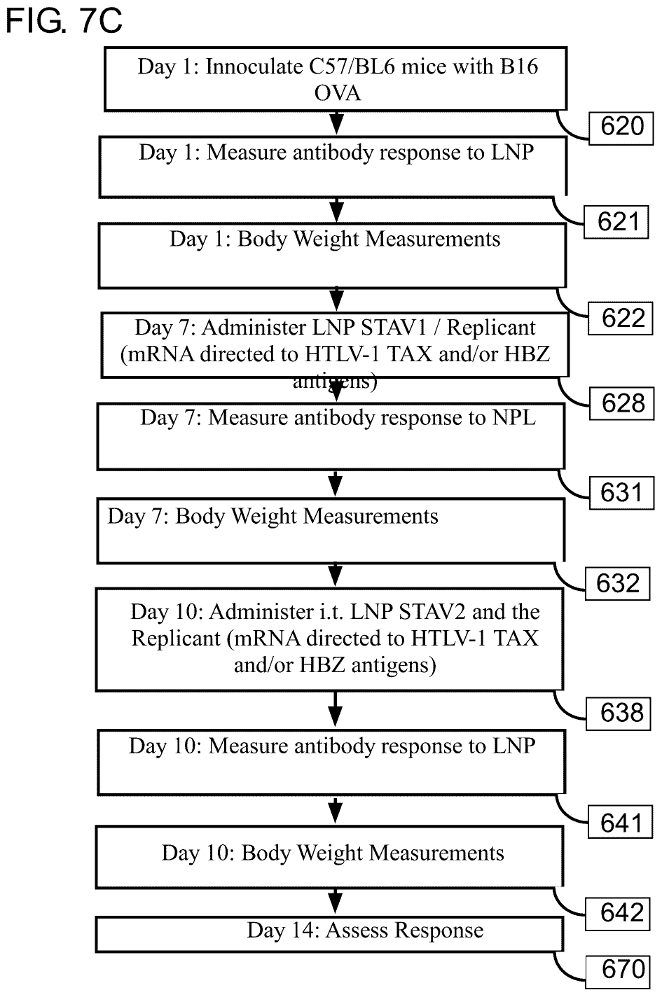

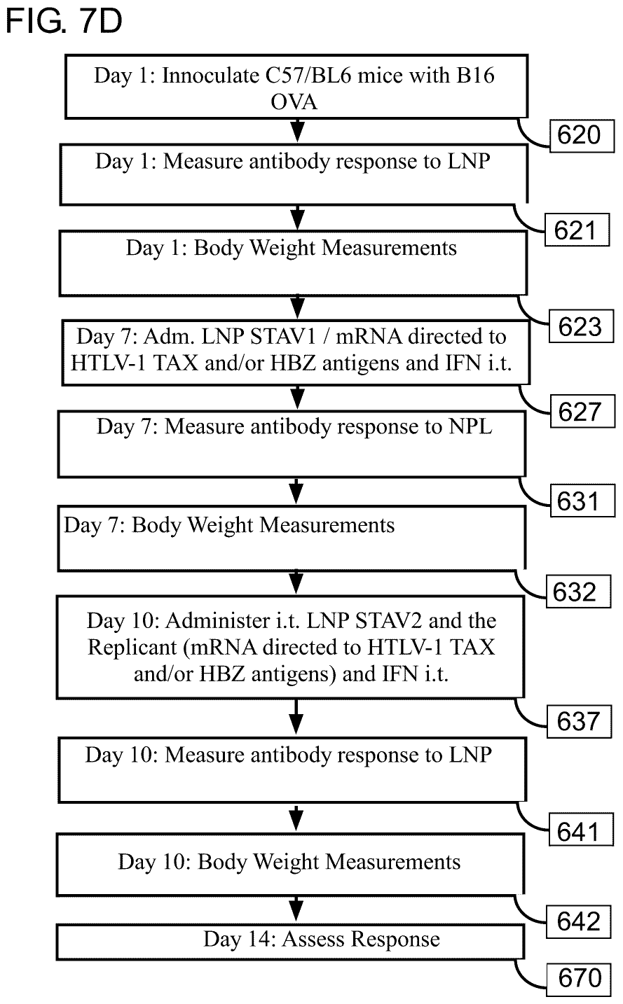

FIGS. 7A and 7B show flow diagrams of the protocol for administration of STAVs and RNA. To examine the importance of dose of STAV C57/BL6 mice (n=10) mice were inoculated on both flanks with B16-OVA (5×10⁵) 620. After seven (7) days, when tumors were 50 mm³ in volume, 25 μl (4 μg/mL; 0.1 ug/mouse) or 25 μL (20 μg/mL; 0.5 μg/mouse for STAV dose escalation examination) of STAVs and RNA (comprising STAV1) was injected (on only one flank) i.t. 630. Three (3) days later 25 μl (4 μg/mL; 0.1 ug/mouse) or 25 μL (20 μg/mL; 0.5 μg/mouse for STAV dose escalation examination) of STAVs and RNA (comprising STAV2) was injected (on the same flank) i.t. 640. Finally, three (3) days later 25 μl (4 μg/mL; 0.1 ug/mouse) or 25 μL (20 μg/mL; 0.5 μg/mouse for STAV dose escalation examination) of STAVs and RNA (comprising STAV3) was injected (on the same flank) i.t. 660. Nanoparticles alone, were used as controls. Body weights were monitored before and after treatment and the tumor volume measured using calipers and calculated with the formula V=(length×width²) 622, 632, 662. The generation of anti-tumor CTL activity (against OVA) was measured using the B16 model 621, 631, 661. Both flanks were monitored. Unexpectedly, STAVs and RNA generate effective anti-tumor T-cell responses which attack the non-injected tumor on the opposite flank. In addition to these studies, serum taken from the mice, before every inoculation, ascertained the antibody response to the nanoparticles themselves (to gauge the immune response to the formulations) 621, 631, 661. 0.5 μg of the particles was used in solid-phase ELISA assays and the serum from immunized animals incubated at 1/100 for 2 hours in PBS/0.1% Tween. Anti-murine conjugates were used to detect any-anti-nano-particle antibody. A significant humoral response was observed to the STAVs and RNA formulations.

FIGS. 7C and 7D show flow diagrams of the protocol for administration of STAVs and RNA with IFN type I. It has recently been shown that IFN type I can facilitate anti-tumor T cell activity, see U.S. Provisional Patent Application No. 63/521,696 entitled METHOD AND SYSTEM OF AUGMENTING CANCER THERAPY inventor Glen N. Barber, filed Jun. 18, 2023, which is herein expressly incorporated by reference in its entirety and for all purposes. STAVs enter the tumor microenvironment (TME) and function by entering and/or adhering to tumor cells. Tumor cells containing STAVs and RNA are engulfed by phagocytes in the TME, to activate extrinsic STING signaling and facilitate the cross-presentation of tumor antigen. Accordingly, stimulating STING signaling while presenting the antigen through the RNA is a key mechanism of cytotoxic T cell generation. IFN type I was used to evaluate whether STAVs and RNA exert synergistic effects with the IFN type I in B16 melanoma model. Sex matched C57/BL6 mice (n=10) were inoculated with B16-OVA (5×10⁵) on both flanks 620. After 7, 10, and 13 days, when tumors are 50 mm³ in volume, 25 μl (4 μg/mL; 0.1 μg/mouse) of STAVs and RNA (comprising three or more of STAV1, STAV2, STAV3, STAV4, STAV5, STAV6 and/or STAV7) will be injected i.t. in presence of IFN type I (50 μg/mouse) 635, 645, 665. Nanoparticles alone, IFN type I alone, PBS, isotype control antibody were used as controls. The tumor volume was measured using calipers and calculated with the formula V=(length×width²). STAVs and RNA exhibit potent anti-tumor activity, increasing CTL infiltration within the TME and augment the efficacy of IFN type I.

### EXAMPLES

### B16 Melanoma Model

Sex matched C57/BL6 mice (n=10) inoculated with B16-OVA (5×10⁵) on the flanks. After 7, 10, and 13 days, when tumors are 50 mm³ in volume, 25 μl (4 μg/mL; 0.1 μg/mouse) of Nano-STAVs (STAV1=(SEQ ID NO:24)+(SEQ ID NO:25); STAV2=(SEQ ID NO:26)+(SEQ ID NO:27); or STAV3=(SEQ ID NO:37)+(SEQ ID NO:38)] were injected i.m., i.v., or i.t. in presence Replicants (+−) IFN (50 μg/mouse). Nanoparticles alone, STAVs alone, Replicants alone, IFN alone, PBS were used as controls. The tumor volume was measured using calipers and calculated with the formula V=(length×width²). The generation of anti-tumor CTL activity (e.g., against OVA) was measured.

In an embodiment of the present invention, enrollment of human subjects of HTLV-1 of each cohort (ATLL, AML, ALL): enroll after the prior subject receives n doses of LNP containing STAV1-STAVn and Replicant (+−IFN) without treatment limiting toxicities (TLTs). An Interim Safety Analysis is undertaken. If there is one patient with TLT, then there is one patient with TLT, continue staggered accrual until 3 straight subjects have no treatment-limiting toxicity (TLT). If two or more subjects have TLT, stop accrual and re-evaluate protocol to adjust for toxicities and fix any other issues. If there are no patients with TLT, then continue injections of STAVn/Replicant (+−IFN) in subjects.

Correlative Studies—Molecular evaluations/analysis in patients with HTLV-1/ATLL: Venous blood can be collected from patients diagnosed with leukemia-type HTLV-1/ATLL at baseline, Day 10, at the ends of Months 1, Month 3, Month, 6, Month, 9, Month 12, an at the end-of-treatment visit after early discontinuation. Collected blood specimens can be processed and PMBCs can be isolated by centrifugation using standard Lymphoprep (ficol) procedure. A portion of fresh or thawed cells can be subjected to magnetic CD4-enrichment by negative selection using commercially available kits. These cells can serve as source for protein and RNA after standard extraction procedures. Non-enriched PBMCs can be used to extract genomic DNA for HTLV-1 pro-viral loads. The extracted cells may be utilized fresh or be cryopreserved in DMSO-liquid nitrogen.

Re-infusion of dead STAVs-loaded HTLV-1/ATLL cells can lead to phagocytosis by APCs in vivo. Such event can result in excess indigestible STAVs that can activate STING dependent signaling within APCs which in turn can facilitate a potent anti-tumor T cell activation. In addition, APCs can present HTLV-1 antigens, such as HBZ (which is always expressed ATLL tumors), which can in turn facilitate CTL priming against HTLV-1 infected cells and eliminate such clones.

CTL assays: To evaluate CTL responses after sequential administrations of STAVs loaded tumor cells and DC vaccinations, venous blood can be collected from patients at baseline, before each DC vaccination on Days 10, 17, 24, 31, 45, and at the end of Months 2, 3, and 6. Collected blood specimens can be processed on the same day. PMBCs can be isolated by centrifugation using standard Lymphoprep (ficol) procedure. The extracted cells may be utilized fresh or be cryopreserved in DMSO-liquid nitrogen.

Methods: HTLV-1 specific CTL responses can be assessed using PBMC isolated from peripheral blood. CD8 T cells can be isolated using human MACS CD8+ T cell isolation kit through negative selection (Miltenyil Biotec, 130-096-495). CD8 T Cells can be plated at 2×105 per well and stimulated with g/ml of tumor cell lysate protein or overlapping 15-aa peptides covering the envelope, TAX or HBZ region of HTLV-1 for ATLL (custom synthesized by GenScript). After 72 hours stimulation Interferon gamma secreting cells can be determined using an ELISPOT assay for human IFNγ and quantitated using a ELISPOT reader system. For flow cytometry, cells can be stimulated for 72 hours. Brefeldin A (3 mg/ml) can be added to the cells 6 h before analysis. Cells can be then washed, stained with cell surface marker (anti-CD3, anti-CD8), permeabilized with Cytofix/Cytoperm (BD Biosciences), and stained with IFNγ. Data can be acquired using an LSR II flow cytometer.

### Example 1. LNP Synthesis

In an embodiment of the present invention, a LNP can include a polyethylene glycol lipid, an ionizable cationic lipid, cholesterol and a surfactant (PEG lipid:ionizable lipid:cholesterol:surfactant in the ratio 50:10:30:2). In an alternative embodiment of the present invention, a LNP can include PEG lipid:ionizable lipid:cholesterol:surfactant in the ratio 50:10:30:1. In an embodiment of the present invention, a LNP can include ethanol solvent. In an alternative embodiment of the present invention, a LNP can include a phosphate-buffered saline (PBS) as the solvent (with 20% (w/v) sucrose). In another embodiment of the present invention, a LNP can include a tris-HCl-buffered saline (TBS; with 8% (w/v) sucrose) solvent. In another alternative embodiment of the present invention, a LNP can include a cryopreservation reagent solvent. In an embodiment of the present invention, the polyethylene glycol lipid is DMG-PEG 2000. In an embodiment of the present invention, the ionizable cationic lipid can be MC3. In an embodiment of the present invention, the surfactant can be distearoylphosphatidylcholine. In an embodiment of the present invention, the LNP dissolved in the solvent can be rapidly mixed with the STAVs and the mRNA in aqueous buffer at a pH where the ionizable lipid can be positively charged (pH approximately 4, where approximately means+−pH 1). The resulting dispersion can be dialyzed against a buffer to remove residual solvent and raise the pH above the pKa of the cationic lipid (pH approximately 7.4 for MC3, where approximately means+−pH 0.5) to produce the finished LNP. Formulations can be analyzed for particle size, polydispersity index (PDI), zeta potential total nucleic content, encapsulation efficiency and concentrations using light scattering, fluorescence and UHPLC. The formulation can be stored at −3 to 3° C. In an embodiment of the invention, the LNP can be stored in RNAse-free PBS containing 10% (w/v) sucrose at −20° C. In an embodiment of the invention, the LNP can be stored in RNAse-free TBS; with 8% (w/v) sucrose at −20° C. In an embodiment of the invention, the LNP can be frozen to −80° C. with a cryopreservation reagent (Bambaker, Portsmouth NH).

### Example 2. Replicant (HTLV1 gp62 Envelope Glycoprotein mRNA)

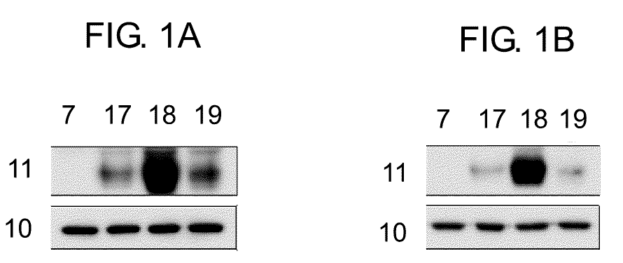

In an embodiment of the present invention, tumor cells loaded with STAVs, RNA directed to for example HTLV1 gp62 envelope glycoprotein (gp62G) can be used to treat autologous aggressive leukemia cells (ATLL, AML, and ALL) concomitant with a personalized dendritic cell (DCs) vaccine. In an alternative embodiment of the present invention, tumor cells loaded with STAVs, and mRNA directed to for example gp62G can be used to generate a vaccine for ATLL, AML, and ALL. In an embodiment of the present invention, the tumor cells loaded with STAVs, with mRNA directed to IFN type I and/or gp62G are able to stimulate antigen presenting cells (APCs) to generate immunity in adult T cell leukemia (ATLL) infected patients. In an embodiment of the present invention, the tumor cells loaded with STAVs, with mRNA directed to IFN type I and gp62G are able to stimulate antigen presenting cells (APCs) to generate immunity in adult T cell leukemia (ATLL) infected patients. FIG. 1A shows an immunoblot of total protein production probed with a gp62 antibody (11) and β-actin antibody (10) in HEK293T cells 24 hours after transfecting with either Mock (7), 2 g gp62 mRNA (17), 5 μg gp62 mRNA (18), or 10 μg gp62 mRNA (19), using a DMRIE-C liposomal transfection reagent (ThermoFisher Scientific, Cat. no. 10459014). FIG. 1B shows an immunoblot of total protein production probed with a gp62 antibody (11) and β-actin antibody (10) in HEK293T cells 32 hours after transfecting with either Mock (7), 2 μg gp62 mRNA (17), 5 μg gp62 mRNA (18), or 10 μg gp62 mRNA (19), using a DMRIE-C liposomal transfection reagent. In an unexpected result, the 5 μg gp62 mRNA transfection was found to be optimal.

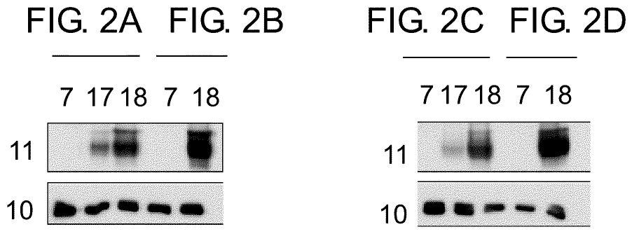

FIG. 2A shows an immunoblot of total protein production probed with a gp62 antibody (11) and β-actin antibody (10) in HEK293T cells 20 hours after transfecting with either Mock (7), 2 μg gp62 mRNA (17), or 5 μg gp62 mRNA (18), using a DMRIE-C liposomal transfection reagent. FIG. 2B shows an immunoblot of total protein production probed with a gp62 antibody (11) and β-actin antibody (10) in HEK293T cells 20 hours after transfecting with either Mock (7), or 5 μg gp62 mRNA (18), using lipofectamine 2000 liposomal transfection reagent (Invitrogen, Cat. no. 11668500). FIG. 2C shows an immunoblot of total protein production probed with a gp62 antibody (11) and β-actin antibody (10) in HEK293T cells 30 hours after transfecting with either Mock (7), 2 μg gp62 mRNA (17), or 5 μg gp62 mRNA (18), using a DMRIE-C liposomal transfection reagent. FIG. 2D shows an immunoblot of total protein production probed with a gp62 antibody (11) and β-actin antibody (10) in HEK293T cells 30 hours after transfecting with either Mock (7), or 5 μg gp62 mRNA (18), using lipofectamine 2000 liposomal transfection reagent. It was found that both DMRIE-C and lipofectamine 2000 resulted in efficient transfection, generating protein product 30 hours after transfection.

### Example 3. Replicant (HTLV-1 TAX HBZ mRNA)

In an embodiment of the present invention, tumor cells loaded with LNP that include STAVs (i.e., DNA) and HBZ-TAX mRNA (SEQ ID NO:54) can render non-immunogenic cells immunogenic. In an embodiment of the present invention, the tumor cells loaded with LNP can include STAV1 (SEQ ID NO:24-25), and HBZ-TAX mRNA (SEQ ID NO:54) to stimulate APCs in vitro and in vivo, in trans. In an embodiment of the present invention, the tumor cells loaded with LNP can include STAV2 (SEQ ID NO:26-27), and HBZ-TAX mRNA (SEQ ID NO:54) to stimulate APCs in vitro and in vivo, in trans. In an embodiment of the present invention, the tumor cells loaded with LNP can include STAV3 (SEQ ID NO:37-38), and HBZ-TAX mRNA (SEQ ID NO:54) to stimulate APCs in vitro and in vivo, in trans. In an embodiment of the present invention, the tumor cells loaded with LNP can include STAV4 (SEQ ID NO:39-40), and HBZ-TAX mRNA (SEQ ID NO:54) to stimulate APCs in vitro and in vivo, in trans. In an embodiment of the present invention, the tumor cells loaded with LNP can include STAV5 (SEQ ID NO:41-42), and HBZ-TAX mRNA (SEQ ID NO:54) to stimulate APCs in vitro and in vivo, in trans. In an embodiment of the present invention, the tumor cells loaded with LNP can include STAV6 (SEQ ID NO:43-44), and HBZ-TAX mRNA (SEQ ID NO:54) to stimulate APCs in vitro and in vivo, in trans. In an embodiment of the present invention, the tumor cells loaded with LNP can include STAV7 (SEQ ID NO:45-46), and HBZ-TAX mRNA (SEQ ID NO:54) to stimulate APCs in vitro and in vivo, in trans.

In an alternative embodiment of the invention, the tumor cells loaded with LNP can include STAVs (SEQ ID NO:24-27 and/or SEQ ID NO:37-46), and fragments of the HBZ-TAX mRNA (SEQ ID NO:54) to stimulate APCs in vitro and in vivo. In another embodiment of the invention, the tumor cells loaded with LNP can include STAVs (SEQ ID NO:24-27 and/or SEQ ID NO:37-46), the HBZ-TAX mRNA (SEQ ID NO:54) and IFN (SEQ ID NO:117) to stimulate APCs in vitro and in vivo.

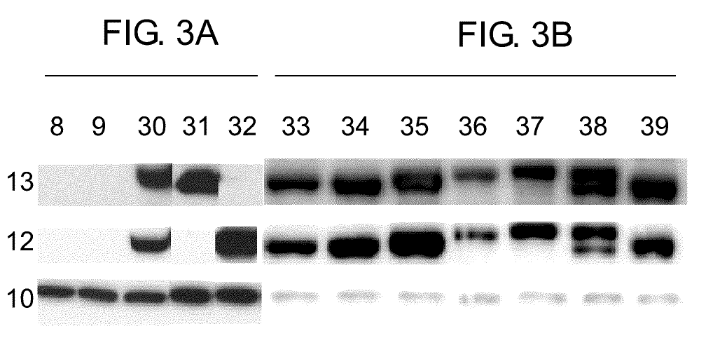

In an embodiment of the present invention, tumor cells loaded with STAVs, RNA directed to for example HTLV-1 TAX (TAX) and/or HTLV-1 basic leucine zipper factor (HBZ) can be used to treat autologous aggressive leukemia cells (ATLL, AML, and ALL) concomitant with a personalized dendritic cell (DCs) vaccine. In an alternative embodiment of the present invention, tumor cells loaded with STAVs, and mRNA directed to for example TAX and/or HBZ can be used to generate a vaccine for ATLL, AML, and ALL. In an embodiment of the present invention, the tumor cells loaded with STAVs, with mRNA directed to TAX and/or HBZ, and IFN type I are able to stimulate antigen presenting cells (APCs) to generate immunity in adult T cell leukemia (ATLL) infected patients. FIG. 3A shows an immunoblot of total protein production (cells are lysed for protein extraction) probed with a HBZ antibody (13), a Tax antibody (12) and j-actin antibody (10) in HEK293T cells 36 hours after transfecting with different constructs cloned in pcDNA3.1 (+) using liposomal transfection reagent either alone (8), empty vector (9), HBZ-Tax mRNA (30), HBZ mRNA (31), Tax mRNA (32), using lipofectamine 2000 liposomal transfection reagent. FIG. 3B shows an immunoblot of total protein production (cells are lysed for protein extraction) probed with a HBZ antibody (13), a Tax antibody (12) and j-actin antibody (10) in HEK293T cells 48 hours after transfecting with different constructs cloned in pcDNA3.1 (+) using liposomal transfection reagent either HBZ-Tax (R114D) mRNA (33), HBZ-Tax (R114D) with the N-terminal endoplasmic reticulum (ER) signalling peptide of human STING (MKLSLVAAMLLLLSAARA, SEQ ID NO:162) mRNA (34), HBZ-Tax (R114D) with the N-terminal endoplasmic reticulum (ER) signaling peptide of human STING (MKLSLVAAMLLLLSAARA, SEQ ID NO:162) mRNA with ER retention sequence (KDEL, SEQ ID NO:166) (35), HBZ-Tax (R114D) with the N-terminal endoplasmic reticulum (ER) signaling peptide of human BiP (MPHSSLHPSIPCPRGHG, SEQ ID NO:165) mRNA (36), HBZ-Tax (R114D) with the N-terminal endoplasmic reticulum (ER) signaling peptide of human BiP (MPHSSLHPSIPCPRGHG, SEQ ID NO:165) mRNA with ER retention sequence (37), Tax mRNA (32), HBZ-Tax (R114D) with the Mitochondrial Targeting signal (MLATRVFSLVGKRAISTSVCVR, SEQ ID NO:165) mRNA (38), or Tax mRNA (32), HBZ-Tax (R114D) with the Myristylation/palmitoylation site (MGCIKSKRKD, SEQ ID NO:164) mRNA (39), using lipofectamine 2000 liposomal transfection reagent. SEQ ID NOS:167-171 are codon optimized sequences corresponding to SEQ ID NOS:162-166, respectively.

### Example 4. Replicant (Ovalbumin Antigen mRNA)

In an embodiment of the present invention, tumor cells can be loaded with LNP that include STAVs (i.e., DNA) and Ovalbumin mRNA. In an embodiment of the present invention, tumor cells loaded with LNP that include STAVs (i.e., DNA) and mRNA encoding ovalbumin (SEQ ID NO:47) can render non-immunogenic cells immunogenic. In an embodiment of the present invention, the tumor cells loaded with LNP can include STAV1 (SEQ ID NO:24-25), and ovalbumin mRNA (SEQ ID NO: 47) to stimulate APCs in vitro and in vivo, in trans. In an embodiment of the present invention, the tumor cells loaded with LNP can include STAV2 (SEQ ID NO:26-27), and ovalbumin mRNA (SEQ ID NO: 47) to stimulate APCs in vitro and in vivo, in trans. In an embodiment of the present invention, the tumor cells loaded with LNP can include STAV3 (SEQ ID NO:37-38), and ovalbumin mRNA (SEQ ID NO: 47) to stimulate APCs in vitro and in vivo, in trans. In an embodiment of the present invention, the tumor cells loaded with LNP can include STAV4 (SEQ ID NO:39-40), and ovalbumin mRNA (SEQ ID NO: 47) to stimulate APCs in vitro and in vivo, in trans. In an embodiment of the present invention, the tumor cells loaded with LNP can include STAV5 (SEQ ID NO:41-42), and ovalbumin mRNA (SEQ ID NO: 47) to stimulate APCs in vitro and in vivo, in trans. In an embodiment of the present invention, the tumor cells loaded with LNP can include STAV6 (SEQ ID NO:43-44), and ovalbumin mRNA (SEQ ID NO: 47) to stimulate APCs in vitro and in vivo, in trans. In an embodiment of the present invention, the tumor cells loaded with LNP can include STAV7 (SEQ ID NO:45-46), and ovalbumin mRNA (SEQ ID NO: 47) to stimulate APCs in vitro and in vivo.

In an alternative embodiment of the invention, the tumor cells loaded with LNP can include STAVs (SEQ ID NO:24-27 and/or SEQ ID NO:37-46), and fragments of the ovalbumin mRNA (SEQ ID NO: 47) (e.g., mRNA fragments encoding for residues 47-64, 185-210 and/or 264-292) to stimulate APCs in vitro and in vivo. In another embodiment of the invention, the tumor cells loaded with LNP can include STAVs (SEQ ID NO:24-27 and/or SEQ ID NO:37-46), and the ovalbumin mRNA (SEQ ID NO: 47) and IFN (SEQ ID NO:117) to stimulate APCs in vitro and in vivo.

In an embodiment of the present invention, tumor cells can be loaded with LNP that include STAVs (i.e., DNA) and an optimized Ovalbumin mRNA. In an embodiment of the present invention, the optimized mRNA can be 5′ capped with the modified trimer 7-methyl guanosine-2′-O-methoxy adenosine-guanosine (m7GAG) (CleanCapAG, Trilink Bio Technologies, San Diego, CA). In an embodiment of the present invention, the tumor cells loaded with LNP can include STAV1 (SEQ ID NO:24-25), and m7GAG capped ovalbumin mRNA (SEQ ID NO:48) can render non-immunogenic cells immunogenic. In an embodiment of the present invention, the tumor cells loaded with LNP can include STAV1 (SEQ ID NO:24-25), and m7GAG capped ovalbumin mRNA (SEQ ID NO:48) to stimulate APCs in vitro and in vivo, in trans. In an embodiment of the present invention, the tumor cells loaded with LNP can include STAV2 (SEQ ID NO:26-27), and m7GAG capped ovalbumin mRNA (SEQ ID NO:48) to stimulate APCs in vitro and in vivo, in trans. In an embodiment of the present invention, the tumor cells loaded with LNP can include STAV3 (SEQ ID NO:37-38), and m7GAG capped ovalbumin mRNA (SEQ ID NO:48) to stimulate APCs in vitro and in vivo, in trans. In an embodiment of the present invention, the tumor cells loaded with LNP can include STAV4 (SEQ ID NO:39-40), and m7GAG capped ovalbumin mRNA (SEQ ID NO:48) to stimulate APCs in vitro and in vivo, in trans. In an embodiment of the present invention, the tumor cells loaded with LNP can include STAV5 (SEQ ID NO:41-42), and m7GAG capped ovalbumin mRNA (SEQ ID NO:48) to stimulate APCs in vitro and in vivo, in trans. In an embodiment of the present invention, the tumor cells loaded with LNP can include STAV6 (SEQ ID NO:43-44), and m7GAG capped ovalbumin mRNA (SEQ ID NO:48) to stimulate APCs in vitro and in vivo, in trans. In an embodiment of the present invention, the tumor cells loaded with LNP can include STAV7 (SEQ ID NO:45-46), and m7GAG capped ovalbumin mRNA (SEQ ID NO:48) to stimulate APCs in vitro and in vivo.

In an alternative embodiment of the invention, the tumor cells loaded with LNP can include STAVs (SEQ ID NO:24-27 and/or SEQ ID NO:37-46), and fragments of the m7GAG capped ovalbumin mRNA (SEQ ID NO: 48) (e.g., mRNA fragments encoding for residues 47-64, 185-210 and/or 264-292) to stimulate APCs in vitro and in vivo. In another embodiment of the invention, the tumor cells loaded with LNP can include STAVs (SEQ ID NO:24-27 and/or SEQ ID NO:37-46), the m7GAG capped ovalbumin mRNA (SEQ ID NO: 48) and IFN (SEQ ID NO:117) to stimulate APCs in vitro and in vivo.

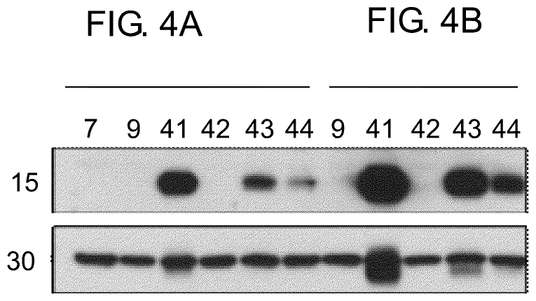

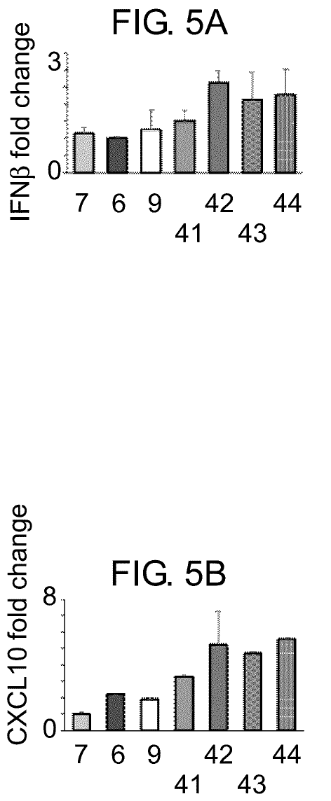

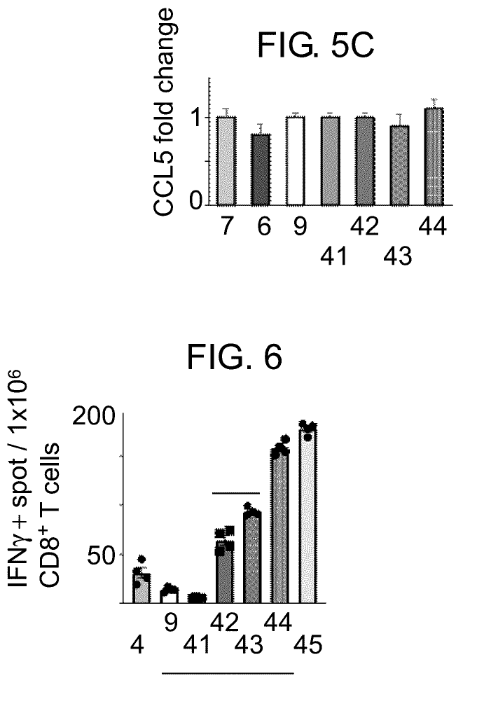

In an embodiment of the present invention, tumor cells loaded with STAV1, Replicon directed to for example OVA can be used to increase cytotoxic CD8+ T cell (CTL) activity. In an alternative embodiment of the present invention, tumor cells loaded with STAV1, and mRNA directed to OVA and IFN type I are able to stimulate antigen presenting cells (APCs) to generate improved immunity in cancer patients. FIG. 4A shows an immunoblot of total protein production (cells are lysed for protein extraction) probed with ovalbumin antibody (15) and β-actin antibody (10) in HEK293T cells 6 hours after reagents (2 μg/mL) in a lipo-nano-particle (LNP) are delivered with either Mock (7), empty vector (9), OVA mRNA (41), STAV1 (42), OVA mRNA+STAV1 (2:1) (43), OVA mRNA+STAV1 (1:2) (44). FIG. 4B shows an immunoblot of total protein production (cells are lysed for protein extraction) probed with ovalbumin antibody (15) and (3-actin antibody (10) in HEK293T cells 24 hours after reagents (2 μg/mL) in a lipo-nano-particle (LNP) are delivered with either empty vector (9), OVA mRNA (41), STAV1 (42), OVA mRNA+STAV1 (2:1) (43), OVA mRNA+STAV1 (1:2) (44). FIG. 5A shows a schematic representation of IFN-β production in HEK293T cells irradiated with UV (120 mJ/cm²) after 6 hours, incubated for 24 hours fed to WT macrophages where either the HEK293T cells are treated with Mock (7), UV light only (6), empty LNP (9), OVA mRNA (41), STAV1 (42), OVA mRNA+STAV1 (2:1) (43), OVA mRNA+STAV1 (1:2) (44). FIG. 5B shows a schematic representation of CXCL10 production in HEK293T cells irradiated with UV (120 mJ/cm²) after 6 hours, incubated for 24 hours fed to WT macrophages where either the HEK293T cells are treated with Mock (7), UV light only (6), empty LNP (9), OVA mRNA (41), STAV1 (42), OVA mRNA+STAV1 (2:1) (43), OVA mRNA+STAV1 (1:2) (44). FIG. 5C shows a schematic representation of CCL5 production in HEK293T cells irradiated with UV (120 mJ/cm²) after 6 hours, incubated for 24 hours fed to WT macrophages where either the HEK293T cells are treated with: Mock (7), UV light only (6), empty LNP (9), OVA mRNA (41), STAV1 (42), OVA mRNA+STAV (2:1) (43), OVA mRNA+STAV1 (1:2) (44). FIG. 6 shows an ELISPOT representation of IFN-γ production in C57/BL6 mice at 5/6 weeks (n=4 per group) intramuscularly injected with PBS or 10 μg of different LNP conditions (for (9), (41), (42), (43) and (44)) per mouse and boosted twice every 3 weeks (where 10 days after the 2nd boost, mice are sacrificed and their spleen collected to measure cytotoxic CD8+ T cell (CTL) activity specific against OVA peptide (SIINFEKL, SEQ ID NO:34) where PBS (7), empty LNP (9), OVA mRNA (41), STAV1 (42), OVA mRNA+STAV1 (2:1) (43), OVA mRNA+STAV1 (1:2) (44), and VSV-OVA (Positive control) (45). Unexpectedly, the Replicant/STAV (1:2) introduced in vivo results in a synergistic increase in IFNγ compared to either the Replicant alone or the STAV alone.

### Example 5. Interferon Administration

In an embodiment of the present invention, a LNP can be administered with mRNA directed to a Gene of Interest (GOI) together with type I Interferon (IFN) (SEQ ID NO:117). In an embodiment of the present invention, the LNP can be mixed with the GOI mRNA and IFN and co-administered into tumor cells. In an alternative embodiment of the present invention, the GOI mRNA plus the IFN can be co-administered into tumor cells and then the GOI mRNA and the LNP can be re-administered into the tumor cells after a time period. In another embodiment of the present invention, the GOI mRNA in the LNP can be administered into tumor cells and then the IFN can be administered intratumorally after a time period. In various embodiments of the invention, the time period can be approximately 10 minutes to 100 minutes. In this range approximately means plus or minus twenty (20) percent. In an embodiment of the present invention, the GOI mRNA can be administered i.m. (intra muscularly), i.t. (intrathecal) or i.v. (intravenous). In an embodiment of the present invention, the IFN can be administered i.m. (intra muscularly), i.t. (intrathecal) or i.v. (intravenous). In an embodiment of the present invention, the IFN can be administered systemically.

FIG. 7A is a flow diagram showing a treatment protocol for administration of STAVs and Replicant (mRNA directed to HPV antigens, or mRNA directed to melanoma antigens or mRNA directed to CRC antigens) to B16 mice. At day 1 620, an antibody response to a blank LNP is measured 621 and body weight 622. On day 7 the STAVn (e.g., STAV1) and Replicant (mRNA directed to HPV antigens, or mRNA directed to melanoma antigens or mRNA directed to CRC antigens) in a LNP is injected into the tumor i.t. 630. An antibody response to LNP is measured 631 and body weight is measured 632. The procedure is repeated on day 10 with STAVn (e.g., STAV2) and Replicant (mRNA directed to HPV antigens, or mRNA directed to melanoma antigens or mRNA directed to CRC antigens) in a LNP is injected into the tumor i.t. 640 where an antibody response to the LNP is measured 641 and body weight is measured 642. On day 14 the response can be assessed 670. The procedure can be repeated with additional STAVn (e.g., STAV3 or STAV4 or STAV5) and Replicant (mRNA directed to HPV antigens, or mRNA directed to melanoma antigens or mRNA directed to CRC antigens) in LNP. In an embodiment of the invention, if the response is sufficient the length of time before the next administration of a STAVn and Replicant (mRNA directed to HPV antigens, or mRNA directed to melanoma antigens or mRNA directed to CRC antigens) in LNP can be extended. In an alternative embodiment of the invention, if the response is insufficient the next administration of a STAVn and Replicant (mRNA directed to HPV antigens, or mRNA directed to melanoma antigens or mRNA directed to CRC antigens) in LNP can be brought forward.

FIG. 7B is a flow diagram showing a treatment protocol for administration of STAVs and Replicant (mRNA directed to HPV antigens, or mRNA directed to melanoma antigens or mRNA directed to CRC antigens) with IFN to B16 mice. At day 1 620, an antibody response to a blank LNP is measured 621 and body weight 622. On day 7 the STAVn (e.g., STAV1) and Replicant (mRNA directed to HPV antigens, or mRNA directed to melanoma antigens or mRNA directed to CRC antigens) in a LNP is injected into the tumor i.t. and thereafter IFN is administered i.t. 629. An antibody response to LNP is measured 631 and body weight is measured 632. The procedure is repeated on day 10 with STAVn (e.g., STAV2) and Replicant (mRNA directed to HPV antigens, or mRNA directed to melanoma antigens or mRNA directed to CRC antigens) in a LNP is injected into the tumor i.t. and thereafter IFN is administered i.t. 639 where an antibody response to the LNP is measured 641 and body weight is measured 642. On day 14 the response can be assessed 670. The procedure can be repeated with additional STAVn (e.g., STAV3 or STAV4 or STAV5) and Replicant (mRNA directed to HPV antigens, or mRNA directed to melanoma antigens or mRNA directed to CRC antigens) in LNP and thereafter IFN is administered i.t. In an embodiment of the invention, if the response is sufficient the length of time before the next administration of a STAVn and Replicant (mRNA directed to HPV antigens, or mRNA directed to melanoma antigens or mRNA directed to CRC antigens) in LNP and thereafter IFN is administered i.t. can be extended. In an alternative embodiment of the invention, if the response is insufficient the next administration of a STAVn and Replicant (mRNA directed to HPV antigens, or mRNA directed to melanoma antigens or mRNA directed to CRC antigens) in LNP and thereafter IFN is administered i.t. can be brought forward.

FIG. 7C is a flow diagram showing a treatment protocol for administration of STAVs and Replicant (mRNA directed to TAX and/or HBZ antigens) to B16 mice. At day 1 620, an antibody response to a blank LNP is measured 621 and body weight 622. On day 7 the STAVn (e.g., STAV1) and Replicant (mRNA directed to HPV antigens, or mRNA directed to melanoma antigens or mRNA directed to CRC antigens) in a LNP is injected into the tumor i.t. 628. An antibody response to LNP is measured 631 and body weight is measured 632. The procedure is repeated on day 10 with STAVn (e.g., STAV2) and Replicant (mRNA directed to TAX and/or HBZ antigens) in a LNP is injected into the tumor i.t. 638 where an antibody response to the LNP is measured 641 and body weight is measured 642. On day 14 the response can be assessed 670. The procedure can be repeated with additional STAVn (e.g., STAV3 or STAV4 or STAV5) and Replicant mRNA directed to TAX and/or HBZ antigens) in LNP. In an embodiment of the invention, if the response is sufficient the length of time before the next administration of a STAVn and Replicant (mRNA directed to TAX and/or HBZ antigens) in LNP can be extended. In an alternative embodiment of the invention, if the response is insufficient the next administration of a STAVn and Replicant (mRNA directed to TAX and/or HBZ antigens) in LNP can be brought forward.

FIG. 7D is a flow diagram showing a treatment protocol for administration of STAVs and Replicant (mRNA directed to TAX and/or HBZ antigens) with IFN to B16 mice. At day 1 620, an antibody response to a blank LNP is measured 621 and body weight 622. On day 7 the STAVn (e.g., STAV1) and Replicant mRNA directed to TAX and/or HBZ antigens) in a LNP is injected into the tumor i.t. and thereafter IFN is administered i.t. 627. An antibody response to LNP is measured 631 and body weight is measured 632. The procedure is repeated on day 10 with STAVn (e.g., STAV2) and Replicant (mRNA directed to TAX and/or HBZ antigens) in a LNP is injected into the tumor i.t. and thereafter IFN is administered i.t. 637 where an antibody response to the LNP is measured 641 and body weight is measured 642. On day 14 the response can be assessed 670. The procedure can be repeated with additional STAVn (e.g., STAV3 or STAV4 or STAV5) and Replicant (mRNA directed to TAX and/or HBZ antigens) in LNP and thereafter IFN is administered i.t. In an embodiment of the invention, if the response is sufficient the length of time before the next administration of a STAVn and Replicant (mRNA directed to TAX and/or HBZ antigens) in LNP and thereafter IFN is administered i.t. can be extended. In an alternative embodiment of the invention, if the response is insufficient the next administration of a STAVn and Replicant (mRNA directed to TAX and/or HBZ antigens) in LNP and thereafter IFN is administered i.t. can be brought forward.

Similar therapy regimes as shown in FIGS. 1A-1D, can be developed for human trials. After completion of therapy with humans (approximately 2 months) subjects will be followed at the end months 3, 6, 9, and 12 (±7 days) for clinical assessment (complete physical exam, CBC, CMP, uric acid, phosphorus, and LDH), with response assessments as per Section 9.0 (CT scans, bone marrow biopsy, peripheral blood flow cytometry, and PCR to evaluate for minimal residual disease). Those who remain progression-free after Year 1 and decide to remain on study will be followed approximately every 3-6 months during Year-2 post-treatment for routine monitoring and laboratory tests, and response assessments at the discretion of the investigator. Thereafter, subjects will continue to be followed every 6 months (±1 month) via a telephone call during Years 3 to 5 (at a minimum) for survival only with periodic visits and clinical assessments at the discretion of the investigator. Subjects who discontinue treatment for disease progression will come off treatment and will be followed for survival only every 6 months (±1 month) for up to 5 years from time of treatment initiation. Subjects who withdraw consent will come off study. A study participant is considered to have completed the study once he or she completes all phases of the study treatment and study related laboratory tests. The primary and secondary endpoints will be available for analysis once all patients have met the end points. Therefore, the clinical trial will be considered completed when the last participant has completed all phases of the study including the last visit or the last scheduled procedure, and the clinical endpoints are available for analysis.

### Example 6. Plasmid mRNA Synthesis

In an embodiment of the present invention, a plasmid can be utilized for the synthesis of mRNA. In an embodiment of the present invention, an optimized plasmid can be used (Azenta Life Sciences, San Diego, CA). In an embodiment of the present invention, the plasmid can contain T7 cap1+UTR (SEQ ID NO:49). In an embodiment of the present invention, the plasmid can contain PolyA+UTR+3RE (SEQ ID NO:50). In an embodiment of the present invention, the plasmid can contain 5′UTR+3′UTR. In an embodiment of the present invention, the plasmid can contain mHBZ-mTAX_pcDNA3.1(+) (Ampicillin, 7019 bp) (SEQ ID NO:51). In an embodiment of the present invention, the plasmid can contain Intermediate (Ampicillin, 7050 bp, from Step 1) (SEQ ID NO:52). In an embodiment of the present invention, the plasmid can contain Intermediate (Ampicillin, from Step 1) (SEQ ID NO:53). In an embodiment of the present invention, the plasmid can contain mHBZ-mTAX_pcDNA3.1(+) IVT (Ampicillin, from step #2) (SEQ ID NO:54). The DNA sequence was used to generate a plasmid with a T7 promoter, 5′Cap, 5′ UTA, 3′ UTR, and a polyA tail, which was cloned into a vector and expressed, and then purified using the 5′Cap sequence to generate the desired mHBZ-mTAX mRNA (SEQ ID NO:54).

### Example 7. Replicant (mRNA Directed to HTLV-1 mTAX Antigen)

In an embodiment of the present invention, tumor cells loaded with LNP that include STAVs (i.e., DNA) and mRNA encoding HTLV-1 mTAX (SEQ ID NO:118) can render non-immunogenic cells immunogenic. In an embodiment of the present invention, the tumor cells loaded with LNP can include STAV1 (SEQ ID NO:24-25), and HTLV-1 mTAX mRNA (SEQ ID NO:118) to stimulate APCs in vitro and in vivo, in trans. In an embodiment of the present invention, the tumor cells loaded with LNP can include STAV2 (SEQ ID NO:26-27), and HTLV-1 mTAX mRNA (SEQ ID NO:118) to stimulate APCs in vitro and in vivo, in trans. In an embodiment of the present invention, the tumor cells loaded with LNP can include STAV3 (SEQ ID NO:37-38), and HTLV-1 mTAX mRNA (SEQ ID NO:118) to stimulate APCs in vitro and in vivo, in trans. In an embodiment of the present invention, the tumor cells loaded with LNP can include STAV4 (SEQ ID NO:39-40), and HTLV-1 mTAX mRNA (SEQ ID NO:118) to stimulate APCs in vitro and in vivo, in trans. In an embodiment of the present invention, the tumor cells loaded with LNP can include STAV5 (SEQ ID NO:41-42), and HTLV-1 mTAX mRNA (SEQ ID NO:118) to stimulate APCs in vitro and in vivo, in trans. In an embodiment of the present invention, the tumor cells loaded with LNP can include STAV6 (SEQ ID NO:43-44), and HTLV-1 mTAX mRNA (SEQ ID NO:118) to stimulate APCs in vitro and in vivo, in trans. In an embodiment of the present invention, the tumor cells loaded with LNP can include STAV7 (SEQ ID NO:45-46), and HTLV-1 mTAX mRNA (SEQ ID NO:118) to stimulate APCs in vitro and in vivo, in trans.

In an alternative embodiment of the invention, the tumor cells loaded with LNP can include STAVs (SEQ ID NO:24-27 and/or SEQ ID NO:37-46), and fragments of the HTLV-1 mTAX mRNA (SEQ ID NO:118) to stimulate APCs in vitro and in vivo. In another embodiment of the invention, the tumor cells loaded with LNP can include STAVs (SEQ ID NO:24-27 and/or SEQ ID NO:37-46), the HTLV-1 mTAX mRNA (SEQ ID NO:118) and IFN (SEQ ID NO:117) to stimulate APCs in vitro and in vivo.

### Example 8. Replicant (mRNA Directed to HTLV-1 mHBZ Antigen)

In an embodiment of the present invention, tumor cells loaded with LNP that include STAVs (i.e., DNA) and mRNA encoding HTLV-1 mHBZ (SEQ ID NO:119) can render non-immunogenic cells immunogenic. In an embodiment of the present invention, the tumor cells loaded with LNP can include STAV1 (SEQ ID NO:24-25), and HTLV-1 mHBZ mRNA (SEQ ID NO:119) to stimulate APCs in vitro and in vivo, in trans. In an embodiment of the present invention, the tumor cells loaded with LNP can include STAV2 (SEQ ID NO:26-27), and HTLV-1 mHBZ mRNA (SEQ ID NO:119) to stimulate APCs in vitro and in vivo, in trans. In an embodiment of the present invention, the tumor cells loaded with LNP can include STAV3 (SEQ ID NO:37-38), and HTLV-1 mHBZ mRNA (SEQ ID NO:119) to stimulate APCs in vitro and in vivo, in trans. In an embodiment of the present invention, the tumor cells loaded with LNP can include STAV4 (SEQ ID NO:39-40), and HTLV-1 mHBZ mRNA (SEQ ID NO:119) to stimulate APCs in vitro and in vivo, in trans. In an embodiment of the present invention, the tumor cells loaded with LNP can include STAV5 (SEQ ID NO:41-42), and HTLV-1 mHBZ mRNA (SEQ ID NO:119) to stimulate APCs in vitro and in vivo, in trans. In an embodiment of the present invention, the tumor cells loaded with LNP can include STAV6 (SEQ ID NO:43-44), and HTLV-1 mHBZ mRNA (SEQ ID NO:119) to stimulate APCs in vitro and in vivo, in trans. In an embodiment of the present invention, the tumor cells loaded with LNP can include STAV7 (SEQ ID NO:45-46), and HTLV-1 mHBZ mRNA (SEQ ID NO:119) to stimulate APCs in vitro and in vivo, in trans. In an alternative embodiment of the invention, the tumor cells loaded with LNP can include STAVs (SEQ ID NO:24-27 and/or SEQ ID NO:37-46), and fragments of the HTLV-1 mHBZ mRNA (SEQ ID NO:119) to stimulate APCs in vitro and in vivo.

In an alternative embodiment of the invention, the tumor cells loaded with LNP can include STAVs (SEQ ID NO:24-27 and/or SEQ ID NO:37-46), and fragments of the HTLV-1 mHBZ mRNA (SEQ ID NO:119) (e.g., mRNA fragments encoding for residues 10-19) to stimulate APCs in vitro and in vivo. In another embodiment of the invention, the tumor cells loaded with LNP can include STAVs (SEQ ID NO:24-27 and/or SEQ ID NO:37-46), the HTLV-1 mHBZ mRNA (SEQ ID NO:119) and IFN (SEQ ID NO:117) to stimulate APCs in vitro and in vivo.

### Example 9. Replicant (HPV E6 mRNA)

In an embodiment of the present invention, tumor cells loaded with LNP that include STAVs (i.e., DNA) and mRNA encoding human papillomavirus (HPV) E6 (SEQ ID NO:120) can render non-immunogenic cells immunogenic. In an embodiment of the present invention, the tumor cells loaded with LNP can include STAV1 (SEQ ID NO:24-25), and HPV E6 mRNA (SEQ ID NO:120) to stimulate APCs in vitro and in vivo, in trans. In an embodiment of the present invention, the tumor cells loaded with LNP can include STAV2 (SEQ ID NO:26-27), and HPV E6 mRNA (SEQ ID NO:120) to stimulate APCs in vitro and in vivo, in trans. In an embodiment of the present invention, the tumor cells loaded with LNP can include STAV3 (SEQ ID NO:37-38), and HPV E6 mRNA (SEQ ID NO:120) to stimulate APCs in vitro and in vivo, in trans. In an embodiment of the present invention, the tumor cells loaded with LNP can include STAV4 (SEQ ID NO:39-40), and HPV E6 mRNA (SEQ ID NO:120) to stimulate APCs in vitro and in vivo, in trans. In an embodiment of the present invention, the tumor cells loaded with LNP can include STAV5 (SEQ ID NO:41-42), and HPV E6 mRNA (SEQ ID NO:120) to stimulate APCs in vitro and in vivo, in trans. In an embodiment of the present invention, the tumor cells loaded with LNP can include STAV6 (SEQ ID NO:43-44), and HPV E6 mRNA (SEQ ID NO:120) to stimulate APCs in vitro and in vivo, in trans. In an embodiment of the present invention, the tumor cells loaded with LNP can include STAV7 (SEQ ID NO:45-46), and HPV E6 mRNA (SEQ ID NO:120) to stimulate APCs in vitro and in vivo.

In an alternative embodiment of the invention, the tumor cells loaded with LNP can include STAVs (SEQ ID NO:24-27 and/or SEQ ID NO:37-46), and fragments of the HPV E6 mRNA (SEQ ID NO:120) (e.g., mRNA fragments encoding for residues 10-16) to stimulate APCs in vitro and in vivo. In another embodiment of the invention, the tumor cells loaded with LNP can include STAVs (SEQ ID NO:24-27 and/or SEQ ID NO:37-46), the HPV E6 mRNA (SEQ ID NO:120) and IFN (SEQ ID NO:117) to stimulate APCs in vitro and in vivo.

### Example 10. Replicant (HPV E7 mRNA)

In an embodiment of the present invention, tumor cells loaded with LNP that include STAVs (i.e., DNA) and mRNA encoding HPV E7 mRNA (SEQ ID NO:121) can render non-immunogenic cells immunogenic. In an embodiment of the present invention, the tumor cells loaded with LNP can include STAV1 (SEQ ID NO:24-25), and HPV E7 mRNA (SEQ ID NO:121) to stimulate APCs in vitro and in vivo, in trans. In an embodiment of the present invention, the tumor cells loaded with LNP can include STAV2 (SEQ ID NO:26-27), and HPV E7 mRNA (SEQ ID NO:121) to stimulate APCs in vitro and in vivo, in trans. In an embodiment of the present invention, the tumor cells loaded with LNP can include STAV3 (SEQ ID NO:37-38), and HPV E7 mRNA (SEQ ID NO:121) to stimulate APCs in vitro and in vivo, in trans. In an embodiment of the present invention, the tumor cells loaded with LNP can include STAV4 (SEQ ID NO:39-40), and HPV E7 mRNA (SEQ ID NO:121) to stimulate APCs in vitro and in vivo, in trans. In an embodiment of the present invention, the tumor cells loaded with LNP can include STAV5 (SEQ ID NO:41-42), and HPV E7 mRNA (SEQ ID NO:121) to stimulate APCs in vitro and in vivo, in trans. In an embodiment of the present invention, the tumor cells loaded with LNP can include STAV6 (SEQ ID NO:43-44), and HPV E7 mRNA (SEQ ID NO:121) to stimulate APCs in vitro and in vivo, in trans. In an embodiment of the present invention, the tumor cells loaded with LNP can include STAV7 (SEQ ID NO:45-46), and HPV E7 mRNA (SEQ ID NO:121) to stimulate APCs in vitro and in vivo.

In an alternative embodiment of the invention, the tumor cells loaded with LNP can include STAVs (SEQ ID NO:24-27 and/or SEQ ID NO:37-46), and fragments of the HPV E7 mRNA (SEQ ID NO:121) (e.g., mRNA fragments encoding for residues 26-37) to stimulate APCs in vitro and in vivo. In another embodiment of the invention, the tumor cells loaded with LNP can include STAVs (SEQ ID NO:24-27 and/or SEQ ID NO:37-46), the HPV E7 mRNA (SEQ ID NO:121) and IFN (SEQ ID NO:117) to stimulate APCs in vitro and in vivo.

### Example 11. Replicant (TYRP-1 mRNA)

In an embodiment of the present invention, tumor cells loaded with LNP that include STAVs (i.e., DNA) and mRNA encoding TRYP-1 (SEQ ID NO:114-116) can render non-immunogenic cells immunogenic. In an embodiment of the present invention, the tumor cells loaded with LNP can include STAV1 (SEQ ID NO:24-25), and TRYP-1 mRNA (SEQ ID NO:114-116) to stimulate APCs in vitro and in vivo, in trans. In an embodiment of the present invention, the tumor cells loaded with LNP can include STAV2 (SEQ ID NO:26-27), and TRYP-1 mRNA (SEQ ID NO:114-116) to stimulate APCs in vitro and in vivo, in trans. In an embodiment of the present invention, the tumor cells loaded with LNP can include STAV3 (SEQ ID NO:37-38), and TRYP-1 mRNA (SEQ ID NO:114-116) to stimulate APCs in vitro and in vivo, in trans. In an embodiment of the present invention, the tumor cells loaded with LNP can include STAV4 (SEQ ID NO:39-40), and TRYP-1 mRNA (SEQ ID NO:114-116) to stimulate APCs in vitro and in vivo, in trans. In an embodiment of the present invention, the tumor cells loaded with LNP can include STAV5 (SEQ ID NO:41-42), and TRYP-1 mRNA (SEQ ID NO:114-116) to stimulate APCs in vitro and in vivo, in trans. In an embodiment of the present invention, the tumor cells loaded with LNP can include STAV6 (SEQ ID NO:43-44), and TRYP-1 mRNA (SEQ ID NO:114-116) to stimulate APCs in vitro and in vivo, in trans. In an embodiment of the present invention, the tumor cells loaded with LNP can include STAV7 (SEQ ID NO:45-46), and TRYP-1 mRNA (SEQ ID NO:114-116) to stimulate APCs in vitro and in vivo.

In an alternative embodiment of the invention, the tumor cells loaded with LNP can include STAVs (SEQ ID NO:24-27 and/or SEQ ID NO:37-46), and fragments of the TRYP-1 mRNA (SEQ ID NO:114-116) to stimulate APCs in vitro and in vivo. In another embodiment of the invention, the tumor cells loaded with LNP can include STAVs (SEQ ID NO:24-27 and/or SEQ ID NO:37-46), the TRYP-1 mRNA (SEQ ID NO:114-116) and IFN (SEQ ID NO:117) to stimulate APCs in vitro and in vivo.

### Example 12. Replicant (Melan-A mRNA)

In an embodiment of the present invention, tumor cells loaded with LNP that include STAVs (i.e., DNA) and mRNA encoding Melan-A (MLANA) (SEQ ID NO:83) can render non-immunogenic cells immunogenic. In an embodiment of the present invention, the tumor cells loaded with LNP can include STAV1 (SEQ ID NO:24-25), and MLANA mRNA (SEQ ID NO:83) to stimulate APCs in vitro and in vivo, in trans. In an embodiment of the present invention, the tumor cells loaded with LNP can include STAV2 (SEQ ID NO:26-27), and MLANA mRNA (SEQ ID NO:83) to stimulate APCs in vitro and in vivo, in trans. In an embodiment of the present invention, the tumor cells loaded with LNP can include STAV3 (SEQ ID NO:37-38), and MLANA mRNA (SEQ ID NO:83) to stimulate APCs in vitro and in vivo, in trans. In an embodiment of the present invention, the tumor cells loaded with LNP can include STAV4 (SEQ ID NO:39-40), and MLANA mRNA (SEQ ID NO:83) to stimulate APCs in vitro and in vivo, in trans. In an embodiment of the present invention, the tumor cells loaded with LNP can include STAV5 (SEQ ID NO:41-42), and MLANA mRNA (SEQ ID NO:83) to stimulate APCs in vitro and in vivo, in trans. In an embodiment of the present invention, the tumor cells loaded with LNP can include STAV6 (SEQ ID NO:43-44), and MLANA mRNA (SEQ ID NO:83) to stimulate APCs in vitro and in vivo, in trans. In an embodiment of the present invention, the tumor cells loaded with LNP can include STAV7 (SEQ ID NO:45-46), and MLANA mRNA (SEQ ID NO:83) to stimulate APCs in vitro and in vivo.

In an alternative embodiment of the invention, the tumor cells loaded with LNP can include STAVs (SEQ ID NO:24-27 and/or SEQ ID NO:37-46), and fragments of the MLANA mRNA (SEQ ID NO:83) (e.g., mRNA fragments encoding for residues 26-35) to stimulate APCs in vitro and in vivo. In another embodiment of the invention, the tumor cells loaded with LNP can include STAVs (SEQ ID NO:24-27 and/or SEQ ID NO:37-46), the MLANA mRNA (SEQ ID NO:83) and IFN (SEQ ID NO:117) to stimulate APCs in vitro and in vivo.

### Example 13. Replicant (CEA mRNA)

In an embodiment of the present invention, tumor cells loaded with LNP that include STAVs (i.e., DNA) and mRNA encoding Carcinoembryonic Antigen (CEA) (SEQ ID NO:854) can render non-immunogenic cells immunogenic. In an embodiment of the present invention, the tumor cells loaded with LNP can include STAV1 (SEQ ID NO:24-25), and CEA mRNA (SEQ ID NO:58-66) to stimulate APCs in vitro and in vivo, in trans. In an embodiment of the present invention, the tumor cells loaded with LNP can include STAV2 (SEQ ID NO:26-27), and CEA mRNA (SEQ ID NO:58-66) to stimulate APCs in vitro and in vivo, in trans. In an embodiment of the present invention, the tumor cells loaded with LNP can include STAV3 (SEQ ID NO:37-38), and CEA mRNA (SEQ ID NO:58-66) to stimulate APCs in vitro and in vivo, in trans. In an embodiment of the present invention, the tumor cells loaded with LNP can include STAV4 (SEQ ID NO:39-40), and CEA mRNA (SEQ ID NO:58-66) to stimulate APCs in vitro and in vivo, in trans. In an embodiment of the present invention, the tumor cells loaded with LNP can include STAV5 (SEQ ID NO:41-42), and CEA mRNA (SEQ ID NO:58-66) to stimulate APCs in vitro and in vivo, in trans. In an embodiment of the present invention, the tumor cells loaded with LNP can include STAV6 (SEQ ID NO:43-44), and CEA mRNA (SEQ ID NO:58-66) to stimulate APCs in vitro and in vivo, in trans. In an embodiment of the present invention, the tumor cells loaded with LNP can include STAV7 (SEQ ID NO:45-46), and CEA mRNA (SEQ ID NO:58-66) to stimulate APCs in vitro and in vivo.

In an alternative embodiment of the invention, the tumor cells loaded with LNP can include STAVs (SEQ ID NO:24-27 and/or SEQ ID NO:37-46), and fragments of the CEA mRNA (SEQ ID NO:58-66) to stimulate APCs in vitro and in vivo. In another embodiment of the invention, the tumor cells loaded with LNP can include STAVs (SEQ ID NO:24-27 and/or SEQ ID NO:37-46), the CEA mRNA (SEQ ID NO:58-66) and IFN (SEQ ID NO:117) to stimulate APCs in vitro and in vivo.

### Example 14. Replicant (Mucin-1 mRNA)

In an embodiment of the present invention, tumor cells loaded with LNP that include STAVs (i.e., DNA) and mRNA encoding Mucin-1 (CEACAM5) (SEQ ID NO:92-112) can render non-immunogenic cells immunogenic. In an embodiment of the present invention, the tumor cells loaded with LNP can include STAV1 (SEQ ID NO:24-25), and CEACAM5 mRNA (SEQ ID NO:92-112) to stimulate APCs in vitro and in vivo, in trans. In an embodiment of the present invention, the tumor cells loaded with LNP can include STAV2 (SEQ ID NO:26-27), and CEACAM5 mRNA (SEQ ID NO:92-112) to stimulate APCs in vitro and in vivo, in trans. In an embodiment of the present invention, the tumor cells loaded with LNP can include STAV3 (SEQ ID NO:37-38), and CEACAM5 mRNA (SEQ ID NO:92-112) to stimulate APCs in vitro and in vivo, in trans. In an embodiment of the present invention, the tumor cells loaded with LNP can include STAV4 (SEQ ID NO:39-40), and CEACAM5 mRNA (SEQ ID NO:92-112) to stimulate APCs in vitro and in vivo, in trans. In an embodiment of the present invention, the tumor cells loaded with LNP can include STAV5 (SEQ ID NO:41-42), and CEACAM5 mRNA (SEQ ID NO:92-112) to stimulate APCs in vitro and in vivo, in trans. In an embodiment of the present invention, the tumor cells loaded with LNP can include STAV6 (SEQ ID NO:43-44), and CEACAM5 mRNA (SEQ ID NO:92-112) to stimulate APCs in vitro and in vivo, in trans.

In an alternative embodiment of the invention, the tumor cells loaded with LNP can include STAVs (SEQ ID NO:24-27 and/or SEQ ID NO:37-46), and fragments of the CEACAM5 mRNA (SEQ ID NO:92-112) to stimulate APCs in vitro and in vivo. In another embodiment of the invention, the tumor cells loaded with LNP can include STAVs (SEQ ID NO:24-27 and/or SEQ ID NO:37-46), the CEACAM5 mRNA (SEQ ID NO:92-112) and IFN (SEQ ID NO:117) to stimulate APCs in vitro and in vivo.

### Example 15. Replicant (EGFR mRNA)

In an embodiment of the present invention, tumor cells loaded with LNP that include STAVs (i.e., DNA) and mRNA encoding Epidermal Growth Factor Receptor (EGFR) (SEQ ID NO:67-79) can render non-immunogenic cells immunogenic. In an embodiment of the present invention, the tumor cells loaded with LNP can include STAV1 (SEQ ID NO:24-25), and EGFR mRNA (SEQ ID NO:67-79) to stimulate APCs in vitro and in vivo, in trans. In an embodiment of the present invention, the tumor cells loaded with LNP can include STAV2 (SEQ ID NO:26-27), and EGFR mRNA (SEQ ID NO:67-79) to stimulate APCs in vitro and in vivo, in trans. In an embodiment of the present invention, the tumor cells loaded with LNP can include STAV3 (SEQ ID NO:37-38), and EGFR mRNA (SEQ ID NO:67-79) to stimulate APCs in vitro and in vivo, in trans. In an embodiment of the present invention, the tumor cells loaded with LNP can include STAV4 (SEQ ID NO:39-40), and EGFR mRNA (SEQ ID NO:67-79) to stimulate APCs in vitro and in vivo. In an embodiment of the present invention, the tumor cells loaded with LNP can include STAV5 (SEQ ID NO:41-42), and EGFR mRNA (SEQ ID NO:67-79) to stimulate APCs in vitro and in vivo. In an embodiment of the present invention, the tumor cells loaded with LNP can include STAV6 (SEQ ID NO:43-44), and EGFR mRNA (SEQ ID NO:67-79) to stimulate APCs in vitro and in vivo. In an embodiment of the present invention, the tumor cells loaded with LNP can include STAV7 (SEQ ID NO:45-46), and EGFR mRNA (SEQ ID NO:67-79) to stimulate APCs in vitro and in vivo.

In an alternative embodiment of the invention, the tumor cells loaded with LNP can include STAVs (SEQ ID NO:24-27 and/or SEQ ID NO:37-46), and fragments of the EGFR mRNA (SEQ ID NO:67-79) to stimulate APCs in vitro and in vivo. In another embodiment of the invention, the tumor cells loaded with LNP can include STAVs (SEQ ID NO:24-27 and/or SEQ ID NO:37-46), the EGFR mRNA (SEQ ID NO:67-79) and IFN (SEQ ID NO:117) to stimulate APCs in vitro and in vivo.

### Example 16. Replicant (Transmembrane 4 Superfamily Member 5 Protein mRNA)

In an embodiment of the present invention, tumor cells loaded with LNP that include STAVs (i.e., DNA) and mRNA encoding Transmembrane 4 superfamily member 5 protein (TM4SF5) (SEQ ID NO:113) can render non-immunogenic cells immunogenic. In an embodiment of the present invention, the tumor cells loaded with LNP can include STAV1 (SEQ ID NO:24-25), and TM4SF5 mRNA (SEQ ID NO:113) to stimulate APCs in vitro and in vivo, in trans. In an embodiment of the present invention, the tumor cells loaded with LNP can include STAV2 (SEQ ID NO:26-27), and TM4SF5 mRNA (SEQ ID NO:113) to stimulate APCs in vitro and in vivo. In an embodiment of the present invention, the tumor cells loaded with LNP can include STAV3 (SEQ ID NO:37-38), and TM4SF5 mRNA (SEQ ID NO:113) to stimulate APCs in vitro and in vivo. In an embodiment of the present invention, the tumor cells loaded with LNP can include STAV4 (SEQ ID NO:39-40), and TM4SF5 mRNA (SEQ ID NO:113) to stimulate APCs in vitro and in vivo. In an embodiment of the present invention, the tumor cells loaded with LNP can include STAV5 (SEQ ID NO:41-42), and TM4SF5 mRNA (SEQ ID NO:113) to stimulate APCs in vitro and in vivo. In an embodiment of the present invention, the tumor cells loaded with LNP can include STAV6 (SEQ ID NO:43-44), and TM4SF5 mRNA (SEQ ID NO:113) to stimulate APCs in vitro and in vivo. In an embodiment of the present invention, the tumor cells loaded with LNP can include STAV7 (SEQ ID NO:45-46), and TM4SF5 mRNA (SEQ ID NO:113) to stimulate APCs in vitro and in vivo.

In an alternative embodiment of the invention, the tumor cells loaded with LNP can include STAVs (SEQ ID NO:24-27 and/or SEQ ID NO:37-46), and fragments of the TM4SF5 mRNA (SEQ ID NO:113) to stimulate APCs in vitro and in vivo. In another embodiment of the invention, the tumor cells loaded with LNP can include STAVs (SEQ ID NO:24-27 and/or SEQ ID NO:37-46), the TM4SF5 mRNA (SEQ ID NO:113) and IFN (SEQ ID NO:117) to stimulate APCs in vitro and in vivo.

### Example 17. Replicant (Mitotic Centromere-Associated Kinesin mRNA)

In an embodiment of the present invention, tumor cells loaded with LNP that include STAVs (i.e., DNA) and mRNA encoding Mitotic Centromere-Associated Kinesin (MCAK) (SEQ ID NO:84-91) can render non-immunogenic cells immunogenic. In an embodiment of the present invention, the tumor cells loaded with LNP can include STAV1 (SEQ ID NO:24-25), and MCAK mRNA (SEQ ID NO:84-91) to stimulate APCs in vitro and in vivo, in trans. In an embodiment of the present invention, the tumor cells loaded with LNP can include STAV2 (SEQ ID NO:26-27), and MCAK mRNA (SEQ ID NO:84-91) to stimulate APCs in vitro and in vivo. In an embodiment of the present invention, the tumor cells loaded with LNP can include STAV3 (SEQ ID NO:37-38), and MCAK mRNA (SEQ ID NO:84-91) to stimulate APCs in vitro and in vivo. In an embodiment of the present invention, the tumor cells loaded with LNP can include STAV4 (SEQ ID NO:39-40), and MCAK mRNA (SEQ ID NO:84-91) to stimulate APCs in vitro and in vivo. In an embodiment of the present invention, the tumor cells loaded with LNP can include STAV5 (SEQ ID NO:41-42), and MCAK mRNA (SEQ ID NO:84-91) to stimulate APCs in vitro and in vivo. In an embodiment of the present invention, the tumor cells loaded with LNP can include STAV6 (SEQ ID NO:43-44), and MCAK mRNA (SEQ ID NO:84-91) to stimulate APCs in vitro and in vivo. In an embodiment of the present invention, the tumor cells loaded with LNP can include STAV7 (SEQ ID NO:45-46), and MCAK mRNA (SEQ ID NO:84-91) to stimulate APCs in vitro and in vivo.

In an alternative embodiment of the invention, the tumor cells loaded with LNP can include STAVs (SEQ ID NO:24-27 and/or SEQ ID NO:37-46), and fragments of the MCAK mRNA (SEQ ID NO:84-91) to stimulate APCs in vitro and in vivo. In another embodiment of the invention, the tumor cells loaded with LNP can include STAVs (SEQ ID NO:24-27 and/or SEQ ID NO:37-46), the MCAK mRNA (SEQ ID NO:84-91) and IFN (SEQ ID NO:117) to stimulate APCs in vitro and in vivo.

### Example 18. Replicant (Guanylyl Cyclase C mRNA)

In an embodiment of the present invention, tumor cells loaded with LNP that include STAVs (i.e., DNA) and mRNA encoding Guanylyl Cyclase C (GUCY2C) (SEQ ID NO:80-82) can render non-immunogenic cells immunogenic. In an embodiment of the present invention, the tumor cells loaded with LNP can include STAV1 (SEQ ID NO:24-25), and GUCY2C mRNA (SEQ ID NO:80-82) to stimulate APCs in vitro and in vivo, in trans. In an embodiment of the present invention, the tumor cells loaded with LNP can include STAV2 (SEQ ID NO:26-27), and GUCY2C mRNA (SEQ ID NO:80-82) to stimulate APCs in vitro and in vivo. In an embodiment of the present invention, the tumor cells loaded with LNP can include STAV3 (SEQ ID NO:37-38), and GUCY2C mRNA (SEQ ID NO:80-82) to stimulate APCs in vitro and in vivo. In an embodiment of the present invention, the tumor cells loaded with LNP can include STAV4 (SEQ ID NO:39-40), and GUCY2C mRNA (SEQ ID NO:80-82) to stimulate APCs in vitro and in vivo. In an embodiment of the present invention, the tumor cells loaded with LNP can include STAV5 (SEQ ID NO:41-42), and GUCY2C mRNA (SEQ ID NO:80-82) to stimulate APCs in vitro and in vivo. In an embodiment of the present invention, the tumor cells loaded with LNP can include STAV6 (SEQ ID NO:43-44), and GUCY2C mRNA (SEQ ID NO:80-82) to stimulate APCs in vitro and in vivo. In an embodiment of the present invention, the tumor cells loaded with LNP can include STAV7 (SEQ ID NO:45-46), and GUCY2C mRNA (SEQ ID NO:80-82) to stimulate APCs in vitro and in vivo.

In an alternative embodiment of the invention, the tumor cells loaded with LNP can include STAVs (SEQ ID NO:24-27 and/or SEQ ID NO:37-46), and fragments of the GUCY2C mRNA (SEQ ID NO:80-82) (e.g., mRNA fragments encoding for residues 7-15, 74-81, 127-134 and/or 170-185) to stimulate APCs in vitro and in vivo. In another embodiment of the invention, the tumor cells loaded with LNP can include STAVs (SEQ ID NO:24-27 and/or SEQ ID NO:37-46), the GUCY2C mRNA (SEQ ID NO:80-82) and IFN (SEQ ID NO:117) to stimulate APCs in vitro and in vivo.

### Example 19. Replicant (Oncofetal Antigen 5T4 mRNA)

In an embodiment of the present invention, tumor cells loaded with LNP that include STAVs (i.e., DNA) and mRNA encoding oncofetal antigen 5T4 (5T4) (SEQ ID NO:55-57) can render non-immunogenic cells immunogenic. In an embodiment of the present invention, the tumor cells loaded with LNP can include STAV1 (SEQ ID NO:24-25), and 5T4 mRNA (SEQ ID NO:55-57) to stimulate APCs in vitro and in vivo, in trans. In an embodiment of the present invention, the tumor cells loaded with LNP can include STAV2 (SEQ ID NO:26-27), and 5T4 mRNA (SEQ ID NO:55-57) to stimulate APCs in vitro and in vivo. In an embodiment of the present invention, the tumor cells loaded with LNP can include STAV3 (SEQ ID NO:37-38), and 5T4 mRNA (SEQ ID NO:55-57) to stimulate APCs in vitro and in vivo. In an embodiment of the present invention, the tumor cells loaded with LNP can include STAV4 (SEQ ID NO:39-40), and 5T4 mRNA (SEQ ID NO:55-57) to stimulate APCs in vitro and in vivo. In an embodiment of the present invention, the tumor cells loaded with LNP can include STAV5 (SEQ ID NO:41-42), and 5T4 mRNA (SEQ ID NO:55-57) to stimulate APCs in vitro and in vivo. In an embodiment of the present invention, the tumor cells loaded with LNP can include STAV6 (SEQ ID NO:43-44), and 5T4 mRNA (SEQ ID NO:55-57) to stimulate APCs in vitro and in vivo. In an embodiment of the present invention, the tumor cells loaded with LNP can include STAV7 (SEQ ID NO:45-46), and 5T4 mRNA (SEQ ID NO:55-57) to stimulate APCs in vitro and in vivo.

In an alternative embodiment of the invention, the tumor cells loaded with LNP can include STAVs (SEQ ID NO:24-27 and/or SEQ ID NO:37-46), and fragments of the 5T4 mRNA (SEQ ID NO:55-57) (e.g., mRNA fragments encoding for residues 1-10, 21-50, 58-143 and/or 260-285) to stimulate APCs in vitro and in vivo. In another embodiment of the invention, the tumor cells loaded with LNP can include STAVs (SEQ ID NO:24-27 and/or SEQ ID NO:37-46), the 5T4 mRNA (SEQ ID NO:55-57) and IFN (SEQ ID NO:117) to stimulate APCs in vitro and in vivo.

### Example 20. Replicant (HHV8 Herpes Virus Family mRNA)

In an embodiment of the present invention, tumor cells loaded with LNP that include STAVs (i.e., DNA) and mRNA encoding Human Herpesvirus-8 (HHV8) antigens (SEQ ID NO:133-146) can render non-immunogenic cells immunogenic. HHV8 antigen candidates as Replicants include mRNA directed to Kaposi sarcoma Herpesvirus (KSHV) glycoproteins K8.1 (SEQ ID NO:133), ORF8 (gB) (SEQ ID NO:134), ORF22 (gH) (SEQ ID NO:135), ORF 46 (gL) (SEQ ID NO:136), ORF39 (gM) (SEQ ID NO:137), and ORF53 (gN) (SEQ ID NO:138), accessory glycoproteins gK8.1, ORF4 (SEQ ID NO:139), ORF27 (SEQ ID NO:140), ORF28 (SEQ ID NO:141), and ORF68 (SEQ ID NO:142), nonstructural (NS) protein LANA (SEQ ID NO:143), vIRF, K1 protein, vIL-6 (SEQ ID NO:145), viral G protein-coupled receptor (SEQ ID NO:144), vFLIP (SEQ ID NO:146) and viral chemokine. There is no ready or predictive animal model for KSHV-related cancers, although animal models are valuable to assess KSHV infection. Vaccine design can be geared toward safety, avoiding predicted severe adverse events. Since HHV8 is a tumor virus, disabling viral oncoproteins can be required for subunit DNA-based or live virus vaccines. Determining efficacy against tumorigenesis can require multi-year evaluations and/or prohibitively large clinical trials. Depending on the vaccine, either defining smaller high-risk populations likely to develop malignancy (e.g., newly diagnosed AIDS patients without KS in a high KS prevalence area) or surrogate measures that can reasonably predict efficacy, can overcome this limitation. Surrogate measures, for example, could include protection against infection (measured by antibodies against HHV8 antigens not included in the vaccine), high-level immune responses, or prevention of oral shedding. Note that KSHV glycoproteins K8.1 (SEQ ID NO:133), ORF8 (gB) (SEQ ID NO:134), ORF22 (gH) (SEQ ID NO:135), ORF 46 (gL) (SEQ ID NO:136), ORF39 (gM) (SEQ ID NO:137), and ORF53 (gN) (SEQ ID NO:138), are virion-associated and candidates for KSHV attachment, fusion, and entry functions together with accessory glycoproteins gK8.1, ORF4 (SEQ ID NO:139), ORF27 (SEQ ID NO:140), ORF28 (SEQ ID NO:141), and ORF68 (SEQ ID NO:142). Further, the nonstructural (NS) protein LANA (SEQ ID NO:143) is required for viral persistence.

In an embodiment of the present invention, the tumor cells loaded with LNP can include STAV1 (SEQ ID NO:24-25), and mRNA encoding HHV8 antigens (SEQ ID NO:133-146) to stimulate APCs in vitro and in vivo, in trans. In an embodiment of the present invention, the tumor cells loaded with LNP can include STAV2 (SEQ ID NO:26-27), and mRNA encoding HHV8 antigens (SEQ ID NO:133-146) to stimulate APCs in vitro and in vivo. In an embodiment of the present invention, the tumor cells loaded with LNP can include STAV3 (SEQ ID NO:37-38), and mRNA encoding HHV8 antigens (SEQ ID NO:133-146) to stimulate APCs in vitro and in vivo. In an embodiment of the present invention, the tumor cells loaded with LNP can include STAV4 (SEQ ID NO:39-40), and mRNA encoding HHV8 antigens (SEQ ID NO:133-146) to stimulate APCs in vitro and in vivo. In an embodiment of the present invention, the tumor cells loaded with LNP can include STAV5 (SEQ ID NO:41-42), and mRNA encoding HHV8 antigens (SEQ ID NO:133-146) to stimulate APCs in vitro and in vivo. In an embodiment of the present invention, the tumor cells loaded with LNP can include STAV6 (SEQ ID NO:43-44), and mRNA encoding HHV8 antigens (SEQ ID NO:133-146) to stimulate APCs in vitro and in vivo. In an embodiment of the present invention, the tumor cells loaded with LNP can include STAV7 (SEQ ID NO:45-46), and mRNA encoding HHV8 antigens (SEQ ID NO:133-146) to stimulate APCs in vitro and in vivo.

In an alternative embodiment of the invention, the tumor cells loaded with LNP can include STAVs (SEQ ID NO:24-27 and/or SEQ ID NO:37-46), and mRNA of fragments of the HHV8 antigens (SEQ ID NO:133-146) to stimulate APCs in vitro and in vivo. In another embodiment of the invention, the tumor cells loaded with LNP can include STAVs (SEQ ID NO:24-27 and/or SEQ ID NO:37-46), the mRNA of HHV8 antigens (SEQ ID NO:133-146) and IFN (SEQ ID NO:117) to stimulate APCs in vitro and in vivo.

### Example 21. Replicant (CMV Herpes Virus Family Antigen mRNA)

In an embodiment of the present invention, tumor cells loaded with LNP that include STAVs (i.e., DNA) and mRNA encoding Cytomegalovirus (CMV) antigens (SEQ ID NO:123-132) can render non-immunogenic cells immunogenic. CMV antigen candidates as Replicants include mRNA directed to pp65 protein, IE1 protein, glycoprotein M (SEQ ID NO:131), glycoprotein N (SEQ ID NO:132), glycoprotein H (SEQ ID NO:123) and glycoprotein L (SEQ ID NO:124).

In an embodiment of the present invention, the tumor cells loaded with LNP can include STAV1 (SEQ ID NO:24-25), and mRNA encoding CMV antigens (SEQ ID NO:123-132) to stimulate APCs in vitro and in vivo, in trans. In an embodiment of the present invention, the tumor cells loaded with LNP can include STAV2 (SEQ ID NO:26-27), and mRNA encoding CMV antigens (SEQ ID NO:123-132) to stimulate APCs in vitro and in vivo. In an embodiment of the present invention, the tumor cells loaded with LNP can include STAV3 (SEQ ID NO:37-38), and mRNA encoding CMV antigens (SEQ ID NO:123-132) to stimulate APCs in vitro and in vivo. In an embodiment of the present invention, the tumor cells loaded with LNP can include STAV4 (SEQ ID NO:39-40), and mRNA encoding CMV antigens (SEQ ID NO:123-132) to stimulate APCs in vitro and in vivo. In an embodiment of the present invention, the tumor cells loaded with LNP can include STAV5 (SEQ ID NO:41-42), and mRNA encoding CMV antigens (SEQ ID NO:123-132) to stimulate APCs in vitro and in vivo. In an embodiment of the present invention, the tumor cells loaded with LNP can include STAV6 (SEQ ID NO:43-44), and mRNA encoding CMV antigens (SEQ ID NO:123-132) to stimulate APCs in vitro and in vivo. In an embodiment of the present invention, the tumor cells loaded with LNP can include STAV7 (SEQ ID NO:45-46), and mRNA encoding CMV antigens (SEQ ID NO:123-132) to stimulate APCs in vitro and in vivo.

In an alternative embodiment of the invention, the tumor cells loaded with LNP can include STAVs (SEQ ID NO:24-27 and/or SEQ ID NO:37-46), and mRNA of fragments of the CMV antigens (SEQ ID NO:123-132) to stimulate APCs in vitro and in vivo. In another embodiment of the invention, the tumor cells loaded with LNP can include STAVs (SEQ ID NO:24-27 and/or SEQ ID NO:37-46), the mRNA of CMV antigens (SEQ ID NO:123-132) and IFN (SEQ ID NO:117) to stimulate APCs in vitro and in vivo.

### Example 22. Replicant (EBV Herpes Virus Family Antigen mRNA)

In an embodiment of the present invention, tumor cells loaded with LNP that include STAVs (i.e., DNA) and mRNA encoding Epstein-Barr virus (EBV) (HHV5) antigens (SEQ ID NO:147-161) can render non-immunogenic cells immunogenic. EBV antigen candidates as Replicants include mRNA directed to gp350 (SEQ ID NO:147), gH (SEQ ID NO:148)/gL (SEQ ID NO:149), gp42 (SEQ ID NO:150), EBV immediate-early proteins Zta (encoded by BZLF1) (SEQ ID NO:151) and Rta (encoded by BRLF1) (SEQ ID NO:152), BMLF1 (a post-transcriptional regulatory protein) (SEQ ID NO:153), BMRF1 (polymerase-associated processivity factor), BNRF1 (the major tegument protein), BORF1 (DNA packaging protein) (SEQ ID NO:154), BcLF1 (major capsid protein) (SEQ ID NO:155), and BXLF1 (thymidine kinase) (SEQ ID NO:156), EBV nuclear antigen 2 (EBNA-2) and EBNA leader protein (EBNA-LP), EBNA2, EBNA3A, EBNA3B (SEQ ID NO:157), EBNA3C (SEQ ID NO:158), LMP1 (SEQ ID NO:159), LMP2A (SEQ ID NO:160), and LMP2B (SEQ ID NO:161).

In an embodiment of the present invention, the tumor cells loaded with LNP can include STAV1 (SEQ ID NO:24-25), and mRNA encoding EBV (HHV5) antigens (SEQ ID NO:147-161) to stimulate APCs in vitro and in vivo, in trans. In an embodiment of the present invention, the tumor cells loaded with LNP can include STAV2 (SEQ ID NO:26-27), and mRNA encoding EBV (HHV5) antigens (SEQ ID NO:147-161) to stimulate APCs in vitro and in vivo. In an embodiment of the present invention, the tumor cells loaded with LNP can include STAV3 (SEQ ID NO:37-38), and mRNA encoding EBV (HHV5) antigens (SEQ ID NO:147-161) to stimulate APCs in vitro and in vivo. In an embodiment of the present invention, the tumor cells loaded with LNP can include STAV4 (SEQ ID NO:39-40), and mRNA encoding EBV (HHV5) antigens (SEQ ID NO:147-161) to stimulate APCs in vitro and in vivo. In an embodiment of the present invention, the tumor cells loaded with LNP can include STAV5 (SEQ ID NO:41-42), and mRNA encoding EBV (HHV5) antigens (SEQ ID NO:147-161) to stimulate APCs in vitro and in vivo. In an embodiment of the present invention, the tumor cells loaded with LNP can include STAV6 (SEQ ID NO:43-44), and mRNA encoding EBV (HHV5) antigens (SEQ ID NO:147-161) to stimulate APCs in vitro and in vivo. In an embodiment of the present invention, the tumor cells loaded with LNP can include STAV7 (SEQ ID NO:45-46), and mRNA encoding EBV (HHV5) antigens (SEQ ID NO:147-161) to stimulate APCs in vitro and in vivo.

In an alternative embodiment of the invention, the tumor cells loaded with LNP can include STAVs (SEQ ID NO:24-27 and/or SEQ ID NO:37-46), and mRNA of fragments of the EBV (HHV5) antigens (SEQ ID NO:147-161) to stimulate APCs in vitro and in vivo. In another embodiment of the invention, the tumor cells loaded with LNP can include STAVs (SEQ ID NO:24-27 and/or SEQ ID NO:37-46), the mRNA of EBV (HHV5) antigens (SEQ ID NO:147-161) and IFN (SEQ ID NO:117) to stimulate APCs in vitro and in vivo.

### Example 23. Replicant (Custom Tumor Antigen mRNA)

Custom tumor antigens specific to patients can include novel TSAs. TSAs that include Custom tumor antigens specific to a patient can be identified using a three-step algorithm: 1) identifying somatic mutations or productions in DNA or messenger RNA (mRNA) sequences; 2) evaluating the affinity and presentation of MHC I/II molecules with new peptides; 3) determining whether new epitopes can stimulate T-cell proliferation and related immune responses. In an embodiment of the present invention, the tumor cells loaded with STAVs, with RNA directed to coronavirus spike protein to stimulate antigen presenting cells (APCs) to generate an immune response to protect patients from coronavirus infection.

In an embodiment of the invention, a kit for treating or preventing a disease in a mammal comprises a LNP comprising the combination of STAVs+RNA (directed to a specific disease) as therapeutic agents or a pharmaceutically acceptable salt or ester thereof, or a pharmaceutical composition of the application with instructions for the use thereof for reducing metastasis and neoplastic growth in a mammal. In another embodiment of the invention, the application provides a kit including a LNP comprising the combination of STAVs+RNA (directed to a specific disease) capable of modulating a disease in a mammal.

In one aspect, the present application also provides a method of treating or preventing cell proliferative disorders such as hyperplasias, dysplasias, or pre-cancerous lesions. Dysplasia is the earliest form of pre-cancerous lesion recognizable in a biopsy by a pathologist. The compounds of the present application can be administered for the purpose of preventing hyperplasias, dysplasias, or pre-cancerous lesions from continuing to expand or from becoming cancerous. Examples of pre-cancerous lesions can occur in skin, esophageal tissue, breast, and cervical intra-epithelial tissue.

In one embodiment, the disease or disorder includes, but is not limited to, immune disorders, autoinnunity, a cell proliferative disease or disorder, cancer, inflammation, cachexia, neurodegenerative disease or disorders, neurological diseases or disorders, cardiac dysfunction, transplantation, or infection (e.g., viral, bacterial, and/or fungi infection, or infection caused by other microorganism).

In one embodiment, the disease or disorder is a cell proliferative disease or disorder.

As used herein, the term ‘cell proliferative disorder’ refers to conditions in which unregulated or abnormal growth, or both, of cells can lead to the development of an unwanted condition or disease, which may or may not be cancerous. Exemplary cell proliferative diseases or disorders encompass a variety of conditions where cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term ‘rapidly dividing cell’ as used herein is defined as any cell that divides at a rate that exceeds or is greater than what is expected or observed among neighboring or juxtaposed cells within the same tissue. A cell proliferative disease or disorder includes a precancer or a precancerous condition. A cell proliferative disease or disorder includes cancer.

In one embodiment, the proliferative disease or disorder is a non-cancerous. In one embodiment, the non-cancerous disease or disorder includes, but is not limited to, rheumatoid arthritis; inflammation; autoimmune disease; lymphoproliferative conditions; acromegaly; rheumatoid spondylitis; osteoarthritis; gout; other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders; psoriasis; eczema; atopic dermatitis; hyperpigmentation disorders; eye-related hyperproliferative disorders; age-related macular degeneration; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

In one embodiment, the proliferative disease or disorder is cancer. In one embodiment, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors.

The term ‘cancer’ includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colonrectum, large intestine, rectum, brain and central nervous system; chronic myeloid leukemia (CML), and leukemia. The term ‘cancer’ includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term ‘cancer’ also refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which can be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Cancer can also include colon carcinoma, familiarly adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

Cancer can also include colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In one embodiment, the compounds of this application are useful for treating hematopoietic disorders, in particular, acute-myelogenous leukemia (AML), chronic-myelogenous leukemia (CML), acute-promyelocytic leukemia, and acute lymphocytic leukemia (ALL).

Exemplary cancers may also include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A ‘cell proliferative disorder of the hematologic system’ is a cell proliferative disease or disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Compounds and compositions of the present application can be used to treat a cancer selected from the group consisting of a hematologic cancer or a hematologic cell proliferative disorder. A hematologic cancer can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms, and mast cell neoplasms.

A ‘cell proliferative disorder of the lung’ is a cell proliferative disease or disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Compounds and compositions of the present application can be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (‘SCLC’), non-small cell lung cancer (‘NSCLC’), squamous cell carcinoma, adenocarcinoma, small cell carcinoma, large cell carcinoma, adenosquamous cell carcinoma, and mesothelioma. Lung cancer can include ‘scar carcinoma’, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can also include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that can predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A ‘cell proliferative disorder of the colon’ is a cell proliferative disorder involving cells of the colon. A cell proliferative disorder of the colon includes colon cancer. Compounds and compositions of the present application can be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome, and juvenile polyposis.

Cell proliferative disorders of the colon can also include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that can predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A ‘cell proliferative disorder of the pancreas’ is a cell proliferative disorder involving cells of the pancreas. Compounds and compositions of the present application can be used to treat pancreatic cancer or cell proliferative disorders of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A ‘cell proliferative disorder of the prostate’ is a cell proliferative disorder involving cells of the prostate. Compounds and compositions of the present application can be used to treat prostate cancer or cell proliferative disorders of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A ‘cell proliferative disorder of the skin’ is a cell proliferative disorder involving cells of the skin. Compounds and compositions of the present application can be used to treat skin cancer or cell proliferative disorders of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A ‘cell proliferative disorder of the ovary’ is a cell proliferative disorder involving cells of the ovary. Compounds and compositions of the present application can be used to treat ovarian cancer or cell proliferative disorders of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A ‘cell proliferative disorder of the breast’ is a cell proliferative disorder involving cells of the breast. Compounds and compositions of the present application can be used to treat breast cancer or cell proliferative disorders of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

In one embodiment, the disease or disorder includes, but is not limited to, a disease or disorders caused by or associated with Entamoeba histolytica, Pneumocystis carinii, Trypanosoma cruzi, Trypanosoma brucei, Leishmania mexicana, Clostridium histolyticum, Staphylococcus aureus, foot-and-mouth disease virus, or Crithidia fasciculata, as well as disease or disorder associated with osteoporosis, autoimmunity, schistosomiasis, malaria, tumor metastasis, metachromatic leukodystrophy, muscular dystrophy, or amytrophy.

Additional examples of the diseases or disorders include, but are not limited to, diseases of disorders caused by or associated with veterinary and human pathogenic protozoa, intracellular active parasites of the phylum Apicomplexa or Sarcomastigophora, Trypanosoma, Plasmodia, Leishmania, Babesia and Theileria, Cryptosporidia, Sacrocystida, Amoeba, Coccidia, and Trichomonadia. For example, the diseases of disorders include, but are not limited to, Malaria tropica, caused by, for example, Plasmodium falciparum; Malaria tertiana, caused by Plasmodium vivax or Plasmodium ovale. Malaria quartana, caused by Plasmodium malariae; Toxoplasmosis, caused by Toxoplasma gondii; Coccidiosis, caused for instance by Isospora belli; intestinal Sarcosporidiosis, caused by Sarcocystis suihominis; dysentery caused by Entamoeba histolytica; Cryptosporidiosis, caused by Cryptosporidium parvum; Chagas' disease, caused by Trypanosoma cruzi; sleeping sickness, caused by Trypanosoma brucei rhodesiense or gambiense, the cutaneous and visceral as well as other forms of Leishmaniosis; diseases or disorders caused by veterinary pathogenic protozoa, such as Theileria parva, the pathogen causing bovine East coast fever, Trypanosoma congolense congolense or Trypanosoma vivax vivax, Trypanosoma brucei brucei, pathogens causing Nagana cattle disease in Africa, Trypanosoma brucei evansi causing Surra, Babesia bigemina, the pathogen causing Texas fever in cattle and buffalos, Babesia bovis, the pathogen causing European bovine Babesiosis as well as Babesiosis in dogs, cats and sheep, Sarcocystis ovicanis and ovifelis pathogens causing Sarcocystiosis in sheep, cattle and pigs, Cryptosporidia, pathogens causing Cryptosporidioses in cattle and birds, Eimeria and Isospora species, pathogens causing Coccidiosis in rabbits, cattle, sheep, goats, pigs and birds, especially in chickens and turkeys. Rickettsia comprise species such as Rickettsia felis. Rickettsia prowazekii, Rickettsia rickettsii. Rickettsia typhi, Rickettsia conorii. Rickettsia africae and cause diseases such as typhus, rickettsialpox, Boutonneuse fever, African Tick Bite Fever, Rocky Mountain spotted fever. Australian Tick Typhus, Flinders Island Spotted Fever and Queensland Tick Typhus.

In one embodiment, the disease or disorder is caused by, or associated with, one or more bacteria. Examples of the bacteria include, but are not limited to, the Gram positive organisms (e.g., Staphylococcus aureus, Staphylococcus epidermidis, Enterococcus faecalis and E. faecium, Streptococcus pneumoniae) and the Gram negative organisms (e.g., Pseudomonas aeruginosa, Burkholdia cepacia, Xanthomonas maltophila. Escherichia coli, Enterobacter spp, Klebsiella pneumoniae and Salmonella spp).

In one embodiment, the disease or disorder is caused by, or associated with, one or more fungi. Examples of the fungi include, but are not limited to, Candida albicans, Histoplasma neoformans, Coccidioides immitis, and Penicillium marneffei.

In one embodiment, the disease or disorder is a neurological disease or disorder. In one embodiment, the neurological disease or disorder involves the central nervous system (e.g., brain, brainstem and cerebellum), the peripheral nervous system (e.g., cranial nerves), and/or the autonomic nervous system (e.g., parts of which are located in both central and peripheral nervous system).

Examples of the neurological disorders include, but are not limited to, acquired epileptiform aphasia; acute disseminated encephalomyelitis; adrenoleukodystrophy: age-related macular degeneration; agenesis of the corpus callosum: agnosia; Aicardi syndrome; Alexander disease; Alpers disease; alternating hemiplegia; Alzheimer's disease; Vascular dementia; amyotrophic lateral sclerosis; anencephaly: Angelman syndrome; angiomatosis; anoxia; aphasia; apraxia; arachnoid cysts; arachnoiditis; Apronl-Chiari malformation; arteriovenous malformation; Asperger syndrome; ataxia tclegiectasia: attention deficit hyperactivity disorder; autism; autonomie dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury: brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease: chemotherapy-induced neuropathy and neuropathic pain; Chiari malformation; chorea: chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy: diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy: Erb's palsy: essential tremor; Fabry's disease; Fahr's syndrome; fainting; familial spastic paralysis; febrile seizures; Fisher syndrome; Friedreich's ataxia; fronto-temporal dementia and other ‘tauopathies’; Gaucher's disease: Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; beredopathia atactica polyneuritiformis; herpes zoster oticus; herpes zoster; Hirayama syndrome; HIV-associated dementia and neuropathy (also neurological manifestations of AIDS); boloprosencephaly: Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease: infantile spasms; inflammatory myopathy; intracranial cyst; intracranial hypertension: Joubert syndrome: Kearns-Sayre syndrome; Kennedy disease Kinsbourne syndrome; Klippel Feil syndrome; Krabbe disease; Kugelberg-Welander disease; kuru; Lafora disease; Lambert-Baton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly: locked-in syndrome; Lou Gehrig's disease (i.e., motor neuron disease or amyotrophic lateral sclerosis); lumbar disc disease; Lyme disease—neurological sequelae: Machado-Joseph disease; maerencephaly; megalencephaly; Melkersson-Rosenthal syndrome: Menieres disease; meningitis; Menkes disease; metachromatic leukodystrophy; microcephaly: migraine; Miller Fisher syndrome; mini-strokes; mitochondrial myopathies; Mobius syndrome; monomelic amyotrophy; motor neuron disease; Moyamoya disease; mucopolysaccharidoses; multi-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy: neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; parancoplastic diseases; paroxysmal attacks; Parry Romberg syndrome; Pelizacus-Merzbacher disease; periodic paralyses: peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy: pseudotumor cerebri: Ramsay-Hunt syndrome (types I and II): Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity: spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea: syncope; syringomyelia; tardive dyskinesia; Tay-Sachs disease; temporal arteritis; tethered spinal cord syndrome; Thomsen disease; thoracic outlet syndrome: Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis: Von Hippel-Lindau disease: Wallenberg's syndrome; Werdnig-Hoffman disease: West syndrome; whiplash; Williams syndrome; Wildon's disease; and Zellweger syndrome.

Examples of neurodegenerative diseases can also include, without limitation, Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Familial fatal insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and Toxic encephalopathy.

In one embodiment, the disease or disorder is an autoimmune disease. Examples of autoimmune diseases include, but are not limited to, rheumatoid arthris, svstemic lupus erythematosus, inflammatory bowel diseases (IBDs) comprising Crohn disease (CD), and ulcerative colitis (UC) which are chronic inflammatory conditions with polygenic susceptibility.

In one embodiment, the disease or disorder is inflammation, arthritis, rheumatoid arthritis, spondyiarthropathies, gouty arthritis, osteoarthritis, juvenile arthritis, and other arthritic conditions, systemic lupus erthematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamous cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, peripheral neuropathy, or B-Cell Lymphoma.

In one embodiment, the disease or disorder is selected from autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In one embodiment, the disease or disorder is selected from a proliferative disorder and an immune disorder.

The present application provides the combination of STAVs+RNA (directed to a specific disease) as therapeutic agents in the generation of a vaccine to the specific disease, treatment or prevention of the specific disease such as cancer, coronavirus, inflammation, and other immunological disorders. In an alternative embodiment of the present application, the combination of STAVs+RNA (directed to a specific disease) and IFN type I act as therapeutic agents in the generation of a vaccine to the specific disease, treatment or prevention of the specific disease such as cancer, coronavirus, inflammation, and other immunological disorders.

Potency can also be determined by IC₅₀ value. A composition with a lower IC₅₀ value, as determined under substantially similar conditions, is more potent relative to a composition with a higher IC₅₀ value. In some embodiments, the substantially similar conditions comprise determining the level of binding of a known STING ligand to a STING protein, in vitro or in vivo, in the presence of a composition of the application.

In one embodiment, the compositions of the present application are useful as therapeutic agents, and thus may be useful in the treatment of a disease caused by, or associated with, STING expression, activity. and/or function (e.g., deregulation of STING expression, activity, and/or function) or a disease associated with one or more of the intracellular pathways that STING is involved in (e.g. regulation of intracellular DNA-mediated type I interferon activation), such as those described herein.

A ‘selective STING modulator’ can be identified, for example, by comparing the ability of a composition to modulate STING expression/activity/function to its ability to modulate the other proteins or a STING protein from another species. In some embodiments, the selectivity can be identified by measuring the EC₅₀ or IC₅₀ of the compositions.

In certain embodiments, the compositions of the application are STING modulators that exhibit at least 2-fold, 3-fold, 5-fold, 10-fold, 25-fold, 50-fold or 100-fold selectivity over other proteins or a STING protein from another species. In various embodiments, the compositions of the application exhibit 1000-fold selectivity over other proteins or a STING protein from another species.

The compositions of the application are defined herein by their chemical structures and/or chemical names. Where a composition is referred to by both a chemical structure and a chemical name, and the chemical structure and chemical name conflict, the chemical structure is determinative of the composition's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In another aspect, the application provides a method of synthesizing a composition disclosed herein. The synthesis of the compositions of the application can be found herein and in the Examples. Other embodiments are a method of making a composition of any of the formulae herein using any one, or combination of, reactions delineated herein. The method can include the use of one or more intermediates or chemical reagents delineated herein.

The application also provides for a pharmaceutical composition comprising a therapeutically effective amount of a composition of the application, or a pharmaceutically acceptable salt or ester thereof, and a pharmaceutically acceptable carrier.

Another aspect of the present application relates to a kit comprising a composition of the application or a pharmaceutically acceptable salt or ester thereof, or a pharmaceutical composition of the application. In another aspect, the application provides a kit comprising a composition capable of modulating STING activity selected from one or more compositions disclosed herein, or a pharmaceutically acceptable salt or ester thereof, optionally in combination with a second agent and instructions for use.

Another aspect of the present application relates to a composition of the application or a pharmaceutically acceptable salt or ester thereof, or a pharmaceutical composition of the application, for use in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to use of a composition of the application or a pharmaceutically acceptable salt or ester thereof, or a pharmaceutical composition of the application, in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to a composition of the application or a pharmaceutically acceptable salt or ester thereof, or a pharmaceutical composition of the application, for use in modulating (e.g., inhibiting or stimulating) a STING protein, in treating or preventing a disease, wherein the diseases is caused by, or associated with, STING expression, activity, and/or function e.g., deregulation of STING expression, activity, and/or function), or in treating or preventing a disease associated with deregulation of one or more of the intracellular pathways in which a STING protein is involved (e.g., deregulation of intracellular dsDNA mediated type 1 interferon activation).

Another aspect of the present application relates to use of a composition of the application or a pharmaceutically acceptable salt or ester thereof, or a pharmaceutical composition of the application, in modulating (e.g., inhibiting or stimulating) a STING protein, in treating or preventing a disease, wherein the diseases is caused by, or associated with, STING expression. activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated IFN activation).

### FURTHER EMBODIMENTS

Embodiments contemplated herein include Embodiments P1-P110 following.

Embodiment P1. A composition for treating a human subject suffering from HTLV-1 including, a) a STAV ds DNA molecule including a first DNA molecule, and a second DNA molecule, b) a Replicant including a mRNA molecule adapted to express a HTLV-1 mHBZ-mTAX (SEQ ID NO:54), c) an IFN type I protein, and d) a LNP, where the STAV and the Replicant are encapsulated in the LNP.

Embodiment P2. The composition of Embodiment P1, where the first DNA molecule is between a lower limit of approximately forty bases, and

an upper limit of approximately eighty bases.

Embodiment P3. The composition of Embodiment P1, where the first DNA molecule is at least eighty (80) percent complimentary with respect to the second DNA molecule.

Embodiment P4. The composition of Embodiment P1, where one or both the first DNA molecule and the second DNA molecule further comprise at least one modification located at one or both a 5′ end and a 3′ end.

Embodiment P5. The composition of Embodiment P4, where the mRNA molecule includes an exonuclease resistant phosphorothioate backbone moiety.

Embodiment P6. The composition of Embodiment P1, where the Replicant includes a 5′ cap.

Embodiment P7. The composition of Embodiment P6, where the 5′ cap includes a 7-methyl guanosine-2′-O-methoxy adenosine-guanosine.

Embodiment P8. The composition of Embodiment P1, where the LNP includes three (3) or more components selected from the group consisting of a polymer-conjugated lipid, a sterol, a phospholipid, an ionizing lipid and a cationic lipid.

Embodiment P9. The composition of Embodiment P1, where the HTLV-1 mHBZ-mTAX is overexpressed in one or more diseases selected from the group consisting of adult T cell leukemia, acute myeloid leukemia, and B-cell or T-cell acute lymphocytic leukemia.

Embodiment P10. The composition of Embodiment P1, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P11. A composition for treating a human subject suffering from HTLV-1 including, a) a Replicant including a messenger ribonucleic acid molecule adapted to express a HTLV-1 polypeptide antigen, b) a STAV including a ds DNA molecule, where a first DNA molecule of the ds DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45, where a second DNA molecule of the ds DNA molecule is approximately eighty percent complimentary to the first DNA molecule in order to generate the ds DNA molecule, and c) a LNP, where the STAV and the Replicant are encapsulated in the LNP.

Embodiment P12. The composition of Embodiment P11, further including an IFN protein.

Embodiment P13. The composition of Embodiment P11, where the HTLV-1 polypeptide antigen includes amino acids with sequence encompassed by a mHBZ gene (SEQ ID NO:118).

Embodiment P14. The composition of Embodiment P11, where the HTLV-1 polypeptide antigen includes amino acids with sequence encompassed by a mTAX gene (SEQ ID NO:117).

Embodiment P15. The composition of Embodiment P11, where the HTLV-1 polypeptide antigen includes amino acids with sequence encompassed by a mHBZ-mTAX gene (SEQ ID NO:54).

Embodiment P16. The composition of Embodiment P11, where the Replicant includes a 5′ cap.

Embodiment P17. The composition of Embodiment P16, where the 5′ cap includes a 7-methyl guanosine-2′-O-methoxy adenosine-guanosine.

Embodiment P18. The composition of Embodiment P11, where the LNP includes three (3) or more components selected from the group consisting of a polymer-conjugated lipid, a sterol, a phospholipid, an ionizing lipid and a cationic lipid.

Embodiment P19. The composition of Embodiment P11, where the HTLV-1 polypeptide antigen is overexpressed in one or more diseases selected from the group consisting of adult T cell leukemia, acute myeloid leukemia, and B-cell or T-cell acute lymphocytic leukemia.

Embodiment P20. A method for treating a human subject suffering from HTLV-1 including, a) determining whether the human subject is suffering from one or more of Adult T cell leukemia, Acute myeloid leukemia, and acute lymphocytic leukemia, b) if the human subject is suffering from ATLL, AML or ALL, then determining whether the human subject has a defective functional activity of STING by: isolating a sample from the human subject, and performing a PCR assay on the sample to determine if a cell population in the sample has a defective functional activity of STING, c) if the human subject is suffering from ATLL, AML or ALL and has the defective functional activity of STING, then administering a composition including, (i) a Replicant including a mRNA molecule adapted to express a HTLV-1 peptide antigen, ii) a STAV including a double-stranded (ds) DNA molecule including a first DNA molecule and a second DNA molecule, where the first DNA molecule is approximately eighty bases, where the second DNA molecule is approximately eighty percent complimentary to the first DNA molecule, and iii) a LNP, where the STAV and the Replicant are encapsulated in the LNP, and d) internally treating the human subject with the composition.

Embodiment P21. The method of Embodiment P20, the composition further including an IFN protein.

Embodiment P22. The method of Embodiment P21, where the IFN is administered prior to administering the LNP.

Embodiment P23. The method of Embodiment P20, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P24. The method of Embodiment P20, where one or both the first DNA molecule and the second DNA molecule further comprise at least one modification located at one or both a 5′ end and a 3′ end.

Embodiment P25. The method of Embodiment P24, where the mRNA molecule includes an exonuclease resistant phosphorothioate backbone moiety.

Embodiment P26. The method of Embodiment P20, where the Replicant includes a 5′ cap.

Embodiment P27. The method of Embodiment P26, where the 5′ cap includes a 7-methyl guanosine-2′-O-methoxy adenosine-guanosine.

Embodiment P28. The method of Embodiment P20, where the LNP includes three (3) or more components selected from the group consisting of a polymer-conjugated lipid, a sterol, a phospholipid, an ionizing lipid and a cationic lipid.

Embodiment P29. A composition for treating a human subject suffering from a HPV (human papillomavirus) including, a) a STAV ds DNA molecule including a first DNA molecule, and a second DNA molecule, b) a Replicant including a mRNA molecule adapted to express one or both a HPV E6 (SEQ ID NO:119) and a HPV E7 (SEQ ID NO:120), c) an IFN protein, and d) a LNP, where the STAV and the Replicant are encapsulated in the LNP.

Embodiment P30. The composition of Embodiment P29, where the first DNA molecule is between a lower limit of approximately forty bases, and an upper limit of approximately eighty bases.

Embodiment P31. The composition of Embodiment P29, where the first DNA molecule is at least eighty (80) percent complimentary with respect to the second DNA molecule.

Embodiment P32. The composition of Embodiment P29, where one or both the first DNA molecule and the second DNA molecule further comprise at least one modification located at one or both a 5′ end and a 3′ end.

Embodiment P33. The composition of Embodiment P32, where the mRNA molecule includes an exonuclease resistant phosphorothioate backbone moiety.

Embodiment P34. The composition of Embodiment P29, where the Replicant includes a 5′ cap.

Embodiment P35. The composition of Embodiment P34, where the 5′ cap includes a 7-methyl guanosine-2′-O-methoxy adenosine-guanosine.

Embodiment P36. The composition of Embodiment P29, where the LNP includes three (3) or more components selected from the group consisting of a polymer-conjugated lipid, a sterol, a phospholipid, an ionizing lipid and a cationic lipid.

Embodiment P37. The composition of Embodiment P29, where one or both the HPV E6 (SEQ ID NO:119) and the HPV E7 (SEQ ID NO:120) is overexpressed in the human subject.

Embodiment P38. The composition of Embodiment P29, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P39. A composition for treating a human subject suffering from a HPV (human papillomavirus) including, a) a Replicant including a messenger ribonucleic acid molecule adapted to express a HPV antigen, b) a STAV including a ds DNA molecule, where a first DNA molecule of the ds DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45, where a second DNA molecule of the ds DNA molecule is approximately eighty percent complimentary to the first DNA molecule in order to generate the ds DNA molecule, and c) a LNP, where the STAV and the Replicant are encapsulated in the LNP.

Embodiment P40. The composition of Embodiment P39, further including an IFN protein.

Embodiment P41. The composition of Embodiment P39, where the HPV antigen includes amino acids with sequence encompassed by a HPV E6 antigen (SEQ ID NO:119).

Embodiment P42. The composition of Embodiment P39, where the HPV antigen includes amino acids with sequence encompassed by a HPV E7 antigen (SEQ ID NO:120).

Embodiment P43. The composition of Embodiment P39, where the HPV antigen includes amino acids with sequence encompassed by a HPV E6 antigen and a HPV E7 antigen gene (SEQ ID NO:119-120).

Embodiment P44. The composition of Embodiment P39, where the Replicant includes a 5′ cap.

Embodiment P45. The composition of Embodiment P44, where the 5′ cap includes a 7-methyl guanosine-2′-O-methoxy adenosine-guanosine.

Embodiment P46. The composition of Embodiment P39, where the LNP includes three (3) or more components selected from the group consisting of a polymer-conjugated lipid, a sterol, a phospholipid, an ionizing lipid and a cationic lipid.

Embodiment P47. A method for treating a human subject suffering from a HPV including, a) determining whether the human subject is suffering from the HPV, b) if the human subject is suffering from the HPV, then determining whether the human subject has a defective functional activity of STINGby: isolating a sample from the human subject, and performing a PCR assay on the sample to determine if a cell population in the sample has a defective functional activity of STING, c) if the human subject is suffering from the HPV and has the defective functional activity of STING, then administering a composition including, (i) a Replicant including a mRNA molecule adapted to express a HPV antigen, ii) a STAV including a double-stranded (ds) DNA molecule including a first DNA molecule and a second DNA molecule, where the first DNA molecule is approximately eighty bases, where the second DNA molecule is approximately eighty percent complimentary to the first DNA molecule, and iii) a LNP, where the STAV and the Replicant are encapsulated in the LNP, and d) internally treating the human subject with the composition.

Embodiment P48. The method of Embodiment P47, the composition further including an IFN protein.

Embodiment P49. The method of Embodiment P48, where the IFN is administered prior to administering the LNP.

Embodiment P50. The method of Embodiment P47, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P51. The method of Embodiment P47, where one or both the first DNA molecule and the second DNA molecule further comprise at least one modification located at one or both a 5′ end and a 3′ end.

Embodiment P52. The method of Embodiment P51, where the mRNA molecule includes an exonuclease resistant phosphorothioate backbone moiety.

Embodiment P53. The method of Embodiment P47, where the Replicant includes a 5′ cap.

Embodiment P54. The method of Embodiment P53, where the 5′ cap includes a 7-methyl guanosine-2′-O-methoxy adenosine-guanosine.

Embodiment P55. The method of Embodiment P47, where the LNP includes three (3) or more components selected from the group consisting of a polymer-conjugated lipid, a sterol, a phospholipid, an ionizing lipid and a cationic lipid.

Embodiment P56. A composition for treating a human subject suffering from a melanoma including, a) a STAV ds DNA molecule including a first DNA molecule, and a second DNA molecule, b) a Replicant including a mRNA molecule adapted to express one or both a melanoma tumor antigen TYRP (SEQ ID NO:114-116) and MELAN-A (SEQ ID NO:83), and c) an IFN type I protein, and d) a LNP, where the STAV and the Replicant are encapsulated in the LNP.

Embodiment P57. The composition of Embodiment P56, where the first DNA molecule is between a lower limit of approximately forty bases, and an upper limit of approximately eighty bases.

Embodiment P58. The composition of Embodiment P56, where the first DNA molecule is at least eighty (80) percent complimentary with respect to the second DNA molecule.

Embodiment P59. The composition of Embodiment P56, where one or both the first DNA molecule and the second DNA molecule further comprise at least one modification located at one or both a 5′ end and a 3′ end.

Embodiment P60. The composition of Embodiment P59, where the mRNA molecule includes an exonuclease resistant phosphorothioate backbone moiety.

Embodiment P61. The composition of Embodiment P56, where the Replicant includes a 5′ cap.

Embodiment P62. The composition of Embodiment P61, where the 5′ cap includes a 7-methyl guanosine-2′-O-methoxy adenosine-guanosine.

Embodiment P63. The composition of Embodiment P56, where the LNP includes three (3) or more components selected from the group consisting of a polymer-conjugated lipid, a sterol, a phospholipid, an ionizing lipid and a cationic lipid.

Embodiment P64. The composition of Embodiment P56, where one or both the melanoma tumor antigen TYRP (SEQ ID NO:114-116) and MELAN-A (SEQ ID NO:83) is overexpressed in the human subject.

Embodiment P65. The composition of Embodiment P56, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P66. A composition for treating a human subject suffering from a melanoma including, a) a Replicant including a messenger ribonucleic acid molecule adapted to express one or both a melanoma tumor antigen, b) a STAV including a ds DNA molecule, where a first DNA molecule of the ds DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45, where a second DNA molecule of the ds DNA molecule is approximately eighty percent complimentary to the first DNA molecule in order to generate the ds DNA molecule, and
c) a LNP, where the STAV and the Replicant are encapsulated in the LNP.

Embodiment P67. The composition of Embodiment P66, further including an IFN protein.

Embodiment P68. The composition of Embodiment P66, where the melanoma tumor antigen includes amino acids with sequence encompassed by a melanoma tumor TYRP (SEQ ID NO:114-116) antigen.

Embodiment P69. The composition of Embodiment P66, where the melanoma tumor antigen includes amino acids with sequence encompassed by a melanoma tumor MELAN-A antigen (SEQ ID NO:83).

Embodiment P70. The composition of Embodiment P66, where the melanoma tumor antigen includes amino acids with sequence encompassed by a melanoma tumor TYRP (SEQ ID NO:114-116) antigen and a melanoma tumor MELAN-A antigen (SEQ ID NO:83).

Embodiment P71. The composition of Embodiment P66, where the Replicant includes a 5′ cap.

Embodiment P72. The composition of Embodiment P71, where the 5′ cap includes a 7-methyl guanosine-2′-O-methoxy adenosine-guanosine.

Embodiment P73. The composition of Embodiment P66, where the LNP includes three (3) or more components selected from the group consisting of a polymer-conjugated lipid, a sterol, a phospholipid, an ionizing lipid and a cationic lipid.

Embodiment P74. A method for treating a human subject suffering from a melanoma including, a) determining whether the human subject is suffering from the melanoma, b) if the human subject is suffering from the melanoma, then determining whether the human subject has a defective functional activity of STING by: isolating a sample from the human subject, and performing a PCR assay on the sample to determine if a cell population in the sample has a defective functional activity of STING, c) if the human subject is suffering from the melanoma and has the defective functional activity of STING, then administering a composition including, (i) a Replicant including a mRNA molecule adapted to express a melanoma tumor antigen, ii) a STAV including a double-stranded (ds) DNA molecule including a first DNA molecule and a second DNA molecule, where the first DNA molecule is approximately eighty bases, where the second DNA molecule is approximately eighty percent complimentary to the first DNA molecule, and
iii) a LNP, where the STAV and the Replicant are encapsulated in the LNP, and d) internally treating the human subject with the composition.

Embodiment P75. The method of Embodiment P74, the composition further including an IFN protein.

Embodiment P76. The method of Embodiment P75, where the IFN is administered prior to administering the LNP.

Embodiment P77. The method of Embodiment P74, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P78. The method of Embodiment P74, where one or both the first DNA molecule and the second DNA molecule further comprise at least one modification located at one or both a 5′ end and a 3′ end.

Embodiment P79. The method of Embodiment P78, where the mRNA molecule includes an exonuclease resistant phosphorothioate backbone moiety.

Embodiment P80. The method of Embodiment P74, where the Replicant includes a 5′ cap.

Embodiment P81. The method of Embodiment P80, where the 5′ cap includes a 7-methyl guanosine-2′-O-methoxy adenosine-guanosine.

Embodiment P82. The method of Embodiment P74, where the LNP includes four (4) or more components selected from the group consisting of a polymer-conjugated lipid, a sterol, a phospholipid, an ionizing lipid and a cationic lipid.

Embodiment P83. The method of Embodiment P74, where the LNP includes three (3) or more components selected from the group consisting of a polymer-conjugated lipid, a sterol, a phospholipid, an ionizing lipid and a cationic lipid.

Embodiment P84. A composition for treating a human subject suffering from a colorectal cancer (CRC) including, a) a STAV ds DNA molecule including a first DNA molecule, and a second DNA molecule, b) a Replicant including a mRNA molecule adapted to express a CRC antigen, c) an IFN type I protein, and d) a LNP, where the STAV and the Replicant are encapsulated in the LNP.

Embodiment P85. The composition of Embodiment P84, where the first DNA molecule is between a lower limit of approximately forty bases and an upper limit of approximately eighty bases.

Embodiment P86. The composition of Embodiment P84, where the first DNA molecule is at least eighty (80) percent complimentary with respect to the second DNA molecule.

Embodiment P87. The composition of Embodiment P84, where one or both the first DNA molecule and the second DNA molecule further comprise at least one modification located at one or both a 5′ end and a 3′ end.

Embodiment P88. The composition of Embodiment P87, where the mRNA molecule includes an exonuclease resistant phosphorothioate backbone moiety.

Embodiment P89. The composition of Embodiment P84, where the Replicant includes a 5′ cap.

Embodiment P90. The composition of Embodiment P89, where the 5′ cap includes a 7-methyl guanosine-2′-O-methoxy adenosine-guanosine.

Embodiment P91. The composition of Embodiment P84, where the LNP includes three (3) or more components selected from the group consisting of a polymer-conjugated lipid, a sterol, a phospholipid, an ionizing lipid and a cationic lipid.

Embodiment P92. The composition of Embodiment P84, where one or more of Carcinoembryonic Antigen mRNA (SEQ ID NO:58-66), Mucin-1 (CEACAM5) mRNA (SEQ ID NO:92-112), Epidermal Growth Factor Receptor (EGFR) mRNA (SEQ ID NO:67-79), VEGFR1, VEGFR2, Transmembrane 4 superfamily member 5 protein (TM4SF5) mRNA (SEQ ID NO:113), surviving, Mitotic Centromere-Associated Kinesin (MCAK) mRNA (SEQ ID NO:84-91), Guanylyl Cyclase C (GUCY2C) mRNA (SEQ ID NO:80-82) and oncofetal antigen 5T4 (5T4) mRNA (SEQ ID NO:55-57) is overexpressed in the human subject.

Embodiment P93. The composition of Embodiment P84, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P94. A composition for treating a human subject suffering from a melanoma including, a) a Replicant including a messenger ribonucleic acid molecule adapted to express a CRC antigen (colorectal cancer antigen), b) a STAV including a ds DNA molecule, where a first DNA molecule of the ds DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45, where a second DNA molecule of the ds DNA molecule is approximately eighty percent complimentary to the first DNA molecule in order to generate the ds DNA molecule, and c) a LNP, where the STAV and the Replicant are encapsulated in the LNP.

Embodiment P95. The composition of Embodiment P94, further including an IFN protein.

Embodiment P96. The composition of Embodiment P94, where the CRC antigen includes amino acids with sequence encompassed by Carcinoembryonic Antigen mRNA (SEQ ID NO:58-66), Mucin-1 (CEACAM5) mRNA (SEQ ID NO:92-112), Epidermal Growth Factor Receptor (EGFR) mRNA (SEQ ID NO:67-79), and Transmembrane 4 superfamily member 5 protein (TM4SF5) mRNA (SEQ ID NO:113) antigen.

Embodiment P97. The composition of Embodiment P94, where the CRC antigen includes amino acids with sequence encompassed by Mitotic Centromere-Associated Kinesin (MCAK) mRNA (SEQ ID NO:84-91), Guanylyl Cyclase C (GUCY2C) mRNA (SEQ ID NO:80-82) and oncofetal antigen 5T4 (5T4) mRNA (SEQ ID NO:55-57) antigen.

Embodiment P98. The composition of Embodiment P94, where the CRC antigen includes amino acids with sequence encompassed by one or more of surviving, VEGFR1 antigen, and VEGFR2 antigen.

Embodiment P99. The composition of Embodiment P94, where the Replicant includes a 5′ cap.

Embodiment P100. The composition of Embodiment P99, where the 5′ cap includes a 7-methyl guanosine-2′-O-methoxy adenosine-guanosine.

Embodiment P101. The composition of Embodiment P94, where the LNP includes three (3) or more components selected from the group consisting of a polymer-conjugated lipid, a sterol, a phospholipid, an ionizing lipid and a cationic lipid.

Embodiment P102. A method for treating a human subject suffering from colorectal cancer (CRC) including, a) determining whether the human subject is suffering from CRC, b) if the human subject is suffering from CRC, then determining whether the human subject has a defective functional activity of STINGby: isolating a sample from the human subject, and performing a PCR assay on the sample to determine if a cell population in the sample has a defective functional activity of STING, c) if the human subject is suffering from CRC and has the defective functional activity of STING, then administering a composition including, (i) a Replicant including a mRNA molecule adapted to express a melanoma tumor antigen, ii) a STAV including a double-stranded (ds) DNA molecule including a first DNA molecule and a second DNA molecule, where the first DNA molecule is approximately eighty bases, where the second DNA molecule is approximately eighty percent complimentary to the first DNA molecule, and iii) a LNP, where the STAV and the Replicant are encapsulated in the LNP, and d) internally treating the human subject with the composition.

Embodiment P103. The method of Embodiment P102, the composition further including an IFN protein.

Embodiment P104. The method of Embodiment P103, where the IFN is administered prior to administering the LNP.

Embodiment P105. The method of Embodiment P102, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment P106. The method of Embodiment P102, where one or both the first DNA molecule and the second DNA molecule further comprise at least one modification located at one or both a 5′ end and a 3′ end.

Embodiment P107. The method of Embodiment P106, where the mRNA molecule includes an exonuclease resistant phosphorothioate backbone moiety.

Embodiment P108. The method of Embodiment P102, where the Replicant includes a 5′ cap.

Embodiment P109. The method of Embodiment P108, where the 5′ cap includes a 7-methyl guanosine-2′-O-methoxy adenosine-guanosine.

Embodiment P110. The method of Embodiment P102, where the LNP includes three (3) or more components selected from the group consisting of a polymer-conjugated lipid, a sterol, a phospholipid, an ionizing lipid and a cationic lipid.

Embodiments contemplated herein include Embodiments Q1-Q68 following.

Embodiment Q1. A composition for treating a human subject suffering from a cancer including, a) a first DNA molecule comprising a first sequence and a first length, b) a second DNA molecule comprising a second sequence and a second length, where the first DNA molecule and the second DNA molecule are annealed to form a STAV, c) a vector comprising a Replicant, the Replicant comprising at least a first mRNA encoding an OVA antigen (SEQ ID NO:48), where the vector is adapted to express the OVA antigen, and d) a LNP, where the STAV and the vector are encapsulated in the LNP, where administering the composition to the human subject modulates one or both innate and adaptive immunity to treat the cancer.

Embodiment Q2. The composition of Embodiment Q1, further including, an IFN type I protein.

Embodiment Q3. The composition of Embodiment Q1, where the first length is between a lower limit of approximately forty bases, and an upper limit of approximately ninety bases.

Embodiment Q4. The composition of Embodiment Q1, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45.

Embodiment Q5. The composition of Embodiment Q1, where the second length exceeds the first length by not more than between a lower limit of approximately one base, and an upper limit of approximately fifty bases.

Embodiment Q6. The composition of Embodiment Q5, where the Percent Complimentary of the second sequence is at least eighty (80) percent with respect to the first sequence.

Embodiment Q7. The composition of Embodiment Q1, where one or both the first DNA molecule and the second DNA molecule further comprise at least one modification located at one or both a 5′ end and a 3′ end.

Embodiment Q8. The composition of Embodiment Q7, where the vector comprises an exonuclease resistant phosphorothioate backbone moiety.

Embodiment Q9. The composition of Embodiment Q1, where the vector comprises a 5′ cap.

Embodiment Q10. The composition of Embodiment Q9, where the 5′ cap comprises a 7-methyl guanosine-2′-O-methoxy adenosine-guanosine.

Embodiment Q11. The composition of Embodiment Q1, where the LNP comprises three (3) or more components selected from the group consisting of a polymer-conjugated lipid, a sterol, a phospholipid, an ionizing lipid and a cationic lipid.

Embodiment Q12. A composition including, a) a vector including, a Replicant including, at least a first mRNA encoding an OVA antigen (SEQ ID NO:48), where the vector is adapted to express the OVA antigen, b) a first DNA molecule including, a first sequence and a first length, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45, c) a second DNA molecule including, a second sequence and a second length, where the second length exceeds the first length by not more than between a lower limit of approximately one base, and an upper limit of approximately fifty bases, where the second sequence is at least eighty (80) percent complimentary with respect to the first sequence, where the first DNA molecule and the second DNA molecule are annealed to form a STAV, and c) a LNP, where the STAV and the vector are encapsulated in the LNP.

Embodiment Q13. The composition of Embodiment Q12, further including, an IFN protein.

Embodiment Q14. The composition of Embodiment Q12, where the vector comprises a 5′ cap.

Embodiment Q15. The composition of Embodiment Q14, where the 5′ cap comprises a 7-methyl guanosine-2′-O-methoxy adenosine-guanosine.

Embodiment Q16. The composition of Embodiment Q12, where the LNP comprises three (3) or more components selected from the group consisting of a polymer-conjugated lipid, a sterol, a phospholipid, an ionizing lipid and a cationic lipid.

Embodiment Q18. A method for treating a human subject suffering from a malady selected from the group consisting of an immune-related disorder, a cancer, an autoimmunity disease, and an infection including, determining whether the human subject is suffering from the malady including, isolating a sample from the human subject, performing an assay to determine the human subject is suffering from the malady, then administering a composition including, a) a first DNA molecule including, a first sequence and a first length, b) a second DNA molecule including, a second sequence and a second length, where the first DNA molecule and the second DNA molecule are annealed to form a, c) a vector including, a Replicant, the Replicant including, at least a first mRNA encoding an OVA antigen (SEQ ID NO:48), where the vector is adapted to express the OVA antigen, and d) a LNP, where the STAV and the vector are encapsulated in the LNP, where administering the composition to the human subject modulates one or both innate and adaptive immunity to treat the malady.

Embodiment Q19. The method of claim 18, the composition further including, an IFN protein.

Embodiment Q20. The method of Embodiment Q18, where an IFN protein is administered prior to administering the composition.

Embodiment Q21. The method of Embodiment Q18, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment Q22. The method of Embodiment Q18, where one or both the first DNA molecule and the second DNA molecule further comprise at least one modification located at one or both a 5′ end and a 3′ end.

Embodiment Q23. The method of Embodiment Q18, where the vector molecule comprises an exonuclease resistant phosphorothioate backbone moiety.

Embodiment Q24. A composition including, a) a first DNA molecule including, a first sequence and a first length, b) a second DNA molecule including, a second sequence and a second length, where the first DNA molecule and the second DNA molecule are annealed to form a, c) a vector including, a Replicant, the Replicant including, at least a first mRNA encoding an antigen, where the vector is adapted to express the antigen, and d) a LNP, where the STAV and the vector are encapsulated in the LNP.

Embodiment Q25. The composition of Embodiment Q24, further including, an IFN type I protein.

Embodiment Q26. The composition of Embodiment Q24, where the first length is between a lower limit of approximately forty bases, and an upper limit of approximately ninety bases.

Embodiment Q27. The composition of Embodiment Q24, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45.

Embodiment Q28. The composition of Embodiment Q24, where the second length exceeds the first length by not more than between a lower limit of approximately one base, and an upper limit of approximately fifty bases.

Embodiment Q29. The composition of Embodiment Q28, where the Percent Complimentary of the second sequence is at least eighty (80) percent with respect to the first sequence.

Embodiment Q30. The composition of Embodiment Q24, where one or both the first DNA molecule and the second DNA molecule further comprise at least one modification located at one or both a 5′ end and a 3′ end.

Embodiment Q31. The composition of Embodiment Q30, where the vector comprises an exonuclease resistant phosphorothioate backbone moiety.

Embodiment Q32. The composition of Embodiment Q24, where the vector comprises a 5′ cap.

Embodiment Q33. The composition of Embodiment Q32, where the 5′ cap comprises a 7-methyl guanosine-2′-O-methoxy adenosine-guanosine.

Embodiment Q34. The composition of Embodiment Q24, where the LNP comprises three (3) or more components selected from the group consisting of a polymer-conjugated lipid, a sterol, a phospholipid, an ionizing lipid and a cationic lipid.

Embodiment Q35. A composition for treating a human subject suffering from HTLV-1 (human T-cell leukemia virus type 1) including, a) a first DNA molecule including, a first sequence and a first length, b) a second DNA molecule including, a second sequence and a second length, where the first DNA molecule and the second DNA molecule are annealed to form a, c) a vector including, a Replicant, the Replicant including, at least a first mRNA encoding a HTLV-1 antigen selected from the group consisting of HBZ (Human Basic leucine Zipper factor gene) (SEQ ID NO:118), TAX (Trans Activator gene) (SEQ ID NO:117), HBV-TAX (SEQ ID NO:54), and gp62G (gp62 envelope glycoprotein) (SEQ ID NO:122), where the vector is adapted to express the HTLV-1 antigen, and d) a LNP, where the STAV and the vector are encapsulated in the LNP, where administering the composition to the human subject modulates one or both innate and adaptive immunity to treat the cancer.

Embodiment Q36. The composition of Embodiment Q35, further including, an IFN type I protein.

Embodiment Q37. The composition of Embodiment Q35, where the first length is between a lower limit of approximately forty bases, and an upper limit of approximately ninety bases.

Embodiment Q38. The composition of Embodiment Q35, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45.

Embodiment Q39. The composition of Embodiment Q35, where the second length exceeds the first length by not more than between a lower limit of approximately one base, and an upper limit of approximately fifty bases.

Embodiment Q40. The composition of Embodiment Q39, where the Percent Complimentary of the second sequence is at least eighty (80) percent with respect to the first sequence.

Embodiment Q41. The composition of Embodiment Q35, where one or both the first DNA molecule and the second DNA molecule further comprise at least one modification located at one or both a 5′ end and a 3′ end.

Embodiment Q42. The composition of Embodiment Q41, where the vector comprises an exonuclease resistant phosphorothioate backbone moiety.

Embodiment Q43. The composition of Embodiment Q35, where the vector comprises a 5′ cap.

Embodiment Q44. The composition of Embodiment Q43, where the 5′ cap comprises a 7-methyl guanosine-2′-O-methoxy adenosine-guanosine.

Embodiment Q45. The composition of Embodiment Q1, where the LNP comprises three (3) or more components selected from the group consisting of a polymer-conjugated lipid, a sterol, a phospholipid, an ionizing lipid and a cationic lipid.

Embodiment Q46. A composition for treating a human subject suffering from HTLV-1 (human T-cell leukemia virus type 1) including, a) a first DNA molecule including, a first sequence and a first length, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45, b) a second DNA molecule including, a second sequence and a second length, where the first DNA molecule and the second DNA molecule are annealed to form a, c) a vector including, a Replicant, the Replicant including, at least a first mRNA encoding a HTLV-1 antigen selected from the group consisting of HBZ (Human Basic leucine Zipper factor gene) (SEQ ID NO:118), TAX (Trans Activator gene) (SEQ ID NO:117), HBV-TAX (SEQ ID NO:54), and gp62G (gp62 envelope glycoprotein) (SEQ ID NO:122), where the vector is adapted to express the HTLV-1 antigen, and d) a LNP, where the STAV and the vector are encapsulated in the LNP, where administering the composition to the human subject modulates one or both innate and adaptive immunity to treat the cancer.

Embodiment Q47. The composition of Embodiment Q46, further including, an IFN protein.

Embodiment Q48. The composition of Embodiment Q46, where the vector comprises a 5′ cap.

Embodiment Q49. The composition of Embodiment Q47, where the 5′ cap comprises a 7-methyl guanosine-2′-O-methoxy adenosine-guanosine.

Embodiment Q50. The composition of Embodiment Q46, where the LNP comprises three (3) or more components selected from the group consisting of a polymer-conjugated lipid, a sterol, a phospholipid, an ionizing lipid and a cationic lipid.

Embodiment Q51. A method for treating a human subject suffering from a malady associated with HTLV-1 (human T-cell leukemia virus type 1) including, determining whether the human subject is suffering from HTLV-1 including, isolating a sample from the human subject, performing an assay to determine if the human subject is suffering from one or more of HTLV-1, Adult T cell leukemia (ATLL), Acute myeloid leukemia (AML), and acute lymphocytic leukemia (ALL), and then administering a composition including, a) a first DNA molecule including, a first sequence and a first length, b) a second DNA molecule including, a second sequence and a second length, where the first DNA molecule and the second DNA molecule are annealed to form a, c) a vector including, a Replicant, the Replicant including, at least a first mRNA encoding a HTLV-1 antigen selected from the group consisting of HBZ (Human Basic leucine Zipper factor gene) (SEQ ID NO:118), TAX (Trans Activator gene) (SEQ ID NO:117), HBV-TAX (SEQ ID NO:54), and gp62G (gp62 envelope glycoprotein) (SEQ ID NO:122), where the vector is adapted to express the HTLV-1 antigen, and d) a LNP, where the STAV and the vector are encapsulated in the LNP, where administering the composition to the human subject modulates one or both innate and adaptive immunity to treat the malady.

Embodiment Q52. The method of Embodiment Q51, the composition further including, an IFN protein.

Embodiment Q53. The method of Embodiment Q51, where an IFN protein is administered prior to administering the composition.

Embodiment Q54. The method of Embodiment Q51, where the first DNA molecule is selected from the group consisting of SEQ ID NO:24, SEQ ID NO:26, SEQ ID NO:37, SEQ ID NO:39, SEQ ID NO:41, SEQ ID NO:43, and SEQ ID NO:45 and the second DNA molecule is at least eighty (80) percent complimentary to the first DNA molecule.

Embodiment Q55. The method of Embodiment Q51, where one or both the first DNA molecule and the second DNA molecule further comprise at least one modification located at one or both a 5′ end and a 3′ end.

Embodiment Q56. The method of Embodiment Q51, where the vector comprises an exonuclease resistant phosphorothioate backbone moiety.

Embodiment Q57. The method of Embodiment Q51, where the vector comprises a 5′ cap.

Embodiment Q58. The method of Embodiment Q57, where the 5′ cap comprises a 7-methyl guanosine-2′-O-methoxy adenosine-guanosine.

Embodiment Q59. The method of Embodiment Q51, where the LNP comprises three (3) or more components selected from the group consisting of a polymer-conjugated lipid, a sterol, a phospholipid, an ionizing lipid and a cationic lipid.

Embodiment Q60. A composition for treating a human subject suffering from a cancer including, a) a first DNA molecule comprising a first sequence and a first length, b) a second DNA molecule comprising a second sequence and a second length, where the first DNA molecule and the second DNA molecule are annealed to form a STAV, and c) a vector comprising a Replicant, the Replicant comprising at least a first mRNA encoding an OVA antigen (SEQ ID NO:48), where the vector is adapted to express the OVA antigen, where administering the composition to the human subject modulates one or both innate and adaptive immunity to treat the cancer.

Embodiment Q61. The composition of Embodiment Q60, the composition further comprising an IFN type I protein, where the composition is delivered in a LNP, where the LNP comprises three (3) or more components selected from the group consisting of a polymer-conjugated lipid, a sterol, a phospholipid, an ionizing lipid and a cationic lipid, where at least the STAV and the vector are encapsulated in the LNP.

Embodiment Q62. The composition of Embodiment Q60, where the vector comprises a 7-methyl guanosine-2′-O-methoxy adenosine-guanosine 5′ cap.

Embodiment Q63. A method for treating a human subject suffering from a malady selected from the group consisting of an immune-related disorder, a cancer, an autoimmunity disease, and an infection including, determining whether the human subject is suffering from the malady including, isolating a sample from the human subject; and performing an assay to determine the human subject is suffering from the malady, then administering a composition including, a) a first DNA molecule comprising a first sequence and a first length, b) a second DNA molecule comprising a second sequence and a second length, where the first DNA molecule and the second DNA molecule are annealed to form a STAV, c) a vector comprising a Replicant, the Replicant comprising at least a first mRNA encoding an OVA antigen (SEQ ID NO:48), where the vector is adapted to express the OVA antigen, where administering the composition to the human subject modulates one or both innate and adaptive immunity to treat the malady.

Embodiment Q64. The method of Embodiment Q63, the composition further comprising an IFN type I protein, where the composition is delivered in a LNP, where the LNP comprises three (3) or more components selected from the group consisting of a polymer-conjugated lipid, a sterol, a phospholipid, an ionizing lipid and a cationic lipid, where at least the STAV and the vector are encapsulated in the LNP.

Embodiment Q65. The method of Embodiment Q64, where the IFN type I protein is administered prior to administering the LNP.

Embodiment Q66. The method of Embodiment Q64, where an IFN type I protein is administered after administering the LNP.

Embodiment Q67. The method of Embodiment Q63, where one or both the first DNA molecule and the second DNA molecule further comprise one or more modifications located at one or both a 5′ end and a 3′ end.

Embodiment Q68. The method of Embodiment Q67, where the one or more modifications inhibit DNaseII.

Abbreviations include: ALL=acute lymphocytic leukemia; AML=acute myeloid leukemia; ATLL=Adult T cell leukemia; AZT=Zidovudine; B-cell=B-lymphocyte cell; CTL=Cytotoxic T lymphocyte; DC=dendritic cell; DNA=Deoxyribonucleic acid; dsDNA=double stranded DNA; DMSO=dimethyl sulfoxide; HCl=hydrochloric acid; HSCT=Hematopoietic stem cell transplantation; HTLV-1=human T-cell leukemia virus type 1; IFN type I=Interferon type I; LNP=lipid nano particle; mRNA=messenger RNA; MS=medium survival; PBS=phosphate buffered saline; PS=phosphorothioate; PMBC=peripheral blood mononuclear cell; RNA=ribonucleic acid; TBS=tris-HCl-buffered saline; T-cell=T-lymphocyte cell; TLT=treatment limiting toxicities; WT=wild type.

What is claimed:

1. A composition for treating a human subject suffering from a cancer comprising:
a) a first DNA molecule comprising a first sequence and a first length;
b) a second DNA molecule comprising a second sequence and a second length, where the first DNA molecule and the second DNA molecule are annealed to form a STAV (a dsDNA (double stranded DeoxyriboNucleic Acid) molecule); and
c) a vector comprising a Replicant, the Replicant comprising at least a first mRNA (messenger RiboNucleic Acid) encoding an OVA antigen (SEQ ID NO:48), where the vector is adapted to express the OVA antigen, where administering the composition to the human subject modulates one or both innate and adaptive immunity to treat the cancer.

2. The composition of claim 1, the composition further comprising an IFN type I protein, where the composition is delivered in a LNP (a Lipid Nanoparticle), where the LNP comprises three (3) or more components selected from the group consisting of a polymer-conjugated lipid, a sterol, a phospholipid, an ionizing lipid and a cationic lipid, where at least the STAV and the vector are encapsulated in the LNP.

3. The composition of claim 1, where the first length is between:
a lower limit of approximately forty bases; and
an upper limit of approximately ninety bases.

4. The composition of claim 1, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45.

5. The composition of claim 1, where the second length exceeds the first length by not more than between:
a lower limit of approximately one base; and
an upper limit of approximately fifty bases.

6. The composition of claim 5, where the Percent Complimentary of the second sequence is at least eighty (80) percent with respect to the first sequence.

7. The composition of claim 1, where one or both the first DNA molecule and the second DNA molecule further comprise at least one modification located at one or both a 5′ end and a 3′ end.

8. The composition of claim 7, where the vector comprises an exonuclease resistant phosphorothioate backbone moiety.

9. The composition of claim 1, where the vector comprises a 5′ cap.

10. The composition of claim 9, where the 5′ cap comprises a 7-methyl guanosine-2′-O-methoxy adenosine-guanosine.

11. A composition comprising:
a) a vector comprising a Replicant comprising at least a first mRNA (messenger RiboNucleic Acid) encoding an antigen, where the vector is adapted to express the antigen;
b) a first DNA molecule comprising a first sequence and a first length, where the first sequence is selected from the group consisting of SEQ ID NO:24, SEQ ID NO:26, SEQ ID NO:37, SEQ ID NO:39, SEQ ID NO:41, SEQ ID NO:43, and SEQ ID NO:45; and
c) a second DNA molecule comprising a second sequence and a second length, where the second length exceeds the first length by not more than between:
a lower limit of approximately one base, and
an upper limit of approximately fifty bases, where the Percent Complimentary of the second sequence is at least eighty (80) percent with respect to the first sequence, where the first DNA molecule and the second DNA molecule are annealed to form a STAV (a dsDNA (double-stranded DeoxyriboNucleic Acid) molecule).

12. The composition of claim 11, the composition further comprising an IFN type I protein, where the composition is delivered in a LNP (a Lipid Nanoparticle), where the LNP comprises three (3) or more components selected from the group consisting of a polymer-conjugated lipid, a sterol, a phospholipid, an ionizing lipid and a cationic lipid, where at least the STAV and the vector are encapsulated in the LNP.

13. The composition of claim 11, where the vector comprises a 7-methyl guanosine-2′-O-methoxy adenosine-guanosine 5′ cap.

14. The composition of claim 11, where the first mRNA encodes an OVA antigen (SEQ ID NO:48), where the vector is adapted to express the OVA antigen.

15. A method for treating a human subject suffering from a malady selected from the group consisting of an immune-related disorder, a cancer, an autoimmunity disease, and an infection comprising:
determining whether the human subject is suffering from the malady comprising:
i) isolating a sample from the human subject;
ii) performing an assay to determine the human subject is suffering from the malady, then administering a composition comprising:
a) a first DNA molecule comprising a first sequence and a first length;
b) a second DNA molecule comprising a second sequence and a second length, where the first DNA molecule and the second DNA molecule are annealed to form a STAV (a dsDNA (double stranded DeoxyriboNucleic Acid) molecule); and
c) a vector comprising a Replicant, the Replicant comprising at least a first mRNA (messenger RiboNucleic Acid) encoding an OVA antigen (SEQ ID NO:48), where the vector is adapted to express the OVA antigen, where administering the composition to the human subject modulates one or both innate and adaptive immunity to treat the malady.

16. The method of claim 15, the composition further comprising an IFN type I protein, where the composition is delivered in a LNP (a Lipid Nanoparticle), where the LNP comprises three (3) or more components selected from the group consisting of a polymer-conjugated lipid, a sterol, a phospholipid, an ionizing lipid and a cationic lipid, where at least the STAV and the vector are encapsulated in the LNP.

17. The method of claim 16, where the IFN type I protein is administered prior to administering the LNP.

18. The method of claim 16, where the IFN type I protein is administered after administering the LNP.

19. The method of claim 15, where one or both the first DNA molecule and the second DNA molecule further comprise one or more modifications located at one or both a 5′ end and a 3′ end.

20. The method of claim 19, where DNaseII is inhibited by the one or more modifications.


SEQUENCE LISTING (171 sequences)

SEQ ID NO: 1 - DNA, 30 bp, synthetic construct
  misc_feature 1..30: note = Synthetic polynucleotide
  source 1..30: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 28: mod_base = OTHER; note = 2'-deoxyadenosine-5'-O-monophosphorothioate
  modified_base 29: mod_base = OTHER; note = 2'-deoxyadenosine-5'-O-monophosphorothioate
  modified_base 30: mod_base = OTHER; note = 2'-deoxyadenosine-5'-O-monophosphorothioate
aaaaaaaaaaaaaaaaaaaaaaaaaaaaaa

SEQ ID NO: 2 - DNA, 30 bp, synthetic construct
  misc_feature 1..30: note = Synthetic polynucleotide
  source 1..30: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 28: mod_base = OTHER; note = 2'-deoxythymidine-5'-O-monophosphorothioate
  modified_base 29: mod_base = OTHER; note = 2'-deoxythymidine-5'-O-monophosphorothioate
  modified_base 30: mod_base = OTHER; note = 2'-deoxythymidine-5'-O-monophosphorothioate
tttttttttttttttttttttttttttttt

SEQ ID NO: 3 - DNA, 50 bp, synthetic construct
  misc_feature 1..50: note = Synthetic polynucleotide
  source 1..50: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 48: mod_base = OTHER; note = 2'-deoxyadenosine-5'-O-monophosphorothioate
  modified_base 49: mod_base = OTHER; note = 2'-deoxyadenosine-5'-O-monophosphorothioate
  modified_base 50: mod_base = OTHER; note = 2'-deoxyadenosine-5'-O-monophosphorothioate
aaaaaaaaaaaaaaaaaaaaaaaaaaaaaaaaaaaaaaaaaaaaaaaaaa

SEQ ID NO: 4 - DNA, 50 bp, synthetic construct
  misc_feature 1..50: note = Synthetic polynucleotide
  source 1..50: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 48: mod_base = OTHER; note = 2'-deoxythymidine-5'-O-monophosphorothioate
  modified_base 49: mod_base = OTHER; note = 2'-deoxythymidine-5'-O-monophosphorothioate
  modified_base 50: mod_base = OTHER; note = 2'-deoxythymidine-5'-O-monophosphorothioate
tttttttttttttttttttttttttttttttttttttttttttttttttt

SEQ ID NO: 5 - DNA, 60 bp, synthetic construct
  misc_feature 1..60: note = Synthetic polynucleotide
  source 1..60: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 58: mod_base = OTHER; note = 2'-deoxyadenosine-5'-O-monophosphorothioate
  modified_base 59: mod_base = OTHER; note = 2'-deoxyadenosine-5'-O-monophosphorothioate
  modified_base 60: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaa

SEQ ID NO: 6 - DNA, 60 bp, synthetic construct
  misc_feature 1..60: note = Synthetic polynucleotide
  source 1..60: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 58: mod_base = OTHER; note = 2'-deoxythymidine-5'-O-monophosphorothioate
  modified_base 59: mod_base = OTHER; note = 2'-deoxythymidine-5'-O-monophosphorothioate
  modified_base 60: mod_base = OTHER; note = 2'-deoxythymidine-5'-O-monophosphorothioate
tttttttttttttttttttttttttttttttttttttttttttttttttttttttttttt

SEQ ID NO: 7 - DNA, 70 bp, synthetic construct
  misc_feature 1..70: note = Synthetic polynucleotide
  source 1..70: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 68: mod_base = OTHER; note = 2'-deoxyadenosine-5'-O-monophosphorothioate
  modified_base 69: mod_base = OTHER; note = 2'-deoxyadenosine-5'-O-monophosphorothioate
  modified_base 70: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaa

SEQ ID NO: 8 - DNA, 70 bp, synthetic construct
  misc_feature 1..70: note = Synthetic polynucleotide
  source 1..70: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 68: mod_base = OTHER; note = 2'-deoxythymidine-5'-O-monophosphorothioate
  modified_base 69: mod_base = OTHER; note = 2'-deoxythymidine-5'-O-monophosphorothioate
  modified_base 70: mod_base = OTHER; note = 2'-deoxythymidine-5'-O-monophosphorothioate
tttttttttttttttttttttttttttttttttttttttttttttttttttttttttttttttttttttt

SEQ ID NO: 9 - DNA, 80 bp, synthetic construct
  misc_feature 1..80: note = Synthetic polynucleotide
  source 1..80: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 78: mod_base = OTHER; note = 2'-deoxyadenosine-5'-O-monophosphorothioate
  modified_base 79: mod_base = OTHER; note = 2'-deoxyadenosine-5'-O-monophosphorothioate
  modified_base 80: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 80 bp, synthetic construct
  misc_feature 1..80: note = Synthetic polynucleotide
  source 1..80: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 78: mod_base = OTHER; note = 2'-deoxythymidine-5'-O-monophosphorothioate
  modified_base 79: mod_base = OTHER; note = 2'-deoxythymidine-5'-O-monophosphorothioate
  modified_base 80: mod_base = OTHER; note = 2'-deoxythymidine-5'-O-monophosphorothioate
tttttttttttttttttttttttttttttttttttttttttttttttttttttttttttttttttttttttttttttttt

SEQ ID NO: 11 - DNA, 90 bp, synthetic construct
  misc_feature 1..90: note = Synthetic polynucleotide
  source 1..90: mol_type = other DNA
  modified_base 2..4: mod_base = OTHER; note = 2'-deoxyadenosine-5'-O-monophosphorothioate
  modified_base 88..90: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 90 bp, synthetic construct
  misc_feature 1..90: note = Synthetic polynucleotide
  source 1..90: mol_type = other DNA
  modified_base 2..4: mod_base = OTHER; note = 2'-deoxythymidine-5'-O-monophosphorothioate
  modified_base 88..90: mod_base = OTHER; note = 2'-deoxythymidine-5'-O-monophosphorothioate
tttttttttttttttttttttttttttttttttttttttttttttttttttttttttttttttttttttttttttttttttttttttttt

SEQ ID NO: 13 - DNA, 100 bp, synthetic construct
  misc_feature 1..100: note = Synthetic polynucleotide
  source 1..100: mol_type = other DNA
  modified_base 2..4: mod_base = OTHER; note = 2'-deoxyadenosine-5'-O-monophosphorothioate
  modified_base 98..100: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 100 bp, synthetic construct
  misc_feature 1..100: note = Synthetic polynucleotide
  source 1..100: mol_type = other DNA
  modified_base 2..4: mod_base = OTHER; note = 2'-deoxythymidine-5'-O-monophosphorothioate
  modified_base 98..100: mod_base = OTHER; note = 2'-deoxythymidine-5'-O-monophosphorothioate
tttttttttttttttttttttttttttttttttttttttttttttttttttttttttttttttttttttttttttttttttttttttttttttttttttt

SEQ ID NO: 15 - DNA, 110 bp, synthetic construct
  misc_feature 1..110: note = Synthetic polynucleotide
  source 1..110: mol_type = other DNA
  modified_base 2..4: mod_base = OTHER; note = 2'-deoxyadenosine-5'-O-monophosphorothioate
  modified_base 108..110: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 110 bp, synthetic construct
  misc_feature 1..110: note = Synthetic polynucleotide
  source 1..110: mol_type = other DNA
  modified_base 2..4: mod_base = OTHER; note = 2'-deoxythymidine-5'-O-monophosphorothioate
  modified_base 108..110: mod_base = OTHER; note = 2'-deoxythymidine-5'-O-monophosphorothioate
tttttttttttttttttttttttttttttttttttttttttttttttttttttttttttttttttttttttttttttttttttttttttttttttttttttttttttttt

SEQ ID NO: 17 - DNA, 30 bp, synthetic construct
  misc_feature 1..30: note = Synthetic polynucleotide
  source 1..30: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 28: mod_base = OTHER; note = 2'-deoxycytidine-5'-O-monophosphorothioate
  modified_base 29: mod_base = OTHER; note = 2'-deoxyguanosine-5'-O-monophosphorothioate
  modified_base 30: mod_base = OTHER; note = 2'-deoxycytidine-5'-O-monophosphorothioate
gcgcgcgcgcgcgcgcgcgcgcgcgcgcgc

SEQ ID NO: 18 - DNA, 50 bp, synthetic construct
  misc_feature 1..50: note = Synthetic polynucleotide
  source 1..50: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 48: mod_base = OTHER; note = 2'-deoxycytidine-5'-O-monophosphorothioate
  modified_base 49: mod_base = OTHER; note = 2'-deoxyguanosine-5'-O-monophosphorothioate
  modified_base 50: mod_base = OTHER; note = 2'-deoxycytidine-5'-O-monophosphorothioate
gcgcgcgcgcgcgcgcgcgcgcgcgcgcgcgcgcgcgcgcgcgcgcgcgc

SEQ ID NO: 19 - DNA, 60 bp, synthetic construct
  misc_feature 1..60: note = Synthetic polynucleotide
  source 1..60: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 58: mod_base = OTHER; note = 2'-deoxycytidine-5'-O-monophosphorothioate
  modified_base 59: mod_base = OTHER; note = 2'-deoxyguanosine-5'-O-monophosphorothioate
  modified_base 60: mod_base = OTHER; note = 2'-deoxycytidine-5'-O-monophosphorothioate
gcgcgcgcgcgcgcgcgcgcgcgcgcgcgcgcgcgcgcgcgcgcgcgcgcgcgcgcgcgc

SEQ ID NO: 20 - DNA, 70 bp, synthetic construct
  misc_feature 1..70: note = Synthetic polynucleotide
  source 1..70: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 68: mod_base = OTHER; note = 2'-deoxycytidine-5'-O-monophosphorothioate
  modified_base 69: mod_base = OTHER; note = 2'-deoxyguanosine-5'-O-monophosphorothioate
  modified_base 70: mod_base = OTHER; note = 2'-deoxycytidine-5'-O-monophosphorothioate
gcgcgcgcgcgcgcgcgcgcgcgcgcgcgcgcgcgcgcgcgcgcgcgcgcgcgcgcgcgcgcgcgcgcgc

SEQ ID NO: 21 - DNA, 80 bp, synthetic construct
  misc_feature 1..80: note = Synthetic polynucleotide
  source 1..80: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 78: mod_base = OTHER; note = 2'-deoxycytidine-5'-O-monophosphorothioate
  modified_base 79: mod_base = OTHER; note = 2'-deoxyguanosine-5'-O-monophosphorothioate
  modified_base 80: mod_base = OTHER; note = 2'-deoxycytidine-5'-O-monophosphorothioate
gcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 22 - DNA, 90 bp, synthetic construct
  misc_feature 1..90: note = Synthetic polynucleotide
  source 1..90: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 88: mod_base = OTHER; note = 2'-deoxycytidine-5'-O-monophosphorothioate
  modified_base 89: mod_base = OTHER; note = 2'-deoxyguanosine-5'-O-monophosphorothioate
  modified_base 90: mod_base = OTHER; note = 2'-deoxycytidine-5'-O-monophosphorothioate
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 23 - DNA, 100 bp, synthetic construct
  misc_feature 1..100: note = Synthetic polynucleotide
  source 1..100: mol_type = other DNA
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate
  modified_base 98: mod_base = OTHER; note = 2'-deoxycytidine-5'-O-monophosphorothioate
  modified_base 99: mod_base = OTHER; note = 2'-deoxyguanosine-5'-O-monophosphorothioate
  modified_base 100: mod_base = OTHER; note = 2'-deoxycytidine-5'-O-monophosphorothioate
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 24 - DNA, 76 bp, synthetic construct
  misc_feature 1..76: note = Synthetic polynucleotide
  source 1..76: mol_type = other DNA
  modified_base 2..4: mod_base = OTHER; note = 2'-deoxyadenosine-5'-O-monophosphorothioate
  modified_base 74..76: mod_base = OTHER; note = 2'-deoxyadenosine-5'-O-monophosphorothioate
aaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 76 bp, synthetic construct
  misc_feature 1..76: note = Synthetic polynucleotide
  source 1..76: mol_type = other DNA
  modified_base 2..4: mod_base = OTHER; note = 2'-deoxythymidine-5'-O-monophosphorothioate
  modified_base 74..76: mod_base = OTHER; note = 2'-deoxythymidine-5'-O-monophosphorothioate
tttttttttttttttttttttttttttttttttttttttttttttttttttttttttttttttttttttttttttt

SEQ ID NO: 26 - DNA, 76 bp, synthetic construct
  misc_feature 1..76: note = Synthetic polynucleotide
  source 1..76: mol_type = other DNA
  modified_base 2: mod_base = OTHER; note = 2'-deoxycytidine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxycytidine-5'-O-monophosphorothioate
  modified_base 74: mod_base = OTHER; note = 2'-deoxycytidine-5'-O-monophosphorothioate
  modified_base 75: mod_base = OTHER; note = 2'-deoxyadenosine-5'-O-monophosphorothioate
  modified_base 76: mod_base = OTHER; note = 2'-deoxycytidine-5'-O-monophosphorothioate
acacacacacacacacacacacacacacacacacacacacacacacacacacacacacacacacacacacacacac

SEQ ID NO: 27 - DNA, 76 bp, synthetic construct
  misc_feature 1..76: note = Synthetic polynucleotide
  source 1..76: mol_type = other DNA
  modified_base 2: mod_base = OTHER; note = 2'-deoxythymidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxythymidine-5'-O-monophosphorothioate
  modified_base 74: mod_base = OTHER; note = 2'-deoxythymidine-5'-O-monophosphorothioate
  modified_base 75: mod_base = OTHER; note = 2'-deoxyguanosine-5'-O-monophosphorothioate
  modified_base 76: mod_base = OTHER; note = 2'-deoxythymidine-5'-O-monophosphorothioate
gtgtgtgtgtgtgtgtgtgtgtgtgtgtgtgtgtgtgtgtgtgtgtgtgtgtgtgtgtgtgtgtgtgtgtgtgtgt

SEQ ID NO: 28 - DNA, 76 bp, synthetic construct
  misc_feature 1..76: note = Synthetic polynucleotide
  source 1..76: mol_type = other DNA
  modified_base 2: mod_base = OTHER; note = 2'-deoxythymidine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxythymidine-5'-O-monophosphorothioate
  modified_base 74: mod_base = OTHER; note = 2'-deoxythymidine-5'-O-monophosphorothioate
  modified_base 75: mod_base = OTHER; note = 2'-deoxyadenosine-5'-O-monophosphorothioate
  modified_base 76: mod_base = OTHER; note = 2'-deoxythymidine-5'-O-monophosphorothioate
atatatatatatatatatatatatatatatatatatatatatatatatatatatatatatatatatatatatatat

SEQ ID NO: 29 - DNA, 76 bp, synthetic construct
  misc_feature 1..76: note = Synthetic polynucleotide
  source 1..76: mol_type = other DNA
  modified_base 2: mod_base = OTHER; note = 2'-deoxyadenosine-5'-O-monophosphorothioate
  modified_base 3: mod_base = OTHER; note = 2'-deoxythymidine-5'-O-monophosphorothioate
  modified_base 4: mod_base = OTHER; note = 2'-deoxyadenosine-5'-O-monophosphorothioate
  modified_base 74: mod_base = OTHER; note = 2'-deoxyadenosine-5'-O-monophosphorothioate
  modified_base 75: mod_base = OTHER; note = 2'-deoxythymidine-5'-O-monophosphorothioate
  modified_base 76: mod_base = OTHER; note = 2'-deoxyadenosine-5'-O-monophosphorothioate
tatatatatatatatatatatatatatatatatatatatatatatatatatatatatatatatatatatatatata

SEQ ID NO: 30 - DNA, 76 bp, synthetic construct
  misc_feature 1..76: note = Synthetic polynucleotide
  source 1..76: mol_type = other DNA
  modified_base 2: mod_base = OTHER; note = 2'-deoxycytidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxyguanosine-5'-O-monophosphorothioate
  modified_base 74: mod_base = OTHER; note = 2'-deoxycytidine-5'-O-monophosphorothioate
  modified_base 75: mod_base = OTHER; note = 2'-deoxythymidine-5'-O-monophosphorothioate
  modified_base 76: mod_base = OTHER; note = 2'-deoxyguanosine-5'-O-monophosphorothioate
actgactgactgactgactgactgactgactgactgactgactgactgactgactgactgactgactgactgactg

SEQ ID NO: 31 - DNA, 76 bp, synthetic construct
  misc_feature 1..76: note = Synthetic polynucleotide
  source 1..76: mol_type = other DNA
  modified_base 2: mod_base = OTHER; note = 2'-deoxyadenosine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxythymidine-5'-O-monophosphorothioate
  modified_base 74: mod_base = OTHER; note = 2'-deoxyadenosine-5'-O-monophosphorothioate
  modified_base 75: mod_base = OTHER; note = 2'-deoxyguanosine-5'-O-monophosphorothioate
  modified_base 76: mod_base = OTHER; note = 2'-deoxythymidine-5'-O-monophosphorothioate
cagtcagtcagtcagtcagtcagtcagtcagtcagtcagtcagtcagtcagtcagtcagtcagtcagtcagtcagt

SEQ ID NO: 32 - DNA, 76 bp, synthetic construct
  misc_feature 1..76: note = Synthetic polynucleotide
  source 1..76: mol_type = other DNA
  modified_base 2: mod_base = OTHER; note = 2'-deoxyadenosine-5'-O-monophosphorothioate
  modified_base 3..4: mod_base = OTHER; note = 2'-deoxycytidine-5'-O-monophosphorothioate
  modified_base 74: mod_base = OTHER; note = 2'-deoxyadenosine-5'-O-monophosphorothioate
  modified_base 75..76: mod_base = OTHER; note = 2'-deoxycytidine-5'-O-monophosphorothioate
gaccctatcgatacagggcacggggtcgaactgttgggtttcgccatggtaccccctgcatttatatagccagacc

SEQ ID NO: 33 - DNA, 76 bp, synthetic construct
  misc_feature 1..76: note = Synthetic polynucleotide
  source 1..76: mol_type = other DNA
  modified_base 2: mod_base = OTHER; note = 2'-deoxyguanosine-5'-O-monophosphorothioate
  modified_base 3: mod_base = OTHER; note = 2'-deoxythymidine-5'-O-monophosphorothioate
  modified_base 4: mod_base = OTHER; note = 2'-deoxycytidine-5'-O-monophosphorothioate
  modified_base 74: mod_base = OTHER; note = 2'-deoxyguanosine-5'-O-monophosphorothioate
  modified_base 75: mod_base = OTHER; note = 2'-deoxythymidine-5'-O-monophosphorothioate
  modified_base 76: mod_base = OTHER; note = 2'-deoxycytidine-5'-O-monophosphorothioate
ggtctggctatataaatgcagggggtaccatggcgaaacccaacagttcgaccccgtgccctgtatcgatagggtc

SEQ ID NO: 34 - protein, 8 aa, synthetic construct
  REGION 1..8: note = Synthetic polypeptide
SIINFEKL

SEQ ID NO: 35 - DNA, 90 bp, synthetic construct; identical to SEQ ID NO: 11
  misc_feature 1..90: note = Synthetic polynucleotide
  source 1..90: mol_type = other DNA
  modified_base 1: mod_base = OTHER; note = 2'-deoxyadenosine-5'-monophosphoryl 5(6)-carboxyfluorescein phosphoramidite
  modified_base 2..4: mod_base = OTHER; note = 2'-deoxyadenosine-5'-O-monophosphorothioate
  modified_base 88..90: mod_base = OTHER; note = 2'-deoxyadenosine-5'-O-monophosphorothioate

SEQ ID NO: 36 - DNA, 90 bp, synthetic construct; identical to SEQ ID NO: 12
  misc_feature 1..90: note = Synthetic polynucleotide
  source 1..90: mol_type = other DNA
  modified_base 1: mod_base = OTHER; note = 2'-deoxythymidine-5'-monophosphoryl 5(6)-carboxyfluorescein phosphoramidite
  modified_base 2..4: mod_base = OTHER; note = 2'-deoxythymidine-5'-O-monophosphorothioate
  modified_base 88..90: mod_base = OTHER; note = 2'-deoxythymidine-5'-O-monophosphorothioate

SEQ ID NO: 37 - DNA, 76 bp, synthetic construct
  misc_feature 1..76: note = Synthetic polynucleotide
  source 1..76: mol_type = other DNA
  modified_base 2..3: mod_base = OTHER; note = 2'-deoxythymidine-5'-O-monophosphorothioate
  modified_base 4: mod_base = OTHER; note = 2'-deoxyadenosine-5'-O-monophosphorothioate
  modified_base 74..75: mod_base = OTHER; note = 2'-deoxythymidine-5'-O-monophosphorothioate
  modified_base 76: mod_base = OTHER; note = 2'-deoxyadenosine-5'-O-monophosphorothioate
attaattaattaattaattaattaattaattaattaattaattaattaattaattaattaattaattaattaatta

SEQ ID NO: 38 - DNA, 76 bp, synthetic construct
  misc_feature 1..76: note = Synthetic polynucleotide
  source 1..76: mol_type = other DNA
  modified_base 2..3: mod_base = OTHER; note = 2'-deoxyadenosine-5'-O-monophosphorothioate
  modified_base 4: mod_base = OTHER; note = 2'-deoxythymidine-5'-O-monophosphorothioate
  modified_base 74..75: mod_base = OTHER; note = 2'-deoxyadenosine-5'-O-monophosphorothioate
  modified_base 76: mod_base = OTHER; note = 2'-deoxythymidine-5'-O-monophosphorothioate
taattaattaattaattaattaattaattaattaattaattaattaattaattaattaattaattaattaattaat

SEQ ID NO: 39 - DNA, 76 bp, synthetic construct; identical to SEQ ID NO: 30
  misc_feature 1..76: note = Synthetic polynucleotide
  source 1..76: mol_type = other DNA
  modified_base 2: mod_base = OTHER; note = 2'-deoxycytidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxyguanosine-5'-O-monophosphorothioate
  modified_base 74: mod_base = OTHER; note = 2'-deoxycytidine-5'-O-monophosphorothioate
  modified_base 75: mod_base = OTHER; note = 2'-deoxythymidine-5'-O-monophosphorothioate
  modified_base 76: mod_base = OTHER; note = 2'-deoxyguanosine-5'-O-monophosphorothioate

SEQ ID NO: 40 - DNA, 76 bp, synthetic construct; identical to SEQ ID NO: 31
  misc_feature 1..76: note = Synthetic polynucleotide
  source 1..76: mol_type = other DNA
  modified_base 2: mod_base = OTHER; note = 2'-deoxyadenosine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxythymidine-5'-O-monophosphorothioate
  modified_base 74: mod_base = OTHER; note = 2'-deoxyadenosine-5'-O-monophosphorothioate
  modified_base 75: mod_base = OTHER; note = 2'-deoxyguanosine-5'-O-monophosphorothioate
  modified_base 76: mod_base = OTHER; note = 2'-deoxythymidine-5'-O-monophosphorothioate

SEQ ID NO: 41 - DNA, 76 bp, synthetic construct; identical to SEQ ID NO: 32
  misc_feature 1..76: note = Synthetic polynucleotide
  source 1..76: mol_type = other DNA
  modified_base 2: mod_base = OTHER; note = 2'-deoxyadenosine-5'-O-monophosphorothioate
  modified_base 3: mod_base = OTHER; note = 2'-deoxycytidine-5'-O-monophosphorothioate
  modified_base 4: mod_base = OTHER; note = 2'-deoxycytidine-5'-O-monophosphorothioate
  modified_base 74: mod_base = OTHER; note = 2'-deoxyadenosine-5'-O-monophosphorothioate
  modified_base 75: mod_base = OTHER; note = 2'-deoxycytidine-5'-O-monophosphorothioate
  modified_base 76: mod_base = OTHER; note = 2'-deoxycytidine-5'-O-monophosphorothioate

SEQ ID NO: 42 - DNA, 76 bp, synthetic construct; identical to SEQ ID NO: 33
  misc_feature 1..76: note = Synthetic polynucleotide
  source 1..76: mol_type = other DNA
  modified_base 2: mod_base = OTHER; note = 2'-deoxyguanosine-5'-O-monophosphorothioate
  modified_base 3: mod_base = OTHER; note = 2'-deoxythymidine-5'-O-monophosphorothioate
  modified_base 4: mod_base = OTHER; note = 2'-deoxycytidine-5'-O-monophosphorothioate
  modified_base 74: mod_base = OTHER; note = 2'-deoxyguanosine-5'-O-monophosphorothioate
  modified_base 75: mod_base = OTHER; note = 2'-deoxythymidine-5'-O-monophosphorothioate
  modified_base 76: mod_base = OTHER; note = 2'-deoxycytidine-5'-O-monophosphorothioate

SEQ ID NO: 43 - DNA, 90 bp, synthetic construct; identical to SEQ ID NO: 11
  misc_feature 1..90: note = Synthetic polynucleotide
  source 1..90: mol_type = other DNA
  modified_base 2: mod_base = OTHER; note = 2'-deoxyadenosine-5'-O-monophosphorothioate
  modified_base 3: mod_base = OTHER; note = 2'-deoxyadenosine-5'-O-monophosphorothioate
  modified_base 4: mod_base = OTHER; note = 2'-deoxyadenosine-5'-O-monophosphorothioate
  modified_base 88: mod_base = OTHER; note = 2'-deoxyadenosine-5'-O-monophosphorothioate
  modified_base 89: mod_base = OTHER; note = 2'-deoxyadenosine-5'-O-monophosphorothioate
  modified_base 90: mod_base = OTHER; note = 2'-deoxyadenosine-5'-O-monophosphorothioate

SEQ ID NO: 44 - DNA, 90 bp, synthetic construct; identical to SEQ ID NO: 12
  misc_feature 1..90: note = Synthetic polynucleotide
  source 1..90: mol_type = other DNA
  modified_base 2: mod_base = OTHER; note = 2'-deoxythymidine-5'-O-monophosphorothioate
  modified_base 3: mod_base = OTHER; note = 2'-deoxythymidine-5'-O-monophosphorothioate
  modified_base 4: mod_base = OTHER; note = 2'-deoxythymidine-5'-O-monophosphorothioate
  modified_base 88: mod_base = OTHER; note = 2'-deoxythymidine-5'-O-monophosphorothioate
  modified_base 89: mod_base = OTHER; note = 2'-deoxythymidine-5'-O-monophosphorothioate
  modified_base 90: mod_base = OTHER; note = 2'-deoxythymidine-5'-O-monophosphorothioate

SEQ ID NO: 45 - DNA, 90 bp, synthetic construct; identical to SEQ ID NO: 22
  misc_feature 1..90: note = Synthetic polynucleotide
  source 1..90: mol_type = other DNA
  modified_base 88: mod_base = OTHER; note = 2'-deoxycytidine-5'-O-monophosphorothioate
  modified_base 89: mod_base = OTHER; note = 2'-deoxyguanosine-5'-O-monophosphorothioate
  modified_base 90: mod_base = OTHER; note = 2'-deoxycytidine-5'-O-monophosphorothioate
  modified_base 2: mod_base = OTHER; note = 2'-deoxycytidine-5'-O-monophosphorothioate
  modified_base 3: mod_base = OTHER; note = 2'-deoxyguanosine-5'-O-monophosphorothioate
  modified_base 4: mod_base = OTHER; note = 2'-deoxycytidine-5'-O-monophosphorothioate

SEQ ID NO: 46 - DNA, 90 bp, synthetic construct
  misc_feature 1..90: note = Synthetic polynucleotide
  modified_base 2: mod_base = OTHER; note = 2'-deoxyguanosine-5'-O-monophosphorothioate
  source 1..90: mol_type = other DNA
  modified_base 3: mod_base = OTHER; note = 2'-deoxycytidine-5'-O-monophosphorothioate
  modified_base 4: mod_base = OTHER; note = 2'-deoxyguanosine-5'-O-monophosphorothioate
  modified_base 88: mod_base = OTHER; note = 2'-deoxyguanosine-5'-O-monophosphorothioate
  modified_base 89: mod_base = OTHER; note = 2'-deoxycytidine-5'-O-monophosphorothioate
  modified_base 90: mod_base = OTHER; note = 2'-deoxyguanosine-5'-O-monophosphorothioate
cgcgcgcgcgcgcgcgcgcgcgcgcgcgcgcgcgcgcgcgcgcgcgcgcgcgcgcgcgcgcgcgcgcgcgcgcgcgcgcgcgcgcgcgcg

SEQ ID NO: 47 - RNA, 1161 nt, Synthetic construct
  source 1..1161: mol_type = other RNA
atgggcagcatcggcgccgccagcatggagttctgcttcgacgtgttcaaggagctgaaggtgcaccacgccaacgagaacatcttctactgccccatcgccatcatgagcgccctggccatggtgtacctgggcgccaaggacagcacccggacccagatcaacaaggtggtgcggttcgacaagctgcccggcttcggcgacagcatcgaggcccagtgcggcaccagcgtgaacgtgcacagcagcctgcgggacatcctgaaccagatcaccaagcccaacgacgtgtacagcttcagcctggccagccggctgtacgccgaggagcggtaccccatcctgcccgagtacctgcagtgcgtgaaggagctgtaccggggcggcctggagcccatcaacttccagaccgccgccgaccaggcccgggagctgatcaacagctgggtggagagccagaccaacggcatcatccggaacgtgctgcagcccagcagcgtggacagccagaccgccatggtgctggtgaacgccatcgtgttcaagggcctgtgggagaagaccttcaaggacgaggacacccaggccatgcccttccgggtgaccgagcaggagagcaagcccgtgcagatgatgtaccagatcggcctgttccgggtggccagcatggccagcgagaagatgaagatcctggagctgcccttcgccagcggcaccatgagcatgctggtgctgctgcccgacgaggtgagcggcctggagcagctggagagcatcatcaacttcgagaagctgaccgagtggaccagcagcaacgtgatggaggagcggaagatcaaggtgtacctgccccggatgaagatggaggagaagtacaacctgaccagcgtgctgatggccatgggcatcaccgacgtgttcagcagcagcgccaacctgagcggcatcagcagcgccgagagcctgaagatcagccaggccgtgcacgccgcccacgccgagatcaacgaggccggccgggaggtggtgggcagcgccgaggccggcgtggacgccgccagcgtgagcgaggagttccgggccgaccaccccttcctgttctgcatcaagcacatcgccaccaacgccgtgctgttcttcggccggtgcgtgagcccctga

SEQ ID NO: 48 - RNA, 1161 nt, synthetic construct; identical to SEQ ID NO: 47
  source 1..1161: mol_type = other RNA
  modified_base 2: mod_base = mo5u
  modified_base 11: mod_base = mo5u
  modified_base 26: mod_base = mo5u
  modified_base 31..32: mod_base = mo5u
  modified_base 44: mod_base = mo5u
  modified_base 46..47: mod_base = mo5u
  modified_base 56: mod_base = mo5u
  modified_base 34: mod_base = mo5u
  modified_base 37..38: mod_base = mo5u
  modified_base 62: mod_base = mo5u
  modified_base 83: mod_base = mo5u
  modified_base 85..86: mod_base = mo5u
  modified_base 91: mod_base = mo5u
  modified_base 98: mod_base = mo5u
  modified_base 104: mod_base = mo5u
  modified_base 107: mod_base = mo5u
  modified_base 116: mod_base = mo5u
  modified_base 122: mod_base = mo5u
  modified_base 125: mod_base = mo5u
  modified_base 127: mod_base = mo5u
  modified_base 131: mod_base = mo5u
  modified_base 161: mod_base = mo5u
  modified_base 170: mod_base = mo5u
  modified_base 173: mod_base = mo5u
  modified_base 178..179: mod_base = mo5u
  modified_base 188: mod_base = mo5u
  modified_base 196..197: mod_base = mo5u
  modified_base 209: mod_base = mo5u
  modified_base 220: mod_base = mo5u
  modified_base 233: mod_base = mo5u
  modified_base 239: mod_base = mo5u
  modified_base 251: mod_base = mo5u
  modified_base 260: mod_base = mo5u
  modified_base 263: mod_base = mo5u
  modified_base 272: mod_base = mo5u
  modified_base 290: mod_base = mo5u
  modified_base 292: mod_base = mo5u
  modified_base 298..299: mod_base = mo5u
  modified_base 305: mod_base = mo5u
  modified_base 317: mod_base = mo5u
  modified_base 319: mod_base = mo5u
  modified_base 334: mod_base = mo5u
  modified_base 341: mod_base = mo5u
  modified_base 344: mod_base = mo5u
  modified_base 352: mod_base = mo5u
  modified_base 356: mod_base = mo5u
  modified_base 361: mod_base = mo5u
  modified_base 365: mod_base = mo5u
  modified_base 374: mod_base = mo5u
  modified_base 376: mod_base = mo5u
  modified_base 389: mod_base = mo5u
  modified_base 398: mod_base = mo5u
  modified_base 403..404: mod_base = mo5u
  modified_base 434: mod_base = mo5u
  modified_base 437: mod_base = mo5u
  modified_base 445: mod_base = mo5u
  modified_base 449: mod_base = mo5u
  modified_base 470: mod_base = mo5u
  modified_base 473: mod_base = mo5u
  modified_base 482: mod_base = mo5u
  modified_base 485: mod_base = mo5u
  modified_base 500: mod_base = mo5u
  modified_base 518: mod_base = mo5u
  modified_base 521: mod_base = mo5u
  modified_base 524: mod_base = mo5u
  modified_base 527: mod_base = mo5u
  modified_base 536: mod_base = mo5u
  modified_base 539: mod_base = mo5u
  modified_base 541..542: mod_base = mo5u
  modified_base 551: mod_base = mo5u
  modified_base 553: mod_base = mo5u
  modified_base 565..566: mod_base = mo5u
  modified_base 590: mod_base = mo5u
  modified_base 595..596: mod_base = mo5u
  modified_base 602: mod_base = mo5u
  modified_base 626: mod_base = mo5u
  modified_base 632: mod_base = mo5u
  modified_base 635: mod_base = mo5u
  modified_base 637: mod_base = mo5u
  modified_base 644: mod_base = mo5u
  modified_base 650: mod_base = mo5u
  modified_base 652..653: mod_base = mo5u
  modified_base 659: mod_base = mo5u
  modified_base 668: mod_base = mo5u
  modified_base 683: mod_base = mo5u
  modified_base 689: mod_base = mo5u
  modified_base 703..704: mod_base = mo5u
  modified_base 719: mod_base = mo5u
  modified_base 725: mod_base = mo5u
  modified_base 728: mod_base = mo5u
  modified_base 731: mod_base = mo5u
  modified_base 734: mod_base = mo5u
  modified_base 737: mod_base = mo5u
  modified_base 749: mod_base = mo5u
  modified_base 758: mod_base = mo5u
  modified_base 767: mod_base = mo5u
  modified_base 776: mod_base = mo5u
  modified_base 779: mod_base = mo5u
  modified_base 784..785: mod_base = mo5u
  modified_base 794: mod_base = mo5u
  modified_base 802: mod_base = mo5u
  modified_base 818: mod_base = mo5u
  modified_base 821: mod_base = mo5u
  modified_base 836: mod_base = mo5u
  modified_base 842: mod_base = mo5u
  modified_base 844: mod_base = mo5u
  modified_base 848: mod_base = mo5u
  modified_base 857: mod_base = mo5u
  modified_base 863: mod_base = mo5u
  modified_base 874: mod_base = mo5u
  modified_base 881: mod_base = mo5u
  modified_base 890: mod_base = mo5u
  modified_base 893: mod_base = mo5u
  modified_base 896: mod_base = mo5u
  modified_base 902: mod_base = mo5u
  modified_base 908: mod_base = mo5u
  modified_base 917: mod_base = mo5u
  modified_base 919..920: mod_base = mo5u
  modified_base 938: mod_base = mo5u
  modified_base 947: mod_base = mo5u
  modified_base 965: mod_base = mo5u
  modified_base 971: mod_base = mo5u
  modified_base 983: mod_base = mo5u
  modified_base 1004: mod_base = mo5u
  modified_base 1025: mod_base = mo5u
  modified_base 1028: mod_base = mo5u
  modified_base 1049: mod_base = mo5u
  modified_base 1064: mod_base = mo5u
  modified_base 1075..1076: mod_base = mo5u
  modified_base 1093..1094: mod_base = mo5u
  modified_base 1097: mod_base = mo5u
  modified_base 1099..1100: mod_base = mo5u
  modified_base 1102: mod_base = mo5u
  modified_base 1106: mod_base = mo5u
  modified_base 1115: mod_base = mo5u
  modified_base 1130: mod_base = mo5u
  modified_base 1133: mod_base = mo5u
  modified_base 1135..1136: mod_base = mo5u
  modified_base 1138..1139: mod_base = mo5u
  modified_base 1147: mod_base = mo5u
  modified_base 1147: mod_base = mo5u
  modified_base 1151: mod_base = mo5u
  modified_base 1159: mod_base = mo5u

SEQ ID NO: 49 - RNA, 118 nt, Synthetic construct
  source 1..118: mol_type = other RNA
gagctctctggctaactagagaacccactgcttactggcttatcgaaattaatacgactcactataaggcttgttctttttgcagaagctcagaataaacgctcaactttggctcgag

SEQ ID NO: 50 - RNA, 236 nt, Synthetic construct
  source 1..236: mol_type = other RNA
tctagaaaccagcctcaagaacacccgaatggagtctctaagctacataataccaacttacactttacaaaatgttgtcccccaaaatgtagccattcgtatctgctcctaataaaaagaaagtttcttcacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagctt

SEQ ID NO: 51 - RNA, 7019 nt, Synthetic construct
  source 1..7019: mol_type = other RNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgctcgaggccaccatggccgcttctggcctgttcagatgcctgcctgtgtcctgccctgaagatctgctggtggaagaactggtcgacggagccgccgccgccgccgccgaactgaaagacaaggaggaggaaaaagccgtgctggacggcctcctgagcctagaggaagagagcagaggcagactgcggcggggcccccctggcgagaaagcccctcctagaggcgagacccaccgggacagacagcggcgcgctgaggagaagcggaagcgaaaaaaggaaagggagaaggaagaagagaagcagatcgccgagtatctgaagcgcaaggaggaagaaaaagctagaagacggagaagagccgagaagaaggccgccgacgtggccagaagaaagcaggaggagcaggagcggcgggaaagaaagtggcggcagggcgccgagaaggccaagcagcacagcgctagaaaggaaaagatgcaggagctgggcatcgacggctataccagacagctggagggcgaggtcgaaagcctggaggccgagcggcggaagctgctgcaggagaaagaggacctgatgggagaggtgaactactggcagggcagactggaagccatgtggctgcagatggcccacttccccggatttggacagagcctgctgttcggctaccccgtgtacgtgttcggcgactgcgtgcagggagattggtgccctatctctggcggcttgtgcagcgcccggctgcatagacacgccctgctcgctacatgtccagaacaccagatcacctgggaccctattgatggaagagtgatcggctctgccctgcaatttctgatccccagactgccatctttccctactcagagaaccagcaagaccctcaaagtgttgaccccacctatcacccacacaacccctaatatcccccccagcttcctgcaggccatgagaaagtacagccctttcagaaacggctacatggaacctaccctgggccagcatctgcctacactgtcttttccagaccccggcctgagaccccaaaatctgtacaccctgtggggcggatctgtggtgtgcatgtacctgtaccagctgtcccctccaatcacatggcctctgcttcctcacgttatcttctgccaccctggacaactgggcgccttcctgacaaacgtgccctacaagagaatcgagaagctgctgtacaagatcagcctgaccacaggcgctctgatcattctgcctgaggactgtctgcccacaacactgtttcagcctgccagagccccggtgaccctgaccgcctggcagaacggcctgctgcctttccactccaccctgaccacccctggcctgatctggacattcaccgacggtacacccatgatctctggcccctgtcctaaggacggccaaccttccctggtgctgcaaagcagcagcttcatcttccacaagttccagaccaaggcctaccaccccagcttcctgctgagccacggcctgatccagtacagcagcttccacaacctgcacctgctgttcgaggagtacacgaacatccctgccgccgccgccgccgccgagaaagaggccgatgataacgaccacgagcctcagatcagccctggcggcctggaacccctgtctgaaaagcacttccgggaaaccgaggtgtgagctagctctagaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 52 - RNA, 7050 nt, Synthetic construct
  source 1..7050: mol_type = other RNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggcttgttctttttgcagaagctcagaataaacgctcaactttggctcgaggccaccatggccgcttctggcctgttcagatgcctgcctgtgtcctgccctgaagatctgctggtggaagaactggtcgacggagccgccgccgccgccgccgaactgaaagacaaggaggaggaaaaagccgtgctggacggcctcctgagcctagaggaagagagcagaggcagactgcggcggggcccccctggcgagaaagcccctcctagaggcgagacccaccgggacagacagcggcgcgctgaggagaagcggaagcgaaaaaaggaaagggagaaggaagaagagaagcagatcgccgagtatctgaagcgcaaggaggaagaaaaagctagaagacggagaagagccgagaagaaggccgccgacgtggccagaagaaagcaggaggagcaggagcggcgggaaagaaagtggcggcagggcgccgagaaggccaagcagcacagcgctagaaaggaaaagatgcaggagctgggcatcgacggctataccagacagctggagggcgaggtcgaaagcctggaggccgagcggcggaagctgctgcaggagaaagaggacctgatgggagaggtgaactactggcagggcagactggaagccatgtggctgcagatggcccacttccccggatttggacagagcctgctgttcggctaccccgtgtacgtgttcggcgactgcgtgcagggagattggtgccctatctctggcggcttgtgcagcgcccggctgcatagacacgccctgctcgctacatgtccagaacaccagatcacctgggaccctattgatggaagagtgatcggctctgccctgcaatttctgatccccagactgccatctttccctactcagagaaccagcaagaccctcaaagtgttgaccccacctatcacccacacaacccctaatatcccccccagcttcctgcaggccatgagaaagtacagccctttcagaaacggctacatggaacctaccctgggccagcatctgcctacactgtcttttccagaccccggcctgagaccccaaaatctgtacaccctgtggggcggatctgtggtgtgcatgtacctgtaccagctgtcccctccaatcacatggcctctgcttcctcacgttatcttctgccaccctggacaactgggcgccttcctgacaaacgtgccctacaagagaatcgagaagctgctgtacaagatcagcctgaccacaggcgctctgatcattctgcctgaggactgtctgcccacaacactgtttcagcctgccagagccccggtgaccctgaccgcctggcagaacggcctgctgcctttccactccaccctgaccacccctggcctgatctggacattcaccgacggtacacccatgatctctggcccctgtcctaaggacggccaaccttccctggtgctgcaaagcagcagcttcatcttccacaagttccagaccaaggcctaccaccccagcttcctgctgagccacggcctgatccagtacagcagcttccacaacctgcacctgctgttcgaggagtacacgaacatccctgccgccgccgccgccgccgagaaagaggccgatgataacgaccacgagcctcagatcagccctggcggcctggaacccctgtctgaaaagcacttccgggaaaccgaggtgtgagctagctctagaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 53 - RNA, 7050 nt, Synthetic construct; identical to SEQ ID NO: 52
  source 1..7050: mol_type = other RNA

SEQ ID NO: 54 - RNA, 7280 nt, Synthetic construct
  source 1..7280: mol_type = other RNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggcttgttctttttgcagaagctcagaataaacgctcaactttggctcgaggccaccatggccgcttctggcctgttcagatgcctgcctgtgtcctgccctgaagatctgctggtggaagaactggtcgacggagccgccgccgccgccgccgaactgaaagacaaggaggaggaaaaagccgtgctggacggcctcctgagcctagaggaagagagcagaggcagactgcggcggggcccccctggcgagaaagcccctcctagaggcgagacccaccgggacagacagcggcgcgctgaggagaagcggaagcgaaaaaaggaaagggagaaggaagaagagaagcagatcgccgagtatctgaagcgcaaggaggaagaaaaagctagaagacggagaagagccgagaagaaggccgccgacgtggccagaagaaagcaggaggagcaggagcggcgggaaagaaagtggcggcagggcgccgagaaggccaagcagcacagcgctagaaaggaaaagatgcaggagctgggcatcgacggctataccagacagctggagggcgaggtcgaaagcctggaggccgagcggcggaagctgctgcaggagaaagaggacctgatgggagaggtgaactactggcagggcagactggaagccatgtggctgcagatggcccacttccccggatttggacagagcctgctgttcggctaccccgtgtacgtgttcggcgactgcgtgcagggagattggtgccctatctctggcggcttgtgcagcgcccggctgcatagacacgccctgctcgctacatgtccagaacaccagatcacctgggaccctattgatggaagagtgatcggctctgccctgcaatttctgatccccagactgccatctttccctactcagagaaccagcaagaccctcaaagtgttgaccccacctatcacccacacaacccctaatatcccccccagcttcctgcaggccatgagaaagtacagccctttcagaaacggctacatggaacctaccctgggccagcatctgcctacactgtcttttccagaccccggcctgagaccccaaaatctgtacaccctgtggggcggatctgtggtgtgcatgtacctgtaccagctgtcccctccaatcacatggcctctgcttcctcacgttatcttctgccaccctggacaactgggcgccttcctgacaaacgtgccctacaagagaatcgagaagctgctgtacaagatcagcctgaccacaggcgctctgatcattctgcctgaggactgtctgcccacaacactgtttcagcctgccagagccccggtgaccctgaccgcctggcagaacggcctgctgcctttccactccaccctgaccacccctggcctgatctggacattcaccgacggtacacccatgatctctggcccctgtcctaaggacggccaaccttccctggtgctgcaaagcagcagcttcatcttccacaagttccagaccaaggcctaccaccccagcttcctgctgagccacggcctgatccagtacagcagcttccacaacctgcacctgctgttcgaggagtacacgaacatccctgccgccgccgccgccgccgagaaagaggccgatgataacgaccacgagcctcagatcagccctggcggcctggaacccctgtctgaaaagcacttccgggaaaccgaggtgtgagctagctctagaaaccagcctcaagaacacccgaatggagtctctaagctacataataccaacttacactttacaaaatgttgtcccccaaaatgtagccattcgtatctgctcctaataaaaagaaagtttcttcacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcttccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 55 - RNA, 3618 nt, Homo sapiens
  source 1..3618: mol_type = mRNA
actcctcatccttggagctttctccacagaagtgacttgacacaccgcgctacatctggggaggggggcgggtccgctttccgggagactcaagtctttgctatttgccttttgtttctgggatgcctttggctgcctttctcttccatctgcttttaattttggtaagagaaaagctaatgaatcaggaatgaatctcctctccactaatcttttttttttctttctctctctctctctcttaaagttggcgcgctctcttcacaagttccatcactacccaagaagtgagggggcgggggggacaggcgggaaggcattaattacacctaaatctgaagtttgcggcctcaagtaccactttgatggggagagcttctgaagcttccatacaacctgccgtgcctttgctgctgccgcagtagattttcagctcatttctttctgctgctgcttcgcgctgctctgtcatgcctaaatctatctcgtcactgccaaaggtgcctgaatcagggtgaattccacgagattcaccagcggttttgctccagtccaaggaggaggcgcgcagcccagctcccctagtctctcttcttaaatccccctacctacggctgccgaggttggccgcgcgtcgggaatctccccgacagtcctggccctcccccgccccccgggttggtttttcccagctgcggaggttgggaggttgggccaggggctggggtgcgaagagagtcggcgcccgcaacgcggagccgggaagtcgtcgctactctggtggaactcagagttggttctggaggcggcggacgcggaggtccctcagaggatccagcgaggggcgccaacaagaggcgaagaggtggcaccagggcggcggcaggaagaggagcgggagcaggagcgcggagcggagcgtcccgacccgccgtgcgtactttctggagggaaggggcgggggaatcggcccctgagggaagcgcccggtggcgagggggttagccaagttccggctgcggcgccactccctcggttccacgagaggaaagttttttttttccagacgcttccgccggctcgcgccctccgggcccagcctcccgagccttcggagcgggcgccgtcccagcccagctccggggaaacgcgagccgcgatgcctggggggtgctcccggggccccgccgccggggacgggcgtctgcggctggcgcgactagcgctggtactcctgggctgggtctcctcgtcttctcccacctcctcggcatcctccttctcctcctcggcgccgttcctggcttccgccgtgtccgcccagcccccgctgccggaccagtgccccgcgctgtgcgagtgctccgaggcagcgcgcacagtcaagtgcgttaaccgcaatctgaccgaggtgcccacggacctgcccgcctacgtgcgcaacctcttccttaccggcaaccagctggccgtgctccctgccggcgccttcgcccgccggccgccgctggcggagctggccgcgctcaacctcagcggcagccgcctggacgaggtgcgcgcgggcgccttcgagcatctgcccagcctgcgccagctcgacctcagccacaacccactggccgacctcagtcccttcgctttctcgggcagcaatgccagcgtctcggcccccagtccccttgtggaactgatcctgaaccacatcgtgccccctgaagatgagcggcagaaccggagcttcgagggcatggtggtggcggccctgctggcgggccgtgcactgcaggggctccgccgcttggagctggccagcaaccacttcctttacctgccgcgggatgtgctggcccaactgcccagcctcaggcacctggacttaagtaataattcgctggtgagcctgacctacgtgtccttccgcaacctgacacatctagaaagcctccacctggaggacaatgccctcaaggtccttcacaatggcaccctggctgagttgcaaggtctaccccacattagggttttcctggacaacaatccctgggtctgcgactgccacatggcagacatggtgacctggctcaaggaaacagaggtagtgcagggcaaagaccggctcacctgtgcatatccggaaaaaatgaggaatcgggtcctcttggaactcaacagtgctgacctggactgtgacccgattcttcccccatccctgcaaacctcttatgtcttcctgggtattgttttagccctgataggcgctattttcctcctggttttgtatttgaaccgcaaggggataaaaaagtggatgcataacatcagagatgcctgcagggatcacatggaagggtatcattacagatatgaaatcaatgcggaccccagattaacgaacctcagttctaactcggatgtctgagaaatattagaggacagaccaaggacaactctgcatgagatgtagacttaagctttatccctactaggcttgctccactttcatcctccactatagatacaacggactttgactaaaagcagtgaaggggatttgcttccttgttatgtaaagtttctcggtgtgttctgttaatgtaagacgatgaacagttgtgtatagtgttttaccctcttctttttcttggaactcctcaacacgtatggagggatttttcaggtttcagcatgaacatgggcttcttgctgtctgtctctctctcagtacagttcaaggtgtagcaagtgtacccacacagatagcattcaacaaaagctgcctcaactttttcgagaaaaatactttattcataaatatcagttttattctcatgtacctaagttgtggagaaaataattgcatcctataaactgcctgcagacgttagcaggctcttcaaaataactccatggtgcacaggagcacctgcatccaagagcatgcttacattttactgttctgcatattacaaaaaataacttgcaacttcataacttctttgacaaagtaaattacttttttgattgcagtttatatgaaaatgtactgatttttttttaataaactgcatcgagatccaaccgactgaattgttaaaaaaaaaaaaaataaagattcttaaaagaattacagtgtgtgcaagtttgctttgaaaaaaggatgaagggcaggagtattcaatggtcttggttccgatgataattattacttaacatagctgagaatcaaactgtagcaatgtctaatataaatagacttgggagatcgttttgaaatggtggatccttttagtatttaaatggagagtttgagtattcatgtgactgtatggtcccaaagttaatgctatttatgaaacagttttgtaagaacataatgaagttggtaaaagctaagcgttctgcacaataaacagttcccagtttggggctttttcaaggctaatgaagttattgttggtctagaaaaatagtatttacttaataaaactctgtttgaaattgttctgtggatttttagagcagaatttgaaacaaatgtaatgcaaataaatataaagataaagtgtcttaaagacaatattctgaaataaaagatataaaatataatttaaaaaa

SEQ ID NO: 56 - RNA, 2884 nt, Homo sapiens
  source 1..2884: mol_type = mRNA
acccggaggccgaccttccgccgaggcggtgcaggaaggaacgggagagggagaagtttggtagggaaggaattggggattaagcggtccctcagaggatccagcgaggggcgccaacaagaggcgaagaggtggcaccagggcggcggcaggaagaggagcgggagcaggagcgcggagcggagcgtcccgacccgccgtgcgtactttctggagggaaggggcgggggaatcggcccctgagggaagcgcccggtggcgagggggttagccaagttccggctgcggcgccactccctcggttccacgagaggaaagttttttttttccagacgcttccgccggctcgcgccctccgggcccagcctcccgagccttcggagcgggcgccgtcccagcccagctccggggaaacgcgagccgcgatgcctggggggtgctcccggggccccgccgccggggacgggcgtctgcggctggcgcgactagcgctggtactcctgggctgggtctcctcgtcttctcccacctcctcggcatcctccttctcctcctcggcgccgttcctggcttccgccgtgtccgcccagcccccgctgccggaccagtgccccgcgctgtgcgagtgctccgaggcagcgcgcacagtcaagtgcgttaaccgcaatctgaccgaggtgcccacggacctgcccgcctacgtgcgcaacctcttccttaccggcaaccagctggccgtgctccctgccggcgccttcgcccgccggccgccgctggcggagctggccgcgctcaacctcagcggcagccgcctggacgaggtgcgcgcgggcgccttcgagcatctgcccagcctgcgccagctcgacctcagccacaacccactggccgacctcagtcccttcgctttctcgggcagcaatgccagcgtctcggcccccagtccccttgtggaactgatcctgaaccacatcgtgccccctgaagatgagcggcagaaccggagcttcgagggcatggtggtggcggccctgctggcgggccgtgcactgcaggggctccgccgcttggagctggccagcaaccacttcctttacctgccgcgggatgtgctggcccaactgcccagcctcaggcacctggacttaagtaataattcgctggtgagcctgacctacgtgtccttccgcaacctgacacatctagaaagcctccacctggaggacaatgccctcaaggtccttcacaatggcaccctggctgagttgcaaggtctaccccacattagggttttcctggacaacaatccctgggtctgcgactgccacatggcagacatggtgacctggctcaaggaaacagaggtagtgcagggcaaagaccggctcacctgtgcatatccggaaaaaatgaggaatcgggtcctcttggaactcaacagtgctgacctggactgtgacccgattcttcccccatccctgcaaacctcttatgtcttcctgggtattgttttagccctgataggcgctattttcctcctggttttgtatttgaaccgcaaggggataaaaaagtggatgcataacatcagagatgcctgcagggatcacatggaagggtatcattacagatatgaaatcaatgcggaccccagattaacgaacctcagttctaactcggatgtctgagaaatattagaggacagaccaaggacaactctgcatgagatgtagacttaagctttatccctactaggcttgctccactttcatcctccactatagatacaacggactttgactaaaagcagtgaaggggatttgcttccttgttatgtaaagtttctcggtgtgttctgttaatgtaagacgatgaacagttgtgtatagtgttttaccctcttctttttcttggaactcctcaacacgtatggagggatttttcaggtttcagcatgaacatgggcttcttgctgtctgtctctctctcagtacagttcaaggtgtagcaagtgtacccacacagatagcattcaacaaaagctgcctcaactttttcgagaaaaatactttattcataaatatcagttttattctcatgtacctaagttgtggagaaaataattgcatcctataaactgcctgcagacgttagcaggctcttcaaaataactccatggtgcacaggagcacctgcatccaagagcatgcttacattttactgttctgcatattacaaaaaataacttgcaacttcataacttctttgacaaagtaaattacttttttgattgcagtttatatgaaaatgtactgatttttttttaataaactgcatcgagatccaaccgactgaattgttaaaaaaaaaaaaaataaagattcttaaaagaattacagtgtgtgcaagtttgctttgaaaaaaggatgaagggcaggagtattcaatggtcttggttccgatgataattattacttaacatagctgagaatcaaactgtagcaatgtctaatataaatagacttgggagatcgttttgaaatggtggatccttttagtatttaaatggagagtttgagtattcatgtgactgtatggtcccaaagttaatgctatttatgaaacagttttgtaagaacataatgaagttggtaaaagctaagcgttctgcacaataaacagttcccagtttggggctttttcaaggctaatgaagttattgttggtctagaaaaatagtatttacttaataaaactctgtttgaaattgttctgtggatttttagagcagaatttgaaacaaatgtaatgcaaataaatataaagataaagtgtcttaaagacaatattctgaaataaaagatataaaatataatttaaaaaa

SEQ ID NO: 57 - RNA, 2890 nt, Homo sapiens
  source 1..2890: mol_type = mRNA
gcgaagagagtcggcgcccgcaacgcggagccgggaagtcgtcgctactctggtggaactcagagttggttctggaggcggcggacgcggaggtccctcagaggatccagcgaggggcgccaacaagaggcgaagaggtggcaccagggcggcggcaggaagaggagcgggagcaggagcgcggagcggagcgtcccgacccgccgtgcgtactttctggagggaaggggcgggggaatcggcccctgagggaagcgcccggtggcgagggggttagccaagttccggctgcggcgccactccctcggttccacgagaggaaagttttttttttccagacgcttccgccggctcgcgccctccgggcccagcctcccgagccttcggagcgggcgccgtcccagcccagctccggggaaacgcgagccgcgatgcctggggggtgctcccggggccccgccgccggggacgggcgtctgcggctggcgcgactagcgctggtactcctgggctgggtctcctcgtcttctcccacctcctcggcatcctccttctcctcctcggcgccgttcctggcttccgccgtgtccgcccagcccccgctgccggaccagtgccccgcgctgtgcgagtgctccgaggcagcgcgcacagtcaagtgcgttaaccgcaatctgaccgaggtgcccacggacctgcccgcctacgtgcgcaacctcttccttaccggcaaccagctggccgtgctccctgccggcgccttcgcccgccggccgccgctggcggagctggccgcgctcaacctcagcggcagccgcctggacgaggtgcgcgcgggcgccttcgagcatctgcccagcctgcgccagctcgacctcagccacaacccactggccgacctcagtcccttcgctttctcgggcagcaatgccagcgtctcggcccccagtccccttgtggaactgatcctgaaccacatcgtgccccctgaagatgagcggcagaaccggagcttcgagggcatggtggtggcggccctgctggcgggccgtgcactgcaggggctccgccgcttggagctggccagcaaccacttcctttacctgccgcgggatgtgctggcccaactgcccagcctcaggcacctggacttaagtaataattcgctggtgagcctgacctacgtgtccttccgcaacctgacacatctagaaagcctccacctggaggacaatgccctcaaggtccttcacaatggcaccctggctgagttgcaaggtctaccccacattagggttttcctggacaacaatccctgggtctgcgactgccacatggcagacatggtgacctggctcaaggaaacagaggtagtgcagggcaaagaccggctcacctgtgcatatccggaaaaaatgaggaatcgggtcctcttggaactcaacagtgctgacctggactgtgacccgattcttcccccatccctgcaaacctcttatgtcttcctgggtattgttttagccctgataggcgctattttcctcctggttttgtatttgaaccgcaaggggataaaaaagtggatgcataacatcagagatgcctgcagggatcacatggaagggtatcattacagatatgaaatcaatgcggaccccagattaacgaacctcagttctaactcggatgtctgagaaatattagaggacagaccaaggacaactctgcatgagatgtagacttaagctttatccctactaggcttgctccactttcatcctccactatagatacaacggactttgactaaaagcagtgaaggggatttgcttccttgttatgtaaagtttctcggtgtgttctgttaatgtaagacgatgaacagttgtgtatagtgttttaccctcttctttttcttggaactcctcaacacgtatggagggatttttcaggtttcagcatgaacatgggcttcttgctgtctgtctctctctcagtacagttcaaggtgtagcaagtgtacccacacagatagcattcaacaaaagctgcctcaactttttcgagaaaaatactttattcataaatatcagttttattctcatgtacctaagttgtggagaaaataattgcatcctataaactgcctgcagacgttagcaggctcttcaaaataactccatggtgcacaggagcacctgcatccaagagcatgcttacattttactgttctgcatattacaaaaaataacttgcaacttcataacttctttgacaaagtaaattacttttttgattgcagtttatatgaaaatgtactgatttttttttaataaactgcatcgagatccaaccgactgaattgttaaaaaaaaaaaaaataaagattcttaaaagaattacagtgtgtgcaagtttgctttgaaaaaaggatgaagggcaggagtattcaatggtcttggttccgatgataattattacttaacatagctgagaatcaaactgtagcaatgtctaatataaatagacttgggagatcgttttgaaatggtggatccttttagtatttaaatggagagtttgagtattcatgtgactgtatggtcccaaagttaatgctatttatgaaacagttttgtaagaacataatgaagttggtaaaagctaagcgttctgcacaataaacagttcccagtttggggctttttcaaggctaatgaagttattgttggtctagaaaaatagtatttacttaataaaactctgtttgaaattgttctgtggatttttagagcagaatttgaaacaaatgtaatgcaaataaatataaagataaagtgtcttaaagacaatattctgaaataaaagatataaaatataatttaaaaaa

SEQ ID NO: 58 - RNA, 3501 nt, Homo sapiens
  source 1..3501: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcacacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatctgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 59 - RNA, 3475 nt, Homo sapiens
  source 1..3475: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcacacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatctgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 60 - RNA, 2248 nt, Homo sapiens
  source 1..2248: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactg

SEQ ID NO: 61 - RNA, 3498 nt, Homo sapiens
  source 1..3498: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatttgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 62 - RNA, 3246 nt, Homo sapiens
  source 1..3246: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatttgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 63 - RNA, 2967 nt, Homo sapiens
  source 1..2967: mol_type = mRNA
agagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaatacatttgaaaacattggttatattaccaagactttgactagaatgtcgtatttgaggatataaacccataggtaataaacccacaggtactacaaacaaagtctgaagtcagccttggtttggcttcctagtgtcaattaaacttctaaaagtttaatttgagattccttataaaaacttccagcaaagcaactttaaaaaagtctgtgtgggccgggcgcggtggctcacgcctgtaatcccagcactttgatccgccgaggcgggcggatcacgaggtcaggagatccagaccatcctggctaacacagtgaaaccccgtctctactaaaaatacaaaaaaagttagccgggcgtggtggtgggggcctgtagtcccagctactcaggaggctgaggcaggagaacggcatgaacccgggaggcagggcttgcagtgagccaagatcatgccgctgcactccagcctgggagacaaagtgagactccgtcaaaaaaaaaaaaaa

SEQ ID NO: 64 - RNA, 3498 nt, Homo sapiens; identical to SEQ ID NO: 61
  source 1..3498: mol_type = mRNA

SEQ ID NO: 65 - RNA, 3498 nt, Homo sapiens; identical to SEQ ID NO: 61
  source 1..3498: mol_type = mRNA

SEQ ID NO: 66 - RNA, 3246 nt, Homo sapiens; identical to SEQ ID NO: 62
  source 1..3246: mol_type = mRNA

SEQ ID NO: 67 - RNA, 9905 nt, Homo sapiens
  source 1..9905: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 68 - RNA, 9770 nt, Homo sapiens
  source 1..9770: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 69 - RNA, 9104 nt, Homo sapiens
  source 1..9104: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 70 - RNA, 3983 nt, Homo sapiens
  source 1..3983: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtgtgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccaga

SEQ ID NO: 71 - RNA, 3848 nt, Homo sapiens
  source 1..3848: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtgtgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccaga

SEQ ID NO: 72 - RNA, 2872 nt, Homo sapiens
  source 1..2872: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactgagcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaa

SEQ ID NO: 73 - RNA, 2254 nt, Homo sapiens
  source 1..2254: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtcctaataaatcttcactgtctgactttagtctcccactaaaactgcatttcctttctacaatttcaatttctccctttgcttcaaataaagtcctgacactattcatttga

SEQ ID NO: 74 - RNA, 1575 nt, Homo sapiens
  source 1..1575: mol_type = mRNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaa

SEQ ID NO: 75 - RNA, 10631 nt, Homo sapiens
  source 1..10631: mol_type = mRNA
actccttcatggaatctaaaaaattgtattcagagaagcagagagtggaatggtggttaccaggggctgggaaggtgtgagcttggggagatttggtgaaaggacatagaatctcagttagacaggaggaataagttaaagagatctattgcacatcatggtaactgtagttagtgacaatgtattgtatacatgaaaattgctaagagagtagattttaagtgttctcaccacaccaaaaaaaggtatgtgcagtaatacagtcattaattagcttgatgtagccattccacaatggatacatatatcaaaacatcatgttgtataccataaatatatactgtctctttatgtaaatttaaaaataagataaaataaatgttattcacttgtcgtggatgtggtggggacaggtgtgggatagccctccctgtacaactaggacccaggggtgatctagtgacactagccatttatcaggacgtatgggtgccagtcaggatgataaagcttccttttggccactatactacttagaaatgccctgcaaaaggtgcacatcaaagattgaaagctcaatcctggattttaagtgcttcaaaagtgcacttaattgccacatttttgtcaaacattttcccaggtagtatttttcctcatgtaaaacaacagcaatttaatttgaacagaaagcattttgaaacatacttttggcagggttccttgcagatcagaatggaaatgattaacagggcaattatcaatcatggacttttggcggcagaaggaactgtattgtttggtacagtctgggccagggccacacaccgtaacggagatactctattctgtggacggttggagggggctgtgctgagcagggtaactgcatcttttcctagactgttcacactgctgccacgaaggagtcttgtttagactggacctggctttcttcttcgcaatgagtgttgcagactcccgacaaaggccaggtggtaaagtgtggtgtctgtgagcgagagcctgagatgcctgagctgacctgtcctcagccacctgccatcgtgcagagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 76 - RNA, 48454 nt, Homo sapiens
  source 1..48454: mol_type = mRNA
agtttcgcctttagaaccacaattctctgatatgtgcaatgagagaattaattatagactcaaaggattgcatgcagacacacacagatacaaacacatacacacaacacacagagttacacacagacatgctcacaatacacagaaatacacacagacacacgcacacagcacacagagatacacacagacacacacacacacacacacagacatacgcacagatgggcacacacagagacacactcacagagacacacagatacacacaggcacacacacagagagacatacacacagcccacagggatacacacagacacacagagacatacctacaacacacagagatacacacagtcacacacagagagacatacatacaatacacagagatacacacagagacacagatacagacacagacagacatacacacagacacgggcacacacagagacacacagacacacacaggcacacacgtgcagataaggtaatattagctagttcaggaggagaaagagataaagataaagtaatattagctagttcaggaggagtgaaagaagccttgtttttctccactttttatagaagagaaagtgaagattcgatttgaggtgagttcagcacaaaagcgtatcccaggccctctggctccaactgcagccctttctacctcattcccagaccccacctaagccttttctcttcaaaatcttctcaggcacactgatacacatacctcagatttttaattctccggttgtgttcaccaggtgcttggtcatgattaagaattccgtgatgtgtaccccatgtgtttaaatttgctgctgagttaactttgtggcggcctgtggactagacctctgcacatgcaatgcagaacggcagggccagatttgaaatcctgctatcttttcggctgccttgtaaaaataacatcaggcgatggggatacgatgccagaggtcacctgtgataagttctgtttatggccattttacttctaggaagacaggaagtgtcaggatctcagggatctaggaagccaaaatgtttttccactctgaaataaagtgactgaccaggagttcccggccacgcagccctgtgggaactgccgcacggccacttttatgaagtggacacgtgttggtcccactgaaaagaaactccccacccatggctccctcacgctgcagcagaggccctgccacagcacctgtcagcccctgccagcttgcaggggcgcaggcgcagagcggtttgtgcccttgctggagccagggaagggcacagggtccctcctggagtcatgggaggtgcagccgaggttctatattaaaatacagaggctagcacatgtgcttggggaatgcagctacagtagtggaatgaaagtgctgtccgttccttacccccccagctcctcacctgtcctccacacgcatatccctggctccctttccctagtaaggagactgaattgaaattgtggcttgcccgaggctgcatacctgtgctctttctgaagcccaagtcactggctctagaattctaacctgtgaggaagccactgaggatgtttgtcaaaatacatatttctgtgccttgccccagttccacggcccaggaatctgcagttttcacaagcacccccaggtgattctggtggtgtctttgcacttcttcaaggcagtactgcctggaacgcagaatcccagcctcctctatcctccttgcctaatggcctggatgctctcagatctacaggggaagggaaggtcacacagtcatcgcaatagtaacctcagctgataaatcctcccccataaaacttattccccagtgttttttaataggaaacaataaaactgtaaccagcccaaatatccatcaaagagaaaatggagaagtaaatcatcgcacattcacctggaccagatctattgtaaagccaataatactgaagccccttccaaggccctgggagtcctaacagtgcactggcagtgtctataatttatattatgaaatttgcataaggaaaacattttgtctcatttgtgcaatttctccttctaaatatacgtgtcactttgtacctgatttctataagacccaggacctacaaaccctgtgtctgcccctgcagccacccagggaaggactgcacagcagcaagacagattgccatggagcatgttgtgcccaactagggacagcgcagatagattctgtaatttgcctaacaatgtctataggatgatcccatttgtcaaaaaaaaaaaagaactgggctttattgatgtcacctaaatgcacctaaacttcttttttgccccatgctcttctgtactcttgatctttccccaaatttttaaaaacatgacactcattcccttatttttcctacttagaaaagtgtagatggttttatcataggaagttcaaaaaaattaaaatataatgaaaaatactcaaatagtgcctcacaacagtaactactgctaacataaataaaatccatatttcctctcatacagaccccagagttgctttgcctgacagtgtagttgatggagaaaataatctttatccttagcctccatctggttgcagaccataaagacagggaaaaaatgagggtgttggtagcttcgttagaaactgaaagctcactgattttttcaaaacctaaatagcctgtgtttctccaaataactaatttgcagccttcggcagccaggactggcagggatggggctagggggactggggagaactgctctctcctgagggtggtctgacccgacagcacgcatgaccttcccacagtcaggaactgctcagagacgtgatggcaactccatagaatgaaatactcttcagccagtaaaatgtatttttggataaatatttgctttaaaaaactttactatatgttgttaaatgaaaaaaaaaccttaaggcatcagaaattatgtgcagtaaaatctcacttttgtaaataaatatacctgtttactacgtatgcataaaaagaatcctgagaaatataagtactgtatgcatattgttgttaagtattttttctgtttgcttatctataattctaattttgcttcaaagaacaagttactccggcaatataaaaataaaataactaatttgtcttgtcatcaaacagatagtaagaacaggcaaacctggccctccacactgccagccttttgtgattcaaggcttcagtttcctccacttgttaaaaagattcaacaaagtagttgaaatagtatgtgaaccagtaaaccctaaaaggtgtccagtgttgtctgtgagctaattaagtgatttgattctgactccccgagtcttctgatttcgaagcagtggggagtcagacaggagcctcaggtggcctctcctgagaggccctggaaagtgatgagaacctggcctctggcagctcttcataaacgtccatgttttccctctactctctcactcttttcccagggcctcaaacagaagatgaaaatcaatttctaaaacagccctctgtgtgctctctcgtatctctccttttcacacatcgtggtggtggctttctctgtgttcctctgttgattcagtctctggaattaacggatcaggattccatgcccagaatgctacaaagactgtgcttgagttctcccacatctcactcaattacacagaagtttcagattatgtaacagatgctgtgctgggttaggcagagccatctgacttgttttgctttattttagaccatgagatgggtgagtttttctttttaatgccacattcttttaagaattaaaaacctccacttggctgtcagcattggaaatcagagtgatggtgcaagccctgatgaggacaatgtccttgtctatgaaaaggtgaaatcattgcttgaaatcgctaagcaggacatgcagtcccagatggaggggggaattcgggagctggttggaaaagagtatttggcactttgcagccttgagaggtgcagaagagacaccgaggggttcaccaccagagccaccattgtcagagaggcgtccagctgtgtccacctgggactctgccttcagggcttcttgcctggctgggagctgcacaggcagactcctgggacggtgtgccgacagctctgggcacccccttctaggatctgattcctgaggaatcacaatgtggatttcacaatcacttccagtgtcttttgccaacctctgtgaacagatgtgcaattaaaaaaaaaaaaagaaaggggcccaattctcaacactgtaagtggaaactttttaatggaaaaggataggctaatgaattgaatttgaaatctgagacagaaccgatgcatcaaatgtgctggtgtttacagataatacaaggggggctgcatcttatggtttcaatccttttttaaatttttgttctgagagacccagccagcagactgccgccagtcttgtcagagatgtcagtggtggccactctgaatggaaagcagcatctctcagcatctctgaggcactgctcctcagcggagactgtggtggctttgcctttcagcacgcatcctttctacgatgcctgacagtgcccagggaatgggcagagctgggagctctgaagccctttcacctaaaccaccctgggtcacctgacctagttttcctcccaattttaattatgtcaggcacttcacaaaggcctccttggggacaccatgagctcactgtcatcagattgctccaatcacagctgtggcttgcacacaaccgccatctctgccccagcagatgctgtgtgtaaacagttgtattaattacatctcaaaaacatggttcttgccagatcctcaggatttgggtgcagcctctgaggtgggtgggaggccctcgagggagaaatgtctgcaggaaattcttcccctacgagaggtctgttttctaagttatctaagagctactgcagctgtttactgcagagtgaccctgctcaaagctgtggtcacccaaggctttgaaaggggacctccacttccgccctgggtggagcaccgtgctggagacccacgcctgccaaggcctcattgtcatctccacacgccgtccttggggtgggccactcctgggacacgcagacaggaagccggccacctgagccactcggaggctctatccagagtcagctgccaagcctcacgtcacacatcactgttagtcttggagggctggcggggccctgaagtcaattgaacacttggatgacagggaacttgccactgccagaggcaatatgctccatttttttgacagttccaacaatttttctttaaactgtcataaaaaattgctgctgtgaataccagtgtcggcgtccctgcctcacctttacctggtgcttttccaccacacaaaactgtttctcctcgtgctggccttgggcttgcagacagctgattcttctcctcccgcggctgagcagcctcctccgagcaaccctctgacaactctgctccttctgacaacctctgcaagggctgccagatgtgaacaaggggcccgggcagaaggtatccaggaagactggaaactcgaggaagcctgccctgtcctgtccaccagactttacgcttgcgtcactgggctttgggacctaagtcctcgtcatttgttccttttgcagttcctactgttctcagcacttccttccagcttactgaggtacactcagatgtgatatgccatcggtacagacacagttctgctccagcatttccccgtgttctttctgtcgctctatttactgaattaccgtgaggatgtggagcgaggctgagttctgtattttaacaccattttaattctcacctactgagaaatccatcctcttatcactgtgctttttttaacctgtcacgaatccatgaaatcctatcagccagcctgcatacttccttttaaggtgcagttgaatcaggagaaacttgccgcacatgctgcgtccgggcacagcattggctgaggctgctgccctgacctgtccgctttgtagtactgcccagctatgaaacaggttagccacacatgacctgcatttaggagtaacaagtctgtctgtacatgcacatacagcaacttttttaaactgtctatattttttcctgagataggtatttataatatctccatcttctttcccattttgaaacttagaacaagtttgcctgtcaacagttctccacagcatactgtgtattctaggattttctaaggttgagcaacggaggttcagcaattttgacttaatttcttcccatcccttttccacgcagcccagaagccttggatcacgtggtgaggggaagaggttgtgctatgtcgggaaactctgtatcgaagctcggctcagatcatgacattctcttgactaaaaccctcagtttccatcaaacttgtcactctggcattaaagcctgtcactgtgtggctctgaaaacctctctgaacgtgttccctgcctctgccctgcaggtccctgtgctccacagaagcccacttatgtgacccacccccactcatcaccaccttccctcacccagagcctcagctccccactcccacctgtaagacccctactggaaagattcccacctgcccctcaagattaatctccaaggacatttccaaattcctctccccatctctcagccagatggctttgctccctccaggaaccccagccaccttcgacctccagcagggcactccactccacattctcctggtctgtctggctcatcttacctgagccatgctctccaggtgaaggactatgtctaactcaactctgctttaaaagcagctaacacattgctctttgcatattgttcactcactaagttgaactggacttggacatgcacactgaactgcagcgtctgctgcttcttggtggcccagctcgtcaaaagaataagatttcagcaaaacaatgtaacaattttttttaccaaaagtaatgttaacaatatatggttttcccctgatgtttgcgtcaaaatgctttttggaaaaaacatttttcaactctttagggtcagaattaagcaatgaaatttatataccacatgtataatgtgtatgtttatctaagtatctgttcatttatatatcttaaatagaaattttaaaaatttttttaaaactcctgataaacattctcaggaggcacactatgtaactgttggttgatatacctagctagatggtgaaatcagattttgtttaaagcatggaggagagggaaaaattaaatcttgcagattctgcagtccttaacatctttgaaagaggaacatttcagacaatgtaataagaaggccacgtgctttgacttctgtagattttaaaaatacttctgtatagtttcttcttcctttgaagaagtttggggagtttgggaagatggagaaagatataagaatagactccccatatgggtcatgaattatctttttgcatcagaactcttagtgcagtttcagtattttcttcctcaggagggtgagctgcttccgaatgtcctccccttctttgaggcatcctctgttggtgaactttgagagcatccatttatgaagttgatgacctttcccagtctctgcaagcccttcagtgtgtgtcctctctgagcaaatctgaattgtgtgcttaatacatggaaagggatttgggagggttgctttttaaactgatttcttaattaatattatggtttagttaactagacagtctcattgcagaagtgcataaccataatatgtcttcaaatatatctcccttcctaacaccctgtaatatacttttgtaaagatacccttacagaatgtgatccaccatttatgaacctgcagcattgcattcagagactaagtgaaaagctggcagattttcatttaaagcacaagctaaggaagaaagctggtctagaaggagctacagaagggtaatgcttagggagggaatgatgtgcctgtgggtggtggtagttaaatctaaccaaagaatgatgtcgtgggtgtttggatattggatggtccacattgggccacattctttcaaacataagagtctgtagaaatatgacctgtaaaagactcttaaatattctggaaactgtttcttccttgtcacatccttatatatacttgaacctatgcctaccagacatgacatgtgactattcatacagatttcatcatctctggtttaagaataaaggatgctgcatagaaggctcacatcttttaattcacaagactgaaactgttctgaaatgacattgtttctaaaaattcattacttgcattatattcatttttatttttccatgccagaagggtagaagttcctgtgctcatattaagaaacagcaatgtcaatcgaggcccaactcaaatccaatttataggagttataaagggcgtgtgcctgttttgtctagaagcagtgttgggcagcactgagtaggatagaccacctgttgctaccgataaaggagcagcttctcgaatgctcctgtctggtaggcactatcccgagtgctttggcccctcatccacaatctgtgtggcaaaaggcattgcaggcaattcagtgaggagaccgaggcatggagagcaagtgccatggaattccctaaggccgtgcagggagcaggttgccaagctgggttgaaaccgtcctccgtaggctcccaactccgccgtcgctgctactgtgctggatgatgcctggtagatgcagatgtggagccccatggattctgagacaggccgggtttcagtcctgccctagctgcctattggctggatgaccttggcaagttgactttcgtgagcctcatttgtctcatctctcaattaagaaaacctagagcctatctgtgggggttatctgaaggattccagggatgcatatggcactgtctaccgcatgcggtaactgtttcacaaatgatgaggagcgatttatgttcttagtggaaatatgtcggcgtgtgaagtcccaaagctctgccctgcctggcttgatccagtgcctaggcactgcccctcttcccctctctcccaacccactgtaagaggctaggctgcctcagtaactctgaggggcattgactcttttcatccaaaaattcatgttactgccccacattttttctgttgttttacaacgcagtaggaagtgggcagactgtcaggaaaagtgatttatagtcatgtattgcttgtgctttggcttcatttgatccaatgcagatcagctgcactcagaaaactactcaagtgaaagagaaaaagtaactgaagggggaaatctggatgagtaagaattccagggataggaatattaatagcaagctttttgcctgatatagtcactttatgctgcaggggtgcccctttataaagtgcttgtacaatggatgtttgcttttgattttggatttggagtctaatgaatgttctaaattattattagaggagcttgcggttgttacatgtctgcctttattgcttatttttagccatctcccctgatgtcaaatgctcaggcaagaatgatacattcatttataatgtggctccttcagaaatataccacataccttttggtgtggtttgtggctgagaagagtggggaatgcacaagtggaaaactgcagaaagattatgccttcatcacttcaagtatttgagatgaaactagatcatttgctgttgctttttattctcattctaagtgcttttcaaagtcagcgctaagattttaaaatggttttctgttgttggcagagagggaattactctattactttctgataaaacagagtctttcatgatcaaagagaaccaggctctagtagttccagtatcctaacgtggacactaattgtttccctccttttcttcatgaaaacagcttctgcacaaatgatagccttgtgaactagccatgggcacaactggagaagcatttagggagctttagtgcaaattgagaccacctacacatctgactctacagggtttgacaacatccagggtgaatcacaaaacatcagtctaatcagggcttatatagaaagagtgaaagaactctgatttcatcctaaagattatttatattaaccattgttccaaatgcattaactattttaatttagttgttttgattgttaaaaaaaacacatctgtttggtagataagacataatttaagacaaatgttctatttgataagcttttagaaacaacttatttttattctttcctgtgagataactcagatgtggagaatgtgacaaaattttaagcataacatgagaagggctgacacacatagatttctgtgtgcttacttgaaaacaacaaaatttaagaatttggtataggagttgtatcaggtagtgcagagtccccaggagacctagagacccaggtctgggagcctagcggcaagggctgaatgtgggatgacatcagcagaaactcacagccactgctattccaaaaacccagcagcagctcagtgcagggcagtgctgatagtacagtgcctgcaatcctggagtggatttggatgtgtcaggtacgcacacgctcactgctcccccagcagtacgttgaacagtgtgcgtccaggtgtctgtagggcccctcgccctaactcacaaaaccattctgggtcagaagccaccaatattgtcatcatcctcccttttctgagaaccctagtaagtccctccagtggggcaagcccaccttttcccttcattctgtggcaatatgccttcatttcctaatcagttttgccctgctcattcaatgcaaaatggatctgctttccttgggcaccaatatgtccagggattgtttatcaatcttcagttctgtttcctttacatatccctccaaaaatcaggcctgcactgcctgtgcactccacaatccacaggcctgaaggaaatgttatctttgatgtagagacttaaagtaaaactcttcaaattaattatttcatgcaaaaggctagtcctgactctaattctaagacatgtctcctaaactctggaagtctgatgtatcctattatcaacatttatccttaatgtgatggtttatcatttatcctcaaagctgcattgtaaaatgtacactgtaaagtgtacattttaaagtcggttttaaaaaatcatatttagagatcctggtaaaaatctatcaagtcaagacattaccttattacccatggaattgtcttcaactcttacagttcaaatattcctgaattggctttcacaataaacatcctaaatatgtaagtagaaacatatatattgccaactttgtgccttcccaagcaaaattaaaatacaggaaaagtcagtttgttttgcccataaataaatatatgtgtgtgtgtatgtgtgtgtatacacatacacactcagaaaagatagaagcagcagcatattttggcagcatctggtttattggaactcaaacgttctgattgtgcatacagactagttaatgtggtaacaattatgtatttcttccctgctccttgccttctttccctccccagtttttttcttcctgatagtaggtgtgtacttttttcctatttccattggcaagccacatgacaagcaaaacgatcactcgaagaatattgttccctcaatcaagaaaaatgcccattgggttttgttatttgatgttatttgatgacagagacctattgtttttccatttttctttttttgttttccgtggcacctatggaattaagcaatataaaaaatctattatttcagatgttcacgtctaatgaatttcatgtgaaatactggcagtataaccccaaatagaggaaatttgtgaagagtggatgctgcagggcatgagacatctgcacagagttcatctcttccagcatcttgcatgtcccaagcactgccctgccaggcagagaatgctgcagatcacggcagtgaattccagttgttcagagcacatttgacttccaaattctcaaggccacagatttgaggacagaacaatatttgcatttgaaattggaagattattttttgcacaagtgcctatatgctatatagagtttgcccactctgcattatcttccccctgttcccccgttatctggcacaagctattcaaaagacacgcctacttgtaaaataaatggtttgcaaactaaggaaaatacttaaatctcatgtaaatggtactatactatgtataaaaatgtgaagaaacacagaacagctcatgaacacctccactgctgtataaaagaaccatcttttttctggctcctattggatgccttagaaaaatctgtatttcctctttagttattgtgtttgaaagatgaagttgagacaaaagttctattctttttaagttggcagaacttctgaaaggtgatttttagctgcagtgtgactcattccaaatgcagaaatctctgaccctgagttagtctatttgtcatgcaagagcctagaaaagccctgagtgataagaaatggccataggccattcccacagaattttcaacaaaaatagaatcatgcttatgttctagtcatgacttagaacttataactcatgttcggaactgtccatgttcacgcacaggggccgtatcactccgccagagctgccctgggtgccggtgtgcagaggggtccgagagtgactgtctcttcctctgttgtcgaatgtgtgggttatctccataaatggctgccatgagcatccttgttcacacatttttaggtacttgagtgagtgtctgtggaataattttgggaagtgaaatctgtggtcagaggtttgtgagttttacatgctacattttcagaagttgagaaatagcagtaggctgaaggcaagtcgccatgcctggaattcatgaacactagttgaaagaactggcgtgagttagtcatgacaggagagatggggaagggagttgcaggtaggagggccatcttcaaattctcaaagtatagtcactccaaaccaaaattcgatttaatctgtaggactccattctcaaagcacagtcactccaaaccgaaattcgatttaatctgtaggactccaggtggcagaataagaggcaatggatgggtggaagcgaaacagggccaaagtttgacttcatgtgcaacttcctaaggagtgatttgaactccacaaacatgaactaagcacctcaacacaggctgggcaagttgctgttcttttggagcttacatcttagtggggaaagagaaatgcctatgtaaacatataaatcagcaggatacattgtgaggacggtcattgctcagtgagactgcaatagagtgatacgctggagggggctgcaagggagaaggtgggagggacagcatttagcagaatgagcagcacagtcccataggaagaagaatttattgcctccttaggcaaataaattcccaaaccttgaacatcagaaaggaaatagattaatgtgcacagaggattaaattatgtgatctgcaaagtcatttaaaatctatttccacataaaacatattaatgcaacctaaacaaaaggggtctggataccctcatcttcttcccaagcatcaagtctttctatagttaaactgagatgcttttattcttggaaaattttaaggactatctacagcaatggaagaatcgggtgttgggatgtgttcccaggtaataatgactgcaggctgatttggcccttgaggtgtggcctcatggccctctccaaaaaaaatcaaggacctgctacaaagcacaaagccgactgcaatgcttgctgcttactggttagggcagctcctctttgccagcgaccaagcagaaagcaagacaagacaggttctgaagcagtaattcaaagccttcctcgctttcccatgtgagtcattgctagtcagaatattacctttgcagagaggcttaattccaaatttgctcttaaagggatatcctctcctggtttaggtataaacttttgactcacaggacaaattctatcattcctttgggcctaggattgcatttatttccatgacaaaagggcctgtctggtgtttcagcaaatgaaaacaaaaatataaagcccatctccttttgaatgagctctaaaacagttctccactggacttcagaacaagagggagctctgggctgctggctggttgtgcatttgctgtgggttccctccggcaggcgacctctccgcgctgagaaggttatccggataaccaagtaagaaagtacatgaggaggcacagaaagaaaaatgtgagagataacagcataaacacacagtgtatgttgttatgaggcatcacatgatgagatactgctggggagggaagaagtgaggagattcctaggaatcttatgagaatttccagagacaacaagttttgagcttttttttaatttagaaaatttaccttatttttaaaagaatatgtaacatatcccatgctataaaattctagacatagtagatttaaaacagcataatggaaaatataaatatctattttcttttcctatttatgtattctgtgccagtaggaatgtagccaaaaagagagaaaaggggtctctgcagacatggatgtctctgtgacttgatcactgctaacccaagaagataataaagcagaagcatgtatccaggttgctgcagccaagcctgcccggtctgcggggcgtcctcacacatggggcagctctcccaccccacacactgggaaaggcggacagaggctgggcaaagcccccaattttcgttggcactgaccccgatgatttataggcctttgtttcccatgttaaatgtcttacgatcattaaattatttatagctcaattagcatgtgtccaaaaccaggaagttcataggagactgtgtgactgggaattaaggagcaaagcaactttccagtctgtgatttactgggtttccattctgtttcctgttcggatccggaagtagaatttcaaatattgcttttcatgctttatttgggaccgattttagccccgctctcctttctcttgccattcgctggccattagccaccagcctctgcacaatgaccagctggcccctggcagatcttgggcccaggtgtgaagtcgctggagaagcatttcagggccaagatgggagtgatttcattttccattgacactatgcagaaatgaaggggattcaagtgccttcagaaaagcttccttccagcgaatggagttttgggggttttccagacttgcaactgcttttattcttggaagcatcattgttgctttttccccccttccatttatatcccaggaactgattcagaaaccatagaaattggatttggaatcgctgaatgctagcagacagctgactgcactcttcccaagaaaccctgccagctgggttcgggtatcgcgcggtgtgtgctctctctgcctggcccgctgagtcctctaactctaatggattccttcttacaccaaagtgcactagaactaaagtgttttgcttcattctttagacattttgtggtttagggctcaatcagccagggtatgatttgcaatccacagtaaccggtttcagagcagctgcccagcgaggcaggtttcatctcgcttgctagacgttttgtttttttttttttctaaacctcacaccttttatttattagacttggattccagtttcctgagcctgtttgtgccactgattagacaggcttgaagcagaacccaccaggcttcctgaataaaatgcagcagtgattgtattagggggttttaaattgctcaaaatactgtctaaaaaacactaaaaatcatgttactttctagattgaataaaatcctatagaaatgaattcctggacttgatatgtagcaagctggcattggctcgggagtgagtgggctcagttaagtgagctaagatgagatggtgcacaggcgagcacccacctgaggagtgtttggatgttatgatagccagctcctctgtaaagacctgtccttctatgtcagcagcccagcagataaatgacgtgtaaataccacatttaggagggcttatgatgatgccaattaatggagacctttttgaaacaggaaggaggtgaaacatattcctttgcttctacatcactgtgtgccaggcactgtttacagcatctcgtttaaccagcagtcaccacctgacggatggctgatgtggggtggggtcccagggtgggattgcgtgatgggcttggggtctctggctgatgggtgccagagctgggactggaactcctggcgtgactgaggcagacacctgggctacccagcctcacccacgacgccctcactaagtgacccacaggactcaccggaagcagggcagcaaggtccccctacagaggtccccactgcaaaccgatacccagcttagacagcagttctgcagtcggcgtctcaccccttcgggtctcattgtgactcactttgatagccacacgatttaagggtggttcagtagtgatttgatgagtgctgtggctcagggtcattcccctgcccaagcatttcaaattccagaagttcatgccctgcatggtgggtgaaaagtctcaggccaaccatgagcacacagcagccaggcgactgaggcagctgcccggggtggcacgttgctcaaacccatcatttggagtcaaaacaaacagatgattagctggggtggtcactttcaatcaagagttttcacatcgcctagacatggcctcagaatcaggcctggtgtggccaggggctgatctcacagtagacaggaagtgtggcccgagggccatggctgccccctcagaaggccctgtggagtggctggccgagcctcagcagcctcctgtgaagcgaggaagggtcttcctgccggcctctggagatcagtatgggaatgcacaagtaggaaacgctggatgggaatccctctgccctgtgataccaaggcagtgagtttgtagactatggaattgctgtcggagggctctgtaaccggccaaggtcacacaggtagccattggtagagcagggactggaatcccagacccccaacttccaggactgtgcacctttctttatcccatacagccttacagtcaagtgccagtgcaacacctgattcccaggttccagcctttgtcttttataatgggaatcaaccttatcttgacgatccagagatagtcatcaaggaagattaaattatccccttagactcagagtgaccatatcattttccctccacacaaggacacttttgagaatgaaaaggaggagatgtctgtaccagacgctggatgacaggcaccgacaggctgtctgccaggggagcagcgattcctgtatgttgtagaaagtttttcaaaagtcaccttggaaagaggttttgttccttaaccttctgttaaataggaagctccgtgaatgaaaacaactcccttccctaaacattctagtaatgacccaacactgccaagcctgccagctctgcctcatggtcgtgttgactgtgtgagactatgtgagtgcctgctacacagtacgctttcagtaaacatggtattgcctcgataatcccacaaaaatgtcctattcaaatcacctggcacccaggaaatttccttcttttttttcccaggtgaaatatacagttgaaaacacctgacagcaattcccctctcccatgtgtttgcaggatggtggttttggttcctccatctttgatgtgtacaagtgtgatgttttccccccacagacaagtaaaccacattctcttcacattcccaatgttttgtcaatgtacctccttcaatagaggatcgataaggaaaaaaatcattgacaatctcaattagattcactatttcatccaaaagcatagcttagaactctagtttttgttcaacactcttgccctatgagtgcacagaactttaattctgatacaaacatccctgaatgtttagctttgacagagattccaaggtgatttgataagaagcagggctgtgtttgggctctgggagtttttgatatggtttcaagccccatccaaaacccacagacctctagaaagtaggtgcctgccttcctgcagcagccctggagcctgctgggggctttgagcagctgctgccaagccaggcctcacccgacactctgatgggcacggccatggtggcaggggcttggacgctgccaggtgactctaacttgtggccagggtgggaagcactgctccacagaggtgccaaaaccaggttccttcctgtgttctcacatttcacagcctcaatgtaaaaagtaagacatgggcactctggaatattacaaaaatatagaaaagcatgttatagtaaataaaaggctcacagaattttgtcatttaggaacaatgattattaatatattagtgtgtgtttttgctcattaacagtatatcctgagatatttcctataccatttaatattttaaaagatgtttacactggccacagtagctcatacctataatcccaacactttagagggcaaggcaggaggatcacttgaggcttaaaaattagccaggtgtagtggcacatgcctgtagtcccagctactcaggaagctgaggctggaggatcacttgagcccaggagttcaaggctgcagtgagctataattgcaccattgcactccagcctaggtgacacagtgagaccctgtttctaaaataaataataaataaattaaaacatttaaaaatacatgatgtttaattattagaggactcaattttatatctatgtatacaataatttttaagtttcttaatattggacttttagtacctttttaaaaatactatttttaaaaaaatctgtatttctaactttttataacaaggaacctttggctttgagatgactggggaatccattctttcctatagtatccatgtccaatggacttaaagtattaatcaatgtgtttatgttttgttatttttctggcattacaaaaaattctaaatatattgttaccgcctgtataaatatcagcttttgagagaaggacattgtgtagaaataatgaaacactgcaacttgtatttgtattattcttttttttttttttttttttgagatggagtctcgccctgtcacccaggctggagtgcaatggtgcgatctctgctcactgcaagctccgcctcccaggttcacaccattctcctgcctcagcctcctgagtagctgggactacaggtgcccgccaccgcgccgggctaattttttgtatttttagtagagacggggtttcaccatggtctcgatctcctgacctcatgatctgcccgcctcagcctcccaatgcactgggattacaggcattatattattctttaaattcacatgagaatttagtatggcttcaaaaaataccataagttaaaatatcaccaagactctgttcagacaaaagtatcagaaaagtgagccaggcactcacatagtttatagtttataaaagtgagacaggcatgatctcttaacctcactatagtcctgtgaataaggtttatttacatttcattttacctgccaggattattgtaaaaacgccaagcacattgcctacacaaactaaatattcagtcaatggctgctattttcatgagttcgttttaacatatatttattgtcctctactggatttaagaagttatatttattatcatctaagattttagctattccttctcttaaaaatagattttataatcaatggcagtaagggagagtaactcgcagttctctgaatctcaaggggttcctggaagccttcctgaaggtatagtgaaatttcagcttcacattcccatccatgagctccctgcaaatatcccggtctgctctcaggacccagtgacttacctatgcagaggctgtagatagcacctggagcttcctgtgtgccctcctcaaactcagccaatgccgtcatacagtagcaggcaggtgtctttgctgggtagttggactggatgtccctgggattgcagaactggaatggggagtgacatcaggaaactataatcatcaggacaacatggtttgccataactttaagttttaagcgaccgcagattatgcggagagagatgcatgcccacagccatgcttcccatgtaactggagaggggtctgaagtttgaaacaagtgttcctaggcacgggttacagtgtttgttatcatcatacttgatttagaatggggcacaacatgtggattcatggtaactgttacaaccttactcattttaatacctgaaaacatgctttccccatgctgggaatcgaaagattctcctaggaaaagaaaggcttgacaacatcgattcaaaaagggcatgcattttcctcatttaaataactctaatgtgcaagtagatcccctgacctcaagctcagaagagtccaggccttcacaccttctctgcttctgctctggggccagctattgagattcctgtgcccacgcaatgcgcacatcccacccctggccgctgtccacaagaaatccagttgcaccaagcaccccactttttgcacctctcatttatgtactcctaagagcctcaccacaactcccttctaaaaacatgagttcctgactgggaattcgatgctgcccaggcagctttgctcagagggagcagccttctagaaatgtttcaagtaaactttcaagtataactaaattcaaaaaaaacacatacacacacacacacacacacaagtcaaaggtgtgtaatttggccaatatcacaaaccaattagccctttgtaagtggcacccagatcaggacagctgaccataccagcaccctagaagcaccccgtgctgcctcctgggacagggctaccaccatcctaaggccagcacgatgggccagctttgcctgctgttgaattttgcttacatagaatcctccagtaggtactcctttgggtcaggttctttcactcaacattatgtgttgatatttttccatgctgtgctgcaaaattgtatttcttgcattccataactgggcagttccatcataggagaataccacactgcgttcgtccattctaccgccaatggacatatgggttctttctcttttcttgcagttacaagtttatgaatattgtcccacgtgtccctggtgaacttttgtttgcatttctgttgggtacctcagagtggcgttgctgggtcagagggtactggtcgctttagtagctttgaaagatattgccaaaacattttccagcgcagttatagcaaattatacaccaccagcagtagaaaacatctcctaattgctcacagtaaacccccaaagattgccacatacatcttccatatcaattacttaactattcagcaaatttgaagggaaatatatttaatctttttattcaaatagtttataaagtggaatagagatgtgggtaaaagttgtcttgccacctttttagatcggtaaaagtttgttgaatgcaggcaagaaaagatgagaaataatggtacccaatgaaagacatagcagtctacaaggaggggcatttcccggggtgggggggacccacactctgtaactcccacattcaattagcatgttataggtaagctgcagaaaacgaggcagcttgtcaaagaggaacggctcttggccatggttgctgccctaggaggatatttgatactagcagagctggggcaaccctggaggaaaccacctggaatgatgggagaactcctccagggaacatggccctttaatagatctctgttataaaaaataatcccaaagcagccaccagggcatactgctgcgatcaagtcctaggcggtattcccttctgcgccatagaccctgtgcagagtgccctcaacgaaggagcaaggaagaccaagtctcccgagggtttgcatatgtgtatgtgattctgcagtcatggtgaatgacacagtcagggctgcggaaaagcattggtaaagtgtatatttgaggcttcagaagtttgaaaaggctagatttcctaggccaaaacactgaaaatttgcaattagaacttcagtgctgatgctgggaagactggagttagtttgagacatgcacctgtgcagaactgggcccccagaaaaggagaaggaagggaatccagaccagagtagggcctgacaccactcagactcggcgtgtctataaattagaattgcgttacaattacactttgacattttagtggtttttaaagtgcccagcacaagttaatttttcattaatgaatcctttattcataaaatgcttagatggagattacccttttgagcattttgccagtgcttctgaaattaatggggacctcctgttggaggacacagtctgttgcaataggtgaccactgctctgaatctatgtcacctctccaggaccacgggcacaaccatcacctgaggcatgttggagatgcagatggtcaggccctcctagaatctcagaatctgcattttagcaaagtcctgggtaattcctatgtccattggagtttgagaagcactggtaatctcaaatactttaaaagattactagagtaagataggctcagtaggtacctgaaggcaccatcccaaagaccagagtggtagaagcaggtggaccagcctctgaacacatttctcccccactccccggctgtgtggaaggttgccacctttggggtagtcattcaacaaacacgtgtcaactgtccactatgtgtcaggccaccactgggcactggctgtggctagctggatagacaccatttctgccctccagaaatgtcatgtccactggcacatgacaagtcactaagtcattcagagccatgggtgacagctccaggggccgacaaaggagctgtgatctcacagatccacagagaagtgtcccagggcgggcgggaaccaggactgcacagggaggggtgaagtgacacataagaagtcagcccatcagcctgaaatgctcccccaaatcttcccattcagtgttttctcagtagcaaactcgtgggaaaattggttattttacttaaaaaactcatactagaaagctagtttaactttaaaaataaattttaaaaacatttttattaacaaatcctacctttcctccaaagtcaaggagaaaagaatagaagtgaacaatggaccaagtaagcctaaaactctgctctttcccctgctcattttacagttcaagtgccattcaatttatcctggcaagaagaggaaggcatcatcaagaccttaattttctaatacatctgatctgagaagaatgtgaaagctataaaattaatttttgatcaataactacaggccttttgagagagtgccctcctaatgaattgagtacctatttctccatacacagtgtctatcatgacctacaaacccttttcccatgaggtgtaacagagagagattacagccttggaactggatgtcagactctcctggtttaagacaataagccatgacatagagcctgaaaccaacacaatcttccgagtggttccagaaacatataggggataatgttggctctgatgctgtacatccccaacaaccatcaactatttggaaactagaatttcagcataattggagttggtgttaccctagcaaatgctgtgggaagagagtctcactgtgtatcttctcctgtttaaagcctgaatttgttcagaatgtaatatctctgtttagccactctactgaaactgatctaggaaatgttcaaaaaaaggtatcccaaggatccctttgtagctacatctgtgggattcccctcgctctggcgtggcctggcccctctgcatttgacaatacggtcctatgcttttgtcttcctgggctgcgtgaacccaccctgccctggttcacctctcctcttgacccatccttatcagtgtcttgaaaggtccttctattggaggacacattctgttgcagcaggtgaccactgccccaaatctgtttcacctccccagggccatgggcacaaccatccctggagtgtgttagagatgcagttggccaggtcctccaaaatctcagaatctgcatttttgcaaagtcctgggtaactcctatgtccatgagagtttgagaagtactggtctcatgagttcctgacatacaaatagtgctgaggccagtatgctgactgggtagccagatacaagtgaaaaccttcctgttttttgcaaacctggatggacccgaggccgctgacgtgggccaggacaagctactctttttcagtgtttctgttgcatcgctgtgtctctctgtgatcaggtgctgccctccctggcaggaggactgcagacaggatgaccaagagcactctacacagcctgctctccagtgttgggggacgccacccaccctcgtggttcctgttcatctgcctacacgtggagggcccaagagggctaatatgtgactatctccacttcctggtaccctgtgtgaataacttcacttactaaagggatgttgagcaactttattaataatgaagaaagcactttggtttgacaaataatcactccattttttcatttgaaagttaactcttgttagtagagaaagcaatgtattacaaccacaaggacgtttacatggaaatgaaccatctgcaaagcatcccccattttccttttaaatcagccaatgggtggtggtgggagaaatattcaccagagtatttaacatctatcccccttcctagactgtcagctccatccgggcggagactgttggtatctccacagcacacacagggcctggcacacatccggggctcagtgagcacttgctgaatggtgaacagattagctctcctgggaacgttgttgacacatctcataacactggtttggagtggagggcattcatcgggctgcatattcctatttttaattgtattctccactggttacagcacctacagttataaagacattgttaacattgcttataggaagacatttgatggaaatgagtccaaaggcattacggttagaaactggccaggtgtcatttttgagagattagataactgttttccggtagagtgaattgcctgtttgttgcaagttgggactttgctgggctggtttacagggccaaggggaaagagataagtggatcttctagtgagaggtcatctgttttgaaagcctggaagattccatgaactaaatccaagtcttacaacacagggaagtgtgtcatactgtgcagggatgaagtctccaatttagcatgaaaacaagagctcctcacactgtcctcttcagaaagcccatacaatccaaacttctgaatgcttagctgcttacaaccatacatagattgagggataaaactctgatatggaagagaaggtaaacattttttggcagacattcccaggaaaaggcggctctcttctctcattgctgctgctctttcagaatccatttcaacagaggaggagtcaatgggagccccgtgcctctggcagatatcatatggcgtttcagtggcattgtgtgttacccttcttaggtaacagctcagccattagaagaatgtcctacacaccttctcattttctgtgatgagaggaatgtgaggtactgcccttcgagagctgtcatttgtcctagtagccagcagcgtgactgtgctgtcttctgctctgtctccctgtcagccttctgcccagccaccaccactatagttttgttctctccattggaactcctggttcagagaattaccataaaaaacagacccctagacatacaacactctatcacataatggtgactttgtcttctattttggattactgagctttcttgggtaacttccactaaatcgaagttaatattagaagaacttcctcttactagaatcgaaaagcatttaagtgatgcagtcaagtttgtaccataagtaattcagtcatttaacaaatatatatggcctctgtgcgacagtgaccttgactgggaatgaagctgtcccatgtggggcctgttcttcaaaggcagttccctgctgcccagttcagtccagtggatctgggcatctctctttaatccgcattaggggctctttactgattcttcactatccaaaaagacttggaggggagacctgagcccacttctggaaggaaatgataacaatttatttagataatctttgtgcaacaagtcaattcactgaagagatctgctctctaggagcctctgtgaccccaccataactgggaaggctctacctctccagtcttcgggccacatttctctctggcctgctgtcttcccagcactctcagccttgctcatggagcactctagtcctccgtcgaccttggcctttggtaacgtgatttttcacctggcagctcccatctggtctcactccctctttttgtccagtctgcatgacacagcctcacatcgttagtgttccctcactcccctcttactgcccaacctgcaaagtccatgcctgggccagtgcagcatgtgtcctcaatgggctgctggtggcagtggggggaaccgcacagccacgctgtgtgctgctgaagaaatgcacagcctcctaccctcgccctcaagaggcagccatggctgcgcatttctgcccttctgagctccgctcacttttggcagcagccgttccaacctgcatgggatcttcactctctcacagatgtgctgactcctcctgctgcctcccctctctgtgccttctcactctctgttccctttgccctttctccccttttctcctctgcctacctccaagccatccatcacaggacagctcaagcatcagatcctctgggacactttccttagttgttcagtctgatgaggtgtccctcatcctctcttagctgaaaatcagcagctgcctcaacttcttttccagcatgtctcatgagtattgccacaacagcatctgtcacaatgtggggtagtggctgacttgcttttctgccattcaactgagttccctcagtgctggggccagcgtgcagtgtcttgtattcagtatatagctgattaattgatgaattgattaattaatggttcacactagcacagtgcaaccttcaatgcaaagatctcatcaaaataattcacatggtgggatattttagaaggatgaccaggctagtttgtagtaagaaaaaatcaacaagactaggtcaggaattctttttttgtctacaggcttgctatagaagatattgaaaatcatctacctaattacctttattttatcaggttgtgtattaaatatcacgtctgggggaagaaaatgtgatatgtgattacagacctttcctggtacaacatagtacgtttcagattaactcaaggtattgtggtgatattgcggtcaaagccaggtgattaaagagtcattctttgaaacaaatatctgtgcaatcaattaagaaattaatttgcaaattttatttgcttagagtaattgatatatcattccttttacaaacaaatataaagaaaacttaactaaaaatactgcatatctctttcagattatatatcccagaaaggatatatttttctcctttctggtcttcctttttggtgtagcatctgtaggaaatgcatttcttcatagctaagtgtacctccttgtgaaatatcttcagagtctactggtgcacataagcaattgctggcagcagcttgagggtctccatctcacatttatcatatgccttattgcatgaggctttgcaagaggaggtctagagctacaatatctcatggatatgaatgtcaattcaaatcccagtggcagtttatgagggggaaagcctagaagagaagaaacctagaggaatcaagcaggaggggagagtaataaaagactagagcagcaggtttttcttaactcaaactagaattaaatctctgtgtgtgtgtgcatgtgaatgtgcccgtatgtgcatgcatgcacgtgtgtaaatggatgtgtgtgtgtgtgcatgtgtgtgcaagtaagtgtgtatacgtgtgtgggcatgtattgtgtacatgtatgtgtgttttatgcatctgtttgcaagtatgtgtgtatgcacataaaagtgtgaatgtacatgtgtgcttggtgtatgtgtgtgtattaatgtatgcgtgtagttctagagtctagttagagaaagtgcataaagaaatagggaaattaacaagaaagctatagcttaaattataggaaaaacttttctccctatcagtcatggttttaaaatgttcagacttgatatgtttcccagtgctattgtcagaaaatgtccctatgacattccatactacttcaatcaaatctaaaacctttgttccaacatgttttattgatatgagtatatttcaaatttctaccaggtttttggagaggtattttggccataaaattgactaaattattcaaaataaaaaatgaataagcctgggccaaggcttggagacttgcttaactcagttcttaaattttcagattttcaaaattacaaatttaagctctaaaatcatggtgctgtgtatgatattctttgattgcaacttatggttgaaaaactatagagggctttatgctaagagttgtggatcttaggattttcatgaaatctgcattatcatcatctgcaagtttagatggggcataactgatccaaaggatggatccctcgggggcaattcaactggctgattccagccaagatgacaacagtcaggatccgttcccttctgatcatccattgggtgccctgatttcctctacagccctagctgaaagaccagacactatctcaggctggctgccccacatgccttgctccacaccaaattcacagtctataaacctgagcctccagtgctcctactaccatactcactcgaacattcccgattctgacctggagatgtcaacagctacttgatgccactctcttctatctttctgtagctaagccatccccaagtttgtcgattcaccctctttaacccctgtcggggtgtccattgtgccccttcaccctgccatctccctggtgcactgttttgcaaagttcagcatacatgagcgtcacctgggaaccttaataaagtgcagatgttgattcagcaaatctgggatgccctcgggctgcatttccagcaggctcctggggatgtccccgctgctgtgctgcagatgacactctcagtggtgggactccaggctctgctgtcgcctcctaggggtttctccacactccctggaggcctaatgggcccttctccacatggcagtaagatctgtttttgtgtttgtgtttcaagttgggagaaggagattatttaatactaaaatgtgcaacatgggattgagaaaactaattattagtcataagttgagtatgcaacattgaaaccacatgctttaaaaaattataagaaaaaatcatagtatttgaaagttacaagctattatggctaactccatttatctcagttagagaagaagagtcacctgtcaccagggcactgccagaagccaggctcatttccaacagcactgggtgctccagctttggggtgccagctcctcccataaagcaaacacatacctagggatgatatttctttgcaagggctctgccctacagcttgtacatctcaagaagttatgtaattaaactgtctgttttgagaaaattgtagattcacacatactagctgtaagaaatgatgcggataaatccagcgtaccagctttccccacggagacgtcttgcagcgtcacagccaggatgaggcattgacccaggcgaagtccagagcacctgtgcgctacagggccccttgcactgtgctgtcacagacacgcccacttccagatgccatctaggaccccctccaaaaagcagaggcattcttaaaaacacacatctgcacatgttcctcttcatttgaatctgtcagtggcttctcagtgcctttcaaatgaaatctaaagtccttacaagccttgcagcaggaacctctccatcccacttcccctcacactctcagcttcatctctgctaggctctgttcagccaggcagcctttcacagtccctctcctcctgccctgccaggaaggtcccctgcccccaactcttccccacatgtggcggggccccgcttgtccttagaagcccagctgaactgcttcctgaaggaacccctccagaacctctcagaccaggtcaggtttctgcactcttagatcatccccatggcataatcacagttgtgatgttgtgatgattcagtgaatgtctgtctccccactggatggtaagcttcctgagggcaggaacagcattggttccagtcaatgctatgtcccaggactgttcgtttttgcacatactaatcctaaaaggacgatgacaacagcaaccacttacatgacctagatgctcttctgggtgttgtgcaaatattaacaatttaatccttgcaacaatccacgagggaggcattcttctactcccacttaacagacaaggacagtgaagctagtaaagagaagtcatttgcccaaggggaccccactactgttggcagagctgggtgcaaacgcaggcttgtgaagccaggacccatgcattcaaagaccatgccaggtgcccccactgcacacctcatccccacataccagtgagggggagagaaatgctcctgcactgcctctgattaactgctttcctagaagtcacacatataaaagggatttaattctagtgggattgaatctcaatagtttccttattaggttgatttctgttaatagtttaagtactggatatacatgaattagaaaatctagattattagcaaatgcaaactataaagtattttataaatgttatcttgtttgtcaggggatgagtgagatattcattatacaaaaagtagtgtggattttgaggtagaaggtttactaaggatcataccgtagtatgaaatagccacaaacattcagtgaaaccaaacacccccgcttaacctcaaactaacactaaataataaggaatagacttgggggcagtgcaagtgtatttctaatggtgaaaaccattccccagtgaaaactaatgtaccatctagttaataagagctcctctgacccacgcacatcaatacttacatcccaatggtgatgtgacattttgggttttgtatttcttttgcaaattgagctagcatttttgatgagtggcagggctctgctacccaacctttggacagtttccaagcataaaatcacaattccagataattctgtcacaaagatctgggtctcattaggaaggagaggaagctgggagatgatccagtccaacctcccccaaaccaaacatcacggccttctcagttgtttcaccaaccatctaaatgttttagtaattctaaaaattgatgcgctttttccacgaaaggaagtgttaccacattttccaagtgggaggcatctatatccttactccttcatcctctccttcccaccccctcaccccccaccacccacacaacatctgcaattcttaaactaaagcacaaattgttacaaaagttaattgcactttcaaaggaatgcttgtatagaaactttctcggcttcaaggaaaaataatacgctttgaatggctgttcaacagcatagaaattagctgagtagaaggcactcatatagccattaggaccaatcctttctgccgccaacaccccccttataaagacttgacagtgggccagaataaacaacttcaggatgaattcagttgagacacaaagtacacacttccagtttttcccttctctggttactggcctcaataaccaggcagtcaacttaaaaagaaaaacaaaagcttgcttcagattacagattgcagacttcttataatatgtccatttcaccaggccccgctctcagccccgggaaaggccactggaaaccacctcacatggtagggccttgcgggagccagtaataaccttatctccgtcaacatgttctgtcagattgaatggggcagccagagaagccagagttggcacaggaaccaaaacaaaggcttcccatcctcctggagtgagcggttgagcctggattggtgcttagacctataatgggtgcaagcagcgttcattcatagtggctttctagacccagggacttggccccagccctgctgctccactcctcttcttgcttcattaccacgagtctcctagaccaccgaacgatgcctgcatttgaaagacacttctgctgatcaaagcagctgatgtgtccctttgcggttcatttctaattgtccccaaggaggagaaattcaaatagtttattactgagagttaaagaaatccactgaaatattctttggtctaaaattactgtcatggcggagcagcttcaccttagtcattgcccttaaatatgaaagctatttaagaaagtttgcccttaaatatgaaagctattttaaaaagtttaatgaaagaagagaatcacaaaacattttcaaaaagcaaaagaaaacctaagagaaaagttgaaagtaggaattttttaaagaatatacgacgtgtgttctgtgactcacccctgcaagttatttgtgtgtattcccttgcatagtaattaataatgaagcaaagcatggcaatgatatcttttcttgtctagtattctagaagactccatgtttttggaaaatatcactctagttagatctcaaatatattcaatcagaaaatgggttttctacaagattctatatctgtagtcaatagcaaatataattctattaagctagtaggatgtgataggaaactaaaacctaggggagaccaaagcaaggaaaaatacttcctcatccaaacttgagagcaatttaccgtcaggcctactattaatagatggaatacagattccattttcattactcaactgccatattcattattacactgtacagaaaagggaatcacatctgttgaaaacttatatatgatgttcatgcatgcattccagtaattcaacaatttttatttatctttttattgcttgctaatttttcaaaataataagctaaagaaaacaaaatgtttgtgctgttctcagatgacatgttatctctttaaaggacaaaatgtgctgtgaaataatagaatgctttcagcactcaagtgtgagtgagtgctcatacatgagagaaagccgtggggactacagaagccaagaagcagatctagctggggaggcctttgcagaggatgtagttgtgtggagaggccacacacgtggaattcccaggagggctgtggaggcggggaatctgcaggaaagcactggggtgagaaacgtgatgagaaacaattattgtcttaaaatatctgcagggctgtaaggtagagaagcaatacgttgcatctgtgttaagtcaaacaaaattatcaagggactggtttcagcttaacataaggaacaattatgtgatagggttgtcaataacaagagtagactgcttcttcacacactcctagtcactcagaatggtccaggaggagtggacaaccatttggtagagtatgggaaggcaggggccctgggtgggagtggtgagggtagggagtgagtatcccaatctagaagtaaattgtgcccagcacggagctgcaacactgccctgcacacaaacacacacaaataacaatccccagcccctgcatttccctctccggtttcaggaccttgtatcttacttcaattcctttatttagctgatgatgaaataggaagagcttagcactaagaaaatccttttggagtttggccttgggggaaaatgaatcactccaaccaggtctgtcttctagaaagtataggatgaaagggctcctcatcacatacttcctgacctcctgctaggcctttccctaaaacaggggctggcaaagcacaacctgtgggtcacgcctagcctgccacctgtttttgcaaataaagttttattggagcatgactatatgtatttgcttacagtctgtggctgcgttcacactatcccagcagagttgaataattgggacagggaccatatgatgggtgaagctgaaaacatttactctctggctgtattcagaggaggtttactgagcccttctctgagacatggcaagcgctgcttcaggctcatgcttcactagattcaggcctggggcagtaaagagccagctcaggatagcactcccgactcactcattttttcaggcaggggagccatctaatgtcaagtgcctacgtgcaggaactggtctgttaattagcagctctcctcatggaagggataatatattctagaaacaggagtgcggccctattgcaagaatgtcctgagccaaaattaagattcttctatggcagaaacttggctggggcttctcctgagttaacttggtagttgttagtgatttttgagtcagtttttccttgtcaacgaccccaggaatgagtttgggattacagggtagccagggaaagggaaagcttcacgcccgcccccgggacaaggtctgtcttcacactgctacatcccttcacccactttaaaatgaaacttaaaaggaggatttcagttgagtaggaagtgagaagagggctcattttaaaacaagcgttaaatgaaaacccacacacactcagagcacacaaatccaaccacgcttacaaaaccatcacagagggtcaggcgaggcccttttctaaatgaaaaagaacaggggtggagactgttctgagagcatgctgggttccctgaagggaattctcagctgtatgtgccccgcacaggatccctgctagacacaaggccagctgccttcctttcaagccgcagacgcatccctgtgtccaggcgggctggtcagctgcggtcagcaccagcttccccgctccatggtgaggtcatcacaacatgtgagcaggagggcaggccggcaacctctgagtgcttagagaaagggacgggattcctcctgtgcaacccctctagtctcactcagactcaagtctgactaaggggccaggtgctttgaccagggactctcccctctcacttccctcccaggagtcacaggtacatgagtccttgttttacaaatgaagaaaacagacccaacatgattaagatgttgccttcataggggtggcaccaggattccaaaccatggactccactgagcccagtgcccactgacatgtgccagtaacagtgcagctgcctgtggttctgtcgactaaactgccggcagaggctggctttccaccttctttttttttttttcactcttcaaacactttatgacatgaacataaactactggctgcatcgttctgctgacaacatgacatgtttctataacttgaaaaaagcaagcagtggactgctcattggtaaaattgagtcagtaatcttttaggaaggttatttttcttccttttactgcttctcatctgttccccgcagtaaagaggacaagatgacgacgactcagggaacacctccagcctgaagcagcaccatgcgagcttagaccttagggtcggcttagaaaccacaggcggggcggcttgggcccctcggacactccctctcgaagctgcttctccccaagctaccccaaaggcactgagcgccctctgccccccagcaattcaattcactggctgtcctgctcctgtcagtactgagagttgcatgtttgaccctcgggggaaaagtccagaggccctggggtgtccagcatgctctgaggtccctgctgctgaccccttgcgctgtcagcattcagagacattcacacagcacagcctcccaggctaacagctgtcatggaacagtggagcagctagacgtggccattctgtggcccagtgctgcagaggtcaaagggacaagcgcagggagcatctttgctttcagaaaaaaaaaaaaaaaaaagaagcacactggtgcactgacctgctcctggtgtctttgtgattgctcttttctttcgatttttggttgtctttttttttttgaaagaggggcttttatgcttttttcctaatgttcatgggtaaaccaatgtaaatgtgtgtatgtttatagagatggctttaaatcgcaattctgcagtagagattgattttttaaaaaacatgggtaaaaattgaagaaaaattttaaaagaacatttaaaccatcttgggctaggggtggatatgcaccaccccacggaagccaaacaaaatctctctgcagataaacatttgcaaaaagaatttccaatcccaatttttgagtcagagatcttttatttccttgcaaattacatatctgtttcaggatttttgactataagaagaatgaatgaagatgtgtttcttacagataactatgaacaaaccaggaaggataataacttgtatcccccaattcgaatccagaggatgggaaggcataaaaaaaagaaatggaagaaactttatttttagtggtaaatggtgggactatgtattttacgtatggtgaagtcaccaagcccaacacttggcacttgtaggcaaggtagtcttctaatctgaatgtgaagtattatgttttcatttgcttggtaatgaggaatattggtgctttcgtcccagttctcgagctgactgacttctctttctgacgtgtgttcctttagcacacctctacactgcatggctctgagatgtcctgtgactgtttcatgtgtaaagttgcctccccaaaggactcacatattccttcagggcagtgagtacttctgattcatccttagcagctaccttcgcgctactttactagatatgttgtagttgaattaatgaacaaaagaacaagcaactttggtgcctggtgtgcatctcagagcagggtggagtgagcctggccaaagggtcatcatgcaacctctgtggctgactccatctggccacggagcttctcagccatgcttggtattcacatgacttctagggcgacagctcaaccagcaaataaacagcttcatatgggaaatattactcagcctttgtcatcaaggagtgagtcacgggcctgaactgaatagaagatagaggagaaaaggtgtgtggactgggtgagacagcgcccagcgaggtgaactcccggcagccctgcctgtctttacctgcacatcaccttgctagggtgccttcggttgtgagggcctgtctaggaagagaagagttgcaccctggcaggcagcactgagctgtctcatgcaaagctgaggaagaaagagtgagctgcccagtgagcctgctggggtggtggaggctgggctgggctgtgcagtctgcagcccccagcagcccttggcacctttctactgcctggtgctcaccagctctccagtaacaaagagggacgtgaagtcagaggggaagggaggtagcacagggcagtcttgactttgaacaaagagctggcttcctgaagtcagctggccgggttttgaagccgattttccagcagtgatctttgatgccaaccccatttaggaattctgtatctccccctaccttctaccagatgtctctgagctcacctttggtgataatcatgcaatctccgtcatccccacgtccacactgccccattctgtcccaccccgggttctgtggtgctgtcggctccccagcgagccaggaagggagaggccagctctgctggggctcctgccgccctggctctgcactgcccttctctggcaggtctgaggcgccactggaggagccacacggccctgaagcagcaaggcagatgccctggacacagtggaggcacagagtgcaagcaccggcctggcccacagacttttggaggggaagtggtattattcagttcaaaagtatgcctgtgtgtaaagagagagcccctgaacatgagtaagcaaaagtctcagcgcagagattagacaagtagaatgctggcccgagaggaggcgtttactcaccctctgtctaggaaggaaagccaggcccagcacgctcactgctatctatcctctcacacagagggattttgaatcgaagccagcatcctgtcctttctccaatgtcccctgctcaggagtcaggactcagcaaggcccaccccagccacacacagatacagttccaggactcagaactcagcgaggcccaccccagccacatgcaggtccagttccaggattcaggacacagtgaggcccacccgagccacatccaggtccagttccaggactcaggattcagtgaggcccaccccagccacacacaggtccagttccaggactcaggactcagcgaggcccaccccagccacatgcaggtccagttccaggattcaggacacagtgaggcccaccccagccatatccaggttcagttccagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 77 - RNA, 9676 nt, Homo sapiens
  source 1..9676: mol_type = mRNA
agttctccactggacttcagaacaagagggagctctgggctgctggctggttgtgcatttgctgtgggttccctccggcaggcgacctctccgcgctgagaaggttatccggataaccaatttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 78 - RNA, 10066 nt, Homo sapiens
  source 1..10066: mol_type = mRNA
gattgaaagctcaatcctggattttaagtgcttcaaaagtgcacttaattgccacatttttgtcaaacattttcccaggtagtatttttcctcatgtaaaacaacagcaatttaatttgaacagaaagcattttgaaacatacttttggcagggttccttgcagatcagaatggaaatgattaacagggcaattatcaatcatggacttttggcggcagaaggaactgtattgtttggtacagtctgggccagggccacacaccgtaacggagatactctattctgtggacggttggagggggctgtgctgagcagggtaactgcatcttttcctagactgttcacactgctgccacgaaggagtcttgtttagactggacctggctttcttcttcgcaatgagtgttgcagactcccgacaaaggccaggtggtaaagtgtggtgtctgtgagcgagagcctgagatgcctgagctgacctgtcctcagccacctgccatcgtgcagagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaatgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 79 - RNA, 9577 nt, Homo sapiens
  source 1..9577: mol_type = mRNA
catatccaggttcagttccagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaatgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 80 - RNA, 3716 nt, Homo sapiens
  source 1..3716: mol_type = mRNA
gaccagttgggcatgaccagagagaagcgtggggaagagtgggctgagggactccactagaggctgtccatctggattccctgcctccctaggagcccaacagagcaaagcaagtgggcacaaggagtatggttctaacgtgattggggtcatgaagacgttgctgttggacttggctttgtggtcactgctcttccagcccgggtggctgtcctttagttcccaggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtactttgaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcatcctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaaacctaaatgaggtataaggactcacacaaattaaaatacagctgcactgaggcagcgacctcaagtgtcctgaaagcttacattttcctgagacctcaatgaagcagaaatgtacttaggcttggctgccctgtctggaacatggactttcttgcatgaatcagatgtgtgttctcagtgaaataactaccttccactctggaaccttattccagcagttgttccagggagcttctacctggaaaagaaaagaaatgaatagactatctagaacttgagaagattttattcttatttcatttattttttgtttgtttatttttatcgtttttgtttactggctttccttctgtattcataagattttttaaattgtcataattatattttaaatacccatcttcattaaagtatatttaactcataatttttgcagaaaatatgctatatattaggcaagaataaaagctaaaggtttcccaaaa

SEQ ID NO: 81 - RNA, 3858 nt, Homo sapiens
  source 1..3858: mol_type = mRNA
gaccagttgggcatgaccagagagaagcgtggggaagagtgggctgagggactccactagaggctgtccatctggattccctgcctccctaggagcccaacagagcaaagcaagtgggcacaaggagtatggttctaacgtgattggggtcatgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtactttgaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcatcctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaaacctaaatgaggtataaggactcacacaaattaaaatacagctgcactgaggcagcgacctcaagtgtcctgaaagcttacattttcctgagacctcaatgaagcagaaatgtacttaggcttggctgccctgtctggaacatggactttcttgcatgaatcagatgtgtgttctcagtgaaataactaccttccactctggaaccttattccagcagttgttccagggagcttctacctggaaaagaaaagaaatgaatagactatctagaacttgagaagattttattcttatttcatttattttttgtttgtttatttttatcgtttttgtttactggctttccttctgtattcataagattttttaaattgtcataattatattttaaatacccatcttcattaaagtatatttaactcataatttttgcagaaaatatgctatatattaggcaagaataaaagctaaaggtttcccaaaa

SEQ ID NO: 82 - RNA, 3737 nt, Homo sapiens
  source 1..3737: mol_type = mRNA
gctgtgttccacggcccatgtgaccagttgggcatgaccagagagaagcgtggggaagagtgggctgagggactccactagaggctgtccatctggattccctgcctccctaggagcccaacagagcaaagcaagtgggcacaaggagtatggttctaacgtgattggggtcatgaagacgttgctgttggacttggctttgtggtcactgctcttccagcccgggtggctgtcctttagttcccaggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtacttggaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcattctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaaacctaaatgaggtataaggactcacacaaattaaaatacagctgcactgaggcagcgacctcaagtgtcctgaaagcttacattttcctgagacctcaatgaagcagaaatgtacttaggcttggctgccctgtctggaacatggactttcttgcatgaatcagatgtgtgttctcagtgaaataactaccttccactctggaaccttattccagcagttgttccagggagcttctacctggaaaagaaaagaaatgaatagactatctagaacttgagaagattttattcttatttcatttattttttgtttgtttatttttatcgtttttgtttactggctttccttctgtattcataagattttttaaattgtcataattatattttaaatacccatcttcattaaagtatatttaactcataatttttgcagaaaatatgctatatattaggcaagaataaaagctaaaggtttcccaaaa

SEQ ID NO: 83 - RNA, 2328 nt, Homo sapiens
  source 1..2328: mol_type = mRNA
cttcatacacgcggccagccagcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctggaatggtgtcattatttgcatagcaacattgcaatttgttttcctttggctacatcaacgtatcacctacactattatttatctaatatttctctttcaggcagctcatttaaactttttttggctttaattcctattgccatcctgagtggaaaaccagtatcattttccatagtagaggataactataacaacgaagataatgaaagtaatttttttaacctaagaaaatactaatggtcacagaaggctatgaacctgagacctgctctctttgtcaaacagagattaacaaggatcagagaaaagttaaaggctgtctttgacataactggaaggcttacaagagttttaaagaaattactttctcactatatgattcattgctatttaacggtgagtcagtaaaccacctaaaaatcatcttgtctcatcagaaacactagtgtaacataactgcatcattttctactaaagacaaaatttcaccaaaggggaaaaagaaaacacaggataaagagatgtattacacacatcgaaaaaaacaacatttaacaaaatattaaaatatttccccactcttctgatactgtcaagtcttactacttaaaataaggcttccacctctcccacttcaaacaaataaatgaattctaaaccaaagaaacttcatcaggcatttgggtacctcctcccctcaccacgaggtctctgctgaacctcaatcctgtacactgaggaacctgtggcttgaaccctacatccctccagaacccccagactg

SEQ ID NO: 84 - RNA, 2902 nt, Homo sapiens
  source 1..2902: mol_type = mRNA
aaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtgaggagcggggtccctaggtcaaggggactcgtgagcggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 85 - RNA, 2779 nt, Homo sapiens
  source 1..2779: mol_type = mRNA
aaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtgaggagcggggtccctaggtcaaggggactcgtgagcggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 86 - RNA, 2862 nt, Homo sapiens
  source 1..2862: mol_type = mRNA
aaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 87 - RNA, 2739 nt, Homo sapiens
  source 1..2739: mol_type = mRNA
aaactgcggcggtttacgcggcgttaagacttcgtagggttagcgaaattgaggtttcttggtattgcgcgtttctcttccttgctgactctccgaatggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 88 - RNA, 2967 nt, Homo sapiens
  source 1..2967: mol_type = mRNA
aaggcatgggcaggaagccgtaactggggaagtttctgggcccagaagaaaatgtaggtagtcttagggttaggtagcagctgtcaggaacttgcccctgcccataagatcctaaagggcccccatttgactctcaccagacagttagaacttgtttcctcctccgtgtcagccatcaagaggtgcttggggggctgtgcccagcaggacctcactgcccagcagatcagcaggggagccaagtggcctagatctgctgtggagtacccgactgtttgcctgcctgtctgccctcctcttcacctcattctcatcactgacgtctaccattggcttggaaatcagaaacccatatccatctagatgattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 89 - RNA, 2654 nt, Homo sapiens
  source 1..2654: mol_type = mRNA
aaggcatgggcaggaagccgtaactggggaagtttctgggcccagaagaaaatattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgaggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgatcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcggacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 90 - RNA, 2902 nt, Homo sapiens
  source 1..2902: mol_type = mRNA
ccaagcgtaggaagatggttgggaactgcgtctcccccaaggctccgcagggcgccgctgactggcggaagcgagggcggggacagcggaatgacgttttaaggacgcttgcgcgcgggatttaaactgcggcggtttacgcggcgttaagacttcgtagggttagcgaaattgaggtttcttggtattgcgcgtttctcttccttgctgaccctccgaatggccatggactcgtcgcttcaggcccgcctgtttcccggtctcgctatcaagatccaacgcagtaatggtgaggagcggggtccctaggtcaaggggactcgtgagcggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgctcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcagacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 91 - RNA, 3023 nt, Homo sapiens
  source 1..3023: mol_type = mRNA
aagcgtaggaagatggttgggaactgcgtctcccccaaggctccgcagggcgccgctgactggcggaagcgagggcggggacagcggaatgacgttttaaggacgcttgcgcgcgggatttaaactgcggcggtttacgcggcgttaagacttcgtagggttagcgaaattgaggtttcttggtattgcgcgtttctcttccttgctgaccctccgaatggccatggactcgtcgcttcaggcccgcctgtttcccggtctcgctatcaagatccaacgcagtaatggtgaggagcggggtccctaggtcaaggggactcgtgagcggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcctgcccccactaggccttcctgccctgcagtggctgaaataccattgaggatggtcagcgaggagatggaagagcaagtccattccatccgtggcagctcttctgcaaaccctgtgaactcagttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgctcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcagacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttgcgcctggccatgcagctggaagagcaggctagcagacaaataagcagcaagaaacggccccagtgacgactgcaaataaaaatctgtttggtttgacacccagcctcttccctggccctccccagagaactttgggtacctggtgggtctaggcagggtctgagctgggacaggttctggtaaatgccaagtatgggggcatctgggcccagggcagctggggagggggtcagagtgacatgggacactccttttctgttcctcagttgtcgccctcacgagaggaaggagctcttagttacccttttgtgttgcccttctttccatcaaggggaatgttctcagcatagagctttctccgcagcatcctgcctgcgtggactggctgctaatggagagctccctggggttgtcctggctctggggagagagacggagcctttagtacagctatctgctggctctaaaccttctacgcctttgggccgagcactgaatgtcttgtactttaaaaaaatgtttctgagacctctttctactttactgtctccctagagatcctagaggatccctactgttttctgttttatgtgtttatacattgtatgtaacaataaagagaaaaaataaatcagctgtttaagtgtgtggaaa

SEQ ID NO: 92 - RNA, 1124 nt, Homo sapiens
  source 1..1124: mol_type = mRNA
cgctccacctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 93 - RNA, 1118 nt, Homo sapiens
  source 1..1118: mol_type = mRNA
cgctccacctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 94 - RNA, 968 nt, Homo sapiens
  source 1..968: mol_type = mRNA
cgctccacctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 95 - RNA, 1853 nt, Homo sapiens
  source 1..1853: mol_type = mRNA
cgctccacctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 96 - RNA, 1031 nt, Homo sapiens
  source 1..1031: mol_type = mRNA
acctctcaagcagccagcgcctgcctgaatctgttctgccccctccccacccatttcaccaccaccatgacaccgggcacccagtctcctttcttcctgctgctgctcctcacagtgcttacaggtggagaaaaggagacttcggctacccagagaagttcagtgcccagctctactgagaagaatgctatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 97 - RNA, 1070 nt, Homo sapiens
  source 1..1070: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 98 - RNA, 957 nt, Homo sapiens
  source 1..957: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatgtttttgcagatttataaacaagggggttttctgggcctctccaatattaagttcagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 99 - RNA, 989 nt, Homo sapiens
  source 1..989: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatgtttttgcagatttataaacaagggggttttctgggcctctccaatattaagttcagtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 100 - RNA, 830 nt, Homo sapiens
  source 1..830: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 101 - RNA, 1145 nt, Homo sapiens
  source 1..1145: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 102 - RNA, 1077 nt, Homo sapiens
  source 1..1077: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 103 - RNA, 1199 nt, Homo sapiens
  source 1..1199: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 104 - RNA, 1805 nt, Homo sapiens
  source 1..1805: mol_type = mRNA
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 105 - RNA, 1094 nt, Homo sapiens
  source 1..1094: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 106 - RNA, 1016 nt, Homo sapiens
  source 1..1016: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctatcccagcaccgactactaccaagagctgcagagagacatttctgaaatgtttttgcagatttataaacaagggggttttctgggcctctccaatattaagttcagtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 107 - RNA, 857 nt, Homo sapiens
  source 1..857: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 108 - RNA, 1140 nt, Homo sapiens
  source 1..1140: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 109 - RNA, 1172 nt, Homo sapiens
  source 1..1172: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgcttttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 110 - RNA, 911 nt, Homo sapiens
  source 1..911: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 111 - RNA, 1226 nt, Homo sapiens
  source 1..1226: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 112 - RNA, 4070 nt, Homo sapiens
  source 1..4070: mol_type = mRNA
acctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgccccccaagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggagagcaggccggccccgggctccaccgcgcccgcagcccacggtgtcacctcggccccggagagcaggccggccccgggctccaccgcgcccgcagcccacggtgtcacctcggccccggagagcaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggagagcaggccggccccgggctccaccgcccccccagcccacggtgtcacctcggccccggagagcaggccggccccgggctccaccgcgcccgcagcccacggtgtcacctcggccccggagagcaggccggccccgggctccaccgcgcccgcagcccacggtgtcacctcggccccggagagcaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcgcccgcagcccacggtgtcacctcggccccggagagcaggccggccccgggctccaccgcgcccgcagcccacggtgtcacctcggccccggagagcaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcacccccagcccacggtgtcacctcggccccggacaccaggcgggccccgggctccaccccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 113 - RNA, 714 nt, Homo sapiens
  source 1..714: mol_type = mRNA
actttcactcaccgcctgtccttcctgacacctcaccatgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatggtactgtgtccggggattgcagccgttcgggcagggggcaagggctgctgtggtgctgggtgctgtggaaaccgctgcaggatgctgcgctcggtcttctcctcggcgttcggggtgcttggtgccatctactgcctctcggtgtctggagctgggctccgaaatggacccagatgcttaatgaacggcgagtggggctaccacttcgaagacaccgcgggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacacctcactgaggctccactgaccgccgggttacacctgctccttcctggacgctcactcccttgctcgctagaataaactgctttgcgctctc

SEQ ID NO: 114 - RNA, 2896 nt, Homo sapiens
  source 1..2896: mol_type = mRNA
aacagttctgacgtgagaggacaagaaacacaagcaaatataaaacattcaattctaagagaagttcatcagagacatccttcaggattgtgagctggattttcctctacgtgcttcagtcttctctacacaaagagctgcaaaccaggtctttgttttgcactcttatttcaagcagaatgagtgctcctaaactcctctctctgggctgtatcttcttccccttgctactttttcagcaggcccgggctcaattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggaaatgttgcaagagccttctttctcccttccttactggaattttgcaacggggaaaaatgtctgtgatatctgcacggatgacttgatgggatccagaagcaactttgattccactctaataagcccaaactctgtcttttctcaatggcgagtggtctgtgactccttggaagattatgataccctgggaacactttgtaacagcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctgatatatccacatttccattggaaaatgcccctattggacataatagacaatacaacatggtgccattctggcccccagtcaccaacacagaaatgtttgttactgctccagacaacctgggatacacttatgaaattcaatggccaagtcgggagtttagtgtacctgagataattgccatagcagtagttggcgctttgttactggttgcactcatttttgggactgcttcttatctgattcgtgccagacgcagtatggatgaagctaaccagcctctcctcactgatcagtatcaatgctatgctgaagaatatgaaaaactccagaatcctaatcagtctgtggtctaacaaatgccctactctcttatgcattagtatcacaaaaccacctggttgaatataatagattgagttattaactgtattttctttcactttattaccttctttctaatacaagcatatgttagcattaaagttctaggcatacttttcaaagctgggaagaccctttcagaatcttttcaatgggttttaattttcagttctatttaaaatggtgaatgacactaaactccatgatatttaaggatagtgtgaagatctttggcatgatttaaaggttgagtatgtgaagatataagtaagtgaactaccatgctttgtttacgtgtaaaggaaaataatgtttgatagtaaatgtccacttaaaatacatgaatgggcatttctaaaatgttaaaacataaacacatttccattcatggatatttgtcaacagatttaaagaaaaccacagttattaattaaagaaaattaattatgtgtagttataaaccaatgaaattttgattaaccttttcaaattaatgttccagtttgaagaccaatcaaatatattatttagtcaacatatactatttagtctcaggttcaaggctacaacaaaaatcaccatctttgtcaaactttggagagggaaaatcttcactttcttaagcaacaatggatattgcctgtgtttgccactgtgtttccctgcctctcaattcgctgaaaaaggaactacctatccttacatttcacctactaatgtctcttctaacatcttagaggtccatggagaaggcatatggagaacatgttttatactgctctataaatagtattccaatcactgtgcttaatttaaatagcattatcttatcatttatcagccttttatgtattttccaagtaaaatattaacatattatttcattggtcttcttttttatctggttctatatgaatgctattttttcccttctcttctaacatgaaatatattttctctttttgatcttgtgctatgaaacaatcttccaaagaactgtataaggtggtcataagtgaatattttaattaaaattggtaaaaataaataataacagtaataatcatgcactata

SEQ ID NO: 115 - RNA, 1235 nt, Homo sapiens
  source 1..1235: mol_type = mRNA
aacagttctgacgtgagaggacaagaaacacaagcaaatataaaacattcaattctaagagaagttcatcagagacatccttcaggattgtgagctggattttcctctacgtgcttcagtcttctctacacaaagagctgcaaaccaggtctttgttttgcactcttatttcaagcagaatgagtgctcctaaactcctctctctgggctgtatcttcttccccttgctactttttcagcaggcccgggctcaattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctg

SEQ ID NO: 116 - RNA, 1235 nt, Homo sapiens; identical to SEQ ID NO: 115
  source 1..1235: mol_type = tRNA

SEQ ID NO: 117 - protein, 166 aa, Homo sapiens [sequence omitted]

SEQ ID NO: 118 - RNA, 7280 nt, Synthetic construct; identical to SEQ ID NO: 54
  source 1..7280: mol_type = other RNA

SEQ ID NO: 119 - RNA, 7280 nt, Synthetic construct; identical to SEQ ID NO: 54
  source 1..7280: mol_type = other RNA

SEQ ID NO: 120 - RNA, 477 nt, Synthetic construct
  source 1..477: mol_type = other RNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 121 - RNA, 297 nt, Synthetic construct
  source 1..297: mol_type = other RNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 122 - DNA, 1467 bp, Synthetic construct
  source 1..1467: mol_type = other DNA
atgggtaagtttctcgccactttgattttattcttccagttctgccccctcatcttcggtgattacagccccagctgctgtactctcacaattggagtctcctcataccactctaaaccctgcaatcctgcccagccagtttgttcgtggaccctcgacctgctggccctttcagcagatcaggccctacagcccccctgccctaacctagtaagttactccagctaccatgccacctattccctatatctattccctcattggactaagaagccaaaccgaaatggcggaggctattattcagcctcttattcagacccttgttccttaaagtgcccatacctggggtgccaatcatggacctgcccctatacaggagccgtctccagcccctactggaagtttcaacacgatgtcaattttactcaagaagtttcacgcctcaatattaatctccatttttcaaaatgcggttttcccttctcccttctagtcgacgctccaggatatgaccccatctggttccttaataccgaacccagccaactgcctcccaccgcccctcctctactcccccactctaacctagaccacatcctcgagccctctataccatggaaatcaaaactcctgacccttgtccagttaaccctacaaagcactaattatacttgcattgtctgtatcgatcgtgccagcctctccacttggcacgtcctatactctcccaacgtctctgttccatcctcttcttctacccccctcctttacccatcgttagcgcttccagccccccacctgacgttaccatttaactggacccactgctttgacccccagattcaagctatagtctcctccccctgtcataactccctcatcctgccccccttttccttgtcacctgttcccaccctaggatcccgctcccgccgagcggtaccggtggcggtctggcttgtctccgccctggccatgggagccggagtggctggcgggattaccggctccatgtccctcgcctcaggaaagagcctcctacatgaggtggacaaagatatttcccagttaactcaagcaatagtcaaaaaccacaaaaatctactcaaaattgcgcagtatgctgcccagaacagacgaggccttgatctcctgttctgggagcaaggaggattatgcaaagcattacaagaacagtgccgttttccgaatattaccaattcccatgtcccaatactacaagaaagacccccccttgagaatcgagtcctgactggctggggccttaactgggaccttggcctctcacagtgggctcgagaggccttacaaactggaatcacccttgttgcgctactccttcttgttatccttgcaggaccatgcatcctccgtcagctacgacacctcccctcgcgcgtcagatacccccattactctcttataaaacctgagtcatccctgtaa

SEQ ID NO: 123 - DNA, 352 bp, Human betaherpesvirus 5
  source 1..352: mol_type = other DNA
gtagaaatggccttcgcctacgcattagcactgttcgcagcagcccgacaagaagaggccggcgcccaagtctccgtcccacgagccctagaccgccaggccgcactcttacaaatacaagaatttatgatcacctgcctctcacaaacaccaccacgcaccacgttgctactgtatcccacggccgtggacctggccaaacgagccctttggacaccgaatcagatcaccgacatcaccagcctcgtacgcctggtctacatactctctaaacagaatcagcaacatctcatcccccagtgggcactacgacagatcgccgactttgccctaaaactacacaaaacgcacc

SEQ ID NO: 124 - DNA, 837 bp, Human betaherpesvirus 5
  source 1..837: mol_type = genomic DNA
atgtgccgccgcccggattgcggcttctctttctcacctggaccggtggtactgctgtggtgttgccttctgctgcccattgtttcctcagtcgccgtcagcgtcgctcctaccgccgccgagaaagtccccgcggagtgccccgaactaacgcgtcgatgcctgttgggtgaggtgtttcagggtgacaagtatgaaagttggctgcgcccgttggtgaatgttaccagacgcgatggcccgctatcgcaacttattcgttaccgtcccgttacgccggaggccgccaactccgtgctgttggacgatgctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacactcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctaa

SEQ ID NO: 125 - DNA, 1419 bp, Human betaherpesvirus 5
  source 1..1419: mol_type = genomic DNA
atggggaaaaaagagatgataatggtgaaaggcattcctaaaattatgctcctgatctctataacgttcttgctcctttccctcataaattgtaatgtattggtaaactccagaggaacaagacgttcctggccgtataccgtgctatcttatcgaggtaaagagattctgaagaaacagaaggaagatatcttaaaacgattgatgtctacatcatctgacggataccggtttttaatgtaccccagtcagcaaaaatttcatgccatcgttattagcatggataaatttcctcaagactacattttagcgggtcccattagaaatgatagcattacccatatgtggtttgacttttacagtactcaactccgaaaaccagccaagtacgtatattccgaatataatcacacggcccacaaaataacgttacgacccccaccttgcggcacagtgccttctatgaactgcctatccgaaatgttaaatgtttccaaacgcaatgataccggcgaaaaaggttgcggtaatttcaccacgtttaatcctatgtttttcaacgtaccacgttggaacacaaaactgtacataggttccaacaaagtcaacgtggatagtcagacaatctactttttgggcctaaccgccctacttttacgatacgcgcaacgtaactgcactcgcagtttctacctggttaacgccatgagccgaaatttattccgcgttcccaagtatattaacggcaccaagttgaaaaacactatgcgaaaactcaaacgtaaacaagcgcttgtcaaagaacaaccacaaaaaaagaataagaaatctcaaagtactactacgccatatctttcctatacaacgtctaccgctttcaacgtcaccactaacgtgacttatagtgctaccgctgctgtaacgcgggttgccacatctacgacaggttatcgtcctgatagtaactttatgaaatccattatggccacgcagttaagagatctcgcaacatgggtatatactactctgcggtatcggaatgaacccttttgtaaaccagaccgtaaccgtaccgccgtgtcagaatttatgaaaaacacgcacgtactgattcgtaacgaaacgccgtacactatttatggcactcttgacatgagctccttatattacaacgaaaccatgtccgtggaaaacgaaacggcttccgataataacgaaactacacctacgtcaccatcgacgaggtttcagagaacgttcatagatcccctatgggactatctagactcgctgctgtttctagataaaatccgtaactttagcctccagttacccgcgtatggaaatcttaccccgccggaacaccgccgggctgcaaatctatccaccctcaatagcctttggtggtggtcgcagtaa

SEQ ID NO: 126 - DNA, 693 bp, Human betaherpesvirus 5
  source 1..693: mol_type = genomic DNA
catgtctcgtttacgaagacgttactttttcatattcactgttttcgcaacagcttgttacgcgattacctataaggaaacatgctgcactcacattgctctcagggaacaattggaagactgctatgacttcaagaaatgtgatctgtatacaatatcgtacgtatgttaataaaacattctaaatcacatagaaagtgtgataagtcgttcttgattcaatgtaatctgccatccattcaggtttctctgccgcgttggacgactttgcatgtcacctagaaacttcagtaagatgaataatctcattttatctatcaaacagaacattaccaaatttgctcttcatcgtcatgtaccatactgcaactacaacccgtaagtcatacaattttctatctataaacaacgatactatctgataactggtagtaacgatgtatcttgcttcgatatagggcccatgtcatgaacgtaggagaacttcgctgcgcactatatactgacttcgatgatacaccaggcagaatgataggattggtcgatcgactggaaactacctggatgactccgtcctatcacgttaagatattagacttagatacatatttgattatgttaaacaatacatacaacagcacgagcaagacgatggacaccaaaatggcgtgtaataaaacctggttaaattga

SEQ ID NO: 127 - DNA, 645 bp, Human betaherpesvirus 5
  source 1..645: mol_type = genomic DNA
atgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgtttga

SEQ ID NO: 128 - DNA, 498 bp, Human betaherpesvirus 5
  source 1..498: mol_type = genomic DNA
atgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagaggtgagggtacgcgctaaagatgcatgacaacgggaaggtaagggcgaacgggtaacgggtaagtaaccgcatggggtatgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactag

SEQ ID NO: 129 - DNA, 645 bp, Human betaherpesvirus 5; identical to SEQ ID NO: 127
  source 1..645: mol_type = genomic DNA

SEQ ID NO: 130 - DNA, 1476 bp, Human betaherpesvirus 5
  source 1..1476: mol_type = genomic DNA
atggagtcctctgccaagagaaagatggaccctgataatcctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatccctcaaaacctttgaacaagtgaccgaggattgcaacgagaaccccgaaaaagatgtcctgacagaactcgtcaaacagattaaggttcgagtggacatggtgcggcatagaatcaaggagcacatgctgaaaaaatatacccagacggaagaaaaattcactggcgcctttaatatgatgggaggatgtttgcagaatgccttagatatcttagataaggttcatgagcctttcgaggacatgaagtgtattgggctaactatgcagagcatgtatgagaactacattgtacctgaggataagcgggagatgtggatggcttgtattaaggagctgcatgatgtgagcaagggcgccgctaacaagttggggggtgcactgcaggctaaggcccgtgctaaaaaggatgaacttaggagaaagatgatgtatatgtgctacaggaatatagagttctttaccaagaactcagccttccctaagaccaccaatggctgcagtcaggccatggcggcattgcagaacttgcctcagtgctctcctgatgagattatgtcttatgcccagaaaatctttaagattttggatgaggagagagacaaggtgctcacgcacattgatcacatatttatggatatcctcactacatgtgtggaaacaatgtgtaatgagtacaaggtcactagtgacgcttgtatgatgaccatgtacgggggcatctctctcttaagtgagttctgtcgggtgctgtgctgctatgtcttagaggagactagtgtgatgctggccaagcggcctctgataaccaagcctgaggttatcagtgtaatgaagcgccgcattgaggagatctgcatgaaggtctttgcccagtacattctgggggccgatcctttgagagtctgctctcctagtgtggatgacctacgggccatcgccgaggagtcagatgaggaagaggctattgtagcctacactttggccaccgctggtgccagctcctctgattctctggtgtcacctccagagtcccctgtacccgcgactatccctctgtcctcagtaattgtggctgagaacagtgatcaggaagaaagtgaacagagtgatgaggaacaggaggagggtgctcaggaggagcgggaggacactgtgtctgtcaagtctgagccagtgtctgagatagaggaagttgcctcagaggaagaggaggatggtgctgaggaacccaccgcctctggaggcaagagcacccaccctatggtgactagaagcaaggctgaccagtaa

SEQ ID NO: 131 - DNA, 1116 bp, Human betaherpesvirus 5
  source 1..1116: mol_type = genomic DNA
atggccccctcgcacgtggataaggtgaatacacggacatggagcgcttctatcgttttcatggtgctgacttttgtcaacgtcagcgtgcatctagtgctgagcaattttccgcacctgggctacccctgcgtctactatcacgtcgtggactttgaaaggctcaacatgtcggcctacaacgtaatgcacctgcacacgcctatgcttttcttagactcggtgcagttggtgtgctacgccgtgttcatgcagctcgtctttttagccgtgaccatctactacctggtatgctggatcaagatcagcatgcgcaaggacaaaggcatgagcctaaaccagtcgacacgcgacatttcgtacatgggcgacagcctcacagccttcctcttcattctcagcatggacacgttccaactattcacactgaccatgtcatttcggctgcccagcatgatcgccttcatggccgccgtacactttttctgcctgaccattttcaacgtgagcatggtcacgcagtaccgcagctacaaacgctcactctttttcttctcgcgtctgcaccccaagctcaaaggtacggtgcagttccgcacgctcatcgtcaacctggtagaggtagcgcttggtttcaacaccaccgtggtagccatggccctgtgctacggcttcggaaacaactttttcgtgcgtacaggccacatggtgttagccgtcttcgtggtctacgctatcatctccatcatctactttttactgatcgaggccgtcttttttcaatacgtcaaggtgcaattcggctaccacctgggcgccttctttggactctgcggcctcatctaccccatcgtgcagtacgataccttcctcagcaacgaataccgcaccggcatcagctggtcgttcggcatgctctttttcatatgggccatgtttacgacgtgtcgcgccgtccgctactttcgcggacgcggtagcggcagtgtcaagtaccaggcgctggccacagcctccggcgaagaagtcgctgcgctcagtcaccacgacagcttggaaagccgtcgcctccgcgaagaagaggacgacgatgatgaagacttcgaggacgcttaa

SEQ ID NO: 132 - DNA, 408 bp, Human betaherpesvirus 5
  source 1..408: mol_type = genomic DNA
atggagtggaacacactagtattaggtcttttggttttatcggtagtggcaagttccaacaatacgtcgactgctagcacaccgcgtccctctagttctactcacgcctcaacaaccgtgaaggcaacgactgttgcgacaactagtacaactacggcgacaagtacttcatcgacgactagtgccaaacctggttccactactcacgaccccaacgtgatgagaccacatgctcacaatgatttttacaatgcgcattgtacatcgcatatgtatgagctttcactgtccagctttgcagcctggtggactatgcttaacgctctcattctgatgggagctttttgtatcgtattacgacattgctgcttccagaactttactgcgaccaccaccaaaggctattga

SEQ ID NO: 133 - DNA, 687 bp, Human herpesvirus 8
  source 1..687: mol_type = genomic DNA; strain = GK18
atgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgttgaagatacgtctgcctctgggtctggagaagatgcaatagatgaatcggggtcgggggaggaagagcgtcccgtgacctcccacgtgacttttatgacacaaagcgtccaggccaccacagaactgaccgatgccttaatatcagccttttcaggatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaa

SEQ ID NO: 134 - DNA, 2538 bp, synthetic construct
  source 1..2538: mol_type = other DNA
atgactcccaggtctagattggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttaggaccaccgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcgtacctgcgtactataaacaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtga

SEQ ID NO: 135 - DNA, 2193 bp, Human herpesvirus 8
  source 1..2193: mol_type = genomic DNA; strain = GK18
atgcagggtctagccttcttggcggcccttgcatgctggcgatgcatatcgttgacatgtggagccactggcgcgttgccgacaacggcgacgacaataacccgctccgccacgcagctcatcaatgggagaaccaacctctccatagaactggaattcaacggcactagtttttttctaaattggcaaaatctgttgaatgtgatcacggagccggccctgacagagttgtggacctccgccgaagtcgccgaggacctcagggtaactctgaaaaagaggcaaagtctttttttccccaacaagacagttgtgatctctggagacggccatcgctatacgtgcgaggtgccgacgtcgtcgcaaacttataacatcaccaagggctttaactatagcgctctgcccgggcaccttggcggatttgggatcaacgcgcgtctggtactgggtgatatcttcgcatcaaaatggtcgctattcgcgagggacaccccagagtatcgggtgttttacccaatgaatgtcatggccgtcaagttttccatatccattggcaacaacgagtccggcgtagcgctctatggagtggtgtcggaagatttcgtggtcgtcacgctccacaacaggtccaaagaggctaacgagacggcgtcccatcttctgttcggtctcccggattcactgccatctctgaagggccatgccacctatgatgaactcacgttcgcccgaaacgcaaaatatgcgctagtggcgatcctgcctaaagattcttaccagacactccttacagagaattacactcgcatatttctgaacatgacggagtcgacgcccctcgagttcacgcggacgatccagaccaggatcgtatcaatcgaggccaggcgcgcctgcgcagctcaagaggcggcgccggacatattcttggtgttgtttcagatgttggtggcacactttcttgttgcgcggggcattgccgagcaccgatttgtggaggtggactgcgtgtgtcggcagtatgcggaactgtattttctccgccgcatctcgcgtctgtgcatgcccacgttcaccactgtcgggtataaccacaccacccttggcgctgtggccgccacacaaatagctcgcgtgtccgccacgaagttggccagtttgccccgctcttcccaggaaacagtgctggccatggtccagcttggcgcccgtgatggcgccgtcccttcctccattctggagggcattgctatggtcgtcgaacatatgtataccgcctacacttatgtgtacacactcggcgatactgaaagaaaattaatgttggacatacacacggtcctcaccgacagctgcccgcccaaagactccggagtatcagaaaagctactgagaacatatttgatgttcacatcaatgtgtaccaacatagagctgggcgaaatgatcgcccgcttttccaaaccggacagccttaacatctatagggcattctccccctgctttctaggactaaggtacgatttgcatccagccaagttgcgcgccgaggcgccgcagtcgtccgctctgacgcggactgccgttgccagaggaacatcgggattcgcagaattgctccacgcgctgcacctcgatagcttaaatttaattccggcgattaactgttcaaagattacagccgacaagataatagctacggtacccttgcctcacgtcacgtatatcatcagttccgaagcactctcgaacgctgttgtctacgaggtgtcggagatcttcctcaagagtgccatgtttatatctgctatcaaacccgattgctccggctttaacttttctcagattgataggcacattcccatagtctacaacatcagcacaccaagaagaggttgccccctttgtgactctgtaatcatgagctacgatgagagcgatggcctgcagtctctcatgtatgtcactaatgaaagggtgcagaccaacctctttttagataagtcacctttctttgataataacaacctacacattcattatttgtggctgagggacaacgggaccgtagtggagataaggggcatgtatagaagacgcgcagccagtgctttgtttctaattctctcttttattgggttctcgggggttatctactttctttacagactgttttccatcctttattag

SEQ ID NO: 136 - DNA, 1224 bp, Human herpesvirus 8
  source 1..1224: mol_type = genomic DNA; strain = GK18
atggcgatgtttgtgaggacctcgtctagcacacacgatgaagagagaatgcttccaattgaaggagcgcctcgcagacgaccccccgtgaagttcatattcccacctccacctctttcatcacttccaggatttggcaggccgcgcggctatgctggacccacggtgatagatatgtctgccccagacgacgtcttcgccgaggacacgccatcgccgccagcaacccctctggatctacagatatccccggatcagtcgagcggcgaatctgaatatgacgaggatgaggaagatgaagatgaagaagaaaatgacgatgttcaggaggaagacgagccagaggggtaccctgcagacttttttcaacctttatctcacttgcgcccgaggcctctggccagacgggcccatacgcccaaaccggtagcagtggtagcgggccgcgtgcgcagttcaacggacacggcggagtccgaggcgtccatgggatgggttagtcaggatgacggattttcccctgctgggctctcaccttcagacgacgagggggttgctatcctggaaccgatggcggcatacactgggaccggggcatacggactttcacctgcttccagaaatagtgtacctggaacacaaagttcaccatacagcgaccctgatgaagggccctcgtggcgccccctgcgcgccgcacccaccgcgatcgtcgacctgacatcggactctgatagcgatgacagttccaactctccggacgtgaacaatgaggccgcgtttaccgacgcgcgccatttttcccaccagccaccctcgtccgaggaggacggagaagaccaaggggaagtattgagtcagagaatcgggctcatggacgtgggccagaagcgcaaaaggcagtctaccgcctcctctggtagcgaggatgtggtgcgctgccagagacaaccaaacttaagccgcaaagcagtggcgtctgtgataattatatcctcggggagtgacacagacgaggagccctcgtccgccgtgagcgtgatcgtgtctccgtcgagcacaaagggtcacctcccaacccaatctcccagtacttccgcccactcgatttcatcaggaagcacaactaccgcggggtccaggtgcagcgacccaacccgcatcctggcctccacgccacccctgtgtggaaacggtgcatataactggccgtggctggactga

SEQ ID NO: 137 - DNA, 1203 bp, Human herpesvirus 8
  source 1..1203: mol_type = genomic DNA; strain = GK18
atgcgcgcttcaaagagcgaccgttttctcatgtcctcctgggtcaagctgttgtttgtggctgtaattatgtacatttgctctgcagtcgtgcccatggccgccacgtacgagggccttggtttcccctgctacttcaacaatctggtaaactacagcgcgctaaacctcacggtgcgcaactcagcgaagcacctcaccccgaccctgtttttagagaaaccagagatgctggtgtacatattttggacttttatcgtggacggcattgccatagtatactactgtctcgcggcggtggccgtatatcgggccaagcatgttcacgccactaccatgatgagcatgcaatcctggatagcgctacttggatcacatagcgtgctttatgtcgccatcttaaggatgtggagcatgcaactgtttatccatgttctgtcatataaacacgtacttatggcagcctttgtctactgcattcatttttgcatatcctttgcccacatccagtcactgataacatgtaactccgcccagtgggaaatacctctcctggaacagcacgtacctgataataccatgatggagtcactgctgactcggtggaaaccggtctgcgtcaacctgtacctatcaacaaccgccttggaaatgctgctgttttcattgagcaccatgatggccgttggcaacagcttttacgtgctcgtgtcagacgccatttttggtgcggtgaacatgttcctagccttgacggtcgtgtggtacataaacacggaatttttcctggtaaagtttatgagacggcaggtaggcttttacgtgggagtattcgtgggttatctgattctactgctgcctgtcatcaggtatgaaaacgcgttcgtgcaggctaatctccactacatagtggctatcaatatctcctgtatccctatactgtgcatcctagccatcgttatacgcgttataaggagcgactggggactctgtacgccgagtgcggcatacatgcctctggcgacgtcggcgccgacggtcgatagaactccgactgttcaccagaaaccgccgcccctcccagcaaagaccagagcccgcgccaaagtgaaggacatatcgacgcccgccccgaggacccaataccagtccgaccatgaaagtgacagtgaaatcgacgaaacgcaaatgatattcatttag

SEQ ID NO: 138 - DNA, 333 bp, Human herpesvirus 8
  source 1..333: mol_type = genomic DNA; strain = GK18
atgacagcgtccacggtggccctggctttatttgtggcctcgatactaggtcactgctgggttacggccaactcgacaggtgtcgcatcatctacagagcgttctagtcccagtacggctgggctcagcgccaggccttcgcccggtcccacatcagtcactaccccaggattctatgacgttgcttgttccgcggatagttttagcccctccctttctagcttttctagcgtctgggccttgattaacgcgttactagtcgtcgtggccacatttttctatctggtatatctttgttttttcaagtttgtggacgaggtggtccatgcatag

SEQ ID NO: 139 - DNA, 1653 bp, Human herpesvirus 8
  source 1..1653: mol_type = genomic DNA; strain = GK18
atggcctttttaagacaaacactgtggattttatggacatttaccatggtcattggccaggacaatgaaaagtgttcccaaaaaaccttaattggatatagacttaaaatgtctcgtgacggtgacattgcagttggagaaacagtggaattacgttgtagatctggatacactacttatgcccgcaatataacagcaacatgtttacaaggtgggacgtggtctgaaccaacggcaacatgtaacaaaaagtcctgtccaaacccaggtgaaatacaaaatggaaaggttatatttcatggtggacaagatgccttaaaatatggggcaaacatttcatatgtttgtaatgaaggatattttttggttggtcgagaatacgtgcgatattgtatgattggagcatctggccaaatggcgtggtcatcttctcctcctttttgtgaaaaagaaaagtgtcacagaccgaaaatcgaaaatggagattttaagcctgataaagattattatgagtataatgatgcagttcattttgaatgtaatgaaggatatactctagttggaccacattccattgcatgtgcagttaataacacgtggacatctaacatgccaacctgtgaactcgcaggctgtaaatttccatcggtgactcatggttatccaatccaaggtttttctcttacttataaacataagcaaagtgttacttttgcatgcaatgatggatttgttctcagaggatcccccacaattacgtgtaacgttactgaatgggacccaccacttcctaagtgtgttttggaagatatagatgatccaaacaattcaaatcctggacgtttgcatccaacacccaatgaaaaaccaaatggtaatgtctttcaacgctcaaactatacagaacctccaacaaagcctgaagacacccatacagcagctacttgtgataccaactgtgaacagccacctaaaatcctgccaacatccgaaggttttaatgagactaccacatctaatacaattacaaaacaattagaggatgagaaaactacatcccagccaaatacacatattacatctgccttaacatccatgaaagcgaaaggtaactttaccaacaagaccaataactctactgatctacatatagcgtctacacccacttcccaagatgatgctacgccttcaatacctagtgtacagacacccaattataatactaacgcaccgacacgtacactaacgtctctccatattgaagaaggcccatccaattctactacttcagaaaaggccactgcctctactctctcacacaactcacacaaaaatgacaccggaggcatatacacaacattaaacaaaacaacacagttgccatccactaataaacctacaaacagtcaagccaagagttccactaagccacgcgttgagacacacaataaaacaaccagtaatcctgccatttctttaacagattctgcagatgtgcctcagagaccgcgagaaccaacactccctcccattttcaggccaccggcgtctaaaaatcgctatctggaaaagcaactagttattggactactaaccgctgtcgccctaacgtgtggactgattaccttatttcactatctgttctttcgttag

SEQ ID NO: 140 - DNA, 873 bp, Human herpesvirus 8
  source 1..873: mol_type = genomic DNA; strain = GK18
atggcgtcatctgatattctgtcggttgcaaggacggatgacggctccgtctgtgaagtctccctgcgtggaggtaggaaaaaaactaccgtctacctgccggacactgaaccctgggtggtagagaccgacgccatcaaagacgccttcctcagcgacgggatcgtggatatggctcgaaagcttcatcgtggtgccctgccctcaaattctcacaacggcttgaggatggtgcttttttgttattgttacttgcaaaattgtgtgtacctagccctgtttctgtgcccccttaatccttacttggtaactccctcaagcattgagtttgccgagcccgttgtggcacctgaggtgctcttcccacacccggctgagatgtctcgcggttgcgatgacgcgattttctgtaaactgccctataccgtgcctataatcaacaccacgtttggacgcatttacccgaactctacacgcgagccggacggcaggcctacggattactccatggcccttagaagggcttttgcagttatggttaacacgtcatgtgcaggagtgacattgtgccgcggagaaactcagaccgcatcccgtaaccacactgagtgggaaaatctgctggctatgttttctgtgattatctatgccttagatcacaactgtcacccggaagcactgtctatcgcgagcggcatctttgacgagcgtgactatggattattcatctctcagccccggagcgtgccctcgcctaccccttgcgacgtgtcgtgggaagatatctacaacgggacttacctagctcggcctggaaactgtgacccctggcccaatctatccacccctcccttgattctaaattttaaataa

SEQ ID NO: 141 - DNA, 309 bp, Human herpesvirus 8
  source 1..309: mol_type = genomic DNA; strain = GK18
atgagcatgacttccccgtctccagtcaccggaggaatggtggacggctccgtcctggtgcgaatggccaccaagcctcccgtgattggtcttataacagtgctcttcctcctagtcataggcgcctgcgtctactgctgcattcgcgtgttcctggcggctcgactgtggcgcgccaccccactaggcagggccaccgtggcgtatcaggtccttcgcaccctgggaccgcaggccgggtcacatgcaccgccgacggtgggcatagctacccaggagccctaccgtacaatatacatgccagattag

SEQ ID NO: 142 - DNA, 1404 bp, Human herpesvirus 8
  source 1..1404: mol_type = genomic DNA; strain = GK18
atgtttgttccctggcaactcggtacaattacccgtcaccgagatgagctccaaaaactactggcagcctccctgctcccggagcacccggaggagagcctcggtaaccccataatgacacagattcaccagtcgctccaaccatcttccccctgcagggtctgtcagctcctattttctctggtccgcgattcgtccacccccatgggtttcttcgaggactatgcctgcctctgcttcttctgtctatacgccccacactgctggacctcgaccatggcggcagcggcagacctgtgcgagatcatgcatctgcactttccagaagaggaggcgacatacgggctattcggaccgggtcgccttatgggtatcgacttgcagctgcacttctttgttcaaaagtgctttaagaccaccgccgccgaaaaaatactgggaatatccaacctgcaatttttaaaatcagaattcatccggggcatgctcacaggcaccatcacctgcaacttctgcttcaaaacgtcctggcccaggacagacaaggaggaggccaccggccccaccccatgctgccagattacagacaccaccaccgcacccgcgagcggcataccggaactagcccgggccacattctgcggcgcaagtcgccccacaaagcccagcctacttcccgcgctaatagatatctggtccacgagctcagagctccttgacgagccgcgccctcgactgatcgcaagcgacatgagtgaactcaaatccgtggtcgcatcccacgatccgttcttctctcccccgcttcaggcagacacctcacagggtccatgtctgatgcacccaaccctggggctacgatacaaaaacgggactgcatccgtctgcctcctctgcgagtgccttgcggcacacccagaggcacccaaggcgctgcagacccttcagtgcgaggtaatgggccatatagaaaacaacgtaaagctggtagacagaattgcctttgtgttggacaacccattcgccatgccatatgtctcagatccgctacttagagagctgatccggggctgtaccccacaggaaattcacaagcacctgttctgcgacccgctgtgcgccctcaatgctaaggtggtgtcagaggacgtactattccgcctgcccagggagcaggagtataaaaagctcagggcatccgcggccgccggacagctcctcgatgccaacaccctgttcgactgcgaggtcgtgcagactttggtctttctctttaagggtctccaaaacgccagggtggggaaaaccacctcactagacattattcgggagctaaccgcacaactaaaaagacaccgcctagacctggcccacccctcacagacgtcacacttgtacgcttga

SEQ ID NO: 143 - DNA, 3390 bp, Human herpesvirus 8
  source 1..3390: mol_type = genomic DNA; strain = GK18
atggcgcccccgggaatgcgcctgaggtcgggacggagcaccggcgcgcccttaacgagaggaagttgtaggaaacgaaacaggtctccggaaagatgtgaccttggcgatgacctacatctacaaccgcgaaggaagcatgtcgccgactccgtcgacggccgggaatgtggaccccacaccttgcctataccaggaagtcccacagtgttcacatccgggctgccagcatttgtgtctagtcctactttaccggtggctcccattccttcacccgctcccgcaacacctttacctccaccggcactcttaccccccgtaaccacgtcttcctccccaatccctccatcccatcctgtgtctccggggaccacggatactcattctccatctcctgcattgccacccacgcagtctccagagtcttctcaaaggccaccgctttcaagtcctacaggaaggccagactcttcaacacctatgcgtccgccaccctcgcagcagactacacctccacactcacccacgactcctccacccgagcctccctccaagtcgtcaccagactctttagctccgtctaccctgcgtagcctgagaaaaagaaggctatcgtccccccaaggtccctctacactaaacccaatatgtcagtcgcccccagtctctccccctagatgtgacttcgccaaccgtagtgtgtaccccccatgggccacagagtccccgatctacgtgggatcatccagcgatggcgatactccgccacgccaaccgcctacatctcccatctccataggatcatcatccccgtctgagggatcctggggtgatgacacagccatgttggtgctccttgcggagattgcagaagaagcatccaagaatgaaaaagaatgttccgaaaataatcaggctggcgaggataatggggacaacgagattagcaaggaaagtcaggttgacaaggatgacaatgacaataaggatgatgaggaggagcaggagacagatgaggaggacgaggaggatgacgaggaggatgacgaggaggatgacgaggaggatgacgaggaggatgacgaggaggatgacgaggaggatgacgaggaggaggacgaggaggaggacgaggaggaggacgaggaggaggacgaggaggaggaggaggacgaggaggatgacgatgatgaggacaatgaggacgaggaggatgacgaggaggaggacaagaaggaggacgaggaggacgggggcgatggaaacaaaacgttgagcatccaaagttcacaacagcagcaggagccacaacagcaggagccacagcagcaggagccacaacagcaggagccacagcagcaggagccacagcagcaggagccacaacagcaggagccacagcagcaggagccacaacagcgggagccacagcagcgggagccacagcagcgggagccccagcagcgggagccccagcagcgggagccccagcagcgggagccccagcagcgggagccccagcagcgggagccacagcagcgggagccacagcagcgggagccacagcagcgggagccccagcagcgggagccacagcagcaggagccacagcagcaggagccacagcagcaggagccacagcagcaggagccacagcagcaggagccacagcagcaggagccacagcagcaggagccacaacagcaggagccacaacagcaggagccacaacagcaggagccacaacagcaggagccacaacagcaggagccacagcagcaggatgagcagcagcaggatgagcagcagcaggatgagcagcagcaggatgagcagcagcaggatgagcagcagcaggatgagcagcagcaggatgagcagcagcaggatgagcaggagcagcaggatgagcagcagcaggatgagcagcagcagcaggatgaacaggagcagcaggaggagcaggagcagcaggaggagcagcagcaggatgagcagcagcaggatgagcagcagcaggatgagcagcagcaggatgagcaggagcagcaggatgagcagcagcaggatgagcagcagcagcaggatgaacaggagcagcaggaggagcaggagcagcaggaggagcaggagcagcaggaggagcaggagcagcaggaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagttagaggagcaggagcaggagcaggagttagaggaggtggaagagcaagagcaggagcaggaagagcaggaattagaggaggtggaggagcaagagcaggagcaggaggagcaggaggagcaggagttagaggaggtggaagagcaggaagagcaggagttagaggaggtggaagagcaggaagagcaggagttagaggaggtggaagagcaggagcagcagggggtggaacagcaggagcaggagacggtggaagagcccataatcttgcacgggtcgtcatccgaggacgaaatggaagtggattaccctgttgttagcacacatgaacaaattgccagtagcccaccaggagataatacaccagacgatgacccacaacctggcccatctcgcgaataccgctatgtactcagaacatcaccaccccacagacctggagttcgtatgaggcgcgttccagttacccacccaaaaaagccacatccaagataccaacaaccaccggtcccttacagacagatagatgattgtcctgcgaaagctaggccacaacacatcttttatagacgctttttgggaaaggatggaagacgagatccaaagtgtcaatggaagtttgcagtgattttttggggcaatgacccatacggacttaaaaaattatctcaggccttccagtttggaggagtaaaggcaggccccgtgtcctgcttgccccaccctggaccagaccagtcgcccataacttattgtgtatatgtgtattgtcagaacaaagacacaagtaagaaagtacaaatggcccgcctagcctgggaagctagtcaccccctggcaggaaacctacaatcttccatagttaagtttaaaaagcccctgccattaacccagccaggggaaaaccaaggtcctggggactctccacaggaaatgacataa

SEQ ID NO: 144 - DNA, 1029 bp, Human herpesvirus 8
  source 1..1029: mol_type = genomic DNA; strain = GK18
atggcggccgaggatttcctaaccatcttcttagatgatgatgaatcctggaatgaaactctaaatatgagcggatatgactactctggaaacttcagcctagaagtgagcgtgtgtgagatgaccaccgtggtgccttacacgtggaacgttggaatactctctctgattttcctcataaatgttcttggaaatggattggtcacctacattttttgcaagcaccgatcgcgggcaggagcgatagatatactgctcctgggtatctgcctaaactcgctgtgtcttagcatatctctattggcagaagtgttgatgtttttgtttcccaatatcatctccacaggcttgtgcagacttgaaatttttttttactatttatatgtctacttggatatcttcagtgttgtgtgcgtcagtctagtgaggtacctcctggtggcatattctacgcgttcctggcccaagaagcagtccctcggatgggtactgacatccgctgcactgttaattgcattggtgctgtcgggggatgcctgtcggcacaggagcagggtggtcgacccggtcagcaagcaggccatgtgttatgagaacgcgggaaacatgactgcagactggcgactgcatgtcagaaccgtgtcagttactgcaggtttcctgttacccctggccctccttattctgttttatgctctcacctggtgtgtggtgaggaggacaaagctgcaagccaggcggaaggtaaggggggtgattgttgctgtggtgctgctgttttttgtgttttgcttcccttaccacgtactaaatctactggacactctgctaaggcgacgctggatccgggacagctgctatacgcgggggttgataaacgtgggtctggcagtaacctcgttactgcaggcactgtacagcgccgttgttcccctgatatactcctgcctgggatccctctttaggcagaggatgtacggtctcttccaaagcctcaggcagtctttcatgtccggcgccaccacgtag

SEQ ID NO: 145 - DNA, 615 bp, Human herpesvirus 8
  source 1..615: mol_type = genomic DNA; strain = GK18
atgcgctggttcaagttgtggtctatcttgctggtcggttcactgctggtatctggaacgcggggcaagttgccggacgcccccgagtttgaaaaggatcttctcattcagagactcaattggatgctatgggtgatcgatgaatgcttccgcgacctctgttaccgtaccggcatctgcaagggtattctagagcccgctgctatttttcatctgaaactaccagccatcaacgatactgatcactgcgggttaataggatttaatgagactagctgccttaaaaagctcgccgatggctttttcgaattcgaggtgttgtttaagtttttaacgacggagtttggaaaatcagtgataaacgtggacgtcatggagcttctgacgaagaccttaggatgggacatacaggaagagctcaataagctgactaagacgcactacagtccacccaaatttgaccgcggtctattagggaggcttcagggacttaagtattgggtgagacactttgcttcgttttatgttctgagtgcaatggaaaagtttgcaggtcaagcggtgcgtgttttgaactctatcccagacgtgactcctgacgtccacgataagtaa

SEQ ID NO: 146 - DNA, 567 bp, Human herpesvirus 8
  source 1..567: mol_type = genomic DNA; strain = GK18
atggccacttacgaggttctctgtgaggtggcgcggaaactgggcacggatgacagggaagtggtattgttcctcctaaacgtgttcatacctcaacccacactggcccaattaattggagctcttagagctttaaaggaggagggcaggttaacgtttcccctgttagcggaatgtctgtttcgtgcaggtcgcagagacctcttgcgcgacctgcttcacttagacccgcgttttttagagcgccacctagcgggcacaatgagttatttcagcccttatcagctcactgttctccacgtagacggggagctgtgtgcgagggatattaggtctttgatatttttaagcaaggacactatagggtctcgcagcacaccacagacattcttacactgggtgtactgtatggaaaacttagacctactgggtcccactgacgtggatgccctaatgtcaatgcttagatctttgtcaagagtagacctacagcgccaagtgcaaaccctaatgggccttcacctttccggcccatcacactcccaacactatcgccatacaccatag

SEQ ID NO: 147 - DNA, 2724 bp, Human gammaherpesvirus 4
  source 1..2724: mol_type = genomic DNA
atggaggcagccttgcttgtgtgtcagtacaccatccagagcctgatccatctcacgggtgaagatcctggttttttcaatgttgagattccggaattcccattttaccccacatgcaatgtttgcacggcagatgtcaatgtaactatcaatttcgatgtcgggggcaaaaagcatcaacttgatcttgactttggccagctgacaccccatacgaaggctgtctaccaacctcgaggtgcatttggtggctcagaaaatgccaccaatctctttctactggagctccttggtgcaggagaattggctctaactatgcggtctaagaagcttccaattaacgtcaccaccggagaggagcaacaagtaagcctggaatctgtagatgtctactttcaagatgtgtttggaaccatgtggtgccaccatgcagaaatgcaaaaccccgtgtacctgataccagaaacagtgccatacataaagtgggataactgtaattctaccaatataacggcagtagtgagggcacaggggctggatgtcacgctacccttaagtttgccaacgtcagctcaagactcgaatttcagcgtaaaaacagaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatcctgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaagacactcattatcacacgaacggctaccaatgccaccacaacaacccacaaggttatattctccaaggcacccgagagcaccaccacctcccctaccttgaatacaactggatttgctgatcccaatacaacgacaggtctacccagctctactcacgtgcctaccaacctcaccgcacctgcaagcacaggccccactgtatccaccgcggatgtcaccagcccaacaccagccggcacaacgtcaggcgcatcaccggtgacaccaagtccatctccatgggacaacggcacagaaagtaaggcccccgacatgaccagctccacctcaccagtgactaccccaaccccaaatgccaccagccccaccccagcagtgactaccccaaccccaaatgccaccagccccaccccagcagtgactaccccaaccccaaatgccaccagccccaccttgggaaaaacaagtcctacctcagcagtgactaccccaaccccaaatgccaccagccccaccttgggaaaaacaagccccacctcagcagtgactaccccaaccccaaatgccaccagccccaccttgggaaaaacaagccccacctcagcagtgactaccccaaccccaaatgccaccggccctactgtgggagaaacaagtccacaggcaaatgccaccaaccacaccttaggaggaacaagtcccaccccagtagttaccagccaaccaaaaaatgcaaccagtgctgttaccacaggccaacataacataacttcaagttcaacctcttccatgtcactgagacccagttcaaacccagagacactcagcccctccaccagtgacaattcaacgtcacatatgcctttactaacctccgctcacccaacaggtggtgaaaatataacacaggtgacaccagcctctatcagcacacatcatgtgtccaccagttcgccagcaccccgcccaggcaccaccagccaagcgtcaggccctggaaacagttccacatccacaaaaccgggggaggttaatgtcaccaaaggcacgcccccccaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaccgagatacaatgcgaccacctatctacctcccagcacttctagcaaactgcggccccgctggacttttacgagcccaccggttaccacagcccaagccaccgtgccagtcccgccaacgtcccagcccagattctcaaacctctccatgctagtactgcagtgggcctctctggctgtgctgacccttctgctgctgctggtcatggcggactgcgcctttaggcgtaacttgtctacatcccatacctacaccaccccaccatatgatgacgccgagacctatgtataa

SEQ ID NO: 148 - DNA, 2121 bp, Human gammaherpesvirus 4
  source 1..2121: mol_type = genomic DNA
atgcagttgctctgtgttttttgcctggtgttgctatgggaggtgggggctgccagcctcagcgaggttaagctgcacctggacatagaggggcatgcttcgcattacaccatcccatggaccgaactgatggcaaaggtcccaggccttagcccagaggcgctgtggagagaggcaaatgtcaccgaagatttggcgtctatgcttaaccgctacaagttaatttacaagacgtctggtacccttggtattgcgctggccgagcctgtcgatatccctgctgtctctgaaggatccatgcaagtggatgcatctaaggtccatcccggagtcattagcggcctgaattcccctgcctgcatgcttagtgccccccttgagaagcagctcttctactatattggcaccatgctgcccaacacgcggccacacagctatgtcttttatcagctgcgctgtcacttgtcttatgtggccctgtccatcaacggggacaagtttcagtacacgggggccatgacttctaaatttctgatgggcacctacaagcgagtgaccgagaagggagatgagcatgtgttgagcctggtctttggcaagacgaaggacctgccggatctgagggggccttttagttacccatccttaaccagtgcccaaagcggggactattccctggtgattgttacaacctttgtgcattatgccaactttcacaactactttgtacccaacctgaaggatatgttttcccgagccgtcaccatgacagccgccagctacgctcgctacgttctccagaaactggtcctgctggagatgaagggaggctgccgggagccggaactggacacggaaacgctgactaccatgtttgaggtttctgtggccttctttaaggtgggtcatgctgtgggtgagactggcaatggctgcgtggacctccgctggttggccaagagcttctttgagctgactgtcctgaaagacatcatcggcatatgttatggggccactgtcaagggcatgcaatcctacgggctggagcgcttggccgccatgctgatggccacggtcaagatggaggagctgggtcacctgactactgagaaacaggagtacgcgctgaggttagccaccgtcggctaccccaaggccggggtttacagtggcctcattggaggcgccacatctgtgcttctctcggcctacaaccgccacccccttttccagcccctgcataccgtgatgagagagaccctgtttatcggcagccacgtggtgctacgcgagttgcggctgaacgtgactacccaggggcccaaccttgccctataccaactgctgtccaccgccctgtgctcggccctagagattggggaggttttgcgggggctagccctggggacagagagcgggctcttctcaccgtgctacctcagcctacgatttgacctcacacgagacaagctgctgagcatggccccccaggaggcaacgctggaccaggcggccgtttcaaatgctgtggatgggtttcttgggcggctctctttggagcgagaagacagggatgcgtggcatctccccgcctacaaatgcgtggacaggctcgacaaagttctgatgattatcccgctcatcaatgtgacattcataatctctagtgaccgtgaggtccgaggctcggcgctatacgaggccagcaccacctatctcagcagctctctctttctctcccccgttataatgaataaatgttcgcagggtgctgtggctggggagccccgccagattccaaagatccagaattttaccaggacgcagaaatcctgcattttttgtggctttgccctgctcagttatgatgaaaaggaaggcctggaaactacaacctacatcacctcccaggaagtccaaaactccatcttgagctccaactactttgattttgacaacctccacgttcactatctgctgctgaccaccaacgggactgtcatggaaattgcgggcctgtatgaagaaagagcacacgttgttttggcaataatcctgtactttattgcttttgctctgggtatctttctggttcacaagattgttatgtttttcctttag

SEQ ID NO: 149 - DNA, 414 bp, Human gammaherpesvirus 4
  source 1..414: mol_type = genomic DNA
atgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagctggtcatcagccgctgcgcaaacggactcaacgtggtctccttctttatctccatcctgaagcgaagcagctccgccctcacgggccatctccgtgagttgttaaccaccctggagactctttacggttcattctcagtggaagacctgtttggtgccaacttaaacagatacgcatggcatcgcgggggctag

SEQ ID NO: 150 - DNA, 672 bp, Human gammaherpesvirus 4
  source 1..672: mol_type = genomic DNA
atggtttcatttaagcaggtgagggtgccattgtttaccgccatcgcacttgttattgttctactcctggcatactttttgccacccagggtaagaggaggagggcgggtggcagccgcggccatcacctgggtacccaaaccaaatgtagaggtctggccggtggatcctccaccgccggttaactttaacaagacggccgagcaggagtatggggacaaagaggtaaaactgccacattggacacccaccctgcacacatttcaggtaccccaaaactataccaaagctaactgtacatactgcaacaccagagaatacacattttcatataaaggatgctgtttttatttcaccaaaaagaagcacacctggaatgggtgtttccaagcctgtgcagagctatatccatgcacttatttttatgggccaacgcccgatattctacctgtggtaactagaaatctgaatgccattgagtccctttgggtcggggtgtacagggtgggagaagggaactggacatcattagatggggggacttttaaggtttatcaaatttttggctctcattgtacatatgtcagcaaatttagtacagttccagtctcacaccatgagtgttcattccttaaaccatgtttatgtgtcagtcaaagatcaaatagctaa

SEQ ID NO: 151 - DNA, 738 bp, Human gammaherpesvirus 4
  source 1..738: mol_type = genomic DNA
atgatggacccaaactcgacttctgaagatgtaaaatttacacctgacccataccaggtgccttttgtacaagcttttgaccaagctaccagagtctatcaggacctgggagggccatcgcaagctcctttgccttgtgtgctgtggccggtgctgccagagcctctgccacaaggccagctaactgcctatcatgtttcaaccgctccgactgggtcgtggttttctgcccctcagcctgctcctgagaatgcttatcaagcttatgcagcacctcagctgttcccagtctccgacataacccagaatcaacagactaaccaagccgggggagaagcacctcaacctggagacaattctactgttcaaacagcagcagcagtggtgtttgcttgccccggggctaaccaaggacaacagctagcagacattggtgttccacagcctgcaccagtggctgccccggcacgacgcacacggaaaccacaacagccagaatcgctggaggaatgcgattctgaactagaaataaagcgatacaagaatcgggtggcttccagaaaatgccgggccaagtttaagcaactgctgcagcactaccgtgaggtggctgctgccaaatcatctgaaaatgacaggctgcgcctcctgttgaagcagatgtgcccaagcctggatgttgactccattatcccccggacaccagatgttttacacgaggatctcttaaatttctaa

SEQ ID NO: 152 - DNA, 1818 bp, Human gammaherpesvirus 4
  source 1..1818: mol_type = genomic DNA
atgaggcctaaaaaggatggcttggaagactttctgaggctaactcctgaaatcaaaaagcagctgggctctctggtctctgactactgcaacgtcctcaacaaggaatttacagccgggagtgtggagattactctgagatcctacaaaatatgcaaggcatttataaatgaggccaaggcccacgggcgagaatggggcgggctaatggccacgctcaacatctgcaatttttgggccattctccgaaacaacagggtaagaagacgggctgagaatgccggcaacgacgcatgttccatcgcgtgccctatagtgatgcgctacgtgttagaccacctgatagtggtcactgacagattcttcatccaggcccccagcaaccgggtgatgattcctgccaccataggcaccgctatgtacaagctcctaaaacacagtcgggtgcgggcctacacctacagcaaggtgctgggcgtggaccgcgcggccatcatggcctccggcaagcaggtagtggaacacctgaacaggatggagaaggaaggcctcctaagctccaagttcaaggccttttgcaagtgggtgttcacctatcccgtcctcgaggagatgttccagactatggtctcgtccaagacaggccatctgacggacgatgttaaggatgtcagggctctgattaagacactgccccgggcctcctactccagccacgccggacagaggagctacgtgagcggcgtgcttcccgcgtgcctgctgtcaaccaagtccaaggcagtggaaactcctatcctcgtgtccggagccgacaggatggacgaggagctcatggggaatgatgggggtgcctctcacaccgaggcccgctactcggagtccggacagtttcatgcttttacagatgaactcgaaagtctcccgagcccgaccatgcccctgaagcccggtgcccaaagcgccgactgcggtgacagcagttccagcagcagtgactcgggcaacagtgacaccgagcagagcgagcgggaagaggccagggccgaggccccgcgcctgcgggccccaaagtcgcgccggacatccaggcccaaccgtggtcaaactccatgtccttccaacgcggcggaacctgaacagccttggatagcagcggtccaccaagagagcgatgagagacccatattcccccacccctcaaagcccacctttcttcctcccgttaaaaggaagaagggcctcagggatagccgggaaggtatgttcctgccaaagccggaagcgggcagtgccatatctgacgtgttcgaggggcgagaggtgtgtcagccaaagaggatcaggcccttccatccacccggatccccgtgggccaaccggcccctgcctgcctctttggctcccacccccacaggacctgtccatgaaccggtcggatccctaacgccagccccggtgccccagccacttgacccggcccccgcagtaacccccgaggcaagtcatctgttggaggaccctgatgaagaaaccagtcaggccgtgaaggccctaagggagatggctgacactgttattccccagaaggaggaagcagccatatgtggacagatggacctgagccacccgccccctcgtggccatttggacgaactgaccacaacactagagtccatgacagaggatttgaatctggactcccccctgacccccgaacttaatgaaatcttggatacatttctaaatgatgaatgtctgctgcatgccatgcatatttcaactgggctgtctatttttgacaccagcttattttag

SEQ ID NO: 153 - DNA, 1317 bp, Human gammaherpesvirus 4
  source 1..1317: mol_type = genomic DNA
atggagggcagcgaagaacactccacagatggagagatttcatcctcagaggaggaggatgaagatccaactccggcccacgccatacctgcacggccctccagcgtggtcataacccctacctcggcatcgtttgtgattcccagaaagaagtgggacctacaggacaagacagtcacattgcatcgctcacccctgtgcagggacgaggacgagaaggaggagactggcaactcctcttacaccagaggccacaaaaggcgacgcggagaggtccatggctgcaccgatgaaagttatggcaagcgccgacacctgcccccgggagcaagagcgcccagagccccaagggcccccagggtgcctagagcaccgaggtctccaagagctccccgaagcaacagagcaaccagaggtccccggtcagaatctcgaggggccggcaggagcacaaggaagcaggcgaggcaagaacgcagccagaggcccctgccgaacaaaccgtggtttgacatgagtctggttaagcctgtctccaagattacatttgtcaccttgcccagccccctggcctctctgaccctagagcccatccaagacccgttcctacagtcgatgctggcggtggccgcccatccagagattggagcctggcagaaagtgcaacccagacacgagctgcgcaggagctacaagacactacgtgagtttttcaccaagtcaaccaacaaggacacatggctggatgcacgcatgcaggcgatccagaacgcggggctctgcaccctggtggccatgctagaagagaccatcttttggctccaggagatcacctaccacggcgacctgcccctagctcccgcggaagacatcctcctggcctgcgccatgagtcttagcaaggtgatcctgaccaagctcaaagagctggcaccctgcttccttcctaacacgcgagactacaactttgtgaagcaactcttctacatcacctgtgccacggcccgtcaaaacaaggtggtggagaccctgagcagctcatatgtgaagcagcccctctgtctcttggcagcatatgcggcagtagccccagcctacattaacgccaactgcagacggagacacgatgaagttgaattcctgggccactacatcaagaattacaaccctggcacgctaagctcccttttgacagaggccgtggagactcacacacgtgactgccgaagtgcatcatgcagccgacttgtcagggccattctctctccgggcactgggtcactaggactgttttttgttcctggattaaatcaataa

SEQ ID NO: 154 - DNA, 1095 bp, Human gammaherpesvirus 4
  source 1..1095: mol_type = genomic DNA
atgaaggtccaggggtccgtcgatcgccgccgtctgcaacgccgaatcgcggggctgctgccccctccggcccggcgtctaaatatttcccgggggtccgaattcacgcgggacgttcgtgggctggttgaggaacacgcgcaggcctcctcgctgagtgcggcggccgtctggcgcgcagggctgctggccccgggggaggtggcggtcgccgggggtggcagtggaggggggagcttcagctggtctgggtggcggccgccagtctttggggactttctgatacacgccagctccttcaacaacgccgaggccactggaacgccccttttccaattcaagcagagtgacccgttctcgggcgtcgacgcggtattcactcctctctccctgtttatcctaatgaatcacggccggggtgtagccgcccgggtcgaggcaggtgggggcctgacgcggatggccaacctgctgtacgacagccccgcaaccctggctgacctggtcccggactttgggcggctggtggccgaccgccgcttccacaacttcatcacccctgtgggccccctggtggagaatataaagagcacctatctgaataaaatcaccacggtggtccacgggcctgtggtcagcaaggccatccctcgcagcaccgtcaaggtgacggtgccccaggaggcctttgtggatctggacgcgtggctctccggcggcgccgggggtggcggtggagtatgcttcgtcggggggctgggcctgcagccgtgccccgccgatgcgcgcctctatgtcgctctgacctatgaggaagccgggccgcggtttacgtttttccagtcgtcccgcggccactgtcagatcatgaatatcttaagaatttattactcaccatccatcatgcaccgctatgctgtggtccagcccctacatatagaggagctaaccttcggggcggttgcctgtctggggacatttagtgctactgacggttggaggaggtctgccttcaattaccgtggctctagcctccccgtggtggagattgacagcttttattccaacgtctctgactgggaggtgattctctag

SEQ ID NO: 155 - DNA, 4146 bp, Human gammaherpesvirus 4
  source 1..4146: mol_type = genomic DNA
atggcctcaaatgagggtgtggaaaacagacccttcccctatctgacggtggatgccgacctgctctcgaacctgcggcagtcagcggctgaggggttgtttcatagctttgacctgctggtgggcaaggatgccagagaggcgggcatcaagtttgaggtgctactcggggtctacacgaacgccatccaatatgttcgcttcctggagacggcactggccgtgtcctgtgtgaacacggaattcaaagacctgagtcgtatgacggatggcaagattcaatttcgaatctccgtccccaccattgctcacggggacggaaggagacccagcaagcagcggacattcattgtggtcaaaaattgccacaaacaccacattagtacggaaatggaactgtccatgctggatctggagatcctgcatagtatccccgagaccccggtcgagtacgcagagtacgtgggggctgtcaagaccgtggcctcggccctacagtttggggtcgatgccctggagaggggcctcattaacaccgtcctgagtgtgaagcttcgccatgcccctcccatgtttatcctgcagaccctggcggatcccaccttcactgagagggggttctccaagactgtcaagtctgacctcattgccatgttcaagaggcatctgctggagcactccttcttcctggaccgggccgagaacatgggctccgggttttctcagtacgtgcgaagccgtctctctgagatggtagcggccgtgtccggggagagcgtgctcaagggggtcagtacctacacgaccgccaaggggggagagccagtggggggggtgtttattgtcaccgacaacgttctgcgccagctcttgaccttcctgggtgaggaggccgacaaccagattatggggccctcgagctatgcttcttttgtggtacggggggagaacctggtcactgccgtgagctacgggcgcgtgatgcgtacgtttgagcatttcatggctaggatcgtggactcaccggaaaaagccggaagcaccaagtctgacctgccggctgtggccgcaggggtcgaggatcagccccgggtgccgatctcagccgccgtcatcaagctgggcaaccacgcggtggccgtggaaagcctgcaaaagatgtacaatgacactcagtccccataccccctcaacagaagaatgcagtacagctactatttcccagtgggcctgtttatgcccaatcccaagtacacgacctccgccgccatcaaaatgctggacaaccccacacagcagctaccggtggaggcatggatcgtaaacaagaataaccttctcctcgcgtttaacctacagaatgctctcaaggttctctgtcacccccgactccacacacccgcccataccctaaacagcctcaacgcggccccggccccgcgagacaggcgcgagacctactccctgcaacacaggaggcccaatcacatgaatgtgcttgttattgtggacgagttctatgataacaagtatgcagcccccgtgacagatatagctctcaagtgcgggctgcccaccgaagacttcctccacccgtccaattatgacctgctgcggctggagctgcaccccctttatgatatttacattggcagggatgccggggagagggccaggcacagggctgtgcaccggctaatggtgggtaacctgccgactcccctggccccagctgcattccaagaggcccgggggcagcagtttgagaccgccacatctctggcccacgtggtggatcaggccgttattgagactgtgcaggatactgcctatgacactgcctatccagccttcttctacgtagtcgaggctatgatccacgggtttgaggaaaagtttgtcatgaacgtgcctttggtgtccctgtgcatcaacacctactgggaacggtcagggaggcttgcctttgtgaacagcttttccatgatcaagttcatctgccgccacctgggaaataacgccatctccaaggaggcctattccatgtatagaaaaatctatggggaacttatagccctagagcaggccctgatgcgcctggccgggtcagatgttgtgggggatgagagcgtgggtcagtatgtctgcgctctcctggaccctaacctgctccccccggtggcctacacagacattttcacccatcttctcaccgttagtgaccgggccccccagattattatcggaaatgaggtttacgctgacaccctggccgcgccccagtttattgagagggttggaaacatggatgagatggctgcccaatttgtggccttgtacggctaccgggttaacggagaccacgaccacgatttccgtctgcacctaggcccttatgtagatgaggggcatgcggatgtgctggaaaagatcttttactacgttttcctcccaacctgcaccaatgcccacatgtgcggcctcggggtggactttcagcacgtggcccagaccctggcctacaacgggccagccttcagccaccattttaccagggacgaggacatcctcgacaatttggagaatgggacgctcagggatctgctggagatctccgacctccgccccaccgtgggcatgatcagggacctcagcgcctcattcatgacctgccccactttcacccgtgccgtgcgtgtgtcggtggacaatgacgttacgcagcagctggccccgaatcccgccgacaagcggacagagcagactgttttggtgaacgggctggtggcctttgccttctccgagaggacccgggccgtcacccagtgtctctttcacgccattcctttccatatgttttacggggacccgcgagtggctgccaccatgcaccaggatgttgccacctttgttatgcgcaatcctcagcagcgggccgtggaagccttcaaccggccagagcagctctttgcagagtaccgggagtggcaccgctcgcccatgggcaaatacgcggccgaatgtcttccttccctcgtttcaatcagtggaatgaccgccatgcacatcaagatgtcccccatggcctatattgcccaggccaagctcaagatccacccaggggtggccatgaccgtggtcaggaccgatgagatcctctctgaaaacatattgtttagctccagggcctcaacatccatgttcattgggaccccaaatgttagccgccgggaggccagggtggacgcggtaacctttgaggtgcatcacgagatggcctccatcgacaccgggcttagttatagctcgaccatgactccggccagggtggcggccatcactactgacatgggtatccacacccaagacttctttagcgtctttccggccgaggcctttggcaaccagcaagtcaatgactacatcaaggccaaggtgggcgctcagcgcaatgggacgctgcttcgggaccccaggacatacctggcaggtatgactaatgttaatggagctccaggactctgccacggccagcaggccacctgtgagattatcgtaacaccggtcacggcagacgtggcttattttcaaaagtccaactctccaaggggacgggccgcctgtgtggtctcctgtgaaaactacaatcaggaggttgccgaggggctcatctatgaccattctcgcccggatgccgcctatgaataccggagcactgtgaatccctgggcatctcagctgggttctctgggtgacatcatgtacaactcctcctatcgccagacggccgtcccgggcctctacagcccctgccgggcatttttcaacaaggaggagcttctgcgcaacaacaggggactctacaacatggtcaacgagtacagccagcgacttggagggcacccagccaccagcaacacagaggtgcagtttgtagtgattgctggcactgacgtgtttctggagcagccctgcagctttctgcaggaggcattccccgcactctcagcctcctcccgggcactcatcgatgagtttatgtctgtcaaacagacccacgcccccatccattacggacactatataattgaagaggtggcgccggtacgaagaatattaaagtttggaaataaggtggttttttga

SEQ ID NO: 156 - DNA, 1824 bp, Human gammaherpesvirus 4
  source 1..1824: mol_type = genomic DNA
atggctggatttccaggaaaggaggccggcccgcccggcggctggcgaaaatgtcaggaggatgagtcccctgaaaacgaaagacacgaaaacttttacgctgagattgatgactttgccccctcagttcttaccccaaccggtagtgactcaggggcaggtgaggaagatgacgacggcctctaccaggtgcccacccactggccccccttgatggctccgaccggactgtctggggagagggtgccgtgccggacccaggctgccgtgacatctaatactggaaactctccgggtagccggcacacatcgtgcccctttaccctgcccaggggagcccagccaccagcacccgcacaccaaaagcctactgcccctaccccgaagccccgtagccgggagtgtggccccagcaagacgccagaccccttctcctggttccgcaaaacttcgtgcacagaaggaggtgcggactccacgagccggagcttcatgtaccagaaaggctttgaggagggcctggccggccttgggctcgatgacaaatctgactgtgagtctgaggatgagtctaatttccgcaggccctcttctcattccgcgttaaaacaaaagaatggtggcaagggtaagccttctggtctcttcgaacacctggccgcccatgggcgcgagtttagtaagttatccaagcatgctgcccagctgaagcggctaagtgggagtgtgatgaatgttctgaatctggatgatgcccaagacacccgccaggccaaggctcagaggaaggagagcatgcgggttccaattgtgacccacctaacaaatcatgtaccagtaatcaaacctgcctgttccctatttttggaaggtgcccctggtgtgggaaagactactatgctgaatcatttgaaggctgtctttggggacctgaccattgtagtccctgaaccgatgcggtactggactcatgtgtatgaaaatgccattaaggccatgcacaagaatgtgactcgggcgaggcatggaagggaagacacgtcggctgaggttttagcatgccagatgaaatttaccacccctttcagagtcttggcctctaggaagcggagcttgctggttaccgagtctggtgccaggtcagtcgcgcccctggattgttggattttgcatgatcgccatttgctgtcggcctccgtggttttccctctaatgctgctccggagccagcttctctcctatagtgactttattcaagttttggccacgtttaccgcagacccgggggacaccatagtctggatgaagttaaacgtggaggagaacatgcgccgcctgaagaagcgagggaggaaacatgagtcaggactggatgctggctacctgaagagtgtcaacgatgcatatcacgccgtttactgtgcctggctcctaacacagtattttgcccccgaggacattgtgaaggtatgtgccggtctaacaacaatcacgacagtatgtcatcaaagccacacccccataattcgaagtggtgtggccgagaagctgtataagaacagcatctttagtgtacttaaggaagtcatacagcctttccgagccgacgcggtgttgttggaagtttgtcttgcgttcacccgaacgctagcctaccttcagtttgtgcttgtggacctctcagagtttcaggacgatttacctggatgctggacggagatttacatgcaggcattgaagaatccggcaatacgatctcagtttttcgattgggctggtctgagcaaggtgatctctgattttgagaggggaaatcgggactag

SEQ ID NO: 157 - DNA, 2817 bp, Human gammaherpesvirus 4
  source 1..2817: mol_type = genomic DNA
atgaagaaagcgtggctcagcagagcacagcaagccgatgccgggggggcatctggctccgaggacccaccagattatggagatcaaggtaatgtgacacaggtgggatctgagcctatttcacctgagattggcccctttgaactctctgcggccagtgaggatgatcctcaatctgggccagtggaagagaatttagatgccgctgcaagagaggaagaggaacctcatgagcaggagcacaatggtggtgacgatcccttggatgtccatactcgccagcctagatttgtggatgtgaacccaacgcaggctccagtgatccaactagtccacgctgtctatgattccatgttgcaatcggacctccggcccctaggcagtttattccttgagcaaaacctgaacatcgaagaatttatatggatgtgcatgaccgtgcgtcacagatgtcaggccatcagaaaaaaaccattaccaattgttaagcagaggcgttggaagctcctgtcatcttgcagatcctggcgtatgggttaccgcacgcataacctcaaagtaaacagttttgagtcagggggggacaatgtccacccggtccttgtgactgctacgctaggatgtgatgagggcacgcggcatgcaacaacgtacagtgctggcattgtacagataccacgaatatcagaccaaaaccaaaagatcgaaacagccttcctgatggcacgtcgtgctaggtcactttcggcagaaagatatactttgttctttgatttagtatcctccggaaacaccctgtatgctatatggattgggctgggcacgaaaaaccgagtttcatttattgagtttgtaggatggttatgcaagaaggaccacactcatatacgcgaatggttccgccagtgcaccgggagacccaaagcagccaagccatggttaagagcgcatcctgtcgccattccttatgatgatccgttaacaaacgaggagattgatctggcctatgcccgcgggcaggccatgaatattgaggctcctagactgccagatgatcctataattgttgaggatgacgacgaaagtgaggaaattgaagctgaaagcgacgaggaggaagacaagagtggaatggaatctcttaaaaatataccgcaaacactgccgtacaatccaacagtatacggcaggcccgcggtgtttgaccgaaagtcagatgcaaaatcaaccaaaaaatgcagggccatagtaactgactttagtgtaatcaaggccattgaagaggaacacagaaagaagaaggcagccagaacagagcagccaagagccacgcctgaatcccaggcccccacagtggtcctccagcgaccacccacgcagcaagagcctggccccgtcggcccactgagtgtccaggctcggctggagccatggcaacctttgcctgggccccaagtgacagcagttctacttcacgaagaatccatgcagggtgtccaagtacatggttcgatgctagaccttcttgaaaaagacgatgaagtcatggagcagagggttatggcaaccctactgccaccagtaccacaacagccccgggctggcagaagaggcccttgtgtcttcaccggtgacctaggcatagagagtgatgagcccgcttcgacagagccggttcatgatcagctactgcctgccccaggacctgaccctcttgaaatccaaccactaacgtcccccaccacgtctcaacttagcagttcggcaccaagctgcgcacaaactccatggccggtggttcagccaagtcagactccagatgacccaacgaaacagtcccggccaccggaaacagctgccccacgccagtggccaatgcccctgcgacctatccctatgcgccccttgcggatgcagccaatcccatttaatcatccagtgggacccactccccatcagacacctcaagtggaaataacaccatataagcccacttgggctcagatagggcacattccatatcagcctacaccaacgggtcctgctaccatgctgttgcgccagtgggcacccgccaccatgcagacaccaccgagagcgcccactcccatgtcaccacctgaggtgccacccgttccccggcagaggcctcggggggcgcccactcccacgccacctcctcaggtgccgcccgttccccggcagaggcctcggggggcgcccactcccacgccacctcctcaggtgctgcccactcccatgcagctggcactaagggctcctgctggtcagcaggggccgacaaagcaaattttgcgccaattgttaacggggggcgtcaagaaagggagaccatcacttaagttacaggccgcccttgagcgtcaagccgctgcgggctggcagccttcaccagggtccggcaccagtgacaagattgtgcaggcgcctattttctacccacccgttttgcagcccatacaggttatggggcaagggggttccccaacggccatggccgcctcagcggtgacacaggcacccacggaatataccagggaaaggaggggagtggggcctatgcctcccaccgatattccgccgtctaaacgagcgaagatcgaggcctatacagagcccgagatgccgcacgggggggcctcgcactctcccgtcgttatcttggagaatgtcggccaggggcaacagcagactctggagtgcggaggaactgctaaacaggaaagggacatgttggggctgggggacattgcagtttcttccccttcctcttctgaaacatcgaacgatgagtga

SEQ ID NO: 158 - DNA, 2979 bp, Human gammaherpesvirus 4
  source 1..2979: mol_type = genomic DNA
atggaatcatttgaaggacagggggactctagacagtcacccgacaatgagcggggagataatgtacagactaccggcgagcatgatcaggaccctgggccggggcctccatccagtggggcttctgagagattggtaccagaagagtcatactcaagagatcagcaaccttgggggcaaagcaggggtgatgaaaacagaggctggatgcagcgcatcaggcgaaggcggagaagacgggctgccttgtccggccatcttttagacacggaagacaatgtgccgccatggttgcctccacacgacatcacaccatataccgcaaggaatatcagggatgctgcctgccgggctgtcaagcaatcgcacctgcaagcgctatcaaacctgatactcgatagtgggttagacacacaacacatcttgtgcttcgtgatggcagccaggcagcgtcttcaggacattcgacgtggacccttggttgcggagggcggtgtgggttggcgacattggcttctaacatctcccagccaatcctggcccatgggatatcgtacagcaacactacgcacattaactcccgtgcctaacagggttggggctgacagcatcatgttaactgccacatttggatgccaaaatgcggcacgaactctaaacaccttctctgccaccgtgtggacaccaccccatgctggaccaagagagcaagaaagatacgctcgggaagccgaggtacgcttccttcgtggtaaatggcagaggcggtaccgaagaatctatgatttgatagaactgtgtggctctctgcaccacatctggcaaaacttgctccagaccgaggagaaccttttagatttcgtgcgtttcatgggtgtcatgtccagctgcaataatccagctgtgaattactggtttcacaagacaatcggaaactttaagccatattacccgtggaatgcaccacctaatgaaaatccatatcacgcgcggagaggcataaaagaacacgtaatccagaacgcatttcgaaaggcacaaatacagggtttatcaatgttagcaacgggaggtgaacccagaggtgatgctactagtgaaacgagcagtgatgaggacaccggtagacagggttcggacgtggagctagagtcctcggacgatgagctgccatatatcgatcccaatatggagccggttcagcagaggcccgtcatgtttgtgagccgtgtgcctgcaaagaaaccgaggaaactgccttggcccacgcccaagacgcacccagtgaagcgcacaaatgttaagacctctgatagatctgataaggcagaagcacaaagcacccctgaaaggccgggcccttccgaacaatcatcagtgaccgtggagcccgcccacccgaccccggtggagatgccaatggtgattctccatcaaccacctccagtgcccaaaccggttccagtcaagcctacgccaccgccttcccgtaggagaaggggagcgtgtgttgtgtacgacgatgatgtcatagaggtgattgatgttgaaaccaccgaagattcatcgtcagtgtcacagccaaataagccacatcggaaacatcaagacggctttcaacgttcaggccgacgtcaaaaacgagccgcgcctcccaccgtgagtccttcggatactgggcctcctgccgtggggcctcctgccgcggggcctcctgccgcggggcctcctgccgcggggcctcctgccgcggggcctcctgccgcggggcctcctgccgcggggcctcgcatactggcgcctctttccgctgggcctcctgccgcggggcctcacatagtgacgcctccttccgcccggcctcgtataatggcgcctcccgtcgtacgtatgtttatgagggagcgacagctcccccagtccaccggccgtaaacctcagtgcttctgggaaatgcgggctggtcgtgaaattacacaaatgcaacaagaaccaagttcacacctgcagtccgccactcagcctacaacgcctcgcccatcatgggccccatcagtctgcgccctctcggtgatggatgctggtaaggcccagcccatagaaagttcacacttgagttccatgtcgcccacacagccgatatcgcacgaagaacaaccccggtatgaggatcctgacgctcctctggatttaagtttacatccagacgttgctgctcaaccagctccccaggctccataccagggataccaggagccgccggccccccaggctccataccagggataccaggagccgccgcccccccaggctccataccagggataccaggagccgccggcccacgggctccaatcatcttcatatccaggatatgcgggtccctggaccccaaggtctcaacatccatgttataggcacccctgggcaccatggtctcaagatcctgtgcatgggcacacccagggtccatgggatcccagggcaccacatctcccacctcagtgggatggatctgcaggacatggccaggatcaggtctcccagttcccacatctgcaatcggagacaggcccaccacgtcttcaactttcattggtgccactggtctcatcctctgcaccatcatggtcatctccccagccccgagcccccatacgccccattccaacaagattcccccctccccctatgccgttacaagatagcatggccgtggggtgtgactcatcaggtacagcatgcccaagcatgccctttgccagtgattacagtcaaggtgcatttaccccactggacattaatgccaccacgccaaaaaggcctcgagtagaagaaagttctcacggacctgcccggtgttcccaagctactgctgaagcacaggagattctcagtgacaattctgagatctccgtgttcccaaaggacgcgaagcagactgactacgatgcatccactgaaagtgagctagattaa

SEQ ID NO: 159 - DNA, 1161 bp, Human gammaherpesvirus 4
  source 1..1161: mol_type = genomic DNA
atggaacacgaccttgagaggggcccaccgggcccgcgacggccccctcgaggaccccccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgatcaccctcctgctcatcgctctctggaatttgcacggacaggcattgttccttggaattgtgctgttcatcttcgggtgcttacttgtcttaggtatctggatctacttattggagatgctctggcgacttggtgccaccatctggcagcttttggccttcttcctagccttcttcctagacctcatcctgctcattattgctctctatctacaacaaaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggacccccactctgctctcaaaacctaggcgcacctggaggtggtcctgacaatggcccacaggaccctgacaacactgatgacaatggcccacaggaccctgacaacactgatgacaatggcccacatgacccgctgcctcaggaccctgacaacactgatgacaatggcccacaggaccctgacaacactgatgacaatggcccacatgacccgctgcctcatagccctagcgactctgctggaaatgatggaggccctccacaattgacggaagaggttgaaaacaaaggaggtgaccagggcccgcctttgatgacagacggaggcggcggtcatagtcatgattccggccatggcggcggtgatccacaccttcctacgctgcttttgggttcttctggttccggtggagatgatgacgacccccacggcccagttcagctaagctactatgactaa

SEQ ID NO: 160 - DNA, 1494 bp, Human gammaherpesvirus 4
  source 1..1494: mol_type = genomic DNA
atggggtccctagaaatggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaagtatgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtttttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgtataa

SEQ ID NO: 161 - DNA, 1137 bp, Human gammaherpesvirus 4
  source 1..1137: mol_type = genomic DNA
atgaatccagtatgcctgcctgtaattgttgcgccctacctcttttggctggcggctattgccgcctcgtgtttcacggcctcagttagtaccgttgtgaccgccaccggcttggccctctcacttctactcttggcagcagtggccagctcatatgccgctgcacaaaggaaactgctgacaccggtgacagtgcttactgcggttgtcactttctttgcaatttgcctaacatggaggattgaggacccaccttttaattctcttctgtttgcattgctggccgcagctggcggactacaaggcatttacgttctggtgatgcttgtgctcctgatactagcgtacagaaggagatggcgccgtttgactgtttgtggcggcatcatgtttttggcatgtgtacttgtcctcatcgtcgacgctgttttgcagctgagtcccctccttggagctgtaactgtggtttccatgacgctgctgctactggctttcgtcctctggctctcttcgccagggggcctaggtactcttggtgcagcccttttaacattggcagcagctctggcactgctagcgtcactgattttgggcacacttaacttgactacaatgttccttctcatgctcctatggacacttgtggttctcctgatttgctcttcgtgctcttcatgtccactgagcaagatccttctggcacgactgttcctatatgctctcgcactcttgttgctagcctccgcgctaatcgctggtggcagtattttgcaaacaaacttcaagagtttaagcagcactgaatttatacccaatttgttctgcatgttattactgattgtcgctggcatactcttcattcttgctatcctgaccgaatggggcagtggaaatagaacatacggtccagtttttatgtgcctcggtggcctgctcaccatggtagccggcgctgtgtggctgacggtgatgtctaacacgcttttgtctgcctggattcttacagcaggattcctgattttcctcattggctttgccctctttggggtcattagatgctgccgctactgctgctactactgccttacactggaaagtgaggagcgcccaccgaccccatatcgcaacactgtataa

SEQ ID NO: 162 - DNA, 51 bp, Homo sapiens
  source 1..51: mol_type = genomic DNA
atgccccactccagcctgcatccatccatcccgtgtcccaggggtcacggg

SEQ ID NO: 163 - DNA, 30 bp, Homo sapiens
  source 1..30: mol_type = genomic DNA
atgggatgtataaaatcaaaagggaaagac

SEQ ID NO: 164 - DNA, 66 bp, Homo sapiens
  source 1..66: mol_type = genomic DNA
atgttggctaccagggtatttagcctagttggcaagcgagcaatttccacctctgtgtgtgtacga

SEQ ID NO: 165 - DNA, 54 bp, Homo sapiens
  source 1..54: mol_type = genomic DNA
atgaagctctccctggtggccgcgatgctgctgctgctcagcgcggcgcgggcc

SEQ ID NO: 166 - DNA, 12 bp, Homo sapiens
  source 1..12: mol_type = genomic DNA
aaagatgagttg

SEQ ID NO: 167 - DNA, 51 bp, synthetic construct
  source 1..51: mol_type = other DNA
atgcctcactctagcctgcaccctagcatcccctgcccaagaggccacggc

SEQ ID NO: 168 - DNA, 30 bp, synthetic construct
  source 1..30: mol_type = other DNA
atgggctgtatcaagagcaagcggaaagac

SEQ ID NO: 169 - DNA, 66 bp, synthetic construct
  source 1..66: mol_type = other DNA
atgctggccacaagagtgttcagcctggtcggcaagagagccatcagcacctctgtgtgcgtgcgg

SEQ ID NO: 170 - DNA, 54 bp, synthetic construct
  source 1..54: mol_type = other DNA
atgaaactgagcctggtggccgccatgctcctgctgctgtctgccgccagagca

SEQ ID NO: 171 - DNA, 12 bp, synthetic construct
  source 1..12: mol_type = other DNA
aaggacgagctg